(19) United States
(12) Patent Application Publication
(10) Pub. No.: US 2012/0128630 A1
(43) Pub. Date: May 24, 2012

(54) 1-(6 MEMBERS AZO-HETEROCYCLIC)-PYRROLIN-2-ONE COMPOUNDS AS INHIBITORS OF HEPATITIS C NS5B POLYMERASE, THE PHARAMACEUTICAL COMPOSITION THEREOF AND THEIR THERAPEUTIC USE

(75) Inventors: Amaya Berecibar, Coueron (FR); Philippe Guedat, Montenois (FR); Céline Mohamed-Arab, Lyon (FR)

(73) Assignee: VIVALIS, Roussay (FR)

(21) Appl. No.: 13/383,120

(22) PCT Filed: Jul. 9, 2010
(86) PCT No.: PCT/EP2010/059917

§ 371 Date: Jan. 9, 2012

(30) Foreign Application Priority Data
Jul. 10, 2009 (EP) 09305671.1

(51) Int. Cl. A61K 38/21; A61K 31/501; A61K 31/4439; C07D 413/14; A61P 31/12; C07D 403/14; C07D 401/04; C07D 417/14; A61P 31/18; C07D 405/14; A61K 31/5377; C07D 403/04

(52) U.S. Cl. 424/85.4; 514/236.5; 514/252.02; 514/252.03; 514/252.05; 514/343; 544/114; 544/238; 546/278.7

(57) ABSTRACT

The present invention concerns a 1-(6 members azo-heterocyclic)-pyrrolin-2-one compound of the following formula I or a salt, solvate, tautomer, isotope, enantiomer, diastereoisomer or racemic mixture thereof: the pharmaceutical composition thereof and their therapeutic use as inhibitors of Hepatitis C NS5B polymerase.

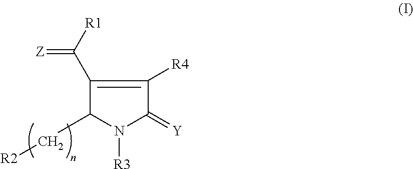

The present invention concerns antiviral compounds, in particular anti hepatitis C compounds.

Viral proteins constitute a group of biologically active proteins with high pharmacological value. Drugs to deal with viral infections are a field of medicine that has been traditionally weak. However since the 1980s, the full genetic sequences of viruses began to be available to researchers, and they began to learn how viruses worked in detail, and to envision what kind of molecules were needed to jam their machinery. The general idea behind modern antiviral drug design is to identify viral proteins, or parts of proteins, that can be disabled. The targets should also be common across many strains of a virus, or even among different species of virus in the same family, so a single drug will have broad effectiveness. Dozens of “antiviral” treatments are now available, and a lot are currently under development. Most of the antivirals now available are designed to help deal with HIV, herpes virus, hepatitis B and C viruses and influenza viruses.

Viral life cycles vary in their precise details depending on the species of virus, but they all share a general pattern:
Attachment to a host cell.
Release of viral genes and possibly enzymes into the host cell.
Replication of viral components using host-cell machinery.
Assembly of viral components into complete viral particles.
Release of viral particles to infect new host cells.

One of the major antivirals development approach is to interfere with the ability of a virus to get into a target cell. The virus has to take a sequence of actions to do this, beginning with binding to a specific receptor molecule on the surface of the host cell and ending with the virus “un-coating” inside the cell and releasing its payload. Viruses that have a lipid envelope must also fuse their envelope with the target cell, or with a vesicle that transports them into the cell, before they can uncoated. All these steps involve the binding of viral proteins with one or more binding partners. Indeed, a number of “entry-inhibiting” or “entry-blocking” drugs are being developed to fight HIV. “Amantine” and “rimantadine” are two entry-blockers that have been developed to combat influenza virus. Amantine and rimantadine are thought to interfere with influenza A virus M2 protein, an ion channel protein, and to inhibit virus uncoating. However, Amantine and rimantadine does not work on influenza B viruses and the two drugs have been associated with gastro-intestinal and central nervous system adverse effects. Pleconaril, another entry-blocker, works against rhinoviruses, which cause most colds, by blocking a pocket on the surface of the virus that controls the un-coating process. This pocket is similar in most strains of rhinoviruses, and the drug also seems to work against “entero-virus”, which can cause diarrhea, meningitis, conjunctivitis, and encephalitis.

A second approach is to target the processes that synthesize virus components after a virus invades a cell. “Nucleotide or nucleoside analogues” are antivirals that will interfere and block the enzymes that synthesize the RNA or DNA once the analogue is incorporated. The first successful antiviral, “acyclovir”, is a nucleoside analogue, and is effective against herpes virus infections. Another nucleoside analogue named “zidovudine” or “AZT” has been approved for treating HIV. Another class of antivirals that has been proven effective is the viral proteases inhibitors. Viral proteases act through binding to a target protein. However, protease inhibitors may have odd side-effects, for example causing fat to build up in unusual places. Then there is a need for improved protease inhibitors.

The final stage in the life cycle of a virus is the release of completed viruses from the host cell, and of course this step has also been targeted by antiviral drug developers. Two drugs named “zanamivir” and “oseltamivir” that have been recently introduced to treat influenza prevent the release of viral particles by blocking a molecule named “neuraminidase” that is found on the surface of flu viruses, and also seems to be constant across a wide range of flu strains. Those two drugs block the active site of the influenza viral enzyme neuraminidase. However Oseltamivir has been associated with adverse effects such as nausea and vomiting. Zanamivir showed adverse respiratory events in persons with chronic pulmonary disease.

Therefore there is a great need to extend the activity, the specificity and the efficacy of current antivirals, but also to extend the range of antivirals to other families of pathogens.

Hepatitis C is a global health problem with 170 million carriers' worldwide, 3 to 4 million new cases each year and a worldwide mortality estimated to 500,000 persons a year. 30% of liver grafts are currently prescribed to patients infected with HCV. HCV is spread primarily by direct contact with human blood. Transmission through blood transfusions that are not screened for HCV infection, through the re-use of inadequately sterilized needles and syringes or other medical equipment or through needle-sharing among drug users, is well documented. Sexual and perinatal transmission may also occur, although less frequently.

The incubation period of HCV infection before the onset of clinical symptoms ranges from 15 to 150 days. About 80% of infected patients progress to develop chronic infection which can also be asymptomatic. Cirrhosis develops in about 10% to 20% of persons with chronic infection and liver cancer develops in 1% to 5% of persons with chronic infection over a period of 20 to 30 years.

The virus responsible for this post transfusion non A non B Hepatitis was identified in 1989. Hepatitis C virus is an enveloped virus from the Flaviviridae family and is the only member of hepacivirus genus. HCV comprises 6 genotypes, more than 45 subtypes and quasi-species patient-specific. Its positive single strand linear RNA has about 9,600 nucleotides. RNA genome is flanked by two untranslated regions (UTR) that play a major role in translation and replication of the viral genome. Upon interaction and fusion of viral and cellular membranes, RNA genome is released into the cytoplasm of a newly infected cell and serves as template for RNA replication. Viral genome replication is a two step process: the positive RNA strand is used as a matrix for the synthesis of a negative polarity RNA which in turn serves as matrix for the synthesis of positive RNA strands that will be incorporated in new virions. Translation of HCV genome depends on an internal ribosome entry site and produces a large polyprotein which is proteolytically cleaved by cellular and viral proteases to produce 10 viral proteins. The amino terminal one third of the polyprotein encodes the structural proteins: core protein glycoproteins E1+E2. After the structural region, comes a small integral protein, P7, which seems to function as an ion chemical. The remainder of the genome encodes the non structural proteins NS2, N3, NS4A, NS4B, NS5A & NS5B which coordinate the intracellular processes of the virus life cycle (Lindenbach et al., 2005). Replication complex is associated with membranes of the endoplasmic reticulum. Viral proteins involved in this complex are the NTPase/helicase/serine protease NS3-4A, NS4B which is involved in the formation of the replication web, NS5A whose function still remains to be elucidated and the RNA-dependent RNA polymerase NS5B. No vaccine is currently available to prevent hepatitis C. The standard treatment consists in a combination between Interferon, a cytokine with immuno-modulatory and antiviral activity (Moussalli et al., 1998) and Ribavirin, a synthetic guanosine nucleoside analogue (Hugle et al., 2003). For patients infected with HCV genotype 1a/1b (the predominant one in USA, Japan and Europe), the sustained viral response (loss of serum HCV RNA following 24 weeks of antiviral therapy) is at best 42-46% (Walker et al. 2002, Gordon et al., 2005; Lake-Bakaar et al., 2003).

Besides its relative inefficacy, this combination therapy yields significant side effects (Fried Michael, 2002). New treatment regimens are needed and, to address inefficiency and specificity issues, investigators have focused in recent years on the identification of drugs that specifically inhibit viral enzymes playing a key role in virus life-cycle.

Although all HCV enzymes are, in theory, equally appropriate for therapeutic intervention, the NS5B RNA polymerase and NS3-4A serine protease are respectively important for genome replication and polyprotein processing and were the most studied. NS5B polymerase is a 66 kD oligomeric, tail-anchored protein (Ivashkina et al., 2002; Schmidt-Mende et al., 2001). Its C-terminal 21 residues form an α-helical transmembrane domain responsible for post-translational targeting to the cytosolic side of the ER, where the functional protein domain is exposed (Moradpour et al., 2004; Schmidt-Mende et al., 2001). The crystal structure of NS5B revealed that the RdRp has a classical “fingers, palm and thumb” structure (Ago et al., 1999; Bressanelli et al., 1999; Lesburg et al., 1999). Unlike many cellular and other viral polymerase, interactions between the fingers and thumb subdomains result in a completely encircled catalytic site that ensures synthesis of positive- and negative-strand HCV RNAs (Lesburg et al., 1999). A unique feature is the presence of a B-harpin in the thumb subdomain that protrudes toward the active site and may thus restrict binding of the template/primer at the active site. NS5B catalyzes de novo, primer-independent initiation of RNA synthesis followed by elongation, termination of polymerization and release of nascent strand.

Therefore, there is a need to find new compounds which can be used in the treatment of hepatitis C, and in particular having a HCV inhibitory activity and more particularly a HCV NS5B polymerase inhibitory activity, without the drawbacks of the prior art.

The present invention concerns a compound of the following formula I or a salt, solvate, tautomer, isotope, enantiomer, diastereoisomer or racemic mixture thereof:

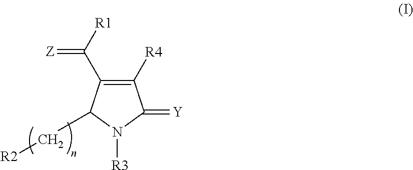

in which
n is an integer chosen between 0, 1 or 2, advantageously n=0;
Y represents an oxygen atom or a sulfur atom, advantageously an oxygen atom
C═Z represents CH₂ or
Z represents an oxygen atom, a —CH—R group or a —N—OR group, in which R represents an hydrogen atom, a C₁-C₆ alkyl group, a C₃-C₆ cycloalkyl group, a 5-6 members heterocyclic group containing one or two heteroatoms selected in the group consisting of oxygen, nitrogen and sulfur atom, a (C₁-C₆ alkyl)COOH group, a (C₁-C₆ alkyl)O(C₁-C₆ alkyl) group or a O-protecting group; advantageously Z represents an oxygen atom or a —N—OR group in which R represent a C₁-C₆ alkyl group, a (C₁-C₆ alkyl)O(C₁-C₆ alkyl) group or a (C₁-C₆ alkyl)COOH group; in particular R represents a CH₂—CH₂—OMe group or a methyl group. More advantageously, Z represents an oxygen atom.
R1 represents a phenyl group or a 5- or 6-members heteroaryl group containing one, two or three heteroatoms selected in the group consisting of oxygen, nitrogen and sulfur atom, advantageously nitrogen and sulfur atom, in particular a thiazol a thiadiazol or pyridine group, the phenyl group and the heteroaryl group being optionally substituted, in particular at the para position for the phenyl and the 6-members heteroaryl group, by a halogen atom; a —CN group; a —SO₂—(C₁-C₆) alkyl group in which the alkyl group is optionally substituted by one or more halogen atom, in particular a fluorine atom; a phenyl group; a 5- or 6-members heteroaryl group containing one, two or three heteroatoms selected in the group consisting of oxygen, nitrogen and sulfur atom, advantageously a nitrogen atom; a C₁-C₆ alkyl group optionally substituted by one or more halogen atom, in particular a fluorine atom; a C₂-C₆ alkenyl group optionally substituted by one or more halogen atom, in particular a fluorine atom; a —O—(C₁-C₆)alkyl group in which the alkyl group is optionally substituted by one or more halogen atom, in particular a fluorine atom; a O—(C₁-C₆)alkyl-O—(C₁-C₆)alkyl group in which the alkyl group is optionally substituted by one or more halogen atom; a —O—(C₁-C₆)alkyl-phenyl-O—(C₁-C₆)alkyl group; a C₃-C₆ cycloalkyl group optionally substituted by one or more halogen atom, in particular a fluorine atom; a 5-, 6- or 7-members heterocyclic group containing one, two or three heteroatoms selected in the group consisting of nitrogen, sulfur and oxygen atom; or a —NR′R″ group in which R′ and R″ represent independently of each other a hydrogen atom or a C₁-C₆ alkyl group.

Advantageously, the phenyl or the heteroaryl group is substituted, more particularly at the para position for the phenyl and the 6-members heteroaryl group, by a halogen atom; a phenyl group; a C₁-C₆ alkyl group optionally substituted by one or more halogen atom, in particular a fluorine atom; a —O—(C₁-C₆)alkyl group in which the alkyl group is optionally substituted by one or more halogen atom, in particular a fluorine atom; a O—(C₁-C₆)alkyl-O—(C₁-C₆)alkyl group in which the alkyl group is optionally substituted by one or more halogen atom; a C₃-C₆ cycloalkyl group optionally substituted by one or more halogen atom, in particular a fluorine atom; a —O—(C₁-C₆)alkyl-phenyl-O—(C₁-C₆)alkyl group; a 5-, 6- or 7-members heterocyclic group containing one, two or three heteroatoms selected in the group consisting of nitrogen, sulfur and oxygen atom; or a —NR′R″ group in which R′ and R″ represent independently of each other a hydrogen atom or a C₁-C₆ alkyl group.

In particular, the phenyl or the heteroaryl group is substituted, more particularly at the para position for the phenyl and the 6-members heteroaryl group, by a C₂-C₆ alkenyl group optionally substituted by one or more halogen atom, in particular a fluorine atom; a C₁-C₆ alkyl group optionally substituted by one or more halogen atom, in particular a fluorine atom; a —O—(C₁-C₆)alkyl group in which the alkyl group is optionally substituted by one or more halogen atom, in particular a fluorine atom; a C₃-C₆ cycloalkyl group optionally substituted by one or more halogen atom, in particular a fluorine atom; or a —NR′R″ group in which R′ and R″ represent independently of each other a hydrogen atom or a C₁-C₆ alkyl group, in particular a C₁-C₆ alkyl group. Advantageously, the phenyl or the heteroaryl group is substituted, more particularly at the para position for the phenyl and the 6-members heteroaryl group, by a C₁-C₆ alkyl group, in particular a methyl, ethyl, tert-butyl, isobutyl or isopropyl group, optionally substituted by one or more halogen atom, in particular a fluorine atom; a C₂-C₆ alkenyl group, in particular a isopropenyl group; a —O—(C₁-C₆) alkyl group, in particular a O-methyl group, in which the alkyl group is optionally substituted by one or more halogen atom, in particular a fluorine atom. More advantageously, the phenyl or the heteroaryl group is substituted, more particularly at the para position for the phenyl and the 6-members heteroaryl group, by a C₁-C₆ alkyl group, in particular a methyl or isopropyl group, or a —OCF₃ group, or a —CF₃ group.

R2 represents a phenyl group, or a 5- or 6-members heteroaryl group containing one two or three heteroatom(s) selected in the group consisting of oxygen, sulfur and nitrogen atom, advantageously nitrogen and sulfur atom, in particular a thiazol a thiadiazol or pyridine group the phenyl group and the heteroaryl group being optionally substituted by one or more groups, advantageously one or two groups, independently selected among a halogen atom; a —OH group; a —CN group; a C₁-C₆ alkyl group optionally substituted by one or more halogen atom, in particular a fluorine atom, or by a 5-members heteroaryl group containing one, two, three or four heteroatom (s) selected in the group consisting of oxygen, sulfur and nitrogen atom, advantageously nitrogen atom;
a —O—(C₁-C₆)alkyl group in which the alkyl group is optionally substituted by one or more halogen atom; a —CO—(C₁-C₆)alkyl group in which the alkyl group is optionally substituted by one or more halogen atom; a —SO₂—(C₁-C₆)alkyl group in which the alkyl group is optionally substituted by one or more halogen atom, in particular a fluorine atom; a —S—(C₁-C₆)alkyl group in which the alkyl group is optionally substituted by one or more halogen atom, in particular a fluorine atom; a C₂-C₆ alkenyl group optionally substituted by one or more halogen atom, in particular a fluorine atom; a C₂-C₆ alkynyl group optionally substituted by one or more halogen atom, in particular a fluorine atom; a C₃-C₆ cycloalkyl group optionally substituted by one or more halogen atom, in particular a fluorine atom; a —O—(C₃-C₆)cycloalkyl group in which the cycloalkyl group is optionally substituted by one or more halogen atom, in particular a fluorine atom; a phenyl group; a —O-phenyl group; a 5-members heteroaryl group containing one, two, three or four heteroatom (s) selected in the group consisting of oxygen, sulfur and nitrogen atom, advantageously nitrogen and oxygen atom, the heteroaryl group being optionally substituted by a C₁-C₆ alkyl group; a —NR′R″ group in which R′ and R″ represent independently of each other a hydrogen atom or a C₁-C₆ alkyl group; a —O-(6-members heterocyclic) group in which the heterocyclic group contains one, two or three heteroatoms selected in the group consisting of nitrogen, sulfur and oxygen atom, advantageously nitrogen and oxygen atom; a —O-(5- or 6-members heteroaryl) group in which the heteroaryl group contains one, two, three or four heteroatom (s) selected in the group consisting of oxygen, sulfur and nitrogen atom, advantageously nitrogen and oxygen atom, the heteroaryl group being optionally substituted by a —(C₁-C₆ alkyl)-phenyl group, a —(C₁-C₆ alkyl) group or a —CH₂—O—CH₂—Si(CH₃)₃ group, the alkyl group being optionally substituted by a halogen atom; a —O—((C₁-C₆)alkyl)-(6-members heterocyclic) group in which the heterocyclic group contains one, two or three heteroatoms selected in the group consisting of nitrogen, sulfur and oxygen atom; a —O—((C₁-C₆)alkyl)-NR′R″ group in which R′ and R″ represent independently of each other a hydrogen atom or a C₁-C₆ alkyl group; and a 6-members heterocyclic group containing one, two or three heteroatoms selected in the group consisting of nitrogen, sulfur and oxygen atom, advantageously nitrogen atom, the heterocyclic group being optionally substituted by a —((C₁-C₆)alkyl)-NR′R″ group in which R′ and R″ represent independently of each other a hydrogen atom or a C₁-C₆ alkyl group.

Advantageously the phenyl or the heteroaryl group is substituted by a halogen atom; a —OH group; a C₁-C₆ alkyl group optionally substituted by one or more halogen atom, in particular a fluorine atom; a —O—(C₁-C₆)alkyl group in which the alkyl group is optionally substituted by one or more halogen atom; a C₃-C₆ cycloalkyl group optionally substituted by one or more halogen atom, in particular a fluorine atom; a phenyl group; a —O-phenyl group; a —NR′R″ group in which R′ and R″ represent independently of each other a hydrogen atom or a C₁-C₆ alkyl group; a —O-(6-members heterocyclic) group in which the heterocyclic group contains one, two or three heteroatoms selected in the group consisting of nitrogen, sulfur and oxygen atom, advantageously nitrogen atom; a —O((C₁-C₆)alkyl)-(6-members heterocyclic) group in which the heterocyclic group contains one, two or three heteroatoms selected in the group consisting of nitrogen, sulfur and oxygen atom; a —O—((C₁-C₆)alkyl)-NR′R″ group in which R′ and R″ represent independently of each other a hydrogen atom or a C₁-C₆ alkyl group; and a 6-members heterocyclic group containing one, two or three heteroatoms selected in the group consisting of nitrogen, sulfur and oxygen atom, advantageously nitrogen atom, the heterocyclic group being optionally substituted by a —((C₁-C₆)alkyl)-NR′R″ group in which R′ and R″ represent independently of each other a hydrogen atom or a C₁-C₆ alkyl group;

In particular the phenyl or the heteroaryl group is substituted by a —O-(5-members heteroaryl) group in which the heteroaryl group contains one, two, three or four heteroatom (s), in particular three heteroatoms, selected in the group consisting of oxygen, sulfur and nitrogen atom, advantageously nitrogen atom, the heteroaryl group being optionally substituted by a —(C₁-C₆ alkyl)-phenyl group, a —(C₁-C₆ alkyl) group or a —CH₂—O—CH₂—Si(CH₃)₃ group, the alkyl group being optionally substituted by a halogen atom, advantageously the heteroaryl group is substituted by a —CH₂—O—CH₂—Si(CH₃)₃ group; a halogen atom; a —S—(C₁-C₆)alkyl group in which the alkyl group is optionally substituted by one or more halogen atom, in particular a fluorine atom; a C₂-C₆ alkenyl group optionally substituted by one or more halogen atom, in particular a fluorine atom; a —O—(C₃-C₆)cycloalkyl group in which the cycloalkyl group is optionally substituted by one or more halogen atom, in particular a fluorine atom; C₁-C₆ alkyl group optionally substituted by one or more halogen atom, more particularly a fluorine atom; a C₃-C₆ cycloalkyl group optionally substituted by one or more halogen atom, more particularly a fluorine atom; a —O—(C₁-C₆)alkyl group in which the alkyl group is optionally substituted by one or more halogen atom; a phenyl group; or a 6-members heterocyclic group containing one, two or three heteroatoms selected in the group consisting of nitrogen, sulfur and oxygen atom, in particular a morpholinyl group. More advantageously the phenyl or heteroaryl group is substituted by a halogen atom, in particular a boron atom; a —S—(C₁-C₆)alkyl group in particular a —SCH(CH₃)₂; a C₂-C₆ alkenyl group, in particular a isopropenyl group; a —O—(C₃-C₆)cycloalkyl group in particular a —O-cyclopropyl; a C₁-C₆ alkyl group, in particular a tert-butyl group, an ethyl group or a isopropyl group, optionally substituted by one or more halogen atom, more particularly a —CF₃ group or a —CF₂CH₃ group, a —O—(C₁-C₆)alkyl group, in particular a O-methyl, O-ethyl or a O-isopropyl group, in which the alkyl group is optionally substituted by one or more halogen atom, in particular a —OCF₃ group; or a phenyl group. Even still more advantageously, the phenyl or the heteroaryl group is substituted by a C₁-C₆ alkyl group, in particular a isopropyl group, optionally substituted by one or more halogen atom, more particularly a —CF₃ group or a —CF₂CH₃ group; or a —O—(C₁-C₆) alkyl group, in particular a O-methyl, in which the alkyl group is optionally substituted by one or more halogen atom, in particular a —OCF₃ group. In an advantageous embodiment, the phenyl or the heteroaryl group is substituted by —CF₃ or a —CF₂CH₃ group; —OCF₃ or an isopropyl group.

R3 represents a 6-members heteroaryl group containing as the only heteroatom(s), one, two or three nitrogen atoms, advantageously two or three nitrogen atoms, the heteroaryl group being optionally substituted by a halogen atom; a C₁-C₆ alkyl group optionally substituted by one or more halogen atom, in particular a fluorine atom, by a —O—(C₁-C₆)alkyl group, by a —O—C₃-C₆ cycloalkyl group, by a —O-aryl group or by a —NR′R″ group in which R′ and R″ represent independently of each other a hydrogen atom, a C₁-C₆ alkyl group, a C₃-C₆ cycloalkyl group or an aryl group; a C₃-C₆ cycloalkyl group optionally substituted by one or more halogen atom, in particular a fluorine atom; a —O—(C₁-C₆)alkyl group in which the alkyl group is optionally substituted by one or more halogen atom, in particular a fluorine atom, or by a phenyl group; a —OH group; a —COOH group; a —COO(C₁-C₆)alkyl group in which the alkyl group is optionally substituted by one or more halogen atom in particular a fluorine atom; a —CN group; a ═O group; a —SO₂-phenyl-NO₂ group; a —S-phenyl-NO₂ group; a —SO₂—(C₁-C₆)alkyl group; a —SO₂-aryl group, a —SO₂—NH—(C₁-C₆)alkyl group; a —SO₂—NH-aryl group; a 6-members heterocyclic group containing one or two heteroatoms selected in the group consisting of nitrogen, sulfur and oxygen atom; or a —NR′R″ group in which R′ and R″ represent independently of each other a hydrogen atom or a C₁-C₆ alkyl group.

In particular the heteroaryl group which is optionally substituted by a C₁-C₆ alkyl group optionally substituted by a —O—(C₁-C₆)alkyl group, by a —O—C₃-C₆ cycloalkyl group, by a —O-aryl group or by a —NR′R″ group in which R′ and R″ represent independently of each other a hydrogen atom, a C₁-C₆ alkyl group, a C₃-C₆ cycloalkyl group or an aryl group, contains as the only heteroatom(s), two or three nitrogen atoms.

Advantageously the heteroaryl group is unsubstituted or substituted by a halogen atom; a C₁-C₆ alkyl group optionally substituted by one or more halogen atom, in particular a fluorine atom; a C₃-C₆ cycloalkyl group optionally substituted by one or more halogen atom, in particular a fluorine atom; a —O—(C₁-C₆)alkyl group in which the alkyl group is optionally substituted by one or more halogen atom, in particular a fluorine atom; a —COOH group; a —COO(C₁-C₆)alkyl group in which the alkyl group is optionally substituted by one or more halogen atom in particular a fluorine atom; a —CN group; a —SO₂-phenyl-NO₂ group; a —SO₂—(C₁-C₆)alkyl group; a —SO₂-aryl group, a —SO₂—NH—(C₁-C₆)alkyl group; a —SO₂—NH-aryl group; a 6-members heterocyclic group containing one or two heteroatoms selected in the group consisting of nitrogen, sulfur and oxygen atom; or a —NR′R″ group in which R′ and R″ represent independently of each other a hydrogen atom or a C₁-C₆ alkyl group.

In particular the heteroaryl group is unsubstituted or substituted by a C₁-C₆ alkyl group optionally substituted by one or more halogen atom, more particularly a fluorine atom; a halogen atom, in particular a chlorine atom; a —O—(C₁-C₆)alkyl group in which the alkyl group is optionally substituted by a phenyl group, in particular —OCH₃ group or a —OCH₂-phenyl group; a —OH group; a —S-phenyl-NO₂ group; a C₃-C₆ cycloalkyl group optionally substituted by one or more halogen atom, more particularly a fluorine atom; a —CN group; or a —NR′R″ group in which R′ and R″ represent independently of each other a hydrogen atom or a C₁-C₆ alkyl group, advantageously a C₁-C₆ alkyl group. More advantageously the heteroaryl group is unsubstituted or substituted by a C₁-C₆ alkyl group, in particular a methyl group, or a —NR′R″ group in which R′ and R″ represent independently of each other a hydrogen atom or a C₁-C₆ alkyl group, advantageously a C₁-C₆ alkyl group. Even still more advantageously, the heteroaryl group is substituted by a C₁-C₆ alkyl group, in particular a methyl group

R4 represents a —OH group or a halogen atom or a —O—C═O—(C₁-C₆alkyl) group or a —O—(C₁-C₆)alkyl group or a —O—(C₁-C₆)alkyl-CO—O—(C₁-C₆)alkyl group or a —SH group, in which the alkyl group is optionally substituted by a phenyl group, advantageously a —O—(CHphenyl)-CO—O—(C₁-C₆)alkyl group or a —NHR₅ group in which R₅ represents an hydrogen or a (C₁-C₆alkyl) group, or a —NHSO₂R₆ group in which R₆ represents a hydrogen atom or a (C₁-C₆alkyl) group.

In particular R₄ represents a —OH group or a —O—C═O—(C₁-C₆alkyl) group, advantageously a —O—C═O—(CH₃) group or a —O—C═O—CH(CH₃)₂ group, or a —O—(C₁-C₆)alkyl group, or a —NHR₅ group in which R₅ represents an hydrogen or a (C₁-C₆alkyl) group, or a —NHSO₂R₆ group in which R₆ represents an hydrogen or a (C₁-C₆alkyl) group, in particular a —NHSO₂CH₃ group;

Advantageously, R4 represents a —OH group or a —O—C═O—(C₁-C₆alkyl) group, in particular a —OH group or a —O—C═O—(CH₃) group.

With the proviso that the compound of formula I does not correspond to the following one:

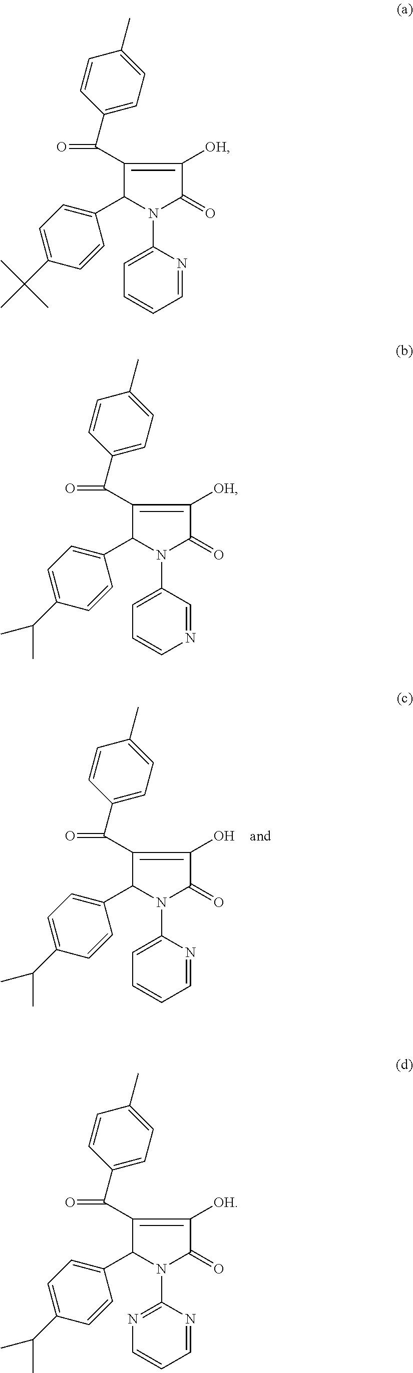

The compounds of formula (a), (b), (c) and (d) are known as such but they have never been described as having a therapeutic activity, in particular an antiviral activity, more particularly an anti HCV activity.

In a particular embodiment, the compound of formula I does not correspond to the following one:

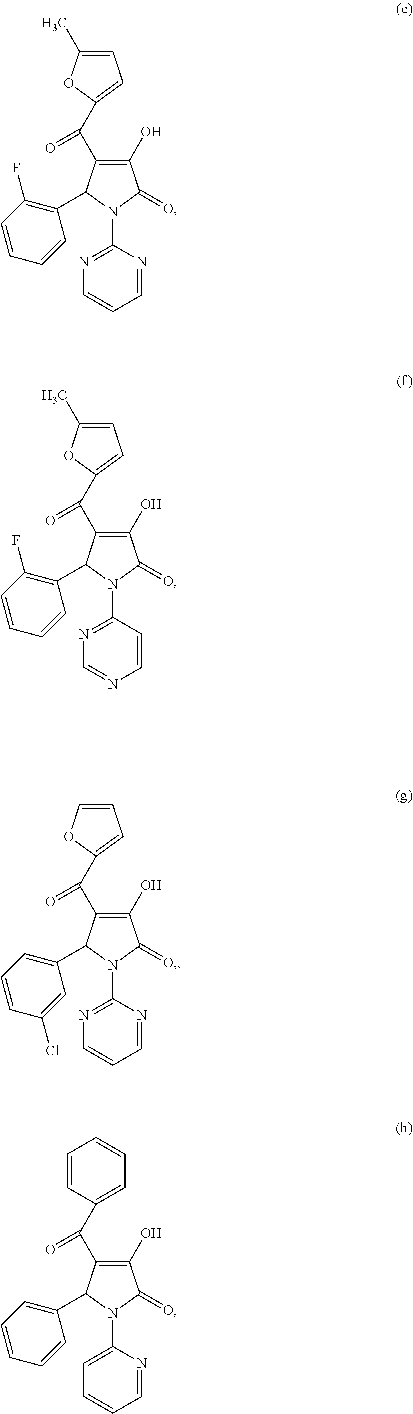

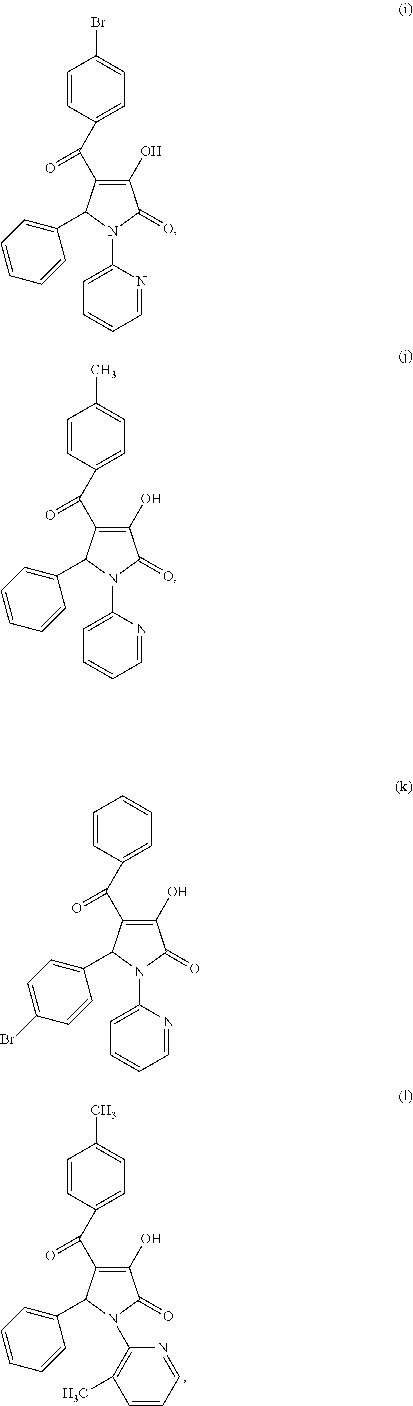

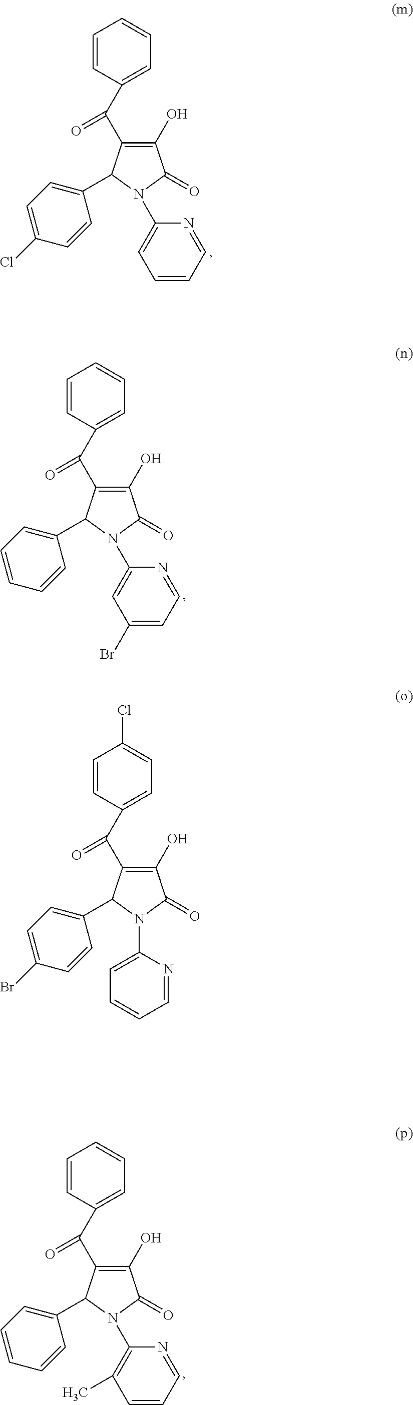

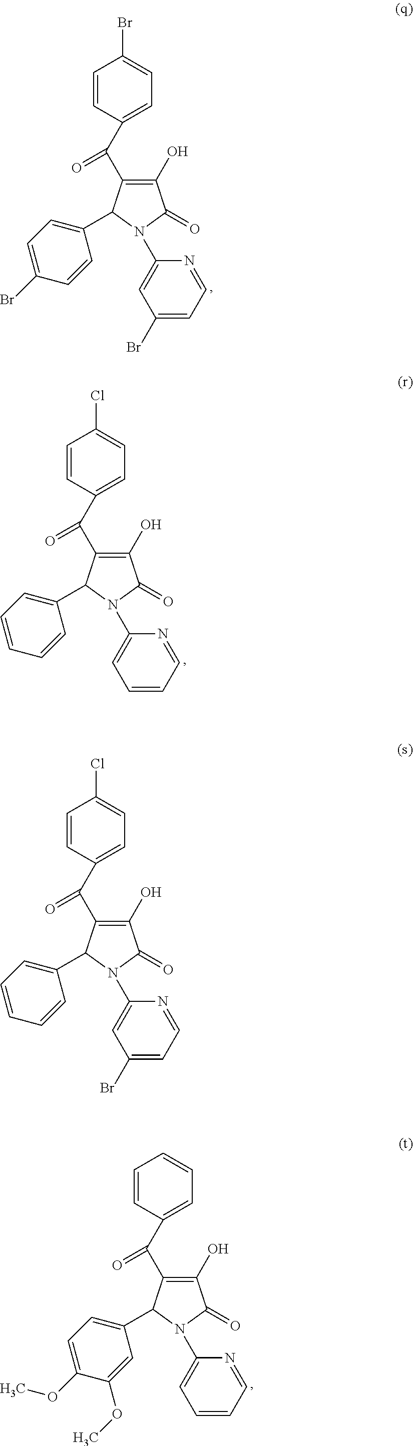

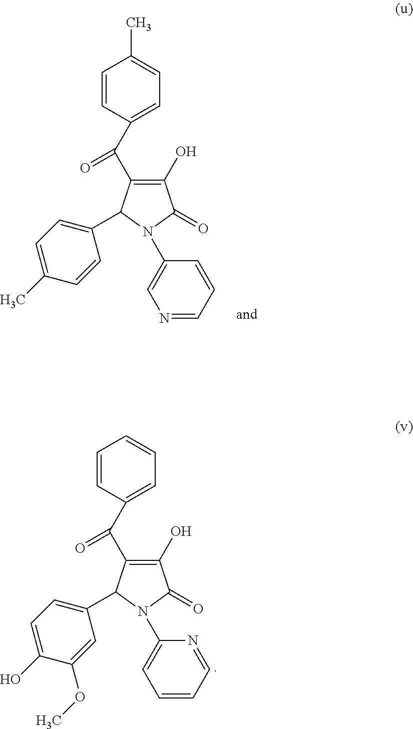

The compounds of formula (g), (u) and (v) are known as such but they have never been described as having a therapeutic activity, in particular an antiviral activity, more particularly an anti HCV activity.

The compounds of formula (e), (f), (h), (i), (j), (k), (l), (m), (n), (O), (p), (q), (r), (s) and (t) are known as having a therapeutic activity, but they have never been described as having an antiviral activity, more particularly an anti HCV activity.

In a particular embodiment, the compound according to the present invention or a salt, solvate, tautomer, isotope, enantiomer, diastereoisomer or racemic mixture thereof is such that R3 represents a group of the following formula II:

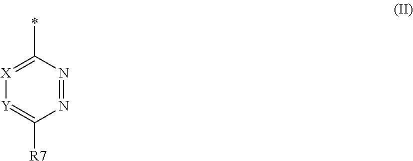

in which X represents a nitrogen atom and Y represents a —C—R8 group, or X represents a —C—R9 group and Y represents a nitrogen atom, or X represents a —C—R9 group and Y represents a —C—R8 group, advantageously X represents a —C—R9 group and Y represents a —C—R8 group; R7, R8 and R9 represent independently of each other a hydrogen atom; a halogen atom; a C₁-C₆ alkyl group optionally substituted by one or more halogen atom, in particular a fluorine atom, by a —O—(C₁-C₆)alkyl group, by a —O—C₃-C₆ cycloalkyl group, by a —O-aryl group or by a —NR′R″ group in which R′ and R″ represent independently of each other a hydrogen atom, a C₁-C₆ alkyl group, a C₃-C₆ cycloalkyl group or an aryl group; a C₃-C₆ cycloalkyl group optionally substituted by one or more halogen atom, in particular a fluorine atom; a —O—(C₁-C₆)alkyl group in which the alkyl group is optionally substituted by one or more halogen atom, in particular a fluorine atom, or by a phenyl group; a —COOH group; a —OH group; a —COO(C₁-C₆)alkyl group in which the alkyl group is optionally substituted by one or more halogen atom in particular a fluorine atom; a —CN group; a —S-phenyl-NO₂ group; a —SO₂-phenyl-NO₂ group; a —SO₂—(C₁-C₆)alkyl group; a —SO₂-aryl group; a —SO₂—NH—(C₁-C₆)alkyl group; a —SO₂—NH-aryl group; a 6-members heterocyclic group containing one or two heteroatoms selected in the group consisting of nitrogen, sulfur and oxygen atom; or a —NR′R″ group in which R′ and R″ represent independently of each other a hydrogen atom or a C₁-C₆ alkyl group, advantageously a C₁-C₆ alkyl group and
* indicates the position involved in binding with another group.

In particular, R7, R8 and R9 represent independently of each other a hydrogen atom; a halogen atom; a C₁-C₆ alkyl group optionally substituted by one or more halogen atom, in particular a fluorine atom; a C₃-C₆ cycloalkyl group optionally substituted by one or more halogen atom, in particular a fluorine atom; a —O—(C₁-C₆)alkyl group in which the alkyl group is optionally substituted by one or more halogen atom, in particular a fluorine atom; a —COOH group; a —COO(C₁-C₆)alkyl group in which the alkyl group is optionally substituted by one or more halogen atom in particular a fluorine atom; a —CN group; a —SO₂-phenyl-NO₂ group; a —SO₂—(C₁-C₆)alkyl group; a —SO₂-aryl group; a —SO₂—NH—(C₁-C₆)alkyl group; a —SO₂—NH-aryl group; a 6-members heterocyclic group containing one or two heteroatoms selected in the group consisting of nitrogen, sulfur and oxygen atom; or a —NR′R″ group in which R′ and R″ represent independently of each other a hydrogen atom or a C₁-C₆ alkyl group, advantageously a C₁-C₆ alkyl group and

Advantageously, R7, R8 and R9 represent independently of each other a hydrogen atom; a halogen atom, in particular a chlorine atom; a —O—(C₁-C₆)alkyl group in which the alkyl group is optionally substituted by a phenyl group, in particular —OCH₃ group or a-OCH₂phenyl group; a —OH group; a —S-phenyl-NO₂ group; a C₁-C₆ alkyl group optionally substituted by one or more halogen atom, in particular a fluorine atom; a C₃-C₆ cycloalkyl group optionally substituted by one or more halogen atom, in particular a fluorine atom; or a —NR′R″ group in which R′ and R″ represent independently of each other a hydrogen atom or a C₁-C₆ alkyl group, advantageously a C₁-C₆ alkyl group. More advantageously R7, R8 and R9 represent independently of each other a hydrogen atom; a C₁-C₆ alkyl group, in particular a methyl group, or a —NR′R″ group in which R′ and R″ represent independently of each other a hydrogen atom or a C₁-C₆ alkyl group, advantageously a C₁-C₆ alkyl group. Still more advantageously R8 and R9 represent a hydrogen atom. Even still more advantageously, R7 represents a C₁-C₆ alkyl group, in particular a methyl group.

In another particular embodiment the compound according to the present invention or a salt, solvate, tautomer, isotope, enantiomer, diastereoisomer or racemic mixture thereof is such that R3 represents a 6-members heteroaryl group containing as the only heteroatom, one nitrogen atom, advantageously a pyridyl group, said heteroaryl group being optionally substituted by a halogen atom; a C₁-C₆ alkyl group optionally substituted by one or more halogen atom, in particular a fluorine atom; a C₃-C₆ cycloalkyl group optionally substituted by one or more halogen atom, in particular a fluorine atom; a —O—(C₁-C₆)alkyl group in which the alkyl group is optionally substituted by one or more halogen atom, in particular a fluorine atom, or by a phenyl group; a —OH group; a —COOH group; a —COO(C₁-C₆)alkyl group in which the alkyl group is optionally substituted by one or more halogen atom in particular a fluorine atom; a —CN group; a ═O group; a —SO₂-phenyl-NO₂ group; a —S-phenyl-NO₂ group; a —SO₂—(C₁-C₆)alkyl group; a —SO₂-aryl group; a —SO₂—NH—(C₁-C₆)alkyl group; a —SO₂—NH-aryl group; a 6-members heterocyclic group containing one or two heteroatoms selected in the group consisting of nitrogen, sulfur and oxygen atom; or a —NR′R″ group in which R′ and R″ represent independently of each other a hydrogen atom or a C₁-C₆ alkyl group, advantageously the 6-members heteroaryl group is substituted by a C₁-C₆ alkyl group or a —CN group.

Advantageously the heteroaryl group is unsubstituted or substituted by a halogen atom; a C₁-C₆ alkyl group optionally substituted by one or more halogen atom, in particular a fluorine atom; a C₃-C₆ cycloalkyl group optionally substituted by one or more halogen atom, in particular a fluorine atom; a —O—(C₁-C₆)alkyl group in which the alkyl group is optionally substituted by one or more halogen atom, in particular a fluorine atom; a —COOH group; a —COO(C₁-C₆)alkyl group in which the alkyl group is optionally substituted by one or more halogen atom in particular a fluorine atom; a —CN group; a —SO₂-phenyl-NO₂ group; a —SO₂—(C₁-C₆)alkyl group; a —SO₂-aryl group; a —SO₂—NH—(C₁-C₆)alkyl group; a —SO₂—NH-aryl group; a 6-members heterocyclic group containing one or two heteroatoms selected in the group consisting of nitrogen, sulfur and oxygen atom; or a —NR′R″ group in which R′ and R″ represent independently of each other a hydrogen atom or a C₁-C₆ alkyl group.

In particular the heteroaryl group is unsubstituted or substituted by a halogen atom, in particular a chlorine atom; a —O—(C₁-C₆)alkyl group in which the alkyl group is optionally substituted by a phenyl group, in particular —OCH₃ group or a —OCH₂-phenyl group; a —OH group; a —S-phenyl-NO₂ group; a C₁-C₆ alkyl group optionally substituted by one or more halogen atom, more particularly a fluorine atom; a C₃-C₆ cycloalkyl group optionally substituted by one or more halogen atom, more particularly a fluorine atom; a —CN group; or a —NR′R″ group in which R′ and R″ represent independently of each other a hydrogen atom or a C₁-C₆ alkyl group, advantageously a C₁-C₆ alkyl group. More advantageously the heteroaryl group is unsubstituted or substituted by a C₁-C₆ alkyl group, in particular a methyl group, or a —CN group. Even still more advantageously, the heteroaryl group is substituted by a C₁-C₆ alkyl group, in particular a methyl group.

In a further particular embodiment, the compound according to the present invention or a salt, solvate, tautomer, isotope enantiomer, diastereoisomer or racemic mixture thereof is such that R2 represents a phenyl group or a pyridyl group, in particular a phenyl group, optionally substituted by one or more groups, advantageously one group, more advantageously at the para position, independently selected among a halogen atom; a —OH group; a —CN group; a C₁-C₆ alkyl group optionally substituted by one or more halogen atom, in particular a fluorine atom, or by a 5-members heteroaryl group containing one, two, three or four heteroatom (s) selected in the group consisting of oxygen, sulfur and nitrogen atom, advantageously nitrogen atom; a C₃-C₆ cycloalkyl group optionally substituted by one or more halogen atom, in particular a fluorine atom; a —O—(C₃-C₆)cycloalkyl group in which the cycloalkyl group is optionally substituted by one or more halogen atom, in particular a fluorine atom; a —O—(C₁-C₆)alkyl group in which the alkyl group is optionally substituted by one or more halogen atom in particular a fluorine atom; a —CO—(C₁-C₆)alkyl group in which the alkyl group is optionally substituted by one or more halogen atom; a —SO₂—(C₁-C₆)alkyl group in which the alkyl group is optionally substituted by one or more halogen atom, in particular a fluorine atom; a —S—(C₁-C₆)alkyl group in which the alkyl group is optionally substituted by one or more halogen atom, in particular a fluorine atom; a C₂-C₆ alkenyl group optionally substituted by one or more halogen atom, in particular a fluorine atom; a C₂-C₆ alkynyl group optionally substituted by one or more halogen atom, in particular a fluorine atom; a phenyl group; a —O-phenyl group; a 5-members heteroaryl group containing one, two, three or four heteroatom (s) selected in the group consisting of oxygen, sulfur and nitrogen atom, advantageously nitrogen and oxygen atom, the heteroaryl group being optionally substituted by a C₁-C₆ alkyl group; a —NR′R″ group in which R′ and R″ represent independently of each other a hydrogen atom or a C₁-C₆ alkyl group, a —O-(6-members heterocyclic) group in which the heterocyclic group contains one, two or three heteroatoms selected in the group consisting of nitrogen, sulfur and oxygen atom, advantageously nitrogen and oxygen atom; a —O-(5- or 6-members heteroaryl) group in which the heteroaryl group contains one, two, three or four heteroatom (s) selected in the group consisting of oxygen, sulfur and nitrogen atom, advantageously nitrogen and oxygen atom, the heteroaryl group being optionally substituted by a —(C₁-C₆ alkyl)-phenyl group, a —(C₁-C₆ alkyl) group or a —CH₂—O—CH₂—Si(CH₃)₃ group, the alkyl group being optionally substituted by a halogen atom; a —O—((C₁-C₆)alkyl)-(6-members heterocyclic) group in which the heterocyclic group contains one, two or three heteroatoms selected in the group consisting of nitrogen, sulfur and oxygen atom; a —O—((C₁-C₆)alkyl)-NR′R″ group in which R′ and R″ represent independently of each other a hydrogen atom or a C₁-C₆ alkyl group, and a 6-members heterocyclic group containing one, two or three heteroatoms selected in the group consisting of nitrogen, sulfur and oxygen atom, advantageously nitrogen atom, the heterocyclic group being optionally substituted by a —((C₁-C₆)alkyl)-NR′R″ group in which R′ and R″ represent independently of each other a hydrogen atom or a C₁-C₆ alkyl group.

Advantageously the phenyl or pyridyl group is substituted by a halogen atom; a —OH group; a C₁-C₆ alkyl group optionally substituted by one or more halogen atom, in particular a fluorine atom; a C₃-C₆ cycloalkyl group optionally substituted by one or more halogen atom, in particular a fluorine atom; a —O—(C₁-C₆)alkyl group in which the alkyl group is optionally substituted by one or more halogen atom in particular a fluorine atom; a phenyl group; a —O-phenyl group; a —NR′R″ group in which R′ and R″ represent independently of each other a hydrogen atom or a C₁-C₆ alkyl group, a —O-(6-members heterocyclic) group in which the heterocyclic group contains one, two or three heteroatoms selected in the group consisting of nitrogen, sulfur and oxygen atom, advantageously nitrogen atom; a —O((C₁-C₆)alkyl)-(6-members heterocyclic) group in which the heterocyclic group contains one, two or three heteroatoms selected in the group consisting of nitrogen, sulfur and oxygen atom; a —O—((C₁-C₆)alkyl)-NR′R″ group in which R′ and R″ represent independently of each other a hydrogen atom or a C₁-C₆ alkyl group, and a 6-members heterocyclic group containing one, two or three heteroatoms selected in the group consisting of nitrogen, sulfur and oxygen atom, advantageously nitrogen atom, the heterocyclic group being optionally substituted by a —((C₁-C₆)alkyl)-NR′R″ group in which R′ and R″ represent independently of each other a hydrogen atom or a C₁-C₆ alkyl group. In particular the phenyl or pyridyl group is substituted by a —O-(5-members heteroaryl) group in which the heteroaryl group contains one, two, three or four heteroatom (s), in particular three heteroatoms, selected in the group consisting of oxygen, sulfur and nitrogen atom, advantageously nitrogen atom, the heteroaryl group being optionally substituted by a —(C₁-C₆ alkyl)-phenyl group, a —(C₁-C₆ alkyl) group or a —CH₂—O—CH₂—Si(CH₃)₃ group, the alkyl group being optionally substituted by a halogen atom, advantageously the heteroaryl group is substituted by a —CH₂—O—CH₂—Si(CH₃)₃ group; a halogen atom; a —S—(C₁-C₆)alkyl group in which the alkyl group is optionally substituted by one or more halogen atom, in particular a fluorine atom; a C₂-C₆ alkenyl group optionally substituted by one or more halogen atom, in particular a fluorine atom; a —O—(C₃-C₆)cycloalkyl group in which the cycloalkyl group is optionally substituted by one or more halogen atom, in particular a fluorine atom; a C₁-C₆ alkyl group optionally substituted by one or more halogen atom, more particularly a fluorine atom; a C₃-C₆ cycloalkyl group optionally substituted by one or more halogen atom, more particularly a fluorine atom; a —O—(C₁-C₆)alkyl group in which the alkyl group is optionally substituted by one or more halogen atom; a phenyl group; or a 6-members heterocyclic group containing one, two or three heteroatoms selected in the group consisting of nitrogen, sulfur and oxygen atom, in particular a morpholinyl group. More advantageously the phenyl or pyridyl group is substituted by a halogen atom, in particular a boron atom; a —S—(C₁-C₆)alkyl group in particular a —SCH(CH₃)₂; a C₂-C₆ alkenyl group, in particular a isopropenyl group; a —O—(C₃-C₆)cycloalkyl group in particular a —O-cyclopropyl; a C₁-C₆ alkyl group, in particular a tert-butyl group, an ethyl group or a isopropyl group, optionally substituted by one or more halogen atom, more particularly a —CF₃ group or a —CF₂CH₃ group, a —O—(C₁-C₆)alkyl group, in particular a O-methyl, O-ethyl or a O-isopropyl group, in which the alkyl group is optionally substituted by one or more halogen atom, in particular a —OCF₃ group; or a phenyl group. Even still more advantageously, the phenyl or pyridyl group is substituted by a C₁-C₆ alkyl group, in particular a isopropyl group, optionally substituted by one or more halogen atom, more particularly a —CF₃ group or a —CF₂CH₃ group; or a —O—(C₁-C₆)alkyl group, in particular a O-methyl, in which the alkyl group is optionally substituted by one or more halogen atom, in particular a —OCF₃ group. In an advantageous embodiment, the phenyl or pyridyl group is substituted by —CF₃; —OCF₃, —CF₂CH₃ or an isopropyl group.

In another particular embodiment, the compound according to the present invention or a salt, solvate, tautomer, isotope enantiomer, diastereoisomer or racemic mixture thereof is such that R1 represents a phenyl or pyridyl group, in particular a phenyl group, optionally substituted, advantageously at the para position, by a halogen atom; a —CN group; a —SO₂—(C₁-C₆) alkyl group in which the alkyl group is optionally substituted by one or more halogen atom, in particular a fluorine atom; a C₁-C₆ alkyl group optionally substituted by one or more halogen atom, in particular a fluorine atom; a C₂-C₆ alkenyl group optionally substituted by one or more halogen atom, in particular a fluorine atom; a C₃-C₆ cycloalkyl group optionally substituted by one or more halogen atom, in particular a fluorine atom; a —O—(C₁-C₆)alkyl group in which the alkyl group is optionally substituted by one or more halogen atom in particular a fluorine atom; a O—(C₁-C₆)alkyl-O—(C₁-C₆)alkyl group in which the alkyl group is optionally substituted by one or more halogen atom; a —O—(C₁-C₆)alkyl-phenyl-β-(C₁-C₆)alkyl group; a phenyl group; a 5- or 6-members heteroaryl group containing one, two or three heteroatoms selected in the group consisting of oxygen, nitrogen and sulfur atom, advantageously a nitrogen atom; a 6-members heterocyclic group containing one, two or three heteroatoms selected in the group consisting of nitrogen, sulfur and oxygen atom; or a —NR′R″ group in which R′ and R″ represent independently of each other a hydrogen atom or a C₁-C₆ alkyl group.

Advantageously, the phenyl or pyridyl group is substituted, more particularly at the para position, by a halogen atom; a C₁-C₆ alkyl group optionally substituted by one or more halogen atom, in particular a fluorine atom; a C₃-C₆ cycloalkyl group optionally substituted by one or more halogen atom, in particular a fluorine atom; a —O—(C₁-C₆)alkyl group in which the alkyl group is optionally substituted by one or more halogen atom in particular a fluorine atom; a O—(C₁-C₆)alkyl-O—(C₁-C₆)alkyl group in which the alkyl group is optionally substituted by one or more halogen atom; a phenyl group; a —O—(C₁-C₆)alkyl-phenyl-O—(C₁-C₆)alkyl group; a 6-members heterocyclic group containing one, two or three heteroatoms selected in the group consisting of nitrogen, sulfur and oxygen atom; or a —NR′R″ group in which R′ and R″ represent independently of each other a hydrogen atom or a C₁-C₆ alkyl group.

In particular, the phenyl or pyridyl group is substituted, more particularly at the para position, by a C₂-C₆ alkenyl group optionally substituted by one or more halogen atom, in particular a fluorine atom; a C₁-C₆ alkyl group optionally substituted by one or more halogen atom, in particular a fluorine atom; a —O—(C₁-C₆)alkyl group in which the alkyl group is optionally substituted by one or more halogen atom, in particular a fluorine atom; a C₃-C₆ cycloalkyl group optionally substituted by one or more halogen atom, in particular a fluorine atom; or a —NR′R″ group in which R′ and R″ represent independently of each other a hydrogen atom or a C₁-C₆ alkyl group, in particular a C₁-C₆ alkyl group. Advantageously, the phenyl or pyridyl group is substituted, more particularly at the para position, by a C₁-C₆ alkyl group, in particular a methyl, ethyl, tert-butyl, isobutyl or isopropyl group, optionally substituted by one or more halogen atom, in particular a fluorine atom; a C₂-C₆ alkenyl group, in particular a isopropenyl group; a —O—(C₁-C₆)alkyl group, in particular a O-methyl group, in which the alkyl group is optionally substituted by one or more halogen atom, in particular a fluorine atom. More advantageously, the phenyl or pyridyl group is substituted, more particularly at the para position, by a C₁-C₆ alkyl group in particular a methyl or isopropyl group, or a —OCF₃ group or a CF₃ group.

Advantageously, the compound according to the present invention, a salt, solvate, tautomer, isotope, enantiomer, diastereoisomer or racemic mixture thereof, is chosen from the group consisting of the compounds of the following formula 1, 3-10, 14-64, 66, 67, 74-80, 82-106 and 108-212.

The particularly advantageous compounds are enantiomers of the compounds according to the present invention having the following formula:

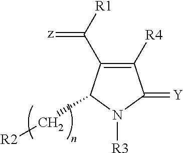

in which R1, R2, R3, R4, Y, Z and n are as defined above.

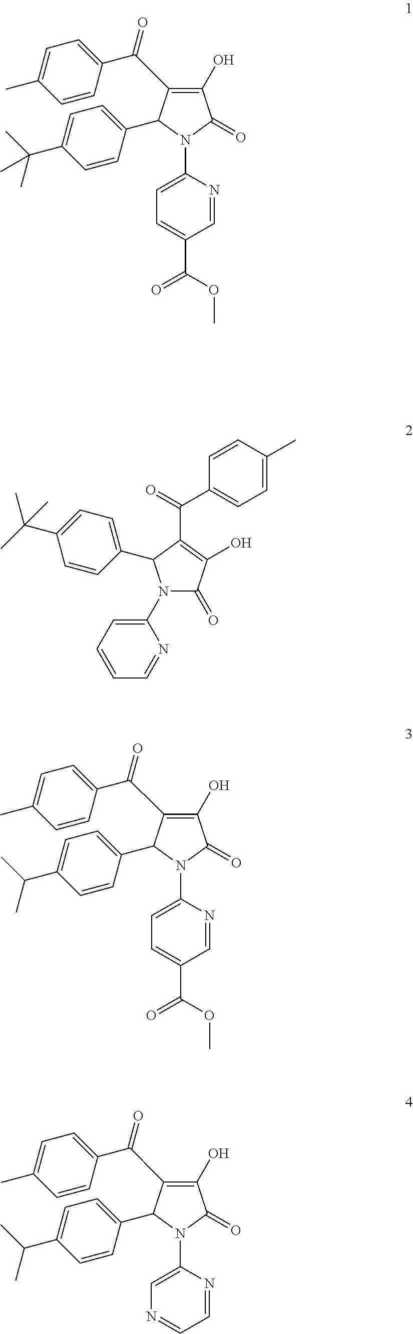

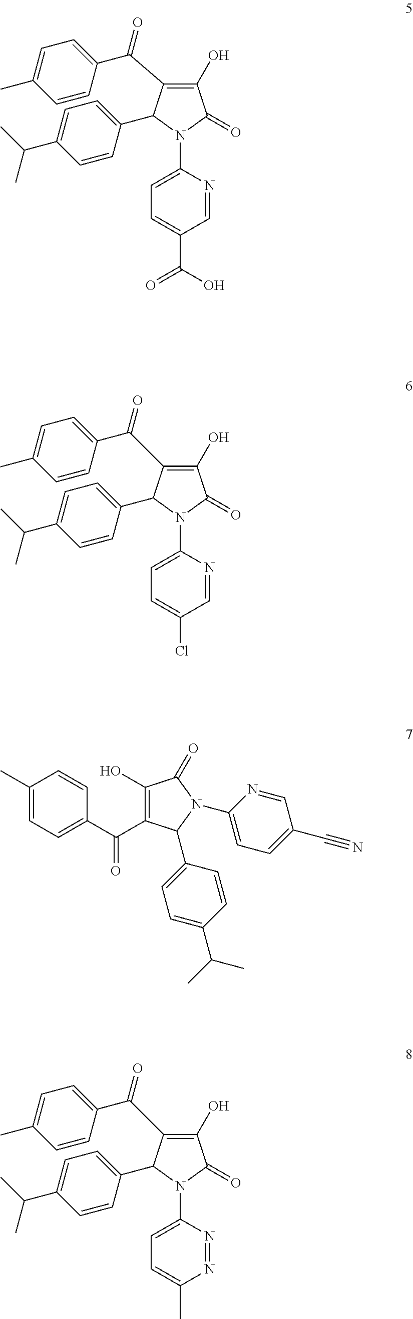

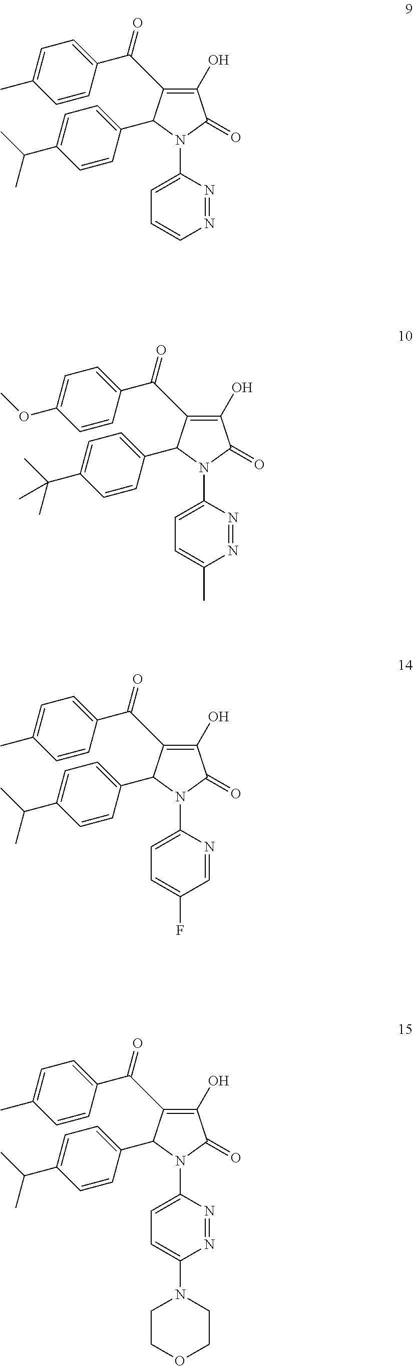

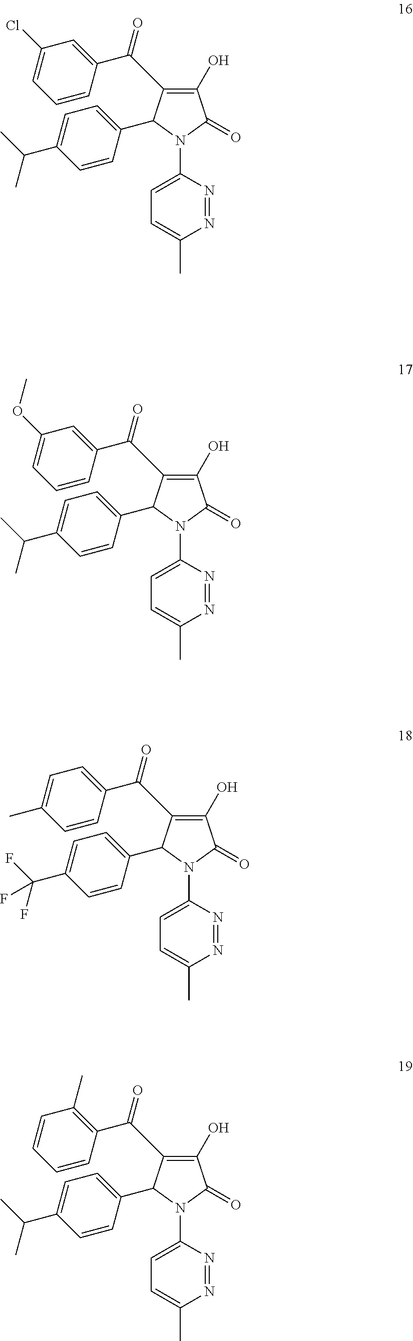

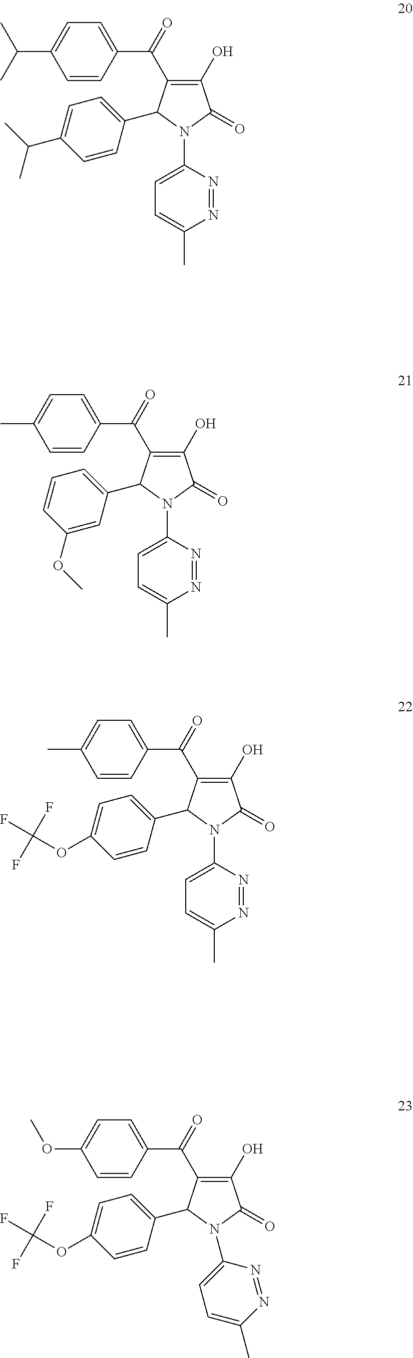

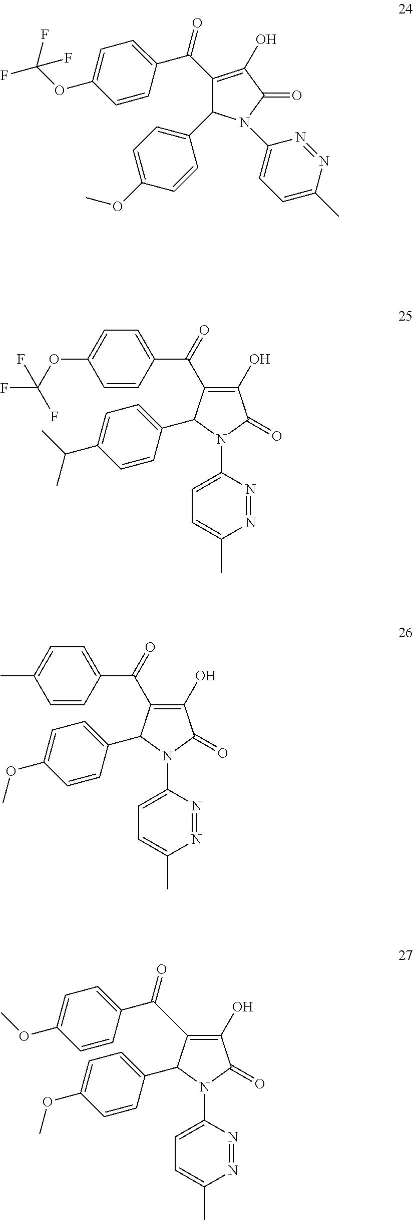

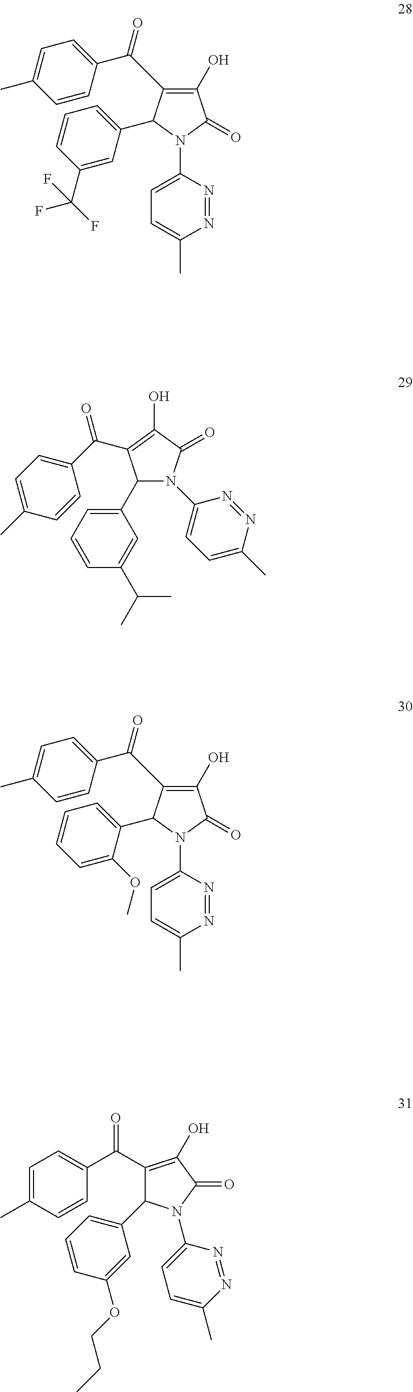

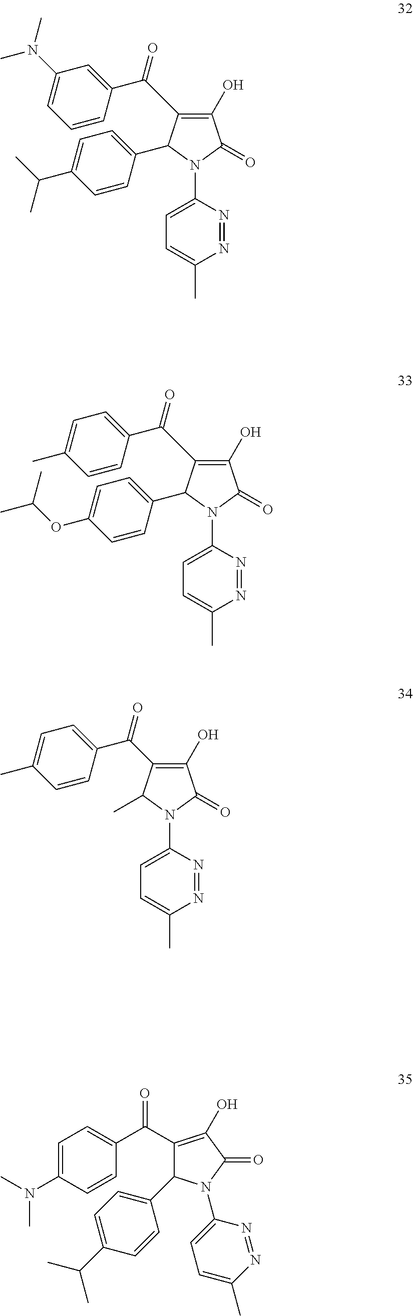

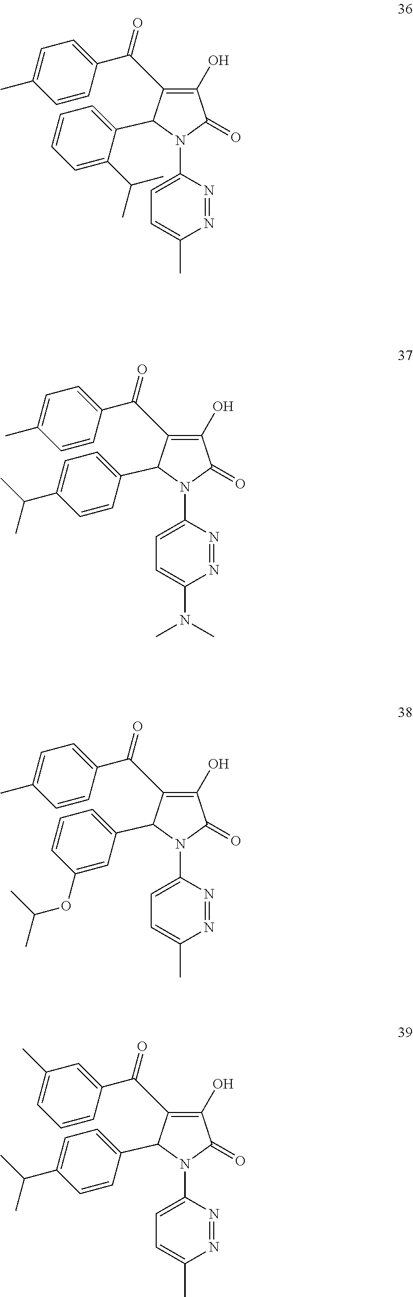

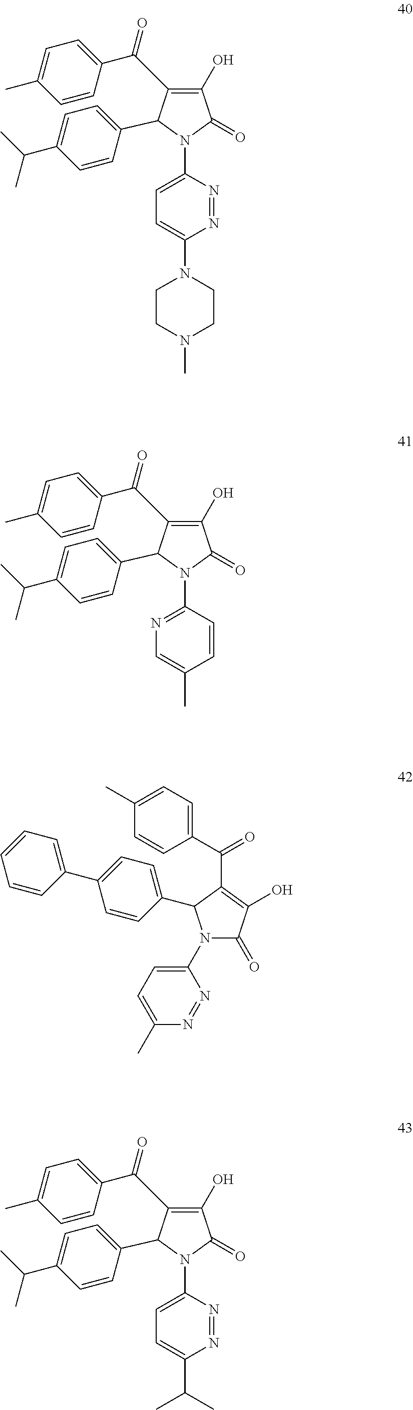

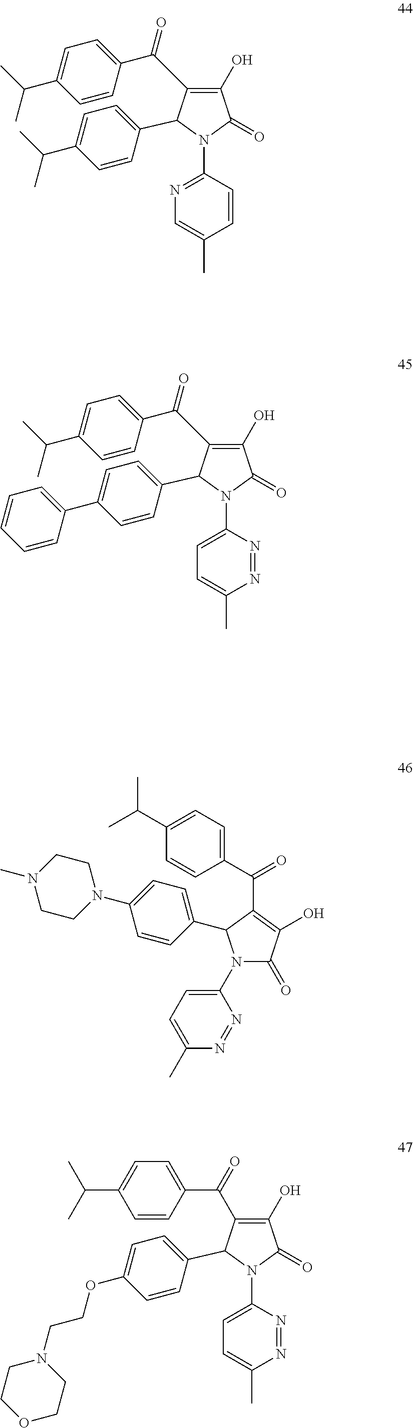

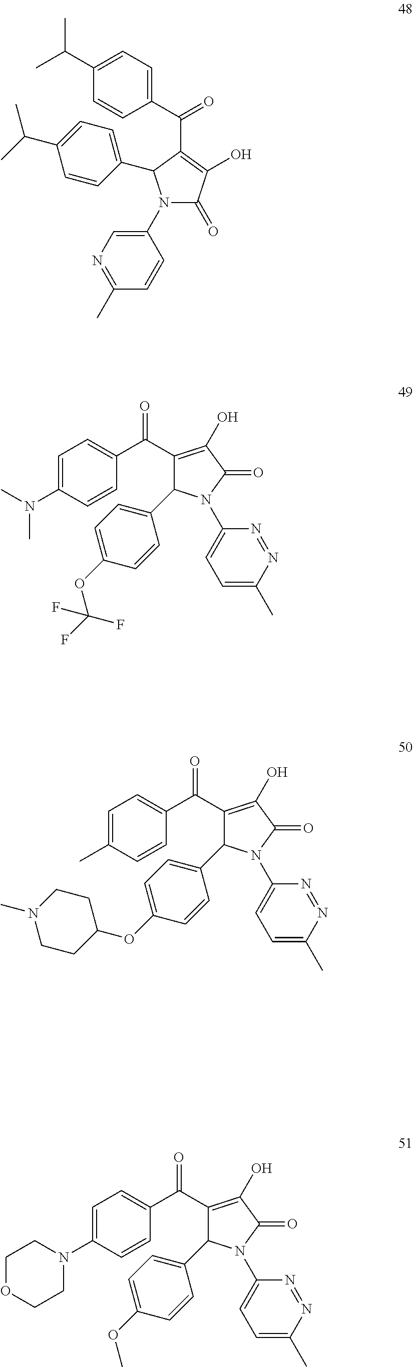

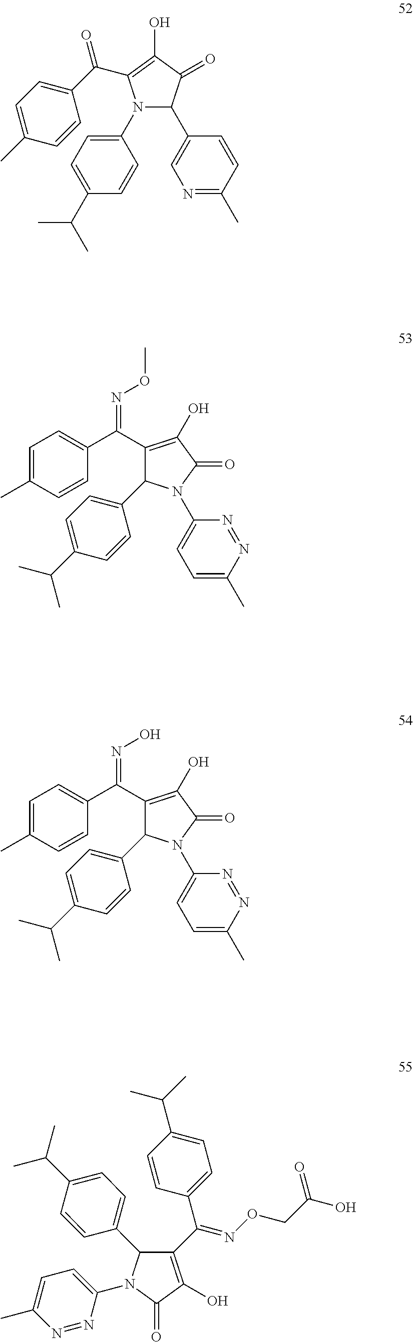

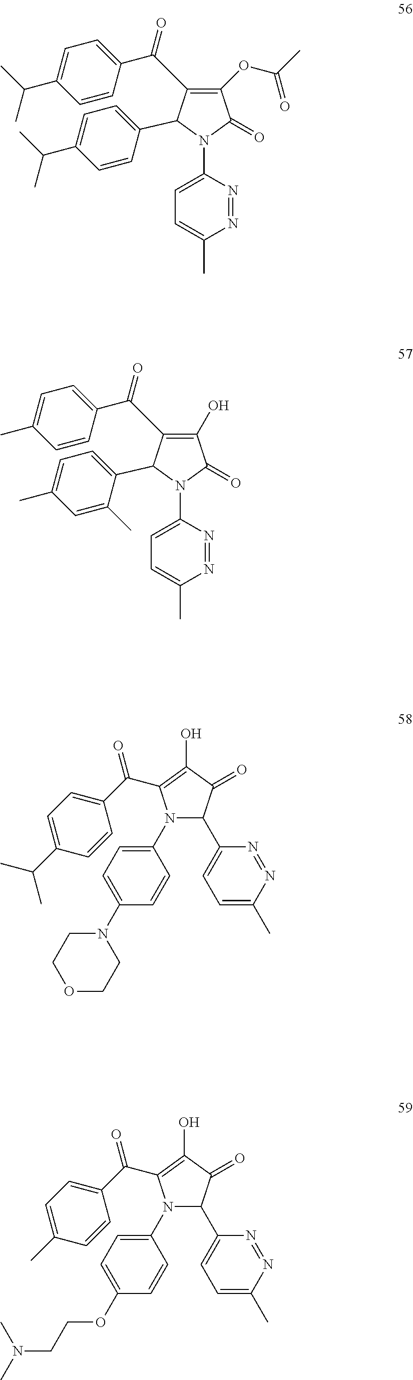

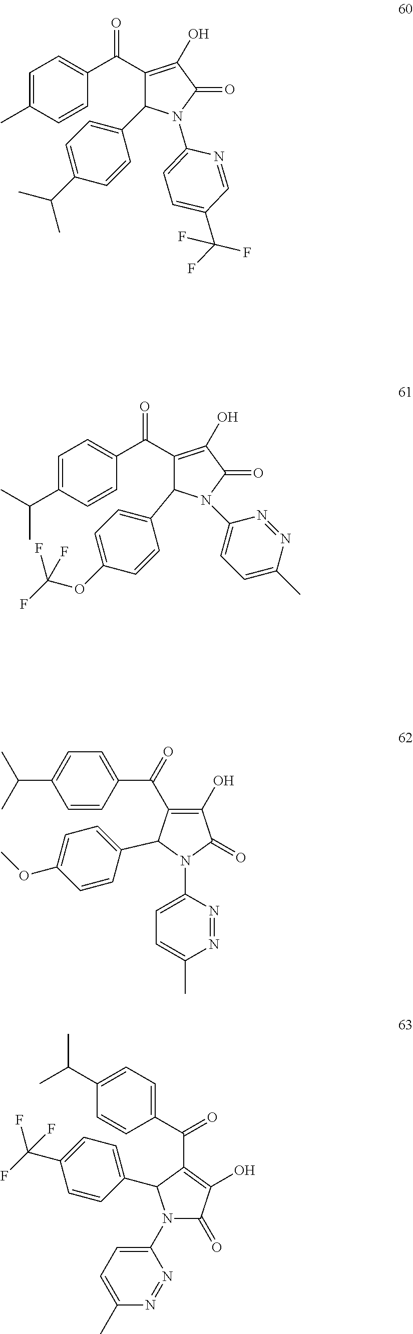

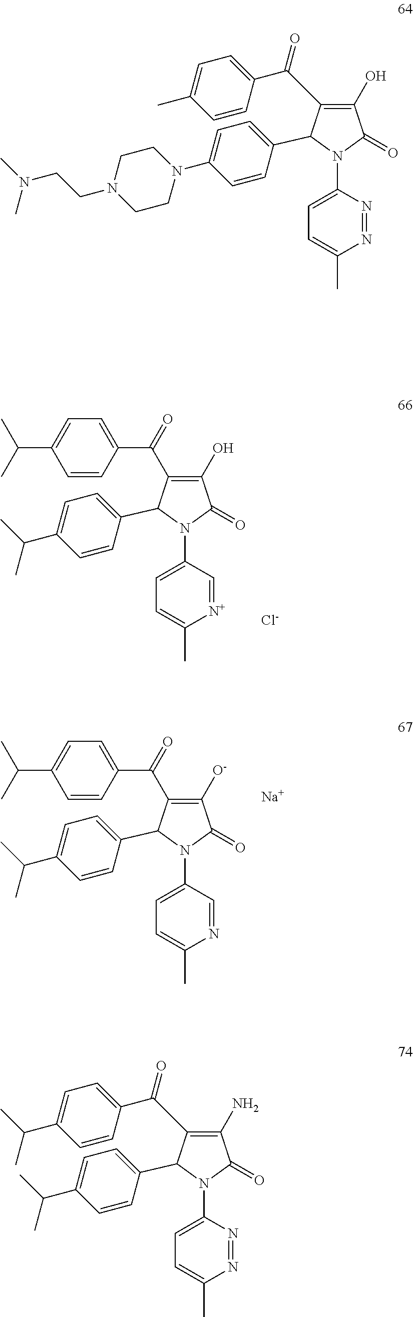

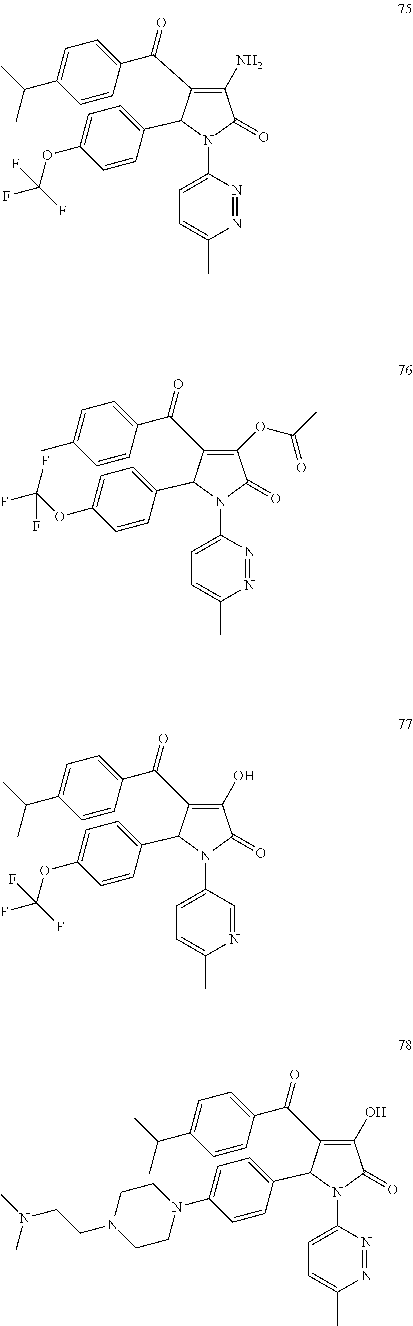

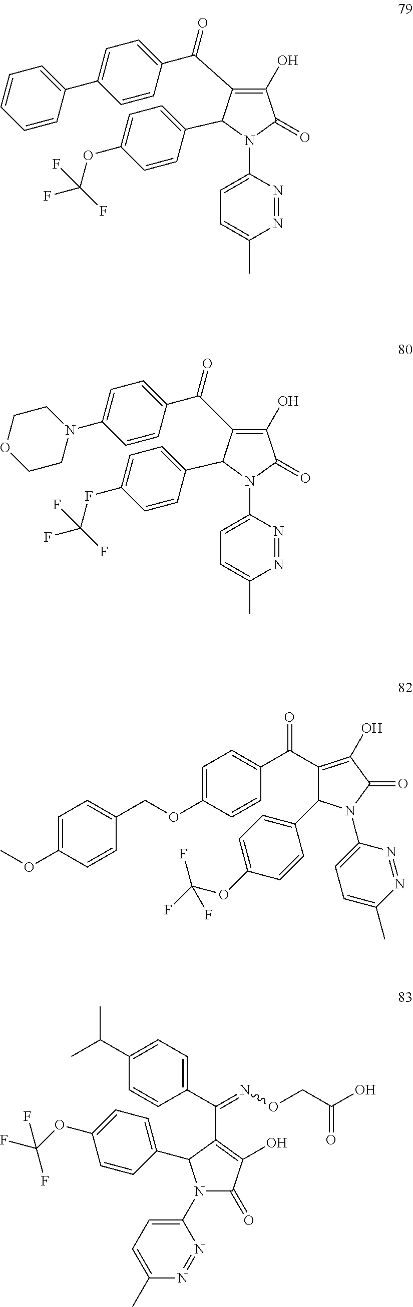

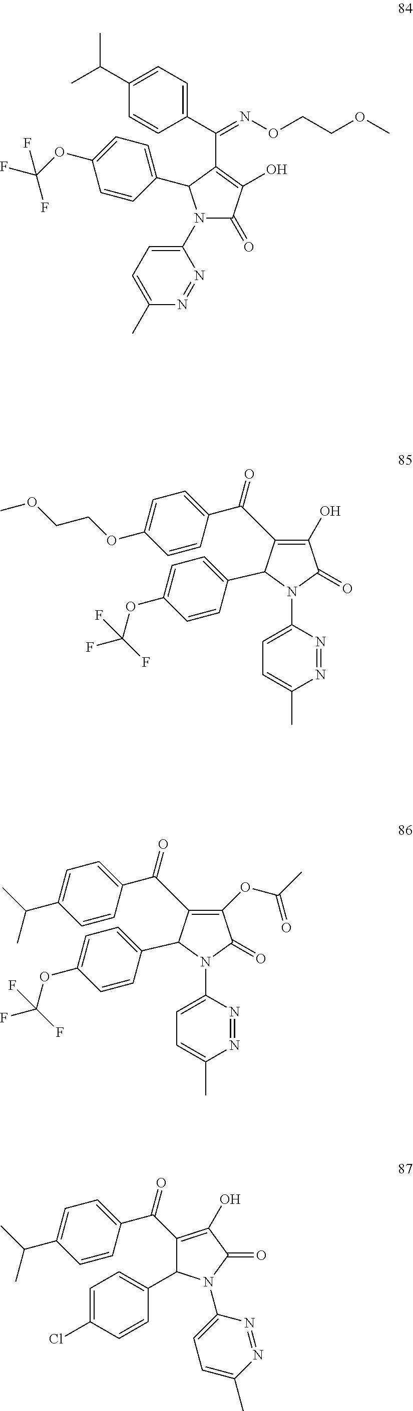

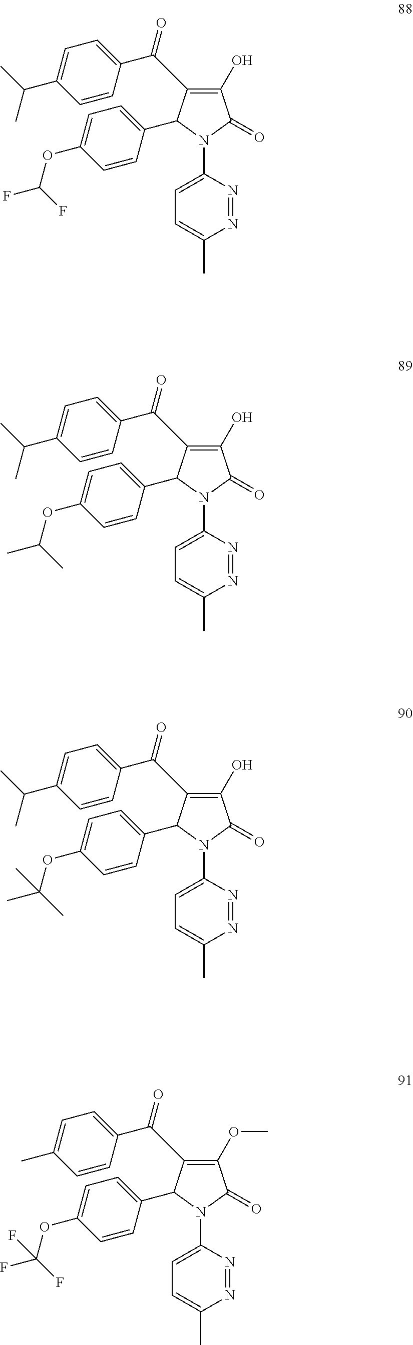

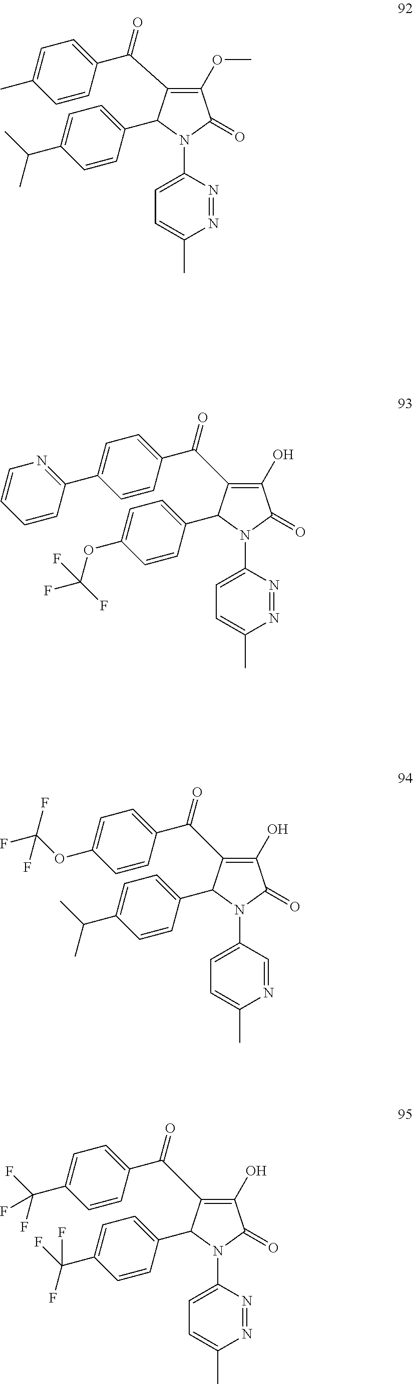

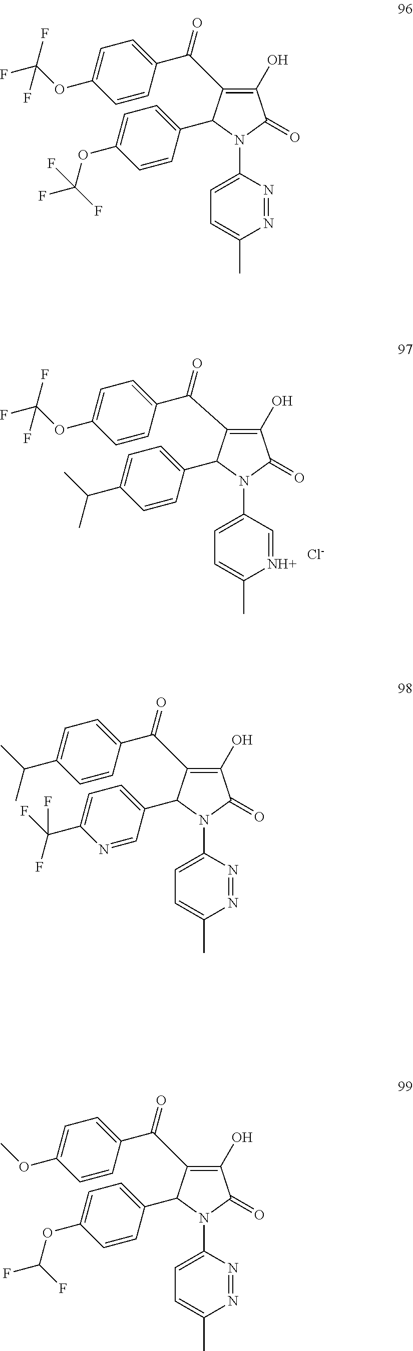

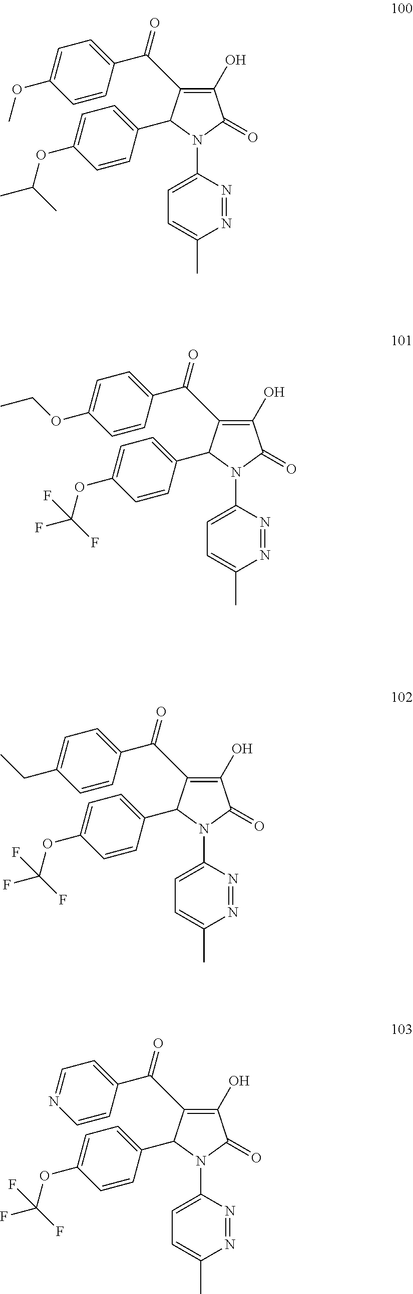

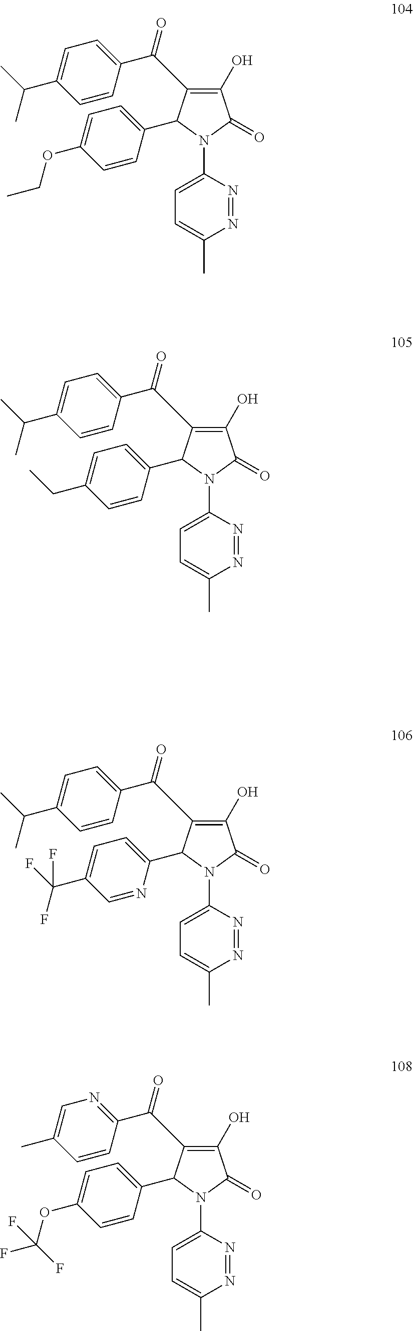

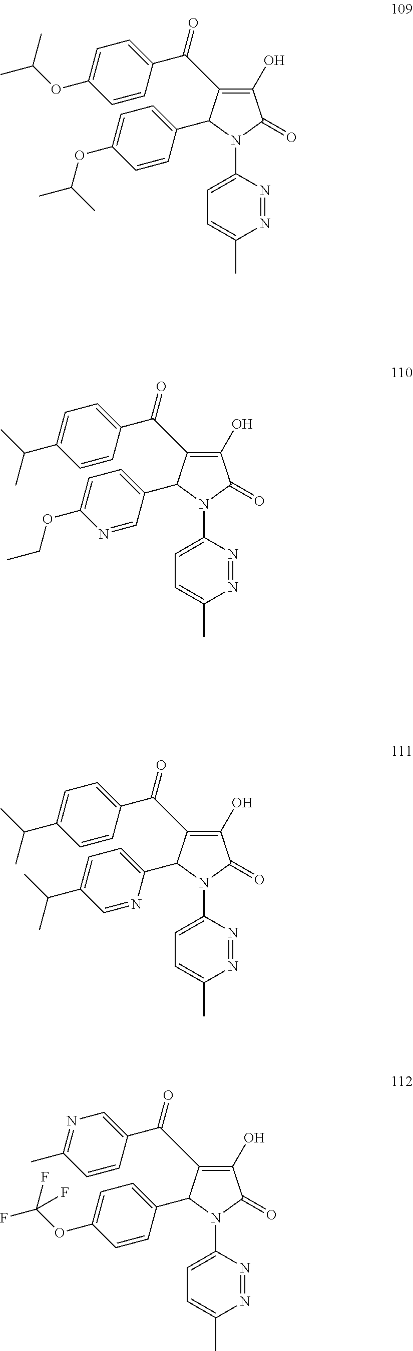

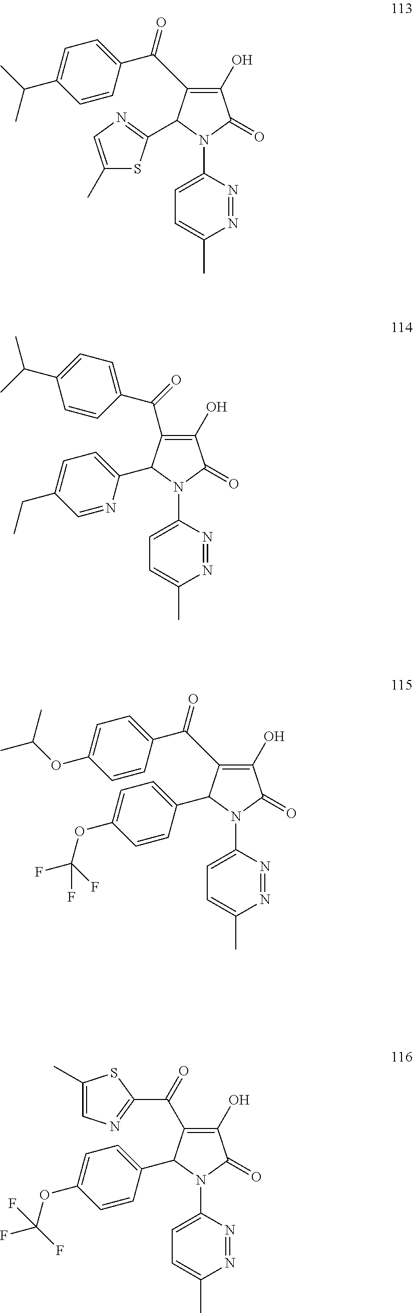

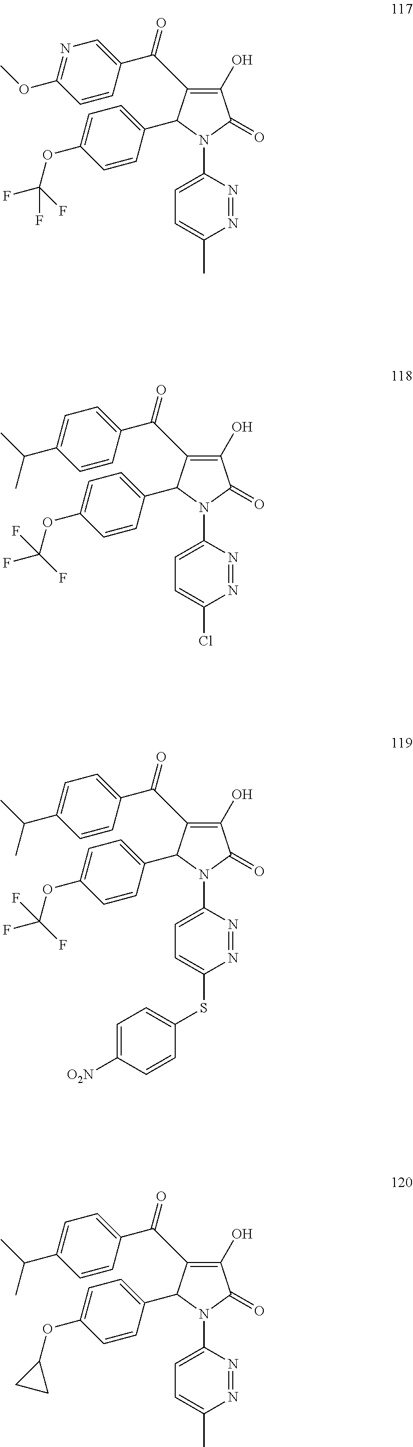

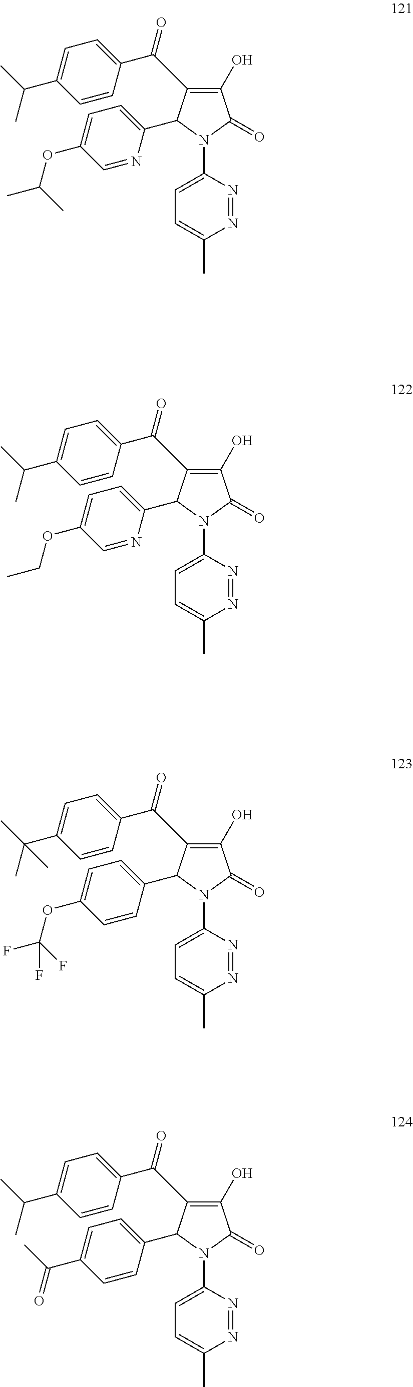

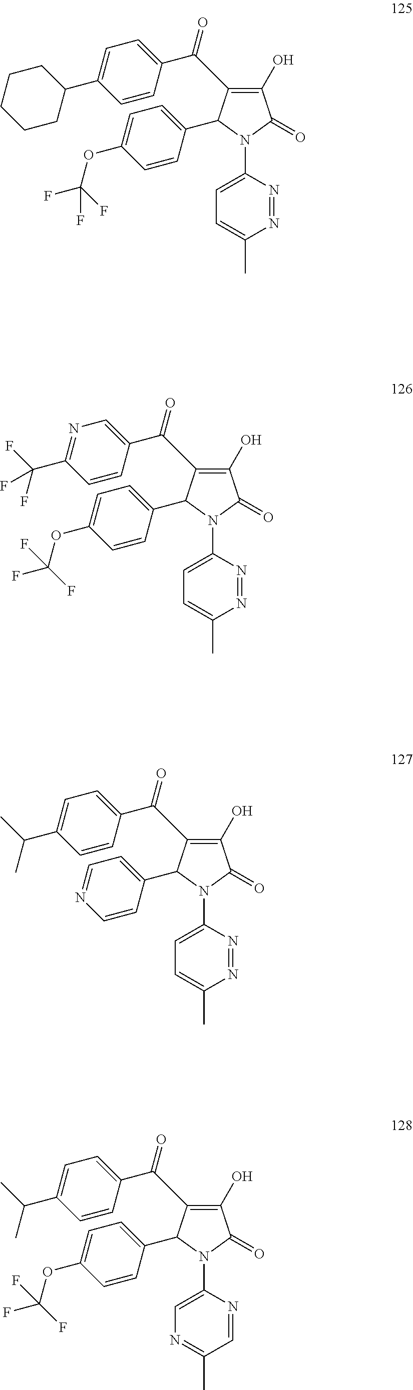

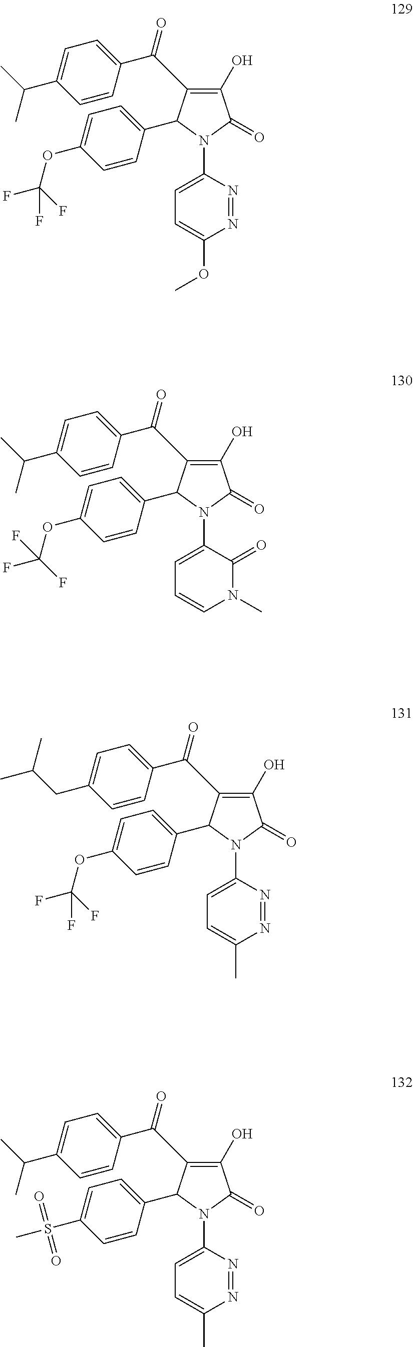

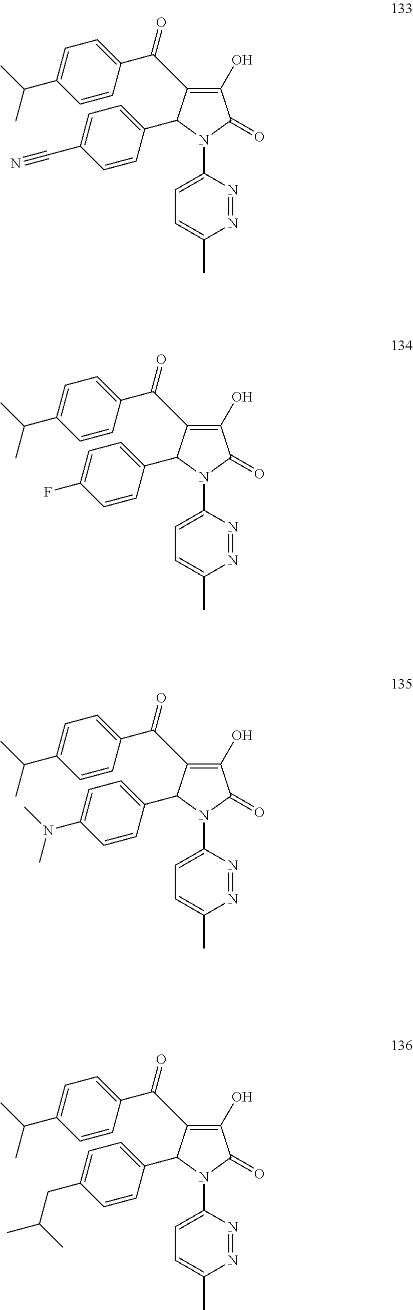

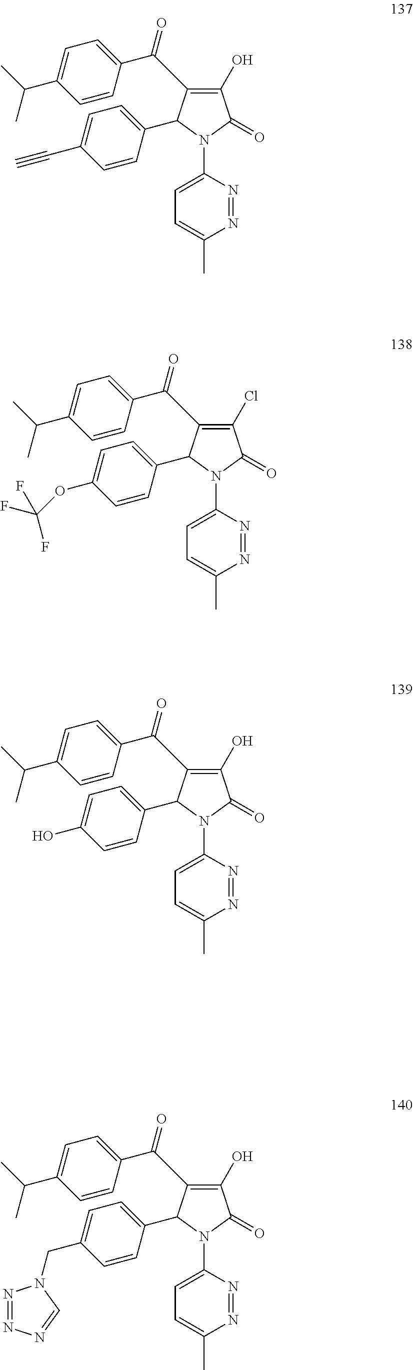

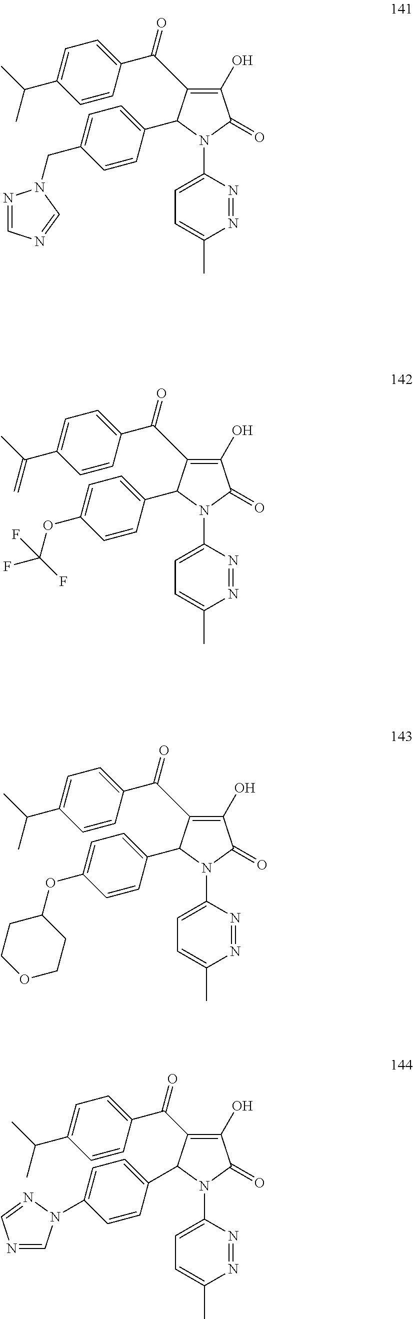

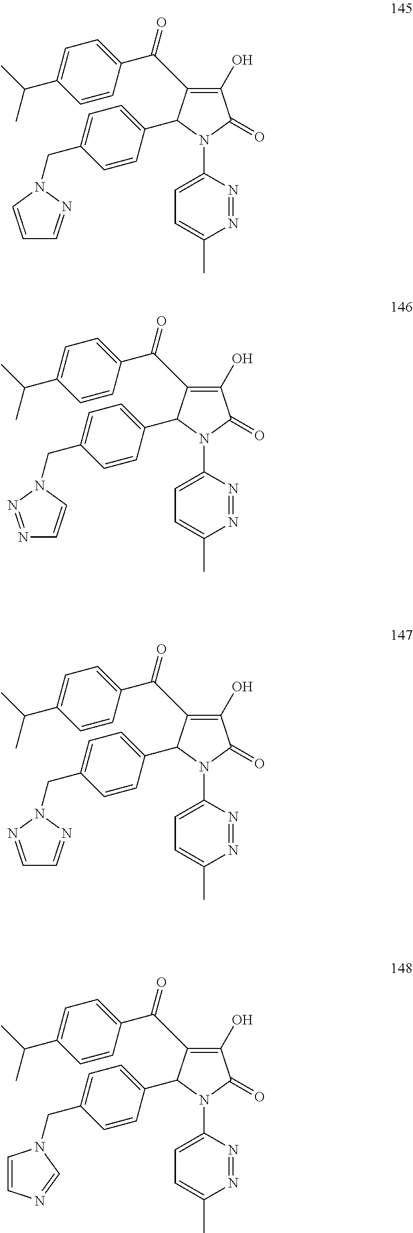

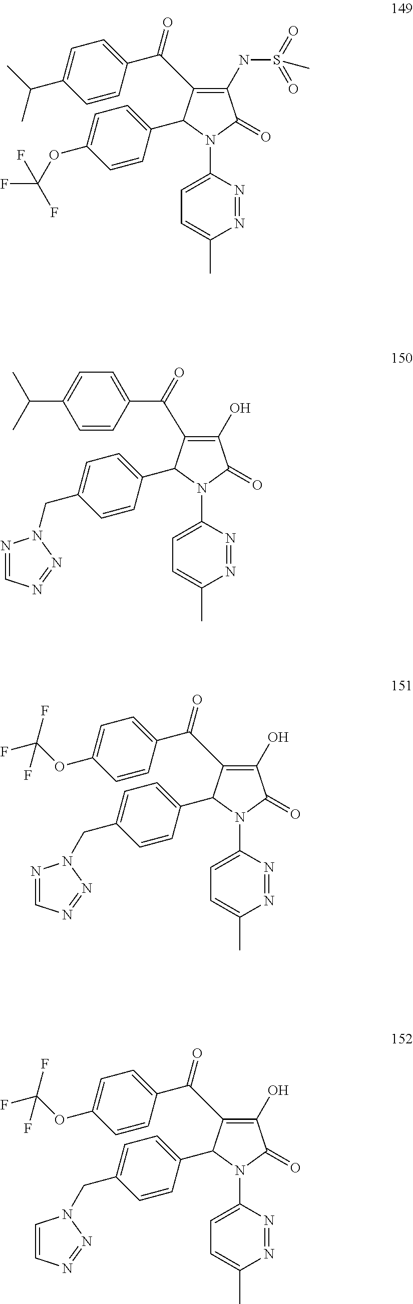

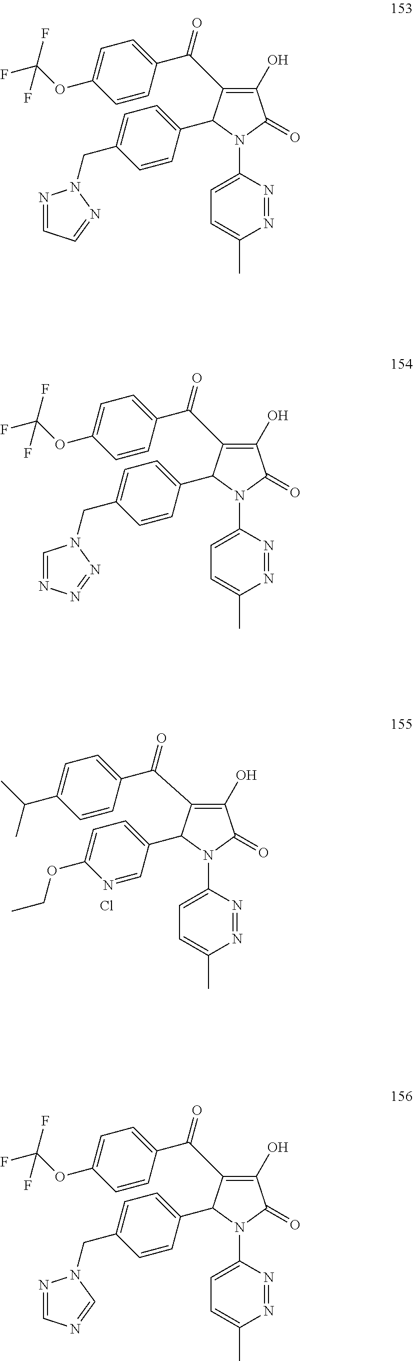

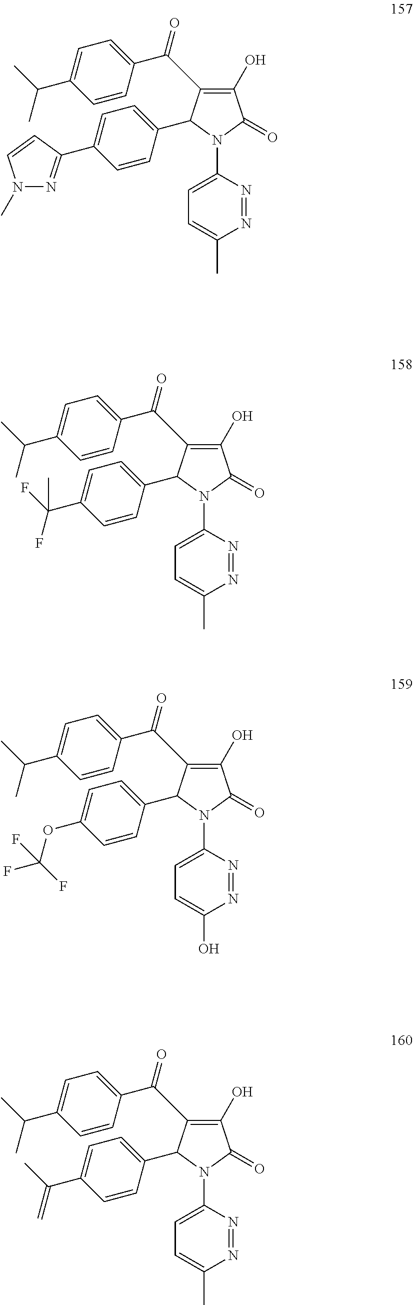

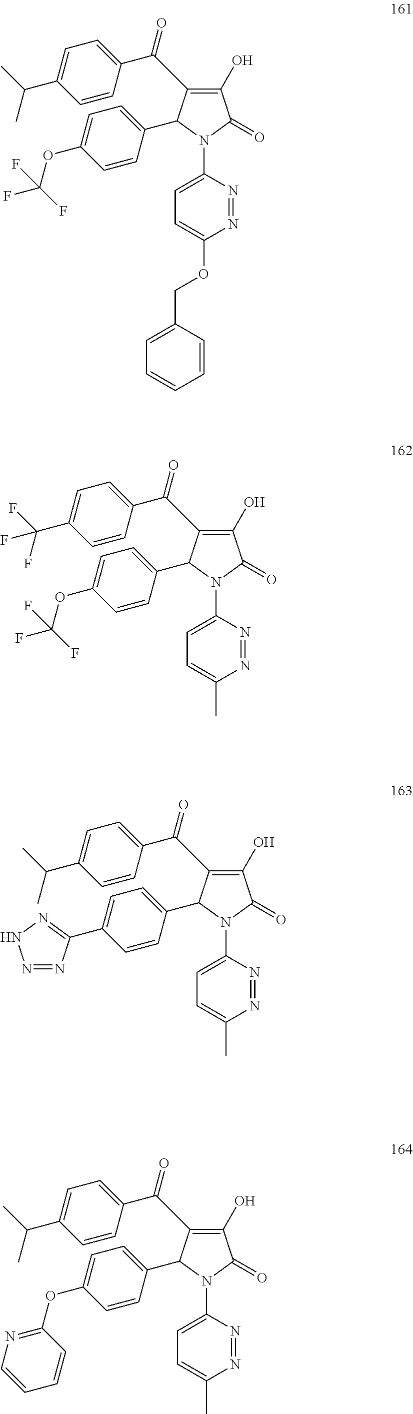

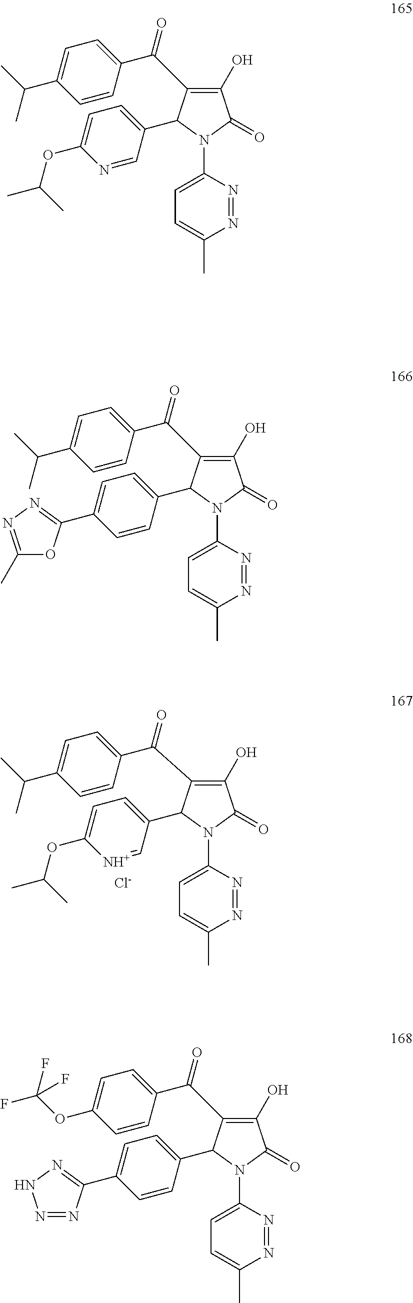

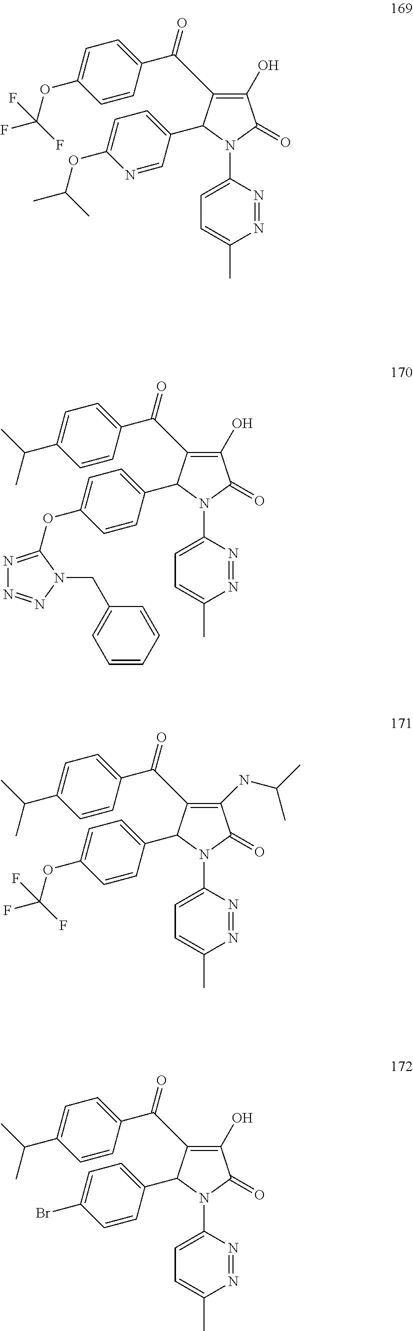

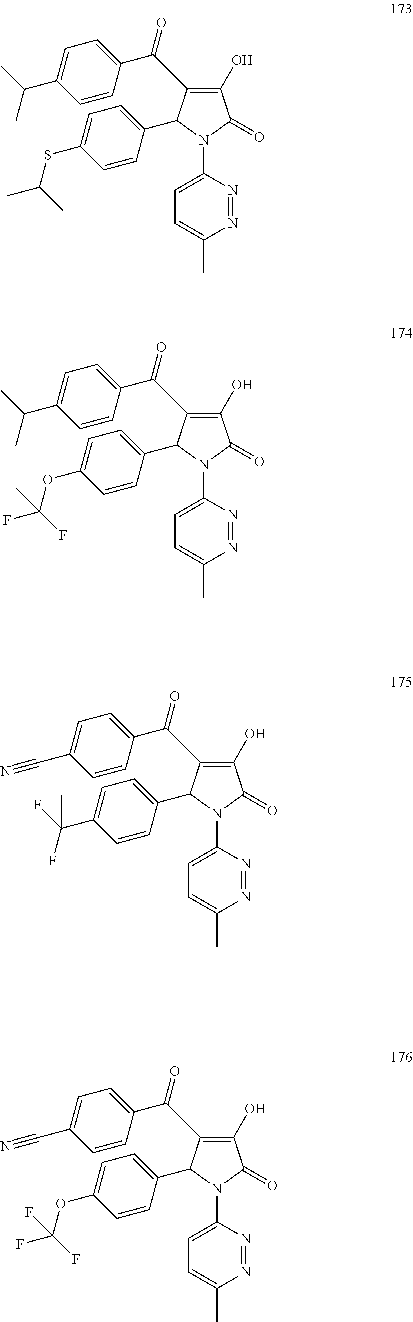

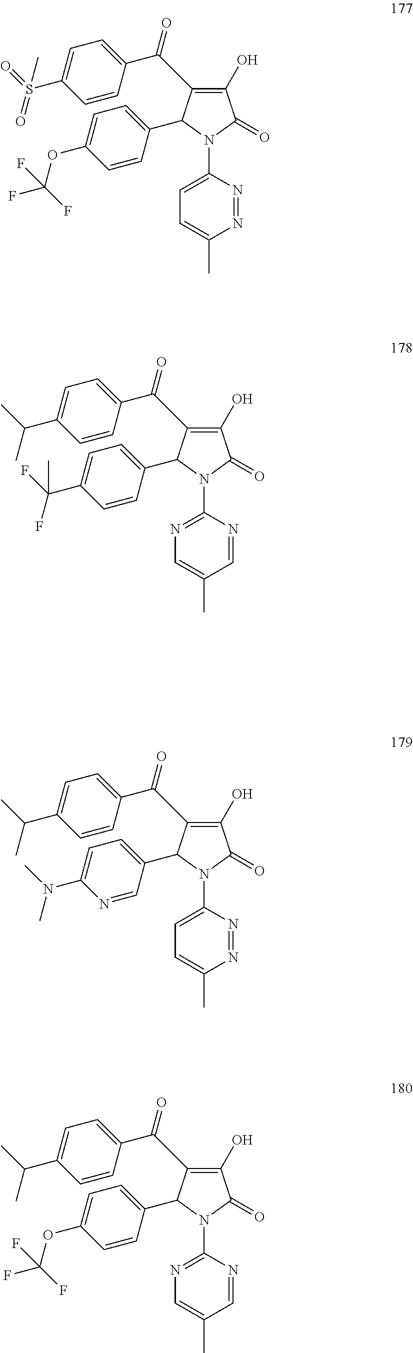

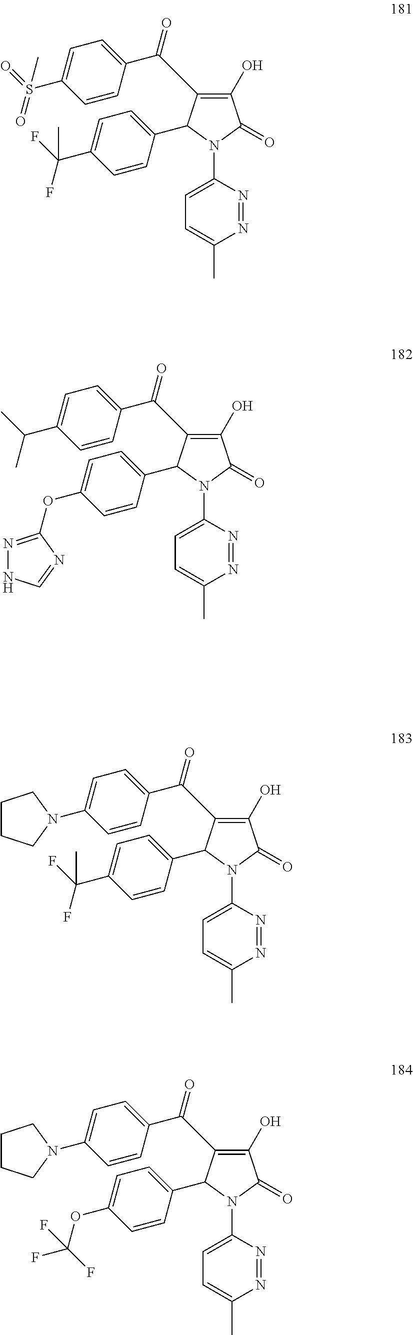

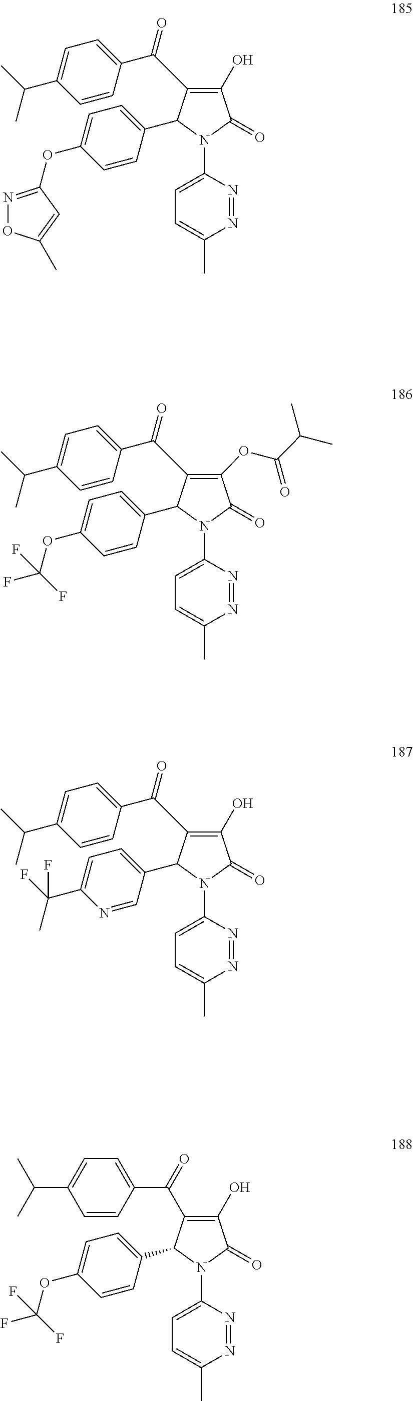

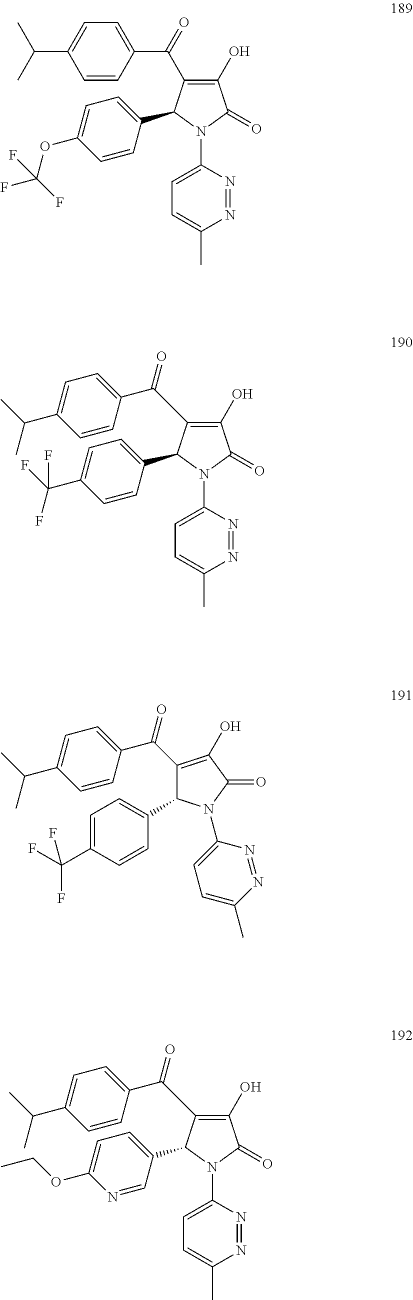

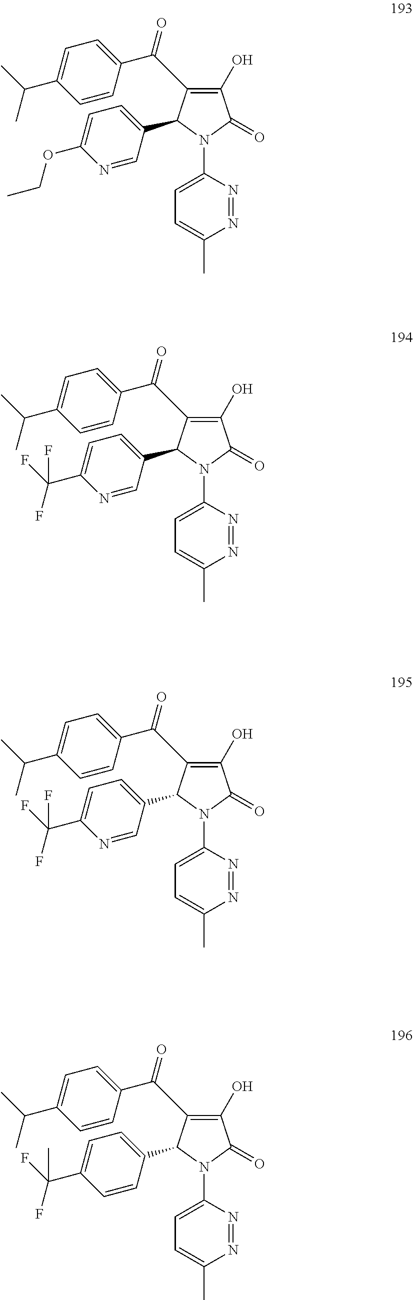

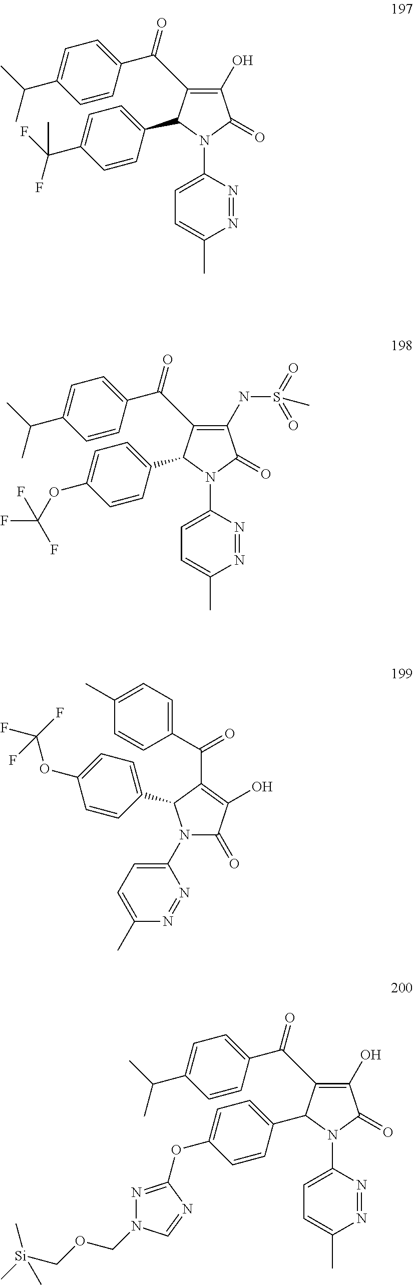

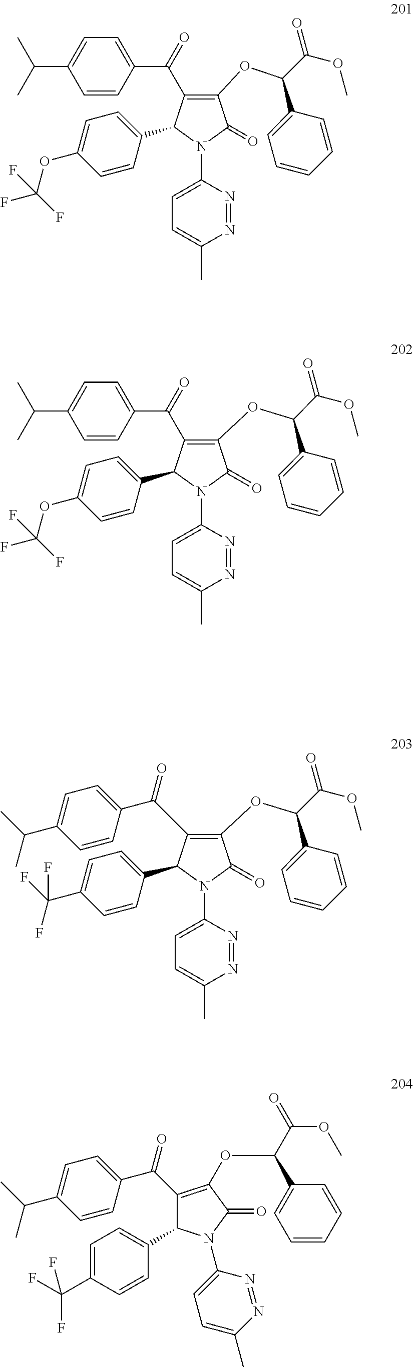

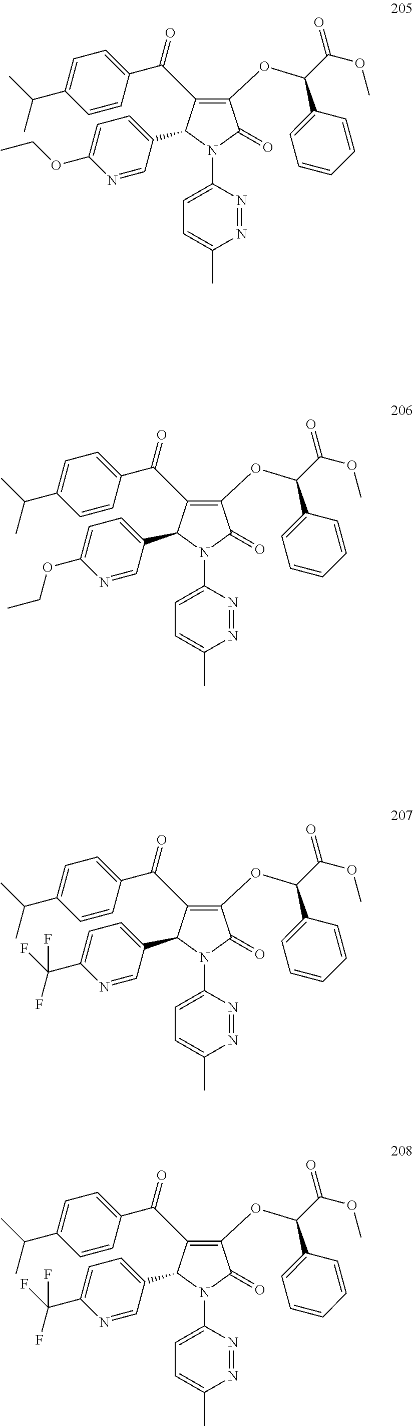

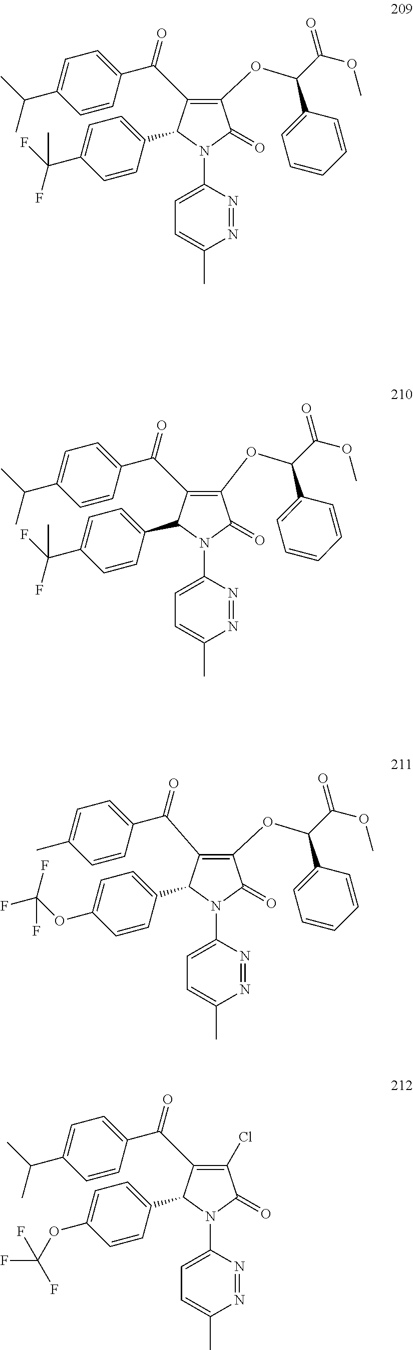

The compounds according to the present invention can be prepared by methods well known in the art. In particular they can be prepared by the general procedure A, C, D, E, G, H, or J as described bellow.

The present invention also concerns a pharmaceutical composition comprising a compound according to the present invention or a salt, solvate, tautomer, isotope, enantiomer, diastereoisomer or racemic mixture thereof and pharmaceutically acceptable excipients.

Advantageously, the pharmaceutical composition according to the present invention contains a further antiviral agent, in particular selected in the group consisting of ribavirin, interferon, inhibitors of HCV helicase, inhibitors of HCV protease, inhibitors of HCV NS4A, inhibitors of HCV NS5B, inhibitors of HCV NS5A, anti-HIV agent and mixture thereof.

The present invention concerns also a composition according to the present invention, a compound according to the present invention, or a compound of formula (a), (b), (c) or (d) as defined above or a salt, solvate, tautomer, isotope, enantiomer, diastereoisomer or racemic mixture thereof for use as a drug, in particular as an antiviral drug, advantageously intended to treat hepatitis, in particular hepatitis C, for example as a hepatitis C polymerase inhibitor.

The present invention also concerns a product containing a compound according to the present invention or a compound of formula (a), (b), (c) or (d) as defined above or a salt, solvate, tautomer, isotope, enantiomer, diastereoisomer or racemic mixture thereof and at least another antiviral agent, in particular selected in the group consisting of ribavirin, interferon, inhibitors of HCV helicase, inhibitors of HCV protease, inhibitors of HCV NS4A, inhibitors of HCV NS5B, inhibitors of HCV NS5A, inhibitors of HCV polymerase, anti-HIV agent and mixture thereof, as a combined preparation for simultaneous, separate or sequential use in hepatitis therapy, in particular in patients having the HIV disease.

Therefore the compound according to the present invention can be used as a bi- or tri-therapy in order to treat hepatitis C with another anti-hepatitis C antiviral agent (ribavirin, interferon, inhibitors of HCV helicase, inhibitors of HCV protease, inhibitors of HCV NS4A, inhibitors of HCV NS5B, inhibitors of HCV NS5A, inhibitors of HCV polymerase or mixture thereof) or even as a bi or tri-therapy with one or several anti-HIV antiviral agent in order to treat hepatitis C in a patient having HIV disease or finally as a tri-therapy with another anti-hepatitis C antiviral agent and an anti-HIV antiviral agent in order to treat hepatitis C in a patient having HIV disease.

### DEFINITIONS

### Antiviral Agent

By antiviral agent it is meant any of several drugs used to treat or prevent viral infections. The drugs act by interfering with a virus's ability to enter a host cell and replicate itself with the host cell's DNA. Some drugs block the virus's attachment or entry into the cell; others inhibit replication or prevent the virus from shedding the protein coat that surrounds the viral DNA.

Antiviral agents or drugs are now available for a wide variety of viral diseases. For example, Ribavirin, available since the mid-1980s, is used to treat respiratory syncytial virus (RSV), a cause of severe childhood respiratory infections. It is thought to inhibit messenger RNA. Amantadine and rimantadine, which are effective against strains of influenza A, act by interfering with viral uncoating.

### Pharmaceutical Compositions

The compounds of the present invention may also be present in the form of pharmaceutically acceptable salts. For use in medicine, the salts of the compounds of this invention refer to non-toxic “pharmaceutically acceptable salts.” Pharmaceutically acceptable salt forms include pharmaceutically acceptable acidic/anionic or basic/cationic salts. Pharmaceutically acceptable salts of the acidic or basic compounds of the invention can of course be made by conventional procedures, such as by reacting the free base or acid with at least a stoichiometric amount of the desired salt-forming acid or base. Pharmaceutically acceptable salts of the acidic compounds of the invention include salts with inorganic cations such as sodium, potassium, calcium, magnesium, zinc, and ammonium, and salts with organic bases. Suitable organic bases include N-methyl-D-glucamine, arginine, benzathine, diolamine, olamine, procaine and tromethamine. Pharmaceutically acceptable salts of the basic compounds of the invention include salts derived from organic or inorganic acids. Suitable anions include acetate, adipate, besylate, bromide, camsylate, chloride, citrate, edisylate, estolate, fumarate, gluceptate, gluconate, glucuronate, hippurate, hyclate, hydrobromide, hydrochloride, iodide, isethionate, lactate, lactobionate, maleate, mesylate, methylbromide, methylsulphate, nap sylate, nitrate, oleate, pamoate, phosphate, polygalacturonate, stearate, succinate, sulphate, sulphosalicylate, tannate, tartrate, terephthalate, tosylate and triethiodide. Hydrochloride salts are particularly preferred.

It is anticipated that the compounds of the invention can be administered by oral or parenteral routes, intestinal, ocular, vaginal, rectal nasal (intranasal), pulmonary or other mucosal, transdermal and topical administration, and inhalation, advantageously by oral route. Primary routes for parenteral administration include intravenous, intramuscular, and subcutaneous administration. Secondary routes of administration include intraperitoneal, intra-arterial, intra-articular, intracardiac, intracisternal, intradermal, intralesional, intraocular, intrapleural, intrathecal, intrauterine, and intraventricular administration. For oral administration, the compounds of the invention will generally be provided in the form of tablets or capsules or as an aqueous solution or suspension. Tablets for oral use may include the active ingredient mixed with pharmaceutically acceptable excipients such as inert diluents, disintegrating agents, binding agents, lubricating agents, sweetening agents, flavouring agents, colouring agents and preservatives. Suitable inert diluents include sodium and calcium carbonate, sodium and calcium phosphate and lactose. Corn starch and alginic acid are suitable disintegrating agents. Binding agents may include starch and gelatine. The lubricating agent, if present, will generally be magnesium stearate, stearic acid or talc. If desired, the tablets may be coated with a material such as glyceryl monostearate or glyceryl distearate, to delay absorption in the gastrointestinal tract. Capsules for oral use include hard gelatine capsules in which the active ingredient is mixed with a solid diluent and soft gelatine capsules wherein the active ingredient is mixed with water or an oil such as peanut oil, liquid paraffin or olive oil.

For intramuscular, intraperitoneal, subcutaneous and intravenous use, the compounds of the invention will generally be provided in sterile aqueous solutions or suspensions, buffered to an appropriate pH and isotonicity. Suitable aqueous vehicles include Ringer's solution and isotonic sodium chloride. Aqueous suspensions according to the invention may include suspending agents such as cellulose derivatives, sodium alginate, polyvinyl-pyrrolidone and gum tragacanth, and a wetting agent such as lecithin. Suitable preservatives for aqueous suspensions include ethyl and n-propyl p-hydroxybenzoate.

The pharmaceutical compositions of the present invention may, in particular, comprise more than one agent (multiple) of the present invention, e.g., two or more agents. The invention also provides a pharmaceutical preparation or system, comprising (a) a first agent, which is an agent of the invention; and (b) a second pharmaceutical agent. Said multiple agents of the invention or said first and second agents are formulated either in admixture or as separate compositions, e.g. for simultaneous though separate, or for sequential administration (see below).

### Modes of Administration

The compositions of the present invention can be delivered directly or in pharmaceutical compositions containing excipients (see above), as is well known in the art. The present methods of treatment involve administration of a therapeutically effective amount of an agent of the present invention to a subject. The term “therapeutically effective amount” as used herein refers to an amount of an agent according to the present invention needed to treat or ameliorate the targeted disease condition, or to exhibit a detectable therapeutic effect or a prolongation of survival in a patient. In general, the therapeutically effective dose can be estimated initially either in cell culture assays or in animal models, for example, in non-human primates, mice, rabbits, dogs, or pigs. The animal model may also be used to determine the appropriate concentration range and route of administration. Such information can then be used to determine useful doses and routes for administration in humans. Effective doses of the compounds of the present invention may be ascertained by conventional methods. The specific dosage level required for any particular patient will depend on a number of factors, including severity of the condition being treated, the route of administration, the general health of the patient (i.e. age, weight and diet) in particular if he is a HIV patient, the gender of the patient, the time and frequency of administration, and tolerance/response to therapy. In general, however, the daily dose (whether administered as a single dose or as divided doses) will be in the range 0.001 to 5000 mg per day, more usually from 1 to 2500 mg per day, and most usually from 10 to 1500 mg per day. Alternatively, dosages can be administered per unit body weight and in this instance a typical dose will be between 0.01 μg/kg and 50 mg/kg, especially between 10 μg/kg and 10 mg/kg, between 100 μg/kg and 2 mg/kg. An advantage of the compounds of the present invention is that they permit administration to be limited to one, two, three or four times weekly or monthly.

The present compositions may, if desired, be presented in a pack or dispenser device containing one or more unit dosage forms containing the active ingredient. Such a pack or device may, for example, comprise metal or plastic foil, such as a blister pack, or glass and rubber stoppers such as in vials. The pack or dispenser device may be accompanied by instructions for administration. Compositions comprising an agent of the invention formulated in a compatible pharmaceutical carrier may also be prepared, placed in an appropriate container, and labelled for treatment of an indicated condition.

### Chemical Definitions

The terms “comprising” and “comprises” means “including” as well as “consisting” e.g. a composition “comprising” X may consist exclusively of X or may include something additional e.g. X+Y.

The word “substantially” does not exclude “completely” e.g. a composition which is “substantially free” from Y may be completely free from Y. Where necessary, the word “substantially” may be omitted from the definition of the invention.

“Optional” or “optionally” means that the subsequently described event or circumstances may or may not occur, and that the description includes instances where said event or circumstance occurs and instances in which it does not.

Where the compounds according to this invention have at least one chiral centre, they may accordingly exist as enantiomers. Where the compounds possess two or more chiral centres, they may additionally exist as diastereomers. Where the processes for the preparation of the compounds according to the invention give rise to mixture of stereoisomers, these isomers may be separated by conventional techniques such as preparative chromatography. The compounds may be prepared in racemic form or individual enantiomers may be prepared by standard techniques known to those skilled in the art, for example, by enantiospecific synthesis or resolution, formation of diastereomeric pairs by salt formation with an optically active acid, followed by fractional crystallization and regeneration of the free base. The compounds may also be resolved by formation of diastereomeric esters or amides, followed by chromatographic separation and removal of the chiral auxiliary. Alternatively, the compounds may be resolved using a chiral HPLC column. It is to be understood that all such isomers and mixtures thereof in all proportion are encompassed within the scope of the present invention.

Where any particular moiety is substituted, for example a phenyl group comprising a substituent on the aryl ring, unless specified otherwise, the term “substituted” contemplates all possible isomeric forms. For example, substituted phenyl includes all of the following ortho-, meta- and para-permutations:

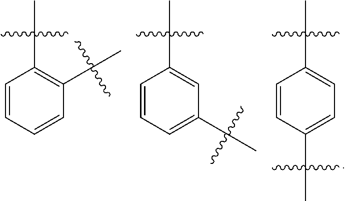

However, in general para substitution is preferred.

As used herein the term <<tautomer>> refers to isomers of the compounds according to the present invention that readily interconvert by a chemical reaction called tautomerization. Commonly this reaction results in the formal migration of a hydrogen atom or proton, accompanied by a switch of a single bond and adjacent double bond. In solutions where tautomerization is possible, a chemical equilibrium of the tautomers will be reached. The exact ratio of the tautomers depends on several factors, including temperature, solvent, and pH. The concept of tautomers that are interconvertible by tautomerizations is called tautomerism.

Common tautomeric pairs are: ketone-enol; amide-imidic acid; lactam-lactim, an amide-imidic acid tautomerism in heterocyclic rings; enamine-imine; enamine-enamine. In particular it can include ring-chain tautomerism which occurs when the movement of the proton is accompanied by a change from an open structure to a ring.

As used herein the term <<isotope>> refers to two molecules which differ only in the isotopic nature of their atoms i.e. their atom have a different atomic mass (mass number). Isotopes of an atom have nuclei with the same number of protons (the same atomic number) but different numbers of neutrons. Therefore, isotopes have different mass numbers, which give the total number of nucleons, the number of protons plus neutrons. In particular in the present invention an isotope of a compound can comprise one deuterium atom in place of a hydrogen atom.

The term “halogen” is used herein to refer to any of fluorine, chlorine, bromine and iodine. Most usually, however, halogen substituents in the compounds of the invention are chlorine, bromine and fluorine substituents, in particular fluorine substituents.

The term “O-Protecting group” as used in the present invention refers to a substituent which protects hydroxyl groups against undesirable reactions during synthetic procedures. O-protecting groups comprise substituted methyl ethers, for example, methoxymethyl (MOM), benzyloxymethyl, 2-methoxyethoxymethyl, 2-(trimethylsilyl)ethoxymethyl, t-butyl, benzyl and triphenylmethyl, tetrahydropyranyl ethers, substituted ethyl ethers, for example, 2,2,2-trichloroethyl, silyl ethers, for example, trimethylsilyl, t-butyldimethylsilyl (TBS) and t-butyldiphenylsilyl; and esters prepared by reacting the hydroxyl group with a carboxylic acid for example, acetate, propionate, benzoate and the like. In particular an allyl or an acetyl group is an “O-Protecting group” according to the present invention.

As used herein, the term “alkyl” refers to a straight or branched saturated monovalent hydrocarbon radical, having the number of carbon atoms as indicated. For example, the term “C₁-C₆-alkyl” includes C₁, C₂, C₃, C₄, C₅ and C₆ alkyl groups. By way of non-limiting example, suitable alkyl groups include methyl, ethyl, propyl, iso-propyl, butyl, iso-butyl, tert-butyl, pentyl and hexyl. In one aspect of the present invention ranges of alkyl groups are: C₁-C₆-alkyl, C₁-C₅-alkyl, C₁-C₄-alkyl, C₁-C₃-alkyl and C₁-C₂-alkyl, in particular C₁-C₃-alkyl.

As used herein, the term “alkenyl” refers to a straight or branched monovalent hydrocarbon radical, having the number of carbon atoms as indicated and at least a double bond. For example, the term “C₂-C₆-alkenyl” includes C₂, C₃, C₄, C₅ and C₆ alkenyl groups. By way of non-limiting example, suitable alkenyl groups include ethenyl, propenyl, isopropenyl, butenyl, iso-butenyl, tert-butenyl, pentenyl and hexenyl. In one aspect of the present invention ranges of alkenyl groups are: C₂-C₆-alkenyl, C₂-C₅-alkenyl, C₂-C₄-alkenyl and C₂-C₃-alkenyl in particular C₂-C₄-alkenyl. As used herein, the term “alkynyl” refers to a straight or branched monovalent hydrocarbon radical, having the number of carbon atoms as indicated and at least a triple bond. For example, the term “C₂-C₆-alkynyl” includes C₂, C₃, C₄, C₅ and C₆ alkynyl groups. By way of non-limiting example, suitable alkynyl groups include ethynyl, propynyl, iso-propynyl, butynyl, iso-butynyl, tert-butynyl, pentynyl and hexynyl. In one aspect of the present invention ranges of alkynyl groups are: C₂-C₆-alkynyl, C₂-C₅-alkynyl, C₂-C₄-alkynyl and C₂-C₃-alkynyl in particular C₂-C₃-alkynyl. As used herein, the term “cycloalkyl” refers to a cyclic saturated hydrocarbon radical, having the number of carbon atoms as indicated. For example, the term “C₃-C₆-cycloalkyl” includes C₃, C₄, C₅ and C₆ cycloalkyl groups. By way of non-limiting example, suitable cycloalkyl groups include cyclopropyl, cyclobutyl, cyclopentyl, cyclohexyl, cyclopropylmethyl, cyclopropylethyl, cyclobutylmethyl, cyclobutylethyl and cyclopentylmethyl. In one aspect of the present invention ranges of alkyl groups are: C₃-C₆-cycloalkyl, C₃-C₅-cycloalkyl and C₃-C₄-cycloalkyl.

As used herein, the term “aryl” refers to monovalent unsaturated aromatic carbocyclic radical having one, two, or three rings, which may be fused or bicyclic. In one aspect of the present invention, the term “aryl” refers to an aromatic monocyclic ring containing 5 or 6 carbon atoms, which may be substituted on the ring with 1, 2, 3, 4 or 5 substituents as defined herein; an aromatic bicyclic or fused ring system containing 7, 8, 9 or 10 carbon atoms, which may be substituted on the ring with 1, 2, 3, 4, 5, 6, 7, 8 or 9 substituents as defined herein; or an aromatic tricyclic ring system containing 10 carbon atoms, which may be substituted on the ring with 1, 2, 3, 4, 5, 6, 7, 8, 9, 10, 11, 12 or 13 substituents as defined herein. By way of non-limiting example, suitable aryl groups include phenyl, biphenyl, indanyl, azulenyl, tetrahydronaphthyl, tolyl, chlorophenyl, dichlorophenyl, trichlorophenyl, methoxyphenyl, dimethoxyphenyl, trimethoxyphenyl, fluorophenyl, difluorophenyl, trifluorophenyl, nitrophenyl, dinitrophenyl, trinitrophenyl, aminophenyl, diaminophenyl, triaminophenyl, cyanophenyl, chloromethylphenyl, tolylphenyl, chloroethylphenyl, trichloromethylphenyl, dihydroindenyl, benzocycloheptyl and trifluoromethylphenyl, advantageously a phenyl. In one aspect of the present invention ranges of aryl groups are: C₃₋₁₀-aryl, C₃₋₆-aryl C₄₋₉-aryl, C₅₋₈-aryl and C₆₋₇-aryl.

As used herein, the term “heteroaryl” refers to monovalent unsaturated aromatic heterocyclic radicals having one ring. Suitably, the term “6-members heteroaryl” encompasses heteroaryl moieties that are aromatic monocyclic ring systems containing six members of which at least one member is a N, O or S atom and which optionally depending of the case can contain one, two or three additional N, O or S atoms, advantageously N atoms. Suitably, the term “5-members heteroaryl” encompasses heteroaryl moieties that are aromatic monocyclic ring systems containing five members of which at least one member is a N, O or S atom and which optionally depending of the case can contain one, two or three additional N, O or S atoms, advantageously N atoms. By way of non-limiting example, suitable heteroaryl groups include furanyl, pyridyl, thiophenyl, pyrrolyl, imidazolyl, pyrazolyl, thiazolyl, isothiazolyl, oxazolyl, oxadiazolyl, isoxazolyl, pyrazinyl, oxazinyl, tetrazol, oxadiazol and triazol.

The term “heterocyclic” refers to a saturated or partially unsaturated ring having five members of which at least one member is a N, O or S atom and which optionally contains one additional O atom or one or two N atoms; a saturated or partially unsaturated ring having six members of which one, two or three members are an N, O or S atom and which optionally contains one additional O atom or one, or two additional N atoms; a saturated or partially unsaturated ring having seven members of which one, two or three members are an N, O or S atom and which optionally contains one additional O atom or one or two additional N atoms. Typically, heterocycles comprising peroxide groups are excluded from the definition of heterocyclic. By way of non-limiting example, suitable heterocyclic groups include pyrrolinyl, pyrrolidinyl, dioxolanyl, tetrahydrofuranyl, morpholinyl, imidazolinyl, imidazolidinyl, pyrazolidinyl, piperidinyl, dihydropyranyl, tetrahydropyranyl and piperazinyl.

### EXPERIMENTAL PART

The compounds according to the present invention have been prepared and tested as described above in a non limiting way

### I Preparation of the Compounds According to the Present Invention

The reactants and commercials compounds were purchased from Acros Organics, Sigma-Aldrich, Alfa Aesar, Interchim and Maybridge.

### General Procedure A:

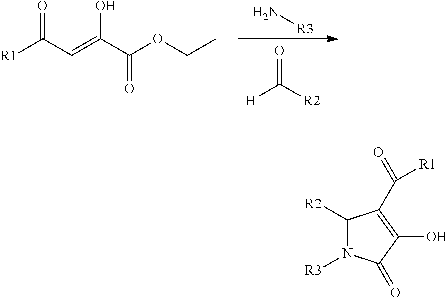

(R1, R2 and R3 are as defined above).

The aldehyde (1 eq) and the amine (1 eq) were dissolved in AcOH (2 ml/mmol). After 15 min at room temperature, the keto-ester (1 eq) in AcOH (2 ml/mmol) was added. The solution was stirred 30 min at 160° C. in micro-waves apparatus (adapted from Silina et al, Chemistry of Heterocyclic Compounds), vol. 34, n° 6, 1998. After filtration of the mixture, the solid was washed with Et₂O or Et₂O/MeOH (99/1) to give the titled compound. When the reaction mixture was homogeneous, it was concentrated under vacuum and the residue was triturated in Et₂O then filtered. When the purity was insufficient, the residue was purified by flash chromatography (silica gel) or HPLC semi-preparative with the appropriate gradient determined by TLC. The following compounds were prepared according general procedure A:

### Example 1

### 6-[2-(4-tert-butyl-phenyl)-4-hydroxy-3-(4-methyl-benzoyl)-5-oxo-2,5-dihydro-pyrrol-1-yl]-nicotinic acid methyl ester

Prepared from 4-tert-butylbenzaldehyde methyl 6-aminonicotinate and 2-hydroxy-4-oxo-4-p-tolyl-but-2-enoic acid ethyl ester in 17% yield. The reaction mixture was refluxed for 4 h. ¹H-NMR (DMSO-d₆): δ (ppm) 1.14 (brs, 9H); 2.35 (s, 3H); 3.82 (s, 3H); 6.39 (s, 1H); 7.19-7.35 (m, 6H); 7.64 (d, 2H); 8.31 (brs, 2H); 8.80 (brs, 1H); MS (ESI+): m/z=485 [M+H]⁺; Melting point: 287-288° C.

### Example 2

### 5-(4-tert-butyl-phenyl)-3-hydroxy-4-(4-methyl-benzoyl)-1-pyridin-2-yl-1,5-dihydro-pyrrol-2-one

Prepared from 4-tert-butylbenzaldehyde, 2-aminopyridine and 2-hydroxy-4-oxo-4-p-tolyl-but-2-enoic acid ethyl ester in 19% yield. ¹H-NMR (DMSO-d₆): δ (ppm) 1.12 (s, 9H); 2.07 (s, 3H); 6.36 (s, 1H); 7.08-7.35 (m, 7H); 7.62 (dd, 2H); 7.80-7.85 (t, 1H); 8.09 (d, 1H); 8.31 (dd, 1H); MS (ESI+): m/z=427 [M+H]⁺

### Example 3

### 6-[3-hydroxy-5-(4-isopropyl-phenyl)-4-(4-methyl-benzoyl)-2-oxo-2,5-dihydro-pyrrol-1-yl]-nicotinic acid methyl ester

Prepared from 4-isopropylbenzaldehyde, methyl-6-aminonicotinate and 2-hydroxy-4-oxo-4-p-tolyl-but-2-enoic acid ethyl ester in 10% yield. ¹H-NMR (DMSO-d₆): δ (ppm) 1.05 (d, 6H); 2.34 (s, 3H); 2.72 (quint, 1H); 3.81 (s, 3H); 6.37 (s, 1H); 7.05 (d, 2H); 7.2-7.35 (m, 4H); 7.63 (d, 2H); 8.3 (brd, 2H); 8.78 (s, 1H); 11.84 (brs, 1H); MS (ESI+): m/z=471 [M+H]⁺; Melting point: 264-266° C.

### Example 4

### 3-hydroxy-5-(4-isopropyl-phenyl)-4-(4-methyl-benzoyl)-1-pyrazin-2-yl-1,5-dihydro-pyrrol-2-one

Prepared from 4-isopropylbenzaldehyde, aminopyrazine and 2-hydroxy-4-oxo-4-p-tolyl-but-2-enoic acid ethyl ester in 6% yield. ¹H-NMR (DMSO-d₆): δ (ppm) 1.04 (d, 6H); 2.33 (s, 3H); 2.65-2.75 (m, 1H); 6.27 (s, 1H); 7.05 (dd, 2H); 7.29-7.32 (m, 4H); 7.63 (dd, 2H); 8.36 (dd, 2H); 9.4 (s, 1H); MS (ESI+): m/z=414 [M+H]⁺

### Example 5

### 6-[3-hydroxy-5-(4-isopropyl-phenyl)-4-(4-methyl-benzoyl)-2-oxo-2,5-dihydro-pyrrol-1-yl]-nicotinic acid

Prepared from 4-isopropylbenzaldehyde, 6-aminonicotinic acid and 2-hydroxy-4-oxo-4-p-tolyl-but-2-enoic acid ethyl ester in 6% yield. ¹H-NMR (DMSO-d₆): δ (ppm) 1.05 (d, 6H); 2.34 (s, 3H); 2.72 (quint, 1H); 6.38 (s, 1H); 7.06 (d, 2H); 7.25-7.33 (m, 4H); 7.64 (d, 2H); 8.29 (brs, 2H); 8.76 (brs, 1H); MS (ESI+): m/z=457 [M+H]⁺

### Example 6

### 1-(5-chloro-pyridin-2-yl)-3-hydroxy-5-(4-isopropyl-phenyl)-4-(4-methyl-benzoyl)-1,5-dihydro-pyrrol-2-one

Prepared from 4-isopropylbenzaldehyde, 2-amino-5-chloropyridine and 2-hydroxy-4-oxo-4-p-tolyl-but-2-enoic acid ethyl ester in 7% yield. After filtration of the mixture, the solid was washed with diisopropyl ether to give the titled compound. ¹H-NMR (DMSO-d₆): δ(ppm) 1.06 (d, 6H); 2.34 (s, 3H); 2.72 (m, 1H); 6.31 (s, 1H); 7.06 (d, 2H); 7.27 (m, 4H); 7.63 (d, 2H); 7.95-7.98 (d, 1H); 8.15-8.19 (d, 1H); 8.37 (brs, 1H); 11.85 (brs, 1H); MS (ESI+): m/z=447 [M+H]⁺

### Example 7

### 6-[3-hydroxy-5-(4-isopropyl-phenyl)-4-(4-methyl-benzoyl)-2-oxo-2,5-dihydro-pyrrol-1-yl]-nicotinonitrile

Prepared from 4-isopropylbenzaldehyde, 2-amino-5-cyanopyridine and 2-hydroxy-4-oxo-4-p-tolyl-but-2-enoic acid ethyl ester in 2% yield. ¹H-NMR (DMSO-d₆): δ(ppm) 1.08 (d, 6H); 2.34 (s, 3H); 2.73 (m, 1H); 6.34 (s, 1H); 7.08-7.67 (m, 8H); 8.35 (brd, 2H); 8.76 (brs, 1H); 11.88 (brs, 1H); MS (ESI+): m/z=438 [M+H]⁺

### Example 8

### 3-hydroxy-5-(4-isopropyl-phenyl)-4-(4-methyl-benzoyl)-1-(6-methyl-pyridazin-3-yl)-1,5-dihydro-pyrrol-2-one

Prepared from 4-isopropylbenzaldehyde, 3-amino-6-methylpyridazine and 2-hydroxy-4-oxo-4-p-tolyl-but-2-enoic acid ethyl ester in 6% yield. ¹H-NMR (DMSO-d₆): δ (ppm) 1.06 (d, 6H); 2.34 (s, 3H); 2.72-2.74 (m, 1H); 6.43 (s, 1H); 7.05 (d, 2H); 7.23-7.28 (m, 4H); 7.56-7.66 (m, 3H); 8.27 (d, 1H); MS (ESI+): m/z=428 [M+H]⁺; Melting point: 279° C.

### Example 9

### 3-hydroxy-5-(4-isopropyl-phenyl)-4-(4-methyl-benzoyl)-1-pyridazin-3-yl-1,5-dihydro-pyrrol-2-one

Prepared from 4-isopropylbenzaldehyde, pyridazin-3-ylamine and 2-hydroxy-4-oxo-4-p-tolyl-but-2-enoic acid ethyl ester in 5% yield. ¹H-NMR (DMSO-d₆): δ (ppm) 1.05 (d, 6H); 2.35 (s, 3H); 2.67-2.77 (m, 1H); 6.48 (s, 1H); 7.06 (dd, 2H); 7.25-7.35 (m, 4H); 7.63-7.76 (m, 3H); 8.40 (dd, 1H); 8.96 (d, 1H); MS (ESI+): m/z=414 [M+H]⁺

### Example 10

### 5-(4-tert-butyl-phenyl)-3-hydroxy-4-(4-methoxy-benzoyl)-1-(6-methyl-pyridazin-3-yl)-1,5-dihydro-pyrrol-2-one

Prepared from 4-tert-butylbenzaldehyde, 3-amino-6-methylpyridazine and 2-hydroxy-4-(4-methoxy-phenyl)-4-oxo-but-2-enoic acid ethyl ester 4% yield. ¹H-NMR (DMSO-d₆): δ (ppm) 1.14 (s, 9H); 2.6 (s, 2H); 3.82 (s, 3H); 6.46 (s, 1H); 7.00 (dd, 2H); 7.19-7.35 (m, 4H); 7.60 (dd, 1H); 7.75 (dd, 2H); 8.28 (dd, 1H); 11.77 (brs, 1H); MS (ESI+): m/z=458 [M+H]⁺

### Example 52

### 3-Hydroxy-5-(4-isopropyl-phenyl)-4-(4-methyl-benzoyl)-1-(6-methyl-pyridin-3-yl)-1,5-dihydro-pyrrol-2-one

Prepared from 4-isopropylbenzaldehyde, 5-amino-2-methylpyridine and 2-hydroxy-4-oxo-4-p-tolyl-but-2-enoic acid ethyl ester in 29% yield. ¹H-NMR (DMSO-d₆): δ (ppm) 1.04 (d, 6H); 2.33 (s, 3H); 2.35 (s, 3H); 2.71 (quint, 1H); 6.28 (s, 1H); 7.06 (d, 2H); 7.18-7.31 (m, 5H); 7.62 (d, 2H); 7.90 (dd, 1H); 8.66 (d, 1H); MS (ESI+): m/z=427 [M+H]⁺

### General Procedure B: Preparation of the Intermediate

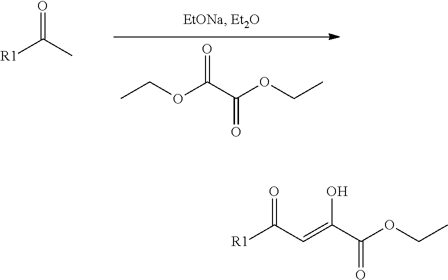

(R1, R2 and R3 are as defined above).

To a solution relevant methyl ketone in diethyl ether (3 ml/mmol) at 0° C. was added EtONa (prepared in situ with Na (1.3 eq)). The mixture was stirred 30 min, then, diethyl oxalate was added drop wise. The mixture was stirred overnight at room temperature. After filtration of the mixture, the solid was washed with Et₂O to give the titled compound. The following intermediates compounds were prepared according general procedure B: 1, 2, 3, 4, 9, 10.

### Intermediate 1

### 4-(3-dimethylamino-phenyl)-2-hydroxy-4-oxo-but-2-enoic acid ethyl ester

Prepared from 3′-dimethylaminoacetophenone and diethyl oxalate in 55% yield. ¹H-NMR (CDCl₃): δ(ppm) 1.05 (t, 3H); 2.83 (s, 6H); 3.82 (q, 2H); 6.37 (s, 1H); 6.63 (dd, 1H); 7.06 (dd, 3H)

### Intermediate 2

### 4-(4-dimethylamino-phenyl)-2-hydroxy-4-oxo-but-2-enoic acid ethyl ester

Prepared from p-dimethylaminoacetophenone and diethyl oxalate in 37% yield. After filtration of the mixture, the solid was diluted in methanol, then, concentrated under vacuum. The methyl ester compound was obtained. ¹H-NMR (DMSO-d₆): δ(ppm) 2.91 (s, 3H); 2.94 (s, 3H); 3.63 (s, 3H); 6.23 (s, 1H); 6.65 (dd, 2H); 7.66 (dd, 2H); MS (ESI+): m/z=250 [M+H]⁺

### Intermediate 3

### 4-(4-dimethylamino-phenyl)-2-hydroxy-4-oxo-but-2-enoic acid ethyl ester

Prepared from p-dimethylaminoacetophenone and diethyl oxalate in 84% yield. ¹H-NMR (DMSO-d₆): δ(ppm) 1.23 (t, 3H); 2.91 (s, 3H); 2.94 (s, 3H); 4.07 (q, 2H); 6.26 (s, 1H); 6.66 (dd, 2H); 7.68 (dd, 2H); MS (ESI+): m/z=264 [M+H]⁺

### Intermediate 4

### 2-hydroxy-4-[4-(2-methoxy-ethoxy)-phenyl]-4-oxo-but-2-enoic acid ethyl ester

Prepared from 1-[4-(2-methoxy-ethoxy)-phenyl]-ethanone and diethyl oxalate. The product was used as such in the next step. ¹H-NMR (DMSO-d₆): δ (ppm) 1.09 (t, 3H); 3.43 (s, 3H); 3.65-3.71 (m, 2H); 3.85-3.98 (m, 4H); 6.39 (s, 1H); 6.70 (d, 2H); 7.67 (d, 2H).

### Intermediate 9

### 2-hydroxy-4-oxo-4-(4-pyridin-2-yl-phenyl)-but-2-enoic acid ethyl ester

Prepared from 1-(4-pyridin-2-yl-phenyl)-ethanone and diethyl oxalate in 86% yield. Note: The mixture was stirred 30 min at room temperature. ¹H-NMR (DMSO-d₆): δ (ppm) 1.24 (t, 3H); 4.10 (q, 2H); 6.33 (s, 1H); 7.36 (t, 1H); 7.80 (d, 1H); 7.88 (d, 2H); 7.96-8.12 (m, 3H), 8.66 (brs, 1H); MS (ESI+): m/z=298 [M+H]⁺

### Intermediate 10

### 2-hydroxy-4-(5-methyl-thiazol-2-yl)-4-oxo-but-2-enoic acid ethyl ester

Prepared, following from 1-(5-methyl-thiazol-2-yl)-ethanone and diethyl oxalate in 48% yield. Note: the crude compound was engaged in the next stage.

### General Procedure C:

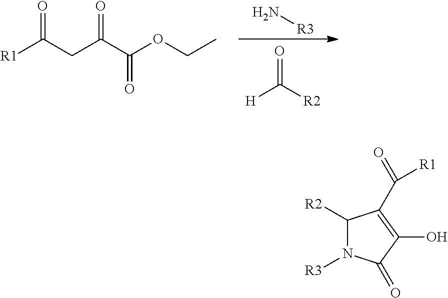

The aldehyde (1 eq) and the amine (1 eq) were dissolved in EtOH (2 ml/mmol)/AcOH cat. After stirring 15 to 30 min at room temperature, the keto-ester (1 eq) in EtOH (2 ml/mmol)/AcOH cat. was added. The reaction mixture was refluxed for 2 to 4 hours. After filtration of the mixture, the solid was washed with Et₂O to give the desired compound. Sometimes, Et₂O needs to be added in the reaction mixture to obtain a precipitate. When some starting amine was recovered, it could be removed by acidic washings. If the purity was insufficient, the residue was purified by flash chromatography (silica gel) or HPLC semi-preparative with the appropriate gradient determined by TLC. The following compounds were prepared according general procedure C:

### Example 14

### 1-(5-fluoro-pyridin-2-yl)-3-hydroxy-5-(4-isopropyl-phenyl)-4-(4-methyl-benzoyl)-1,5-dihydro-pyrrol-2-one

Prepared from 4-isopropylbenzaldehyde, 2-amino-5-fluoropyridine and 2-hydroxy-4-oxo-4-p-tolyl-but-2-enoic acid ethyl ester in 39% yield. ¹H-NMR (DMSO-d₆): δ(ppm) 1.04 (d, 6H); 2.33 (s, 3H); 2.71 (quint, 1H); 6.29 (s, 1H); 7.04 (d, 2H); 7.22-7.28 (m, 4H); 7.62 (d, 2H); 7.74-7.82 (td, 1H); 8.14 (dd, 1H); 8.32 (brs, 1H); 11.79 (brs, 1H); MS (ESI+): m/z=431 [M+H]⁺

### Example 15

### 3-hydroxy-5-(4-isopropyl-phenyl)-4-(4-methyl-benzoyl)-1-(6-morpholin-4-yl-pyridazin-3-yl)-1,5-dihydro-pyrrol-2-one

Prepared from 4-isopropylbenzaldehyde, 6-morpholin-4-yl-pyridazin-3-ylamine and 2-hydroxy-4-oxo-4-p-tolyl-but-2-enoic acid ethyl ester in 10% yield. ¹H-NMR (DMSO-d₆): δ (ppm) 1.06 (d, 6H); 2.34 (s, 3H); 2.73-2.76 (m, 1H); 3.46 (m, 4H); 3.68 (m, 4H); 6.36 (s, 1H); 7.06 (dd, 2H); 7.25-7.38 (m, 5H); 7.63 (dd, 2H); 8.06 (dd, 1H); MS (ESI+): m/z=499 [M+H]⁺

### Example 16

### 4-(3-chloro-benzoyl)-3-hydroxy-5-(4-isopropyl-phenyl)-1-(6-methyl-pyridazin-3-yl)-1,5-dihydro-pyrrol-2-one

Prepared from 4-isopropylbenzaldehyde, 3-amino-6-methylpyridazine and 4-(3-chloro-phenyl)-2-hydroxy-4-oxo-but-2-enoic acid ethyl ester in 15% yield. ¹H-NMR (DMSO-d₆): δ(ppm) 1.06 (d, 6H); 2.71-2.74 (m, 1H); 6.42 (s, 1H); 7.06 (dd, 2H); 7.33 (dd, 2H); 7.46-7.69 (m, 5H); 8.26 (dd, 1H); MS (ESI+): m/z=448 [M+H]⁺; Melting point: 271° C.

### Example 17

### 3-hydroxy-5-(4-isopropyl-phenyl)-4-(3-methoxy-benzoyl)-1-(6-methyl-pyridazin-3-yl)-1,5-dihydro-pyrrol-2-one

Prepared from 4-isopropylbenzaldehyde, 3-amino-6-methylpyridazine and 2-hydroxy-4-(3-methoxy-phenyl)-4-oxo-but-2-enoic acid ethyl ester in 10% yield. ¹H-NMR (DMSO-d₆): δ(ppm) 1.06 (d, 6H); 2.50 (s, 3H); 2.72 (m, 1H); 3.76 (s, 3H); 6.42 (s, 1H); 7.03-7.13 (m, 3H); 7.28-7.35 (m, 5H); 7.57 (dd, 1H); 8.27 (dd, 1H), MS (ESI+): m/z=444 [M+H]⁺

### Example 18

### 3-hydroxy-4-(4-methyl-benzoyl)-1-(6-methyl-pyridazin-3-yl)-5-(4-trifluoromethyl-phenyl)-1,5-dihydro-pyrrol-2-one

Prepared from 4-(trifluoromethyl)benzaldehyde, 3-amino-6-methylpyridazine and 2-hydroxy-4-oxo-4-p-tolyl-but-2-enoic acid ethyl ester in 42% yield. ¹H-NMR (DMSO-d₆): δ(ppm) 2.34 (s, 3H); 2.50 (s, 3H); 6.50 (s, 1H); 7.25 (dd, 2H); 7.58-7.67 (m, 7H); 8.37 (dd, 1H); 12.08 (brs, 1H); MS (ESI+): m/z=454 [M+H]⁺; Melting point: 278° C.

### Example 19

### 3-hydroxy-5-(4-isopropyl-phenyl)-4-(2-methyl-benzoyl)-1-(6-methyl-pyridazin-3-yl)-1,5-dihydro-pyrrol-2-one

Prepared from 4-isopropylbenzaldehyde, 3-amino-6-methylpyridazine and 2-hydroxy-4-oxo-4-o-tolyl-but-2-enoic acid ethyl ester in 20% yield. ¹H-NMR (DMSO-d₆): δ(ppm) 1.07 (d, 6H); 2.07 (s, 3H); 2.50 (s, 3H); 2.75 (quint, 1H); 6.37 (s, 1H); 7.05 (dd, 2H); 7.16-7.36 (m, 6H); 7.57 (dd, 1H); 8.26 (dd, 1H); MS (ESI+): m/z=428 [M+H]⁺; Melting point: 221° C.

### Example 20

### 3-hydroxy-4-(4-isopropyl-benzoyl)-5-(4-isopropyl-phenyl)-1-(6-methyl-pyridazin-3-yl)-1,5-dihydro-pyrrol-2-one

Prepared from 4-isopropylbenzaldehyde, 3-amino-6-methylpyridazine and 2-hydroxy-4-(4-isopropyl-phenyl)-4-oxo-but-2-enoic acid ethyl ester in 10% yield. ¹H-NMR (DMSO-d₆): δ(ppm) 1.05 (d, 6H); 1.18 (d, 6H); 2.50 (s, 3H); 2.71 (quint, 1H); 2.91 (quint, 1H); 6.44 (s, 1H); 7.04 (dd, 2H); 7.3 (m, 4H); 7.58 (dd, 1H); 7.67 (dd, 2H); 8.27 (dd, 1H); MS (ESI+): m/z=456 [M+H]⁺; Melting point: 271° C.

### Example 21

### 3-hydroxy-5-(3-methoxy-phenyl)-4-(4-methyl-benzoyl)-1-(6-methyl-pyridazin-3-yl)-1,5-dihydro-pyrrol-2-one

Prepared from 3-methoxybenzaldehyde, 3-amino-6-methylpyridazine and 2-hydroxy-4-oxo-4-p-tolyl-but-2-enoic acid ethyl ester in 39% yield. ¹H-NMR (DMSO-d₆): δ(ppm) 2.33 (s, 3H); 2.50 (s, 3H); 3.62 (s, 3H); 6.42 (s, 1H); 6.65 (dd, 1H); 6.89 (dd, 2H); 7.08 (dt, 1H); 7.25 (dd, 2H); 7.57-7.65 (m, 3H); 8.27 (d, 1H); MS (ESI+): m/z=416 [M+H]⁺; Melting point: 265° C.

### Example 22

### 3-hydroxy-4-(4-methyl-benzoyl)-1-(6-methyl-pyridazin-3-yl)-5-(4-trifluoromethoxy-phenyl)-1,5-dihydro-pyrrol-2-one

Prepared from 4-(trifluoromethyl)benzaldehyde, 3-amino-6-methylpyridazine and 2-hydroxy-4-oxo-4-p-tolyl-but-2-enoic acid ethyl ester in 40% yield. ¹H-NMR (DMSO-d₆): δ(ppm) 2.34 (s, 3H); 2.50 (s, 3H); 6.46 (s, 1H); 7.18 (dd, 2H); 7.25 (dd, 2H); 7.54-7.65 (m, 5H); 8.32 (d, 1H)); MS (ESI+): m/z=470 [M+H]⁺; Melting point: 269° C.

### Example 23

### 3-hydroxy-4-(4-methoxy-benzoyl)-1-(6-methyl-pyridazin-3-yl)-5-(4-trifluoromethoxy-phenyl)-1,5-dihydro-pyrrol-2-one

Prepared from 4-(trifluoromethyl)benzaldehyde, 3-amino-6-methylpyridazine 2-hydroxy-4-(4-methoxy-phenyl)-4-oxo-but-2-enoic acid ethyl ester in 36% yield. ¹H-NMR (DMSO-d₆): δ(ppm) 2.50 (s, 3H); 3.81 (s, 3H); 6.46 (s, 1H); 6.97 (d, 2H); 7.17 (d, 2H); 7.52-7.62 (m, 3H); 7.74 (d, 2H); 8.32 (dd, 1H); MS (ESI+): m/z=486 [M+H]⁺; Melting point: 265° C.

### Example 24

### 3-hydroxy-5-(4-methoxy-phenyl)-1-(6-methyl-pyridazin-3-yl)-4-(4-trifluoromethoxy-benzoyl)-1,5-dihydro-pyrrol-2-one

Prepared from p-anisaldehyde, 3-amino-6-methylpyridazine and 2-hydroxy-4-oxo-4-(4-trifluoromethoxy-phenyl)-but-2-enoic acid ethyl ester in 23% yield. ¹H-NMR (DMSO-d₆): δ(ppm) 2.50 (s, 3H); 3.60 (s, 3H); 6.40 (s, 1H); 6.71 (dd, 2H); 7.32 (dd, 2H); 7.44 (dd, 2H); 7.58 (dd, 1H); 7.86 (dd, 2H); 8.23 (dd, 1H); MS (ESI+): m/z=486 [M+H]⁺; Melting point: 279° C.

### Example 25

### 3-hydroxy-5-(4-isopropyl-phenyl)-1-(6-methyl-pyridazin-3-yl)-4-(4-trifluoromethoxy-benzoyl)-1,5-dihydro-pyrrol-2-one

Prepared from 4-isopropylbenzaldehyde, 3-amino-6-methylpyridazine and 2-Hydroxy-4-oxo-4-(4-trifluoromethoxy-phenyl)-but-2-enoic acid ethyl ester in 7% yield. ¹H-NMR (DMSO-d₆): δ(ppm) 1.07 (d, 6H); 2.50 (s, 3H); 2.73 (m, 1H); 6.44 (s, 1H); 7.06 (d, 2H); 7.32 (d, 2H); 7.44 (d, 2H); 7.59 (d, 1H); 7.86 (d, 2H); 8.28 (d, 1H); MS (ESI+): m/z=498 [M+H]⁺; Melting point: 269° C.

### Example 26

### 3-hydroxy-5-(4-methoxy-phenyl)-4-(4-methyl-benzoyl)-1-(6-methyl-pyridazin-3-yl)-1,5-dihydro-pyrrol-2-one

Prepared from p-anisaldehyde, 3-amino-6-methylpyridazine and 2-hydroxy-4-oxo-4-p-tolyl-but-2-enoic acid ethyl ester in 25% yield. ¹H-NMR (DMSO-d₆): δ (ppm) 2.35 (s, 3H); 2.50 (s, 3H); 3.61 (s, 3H); 6.41 (s, 1H); 6.72 (d, 2H); 7.25-7.32 (td, 4H); 7.57-7.67 (m, 3H); 8.25 (dd, 1H); MS (ESI+): m/z=416 [M+H]⁺; Melting point: 266° C.

### Example 27

### 3-hydroxy-4-(4-methoxy-benzoyl)-5-(4-methoxy-phenyl)-1-(6-methyl-pyridazin-3-yl)-1,5-dihydro-pyrrol-2-one

Prepared from p-anisaldehyde, 3-amino-6-methylpyridazine and 2-hydroxy-4-(4-methoxy-phenyl)-4-oxo-but-2-enoic acid ethyl ester in 24% yield. ¹H-NMR (DMSO-d₆): δ (ppm) 2.50 (s, 3H); 3.60 (s, 3H); 3.82 (s, 3H); 6.40 (s, 1H); 6.72 (d, 2H); 6.98 (d, 2H); 7.29 (d, 2H); 7.58 (d, 1H); 7.76 (d, 2H); 8.25 (d, 1H); MS (ESI+): m/z=432 [M+H]⁺; Melting point: 259° C.

### Example 28

### 3-hydroxy-4-(4-methyl-benzoyl)-1-(6-methyl-pyridazin-3-yl)-5-(3-trifluoromethyl-phenyl)-1,5-dihydro-pyrrol-2-one

Prepared from 3-(trifluoromethyl)benzaldehyde, 3-amino-6-methylpyridazine and 2-hydroxy-4-oxo-4-p-tolyl-but-2-enoic acid ethyl ester in 16% yield. ¹H-NMR (DMSO-d₆): δ(ppm) 2.34 (s, 3H); 6.50 (s, 1H); 7.25 (dd, 2H); 7.39-7.50 (m, 2H); 7.58-7.76 (m, 5H); 8.35 (dd, 1H); MS (ESI+): m/z=454 [M+H]⁺; Melting point: 232° C.

### Example 29

### 3-hydroxy-5-(3-isopropyl-phenyl)-4-(4-methyl-benzoyl)-1-(6-methyl-pyridazin-3-yl)-1,5-dihydro-pyrrol-2-one

Prepared from 3-(isopropyl)benzaldehyde, 3-amino-6-methylpyridazine and 2-hydroxy-4-oxo-4-p-tolyl-but-2-enoic acid ethyl ester in 3% yield. ¹H-NMR (DMSO-d₆): δ(ppm) 1.03 (d, 3H); 1.05 (d, 3H); 2.33 (s, 3H); 2.71-2.76 (m, 1H); 6.44 (s, 1H); 6.97-7.26 (m, 6H); 7.55-7.63 (m, 3H); 8.25 (d, 1H); MS (ESI+): m/z=428 [M+H]⁺

### Example 30

### 3-hydroxy-5-(2-methoxy-phenyl)-4-(4-methyl-benzoyl)-1-(6-methyl-pyridazin-3-yl)-1,5-dihydro-pyrrol-2-one

Prepared from 2-methoxybenzaldehyde, 3-amino-6-methylpyridazine and 2-hydroxy-4-oxo-4-p-tolyl-but-2-enoic acid ethyl ester in 15% yield. ¹H-NMR (DMSO-d₆): δ(ppm) 2.35 (s, 3H); 2.50 (s, 3H); 3.69 (s, 6H); 6.62 (s, 1H); 6.77-6.84 (dd, 2H); 7.04-7.08 (dt, 1H); 7.26-7.29 (d, 3H); 7.57-7.63 (dt, 3H); 8.25 (dd, 1H); MS (ESI+): m/z=416 [M+H]⁺; Melting point: 211-213° C.

### Example 31

### 3-hydroxy-4-(4-methyl-benzoyl)-1-(6-methyl-pyridazin-3-yl)-5-(3-propoxy-phenyl)-1,5-dihydro-pyrrol-2-one

Prepared from 3-(propoxy)benzaldehyde, 3-amino-6-methylpyridazine and 2-hydroxy-4-oxo-4-p-tolyl-but-2-enoic acid ethyl ester in 11% yield. ¹H-NMR (DMSO-d₆): δ(ppm) 0.87-0.9 (t, 3H); 1.61-1.63 (qd, 2H); 2.34 (s, 3H); 2.50 (s, 3H); 3.74-3.80 (t, 2H); 6.41 (s, 1H); 6.63-6.66 (d, 1H); 6.89 (d, 2H); 7.02-7.06 (t, 1H); 7.24-7.27 (q, 2H); 7.57-7.65 (m, 3H); 8.25 (dd, 1H); 11.77 (brs, 1H); MS (ESI+): m/z=444 [M+H]⁺

### Example 32

### 4-(3-dimethylamino-benzoyl)-3-hydroxy-5-(4-isopropyl-phenyl)-1-(6-methyl-pyridazin-3-yl)-1,5-dihydro-pyrrol-2-one

Prepared from 4-isopropylbenzaldehyde, 3-amino-6-methylpyridazine and 4-(3-dimethylamino-phenyl)-2-hydroxy-4-oxo-but-2-enoic acid ethyl ester (Intermediate 1) in 13% yield. ¹H-NMR (DMSO-d₆): δ (ppm) 1.03 (d, 6H); 2.66 (m, 1H); 2.82 (s, 6H); 6.37 (s, 1H); 6.71-6.74 (dd, 1H); 6.85-6.88 (dd, 2H); 6.97-6.99 (m, 3H); 7.07-7.13 (dd, 1H); 7.46-7.50 (m, 1H); 8.3 (dd, 1H); MS (ESI+): m/z=457 [M+H]⁺

### Example 33

### 3-hydroxy-5-(4-isopropoxy-phenyl)-4-(4-methyl-benzoyl)-1-(6-methyl-pyridazin-3-yl)-1,5-dihydro-pyrrol-2-one

Prepared from 4-isopropoxybenzaldehyde, 3-amino-6-methylpyridazine and 2-hydroxy-4-oxo-4-p-tolyl-but-2-enoic acid ethyl ester in 9% yield. ¹H-NMR (DMSO-d₆): δ(ppm) 1.12 (d, 6H); 2.34 (s, 3H); 2.50 (s, 3H); 4.42 (quint, 1H); 6.40 (s, 1H); 6.68 (d, 2H); 7.26 (d, 4H); 7.60 (m, 3H); 8.24 (dd, 1H); 11.72 (brs, 1H); MS (ESI+): m/z=444 [M+H]⁺; Melting point: 268-272° C.

### Example 34

### 3-hydroxy-5-methyl-4-(4-methyl-benzoyl)-1-(6-methyl-pyridazin-3-yl)-1,5-dihydro-pyrrol-2-one

Prepared from acetaldehyde, 3-amino-6-methylpyridazine and 2-hydroxy-4-oxo-4-p-tolyl-but-2-enoic acid ethyl ester in 12% yield. ¹H-NMR (DMSO-d₆): δ(ppm) 1.39 (d, 3H); 2.39 (s, 3H); 2.62 (s, 3H); 5.40-5.43 (m, 1H); 7.30-7.33 (m, 2H); 7.67-7.76 (m, 3H); 8.37-8.41 (d, 1H); MS (ESI+): m/z=324 [M+H]⁺

### Example 35

### 4-(4-dimethylamino-benzoyl)-3-hydroxy-5-(4-isopropyl-phenyl)-1-(6-methyl-pyridazin-3-yl)-1,5-dihydro-pyrrol-2-one

Prepared from 4-isopropylbenzaldehyde, 3-amino-6-methylpyridazine and 4-(4-dimethylamino-phenyl)-2-hydroxy-4-oxo-but-2-enoic acid ethyl ester (Intermediate 2) in 28% yield. ¹H-NMR (DMSO-d₆): δ(ppm) 1.07-1.14 (dd, 6H); 2.49 (s, 3H); 2.68-2.73 (m, 1H); 2.93 (s, 6H); 6.32 (s, 1H); 6.55-6.58 (dd, 2H); 6.94-6.97 (dd, 2H); 7.21-7.25 (dd, 2H); 7.46-7.49 (dd, 1H); 7.86-7.90 (dd, 2H); 8.31-8.34 (dd, 1H); MS (ESI+): m/z=457 [M+H]⁺

### Example 36

### 3-hydroxy-5-(2-isopropyl-phenyl)-4-(4-methyl-benzoyl)-1-(6-methyl-pyridazin-3-yl)-1,5-dihydro-pyrrol-2-one

Prepared from 2-isopropylbenzaldehyde, 3-amino-6-methylpyridazine and 2-hydroxy-4-oxo-4-p-tolyl-but-2-enoic acid ethyl ester in 8% yield. ¹H-NMR (DMSO-d₆): δ(ppm) 1.26 (dd, 3H); 1.39 (dd, 3H); 2.33 (s, 3H); 2.50 (s, 1H); 3.86-3.92 (m, 1H); 6.88-7.25 (m, 7H); 7.52-7.63 (m, 3H); 8.23-8.27 (dd, 1H); 11.69 (m, 1H); MS (ESI+): m/z=427 [M+H]⁺

### Example 37

### 1-(6-dimethylamino-pyridazin-3-yl)-3-hydroxy-5-(4-isopropyl-phenyl)-4-(4-methyl-benzoyl)-1,5-dihydro-pyrrol-2-one

Prepared from 4-(isopropyl)benzaldehyde, N,N-dimethyl-pyridazine-3,6-diamine and 2-hydroxy-4-oxo-4-p-tolyl-but-2-enoic acid ethyl ester in 9% yield. ¹H-NMR (DMSO-d₆): δ(ppm) 1.07 (d, 6H); 2.34 (s, 3H); 2.77 (quint, 1H); 3.31 (s, 6H); 6.35 (s, 1H); 7.06 (d, 2H); 7.15 (d, 1H); 7.25-7.28 (m, 4H); 7.63 (d, 2H); 7.95 (d, 1H); MS (ESI+): m/z=457 [M+H]⁺; Melting point: 281° C.

### Example 38

### 3-hydroxy-5-(3-isopropoxy-phenyl)-4-(4-methyl-benzoyl)-1-(6-methyl-pyridazin-3-yl)-1,5-dihydro-pyrrol-2-one

Prepared from 3-isopropoxybenzaldehyde, 3-amino-6-methylpyridazine and 2-hydroxy-4-oxo-4-p-tolyl-but-2-enoic acid ethyl ester in 19% yield. ¹H-NMR (DMSO-d₆): δ(ppm) 1.13 (d, 6H); 2.33 (s, 3H); 4.48 (quint, 1H); 6.41 (s, 1H); 6.62 (dd, 1H); 6.80-6.95 (m, 2H); 7.02-7.07 (t, 1H); 7.25 (d, 2H); 7.57-7.65 (m, 3H); 8.27 (d, 1H); 11.82 (brs, 1H); MS (ESI+): m/z=444 [M+H]⁺; Melting point: 243-245° C.

### Example 39

### 3-hydroxy-5-(4-isopropyl-phenyl)-4-(3-methyl-benzoyl)-1-(6-methyl-pyridazin-3-yl)-1,5-dihydro-pyrrol-2-one

Prepared from 4-isopropylbenzaldehyde, 3-amino-6-methylpyridazine and 2-hydroxy-4-oxo-4-m-tolyl-but-2-enoic acid ethyl ester in 8% yield. ¹H-NMR (DMSO-d₆): δ(ppm) 1.06 (d, 6H); 2.33 (s, 3H); 2.5 (s, 3H); 3.27 (quint, 1H); 6.45 (s, 1H); 7.05 (d, 2H); 7.27-7.41 (m, 4H); 7.51-7.54 (m, 2H); 7.57 (s, 1H); 8.27 (d, 1H); MS (ESI+): m/z=428 [M+H]⁺; Melting point: 245-250° C.

### Example 40

### 3-hydroxy-5-(4-isopropyl-phenyl)-4-(4-methyl-benzoyl)-1-[6-(4-methyl-piperazin-1-yl)-pyridazin-3-yl]-1,5-dihydro-pyrrol-2-one

Prepared from 4-isopropylbenzaldehyde, 6-(4-methyl-piperazin-1-yl)-pyridazin-3-ylamine and 2-hydroxy-4-oxo-4-p-tolyl-but-2-enoic acid ethyl ester in 6% yield. ¹H-NMR (DMSO-d₆): δ(ppm) 1.05 (d, 6H); 2.18 (s, 3H); 2.28 (s, 3H); 2.32-2.41 (m, 4H); 2.69 (quint, 1H); 3.42-3.52 (m, 4H); 6.28 (s, 1H); 6.92 (d, 2H); 7.05-7.08 (m, 4H); 7.27 (d, 1H); 7.60 (d, 2H); 8.05 (d, 1H); MS (ESI+): m/z=512 [M+H]⁺

### Example 41

### 3-hydroxy-5-(4-isopropyl-phenyl)-4-(4-methyl-benzoyl)-1-(5-methyl-pyridin-2-yl)-1,5-dihydro-pyrrol-2-one

Prepared from 4-isopropylbenzaldehyde, 2-amino-5-methylpyridine and 2-hydroxy-4-oxo-4-p-tolyl-but-2-enoic acid ethyl ester in 36% yield. ¹H-NMR (DMSO-d₆): δ(ppm) 1.04 (d, 6H); 2.19 (s, 3H); 2.33 (s, 3H); 2.71 (quint, 1H); 6.33 (s, 1H); 7.02 (d, 2H); 7.25 (d, 4H); 7.59-7.64 (m, 3H), 7.95 (d, 1H); 8.13 (brs, 1H); MS (ESI+): m/z=427 [M+H]⁺; Melting point: 259-261° C.

### Example 42

### 5-biphenyl-4-yl-3-hydroxy-4-(4-methyl-benzoyl)-1-(6-methyl-pyridazin-3-yl)-1,5-dihydro-pyrrol-2-one

Prepared from 4-biphenylcarboxaldehyde, 3-amino-6-methylpyridazine and 2-hydroxy-4-oxo-4-p-tolyl-but-2-enoic acid ethyl ester in 26% yield. ¹H-NMR (DMSO-d₆): δ(ppm) 2.33 (s, 3H); 2.50 (s, 3H); 6.50 (s, 1H); 7.24-7.30 (m, 3H); 7.37 (t, 2H); 7.48-7.58 (m, 6H); 7.64 (t, 3H); 8.32 (d, 1H); MS (ESI+): m/z=462 [M+H]⁺

### Example 43

### 3-hydroxy-5-(4-isopropyl-phenyl)-1-(6-isopropyl-pyridazin-3-yl)-4-(4-methyl-benzoyl)-1,5-dihydro-pyrrol-2-one

Prepared from 4-isopropylbenzaldehyde, 6-isopropyl-pyridazin-3-ylamine and 2-hydroxy-4-oxo-4-p-tolyl-but-2-enoic acid ethyl ester in 18% yield. ¹H-NMR (DMSO-d₆) δ(ppm) 1.06 (d, 6H); 1.23 (d, 6H); 2.64-2.82 (m, 1H); 3.10-3.20 (m, 1H); 6.46 (s, 1H); 7.08 (d; 2H); 7.25-7.34 (m, 4H); 7.62-7.66 (m; 3H); 8.33 (d, 1H); MS (ESI+): m/z=456 [M+H]⁺; Melting point: 269-270° C.

### Example 44

### 3-hydroxy-4-(4-isopropyl-benzoyl)-5-(4-isopropyl-phenyl)-1-(5-methyl-pyridin-2-yl)-1,5-dihydro-pyrrol-2-one

Prepared from 4-isopropylbenzaldehyde, 2-amino-5-methylpyridine and 2-hydroxy-4-(4-isopropyl-phenyl)-4-oxo-but-2-enoic acid ethyl ester in 34% yield. ¹H-NMR (DMSO-d₆): δ(ppm) 1.04 (d, 6H); 1.18 (d, 6H); 2.19 (s, 3H); 2.70 (quint, 1H); 2.91 (quint, 1H); 6.33 (s, 1H); 7.03 (d, 2H); 7.28 (dd, 4H); 7.60-7.68 (m, 3H); 7.96 (d, 1H); 8.13 (brs, 1H); 11.72 (brs, 1H); MS (ESI+): m/z=455 [M+H]⁺

### Example 45

### 5-biphenyl-4-yl-3-hydroxy-4-(4-isopropyl-benzoyl)-1-(6-methyl-pyridazin-3-yl)-1,5-dihydro-pyrrol-2-one

Prepared from 4-biphenylcarboxaldehyde, 3-amino-6-methylpyridazine and 2-hydroxy-4-(4-isopropyl-phenyl)-4-oxo-but-2-enoic acid ethyl ester in 37% yield. ¹H-NMR (DMSO-d₆): δ(ppm) 1.19 (d, 6H); 2.50 (s, 3H); 2.94 (quint, 1H); 6.51 (s, 1H); 7.31-7.62 (m, 12H); 7.70 (d, 2H); 8.32 (d; 1H); MS (ESI+): m/z=490 [M+H]⁺

### Example 46

### 3-hydroxy-4-(4-isopropyl-benzoyl)-5-[4-(4-methyl-piperazin-1-yl)-phenyl]-1-(6-methyl-pyridazin-3-yl)-1,5-dihydro-pyrrol-2-one

Prepared from 4-(4-methyl-piperazin-1-yl)-benzaldehyde, 3-amino-6-methylpyridazine and 2-hydroxy-4-(4-isopropyl-phenyl)-4-oxo-but-2-enoic acid ethyl ester in 9% yield. ¹H-NMR (DMSO-d₆): δ(ppm) 1.16 (d, 6H); 2.50 (s, 3H); 2.77-2.93 (m, 5H); 3.10-3.17 (m, 4H); 6.29 (s, 1H); 6.72 (d, 2H); 7.13-7.22 (dd; 4H); 7.51 (d, 1H); 7.67 (d, 2H); 8.29 (d, 1H); MS (ESI+): m/z=512 [M+H]⁺; Melting point: 198-203° C.

### Example 47

### 3-hydroxy-4-(4-isopropyl-benzoyl)-1-(6-methyl-pyridazin-3-yl)-5-[4-(2-morpholin-4-yl-ethoxy)-phenyl]-1,5-dihydro-pyrrol-2-one

Prepared from 4-(2-morpholinoethoxy)benzaldehyde, 3-amino-6-methylpyridazine and 2-hydroxy-4-(4-isopropyl-phenyl)-4-oxo-but-2-enoic acid ethyl ester in 21% yield. ¹H-NMR (DMSO-d₆): δ(ppm) 1.19 (d, 6H); 2.60-2.65 (m, 4H); 2.75-2.96 (m, 4H); 3.57 (m, 5H); 4.00 (t; 2H); 6.37 (s, 1H); 6.73 (d, 2H); 7.26-7.29 (m, 4H); 7.54 (d, 1H); 7.69 (d, 2H); 8.25 (d, 1H); MS (ESI+): m/z=543 [M+H]⁺; Melting point: 256° C.

### Example 48

### 3-hydroxy-4-(4-isopropyl-benzoyl)-5-(4-isopropyl-phenyl)-1-(6-methyl-pyridin-3-yl)-1,5-dihydro-pyrrol-2-one

Prepared from 4-isopropylbenzaldehyde, 5-amino-2-methylpyridine and 2-hydroxy-4-(4-isopropyl-phenyl)-4-oxo-but-2-enoic acid ethyl ester in 17% yield. ¹H-NMR (DMSO-d₆): δ (ppm) 1.04 (d, 6H); 1.18 (d, 6H); 2.35 (s, 3H); 2.71 (quint, 1H); 2.92 (quint, 1H); 6.29 (s, 1H); 7.05 (d, 2H); 7.19 (d, 1H); 7.3 (dd, 4H); 7.66 (d, 2H); 7.90 (dd, 1H); 8.65 (d, 1H); 11.8 (brs, 1H); MS (ESI+): m/z=455 [M+H]⁺; Melting point: 252-254° C.

### Example 49

### 4-(4-dimethylamino-benzoyl)-3-hydroxy-1-(6-methyl-pyridazin-3-yl)-5-(4-trifluoromethoxy-phenyl)-1,5-dihydro-pyrrol-2-one

Prepared from 4-(trifluoromethoxy)benzaldehyde, 3-amino-6-methylpyridazine and 4-(4-dimethylamino-phenyl)-2-hydroxy-4-oxo-but-2-enoic acid ethyl ester (see Intermediate 3) in 8% yield. ¹H-NMR (DMSO-d₆): δ (ppm) 2.50 (s, 3H); 2.99 (s, 6H); 6.45 (s, 1H); 6.65 (d, 2H); 7.16 (d, 2H); 7.48 (d, 2H); 7.63 (t, 3H); 8.33 (d, 1H); MS (ESI+): m/z=499 [M+H]⁺

### Example 50

### 3-hydroxy-4-(4-methyl-benzoyl)-5-[4-(1-methyl-piperidin-4-yloxy)-phenyl]-1-(6-methyl-pyridazin-3-yl)-1,5-dihydro-pyrrol-2-one

Prepared from 4-(1-methyl-piperidin-4-yloxy)-benzaldehyde, 3-amino-6-methylpyridazine and 2-hydroxy-4-oxo-4-p-tolyl-but-2-enoic acid ethyl ester in 21% yield. ¹H-NMR (DMSO-d₆): δ (ppm) 1.65-1.82 (m, 2H); 1.87-2.02 (m, 2H); 2.26 (s, 3H); 2.50 (s, 3H); 2.57 (s, 3H); 2.73-2.91 (m, 2H); 2.95-3.11 (m, 2H); 4.32-4.43 (m, 1H); 6.27 (s, 1H); 6.71 (d, 2H); 7.08 (d, 2H); 7.23 (d, 2H); 7.47 (d, 1H); 7.69 (d, 2H); 8.30 (d, 1H); MS (ESI+): m/z=499 [M+H]⁺; Melting point: 259-260° C.

### Example 51

### 3-hydroxy-5-(4-methoxy-phenyl)-1-(6-methyl-pyridazin-3-yl)-4-(4-morpholin-4-yl-benzoyl)-1,5-dihydro-pyrrol-2-one

Prepared from p-anisaldehyde, 3-amino-6-methylpyridazine and 2-hydroxy-4-(4-morpholin-4-yl-phenyl)-4-oxo-but-2-enoic acid ethyl ester in 21% yield. ¹H-NMR (DMSO-d₆): δ (ppm) 2.50 (s, 3H); 3.11-3.21 (m, 4H); 3.6 (s, 3H); 3.68-3.78 (m, 4H); 6.27 (s, 1H); 6.65 (d, 2H); 6.80 (d, 2H); 7.21 (d, 2H); 7.48 (d, 1H); 7.85 (d, 2H); 8.29 (d, 1H); MS (ESI+): m/z=487 [M+H]⁺; Melting point: 232° C.

### Example 77

### 3-hydroxy-4-(4-isopropyl-benzoyl)-1-(6-methyl-pyridin-3-yl)-5-(4-trifluoromethoxy-phenyl)-1,5-dihydro-pyrrol-2-one

Prepared from 4-isopropylbenzaldehyde, 5-amino-2-methylpyridine and 2-hydroxy-4-(4-isopropyl-phenyl)-4-oxo-but-2-enoic acid ethyl ester in 36% yield; ¹H-NMR (DMSO-d₆): δ (ppm) 1.18 (d, 6H); 2.36 (s, 3H); 2.91 (quint, 1H); 6.39 (s, 1H); 7.2 (t, 3H); 7.3 (d, 2H); 7.54 (d, 2H); 7.68 (d, 2H); 7.90 (dd, 1H); 8.67 (d, 1H); MS (ESI+): m/z=497 [M+H]⁺; Melting point: 214-218° C.

### Example 78

### 5-{4-[4-(2-Dimethylamino-ethyl)-piperazin-1-yl]-phenyl}-3-hydroxy-4-(4-isopropyl-benzoyl)-1-(6-methyl-pyridazin-3-yl)-1,5-dihydro-pyrrol-2-one

Prepared from 4-[4-(2-dimethylamino-ethyl)-piperazin-1-yl]-benzaldehyde (Intermediate 7), 3-amino-6-methylpyridazine and 2-hydroxy-4-(4-isopropyl-phenyl)-4-oxo-but-2-enoic acid ethyl ester in 11% yield. Note: after purification, the residue was diluted in methanol at room temperature for 1 h. The mixture was filtered and the solid was washed with Et₂O to give the desired compound. ¹H-NMR (DMSO-d₆): δ (ppm) 1.18 (d, 6H); 2.5 (s, 3H); 2.50-2.66 (m, 6H); 2.66 (s, 6H); 2.87 (quint, 1H); 2.98-3.19 (m, 6H); 6.24 (s, 1H); 6.68 (d, 2H); 7.15 (t, 4H); 7.47 (d, 1H); 7.72 (d, 2H); 8.28 (d, 1H); MS (ESI+): m/z=569 [M+H]⁺

### Example 79

### 4-(biphenyl-4-carbonyl)-3-hydroxy-1-(6-methyl-pyridazin-3-yl)-5-(4-trifluoromethoxy-phenyl)-1,5-dihydro-pyrrol-2-one

Prepared from 4-(trifluoromethoxy)benzaldehyde, 3-amino-6-methylpyridazine and 4-biphenyl-4-yl-2-hydroxy-4-oxo-but-2-enoic acid ethyl ester in 21% yield; ¹H-NMR (DMSO-d₆): δ (ppm) 2.37 (s, 3H); 6.43 (s, 1H); 6.97 (d, 2H); 7.17 (d, 2H); 7.34-7.95 (m, 5H); 7.70 (t, 3H); 7.85 (d, 2H); 8.37 (d, 1H); MS (ESI+): m/z=532 [M+H]⁺; Melting point: 226-232° C.

### Example 80

### 3-hydroxy-1-(6-methyl-pyridazin-3-yl)-4-(4-morpholin-4-yl-benzoyl)-5-(4-trifluoromethoxy-phenyl)-1,5-dihydro-pyrrol-2-one

Prepared from 4-(trifluoromethoxy)benzaldehyde, 3-amino-6-methylpyridazine and 2-hydroxy-4-(4-morpholin-4-yl-phenyl)-4-oxo-but-2-enoic acid ethyl ester in 24% yield. ¹H-NMR (DMSO-d₆): δ (ppm) 2.50 (s, 3H); 3.30 (brs, 4H); 3.70 (brs, 4H); 6.46 (s, 1H); 6.92 (d, 2H); 7.16 (d, 2H); 7.50 (d, 2H); 7.58-7.68 (m, 3H); 8.32 (d, 1H); MS (ESI+): m/z=541 [M+H]⁺; Melting point: 234° C.

### Example 82

### 3-Hydroxy-4-[4-(4-methoxy-benzyloxy)-benzoyl]-1-(6-methyl-pyridazin-3-yl)-5-(4-trifluoromethoxy-phenyl)-1,5-dihydro-pyrrol-2-one

Prepared from 4-(trifluoromethoxy)benzaldehyde, 3-amino-6-methylpyridazine and 2-hydroxy-4-[4-(4-methoxy-benzyloxy)-phenyl]-4-oxo-but-2-enoic acid ethyl ester (Intermediate 8) in 10% yield; ¹H-NMR (DMSO-d₆): δ (ppm) 2.50 (s, 3H); 3.75 (s, 3H); 5.09 (s, 2H); 6.46 (s, 1H); 6.93 (d, 2H); 7.04 (d, 2H); 7.17 (d, 2H); 7.38 (d, 2H); 7.52-7.62 (m, 3H); 7.74 (d, 2H); 8.33 (d, 1H); 11.92 (brs, 1H); MS (ESI+): m/z=592 [M+H]⁺; Melting point: 206-209° C.

### Example 85

### 3-Hydroxy-4-[4-(2-methoxy-ethoxy)-benzoyl]-1-(6-methyl-pyridazin-3-yl)-5-(4-trifluoromethoxy-phenyl)-1,5-dihydro-pyrrol-2-one

Prepared from 4-(trifluoromethoxy)benzaldehyde, 3-amino-6-methylpyridazine and 2-hydroxy-4-[4-(2-methoxy-ethoxy)-phenyl]-4-oxo-but-2-enoic acid ethyl ester (Intermediate 4) in 13% yield. ¹H-NMR (DMSO-d₆): δ (ppm) 3.64-3.68 (m, 2H); 4.15-4.20 (m, 2H); 6.47 (s, 1H); 7.00 (d, 2H); 7.18 (d, 2H); 7.54 (d, 2H); 7.61 (d, 1H); 7.74 (d, 2H); 8.33 (d, 1H); ¹H-NMR (DMSO-d₆+D₂O): δ (ppm) 3.25 (s, 3H); 3.62-3.65 (m, 2H); 6.44 (s, 1H); 6.95 (d, 2H); 7.13 (d, 2H); 7.49 (d, 2H); 7.56 (d, 1H); 7.71 (d, 2H); 8.28 (d, 1H); MS (ESI+): m/z=530 [M+H]⁺; Melting point: 216-219° C.

### Example 87

### 5-(4-chloro-phenyl)-3-hydroxy-4-(4-isopropyl-benzoyl)-1-(6-methyl-pyridazin-3-yl)-1,5-dihydro-pyrrol-2-one

Prepared from 4-chlorobenzaldehyde, 3-amino-6-methylpyridazine and 2-hydroxy-4-(4-isopropyl-phenyl)-4-oxo-but-2-enoic acid ethyl ester in 9% yield. ¹H-NMR (DMSO-d₆): δ (ppm) 1.19 (d, 6H); 2.92 (quint, 1H); 6.41 (s, 1H); 7.22 (d, 2H); 7.30 (d, 2H); 7.41 (d, 2H); 7.59 (d, 1H); 7.68 (d, 2H); 8.32 (d, 1H); MS (ESI+): m/z=448 [M+H]⁺; Melting point: 258-260° C.

### Example 88

### 5-(4-difluoromethoxy-phenyl)-3-hydroxy-4-(4-isopropyl-benzoyl)-1-(6-methyl-pyridazin-3-yl)-1,5-dihydro-pyrrol-2-one

Prepared from 4-(difluoromethoxy)benzaldehyde, 3-amino-6-methylpyridazine and 2-hydroxy-4-(4-isopropyl-phenyl)-4-oxo-but-2-enoic acid ethyl ester in 24% yield. ¹H-NMR (DMSO-d₆): δ (ppm) 1.20 (d, 6H); 2.50 (s, 3H); 2.94 (quint, 1H); 6.46 (s, 1H); 6.83 (s, 0.2H); 6.97 (d, 2H); 7.12 (s, 0.5H); 7.33 (d, 2H); 7.42 (s, 0.3H): 7.47 (d, 2H); 7.60 (d, 1H); 7.70 (d, 2H); 8.30 (d, 1H); 11.92 (brs, 1H); MS (ESI+): m/z=480 [M+H]⁺; Melting point: 263-265° C.

### Example 57

### 5-(2,4-dimethyl-phenyl)-3-hydroxy-4-(4-methyl-benzoyl)-1-(6-methyl-pyridazin-3-yl)-1,5-dihydro-pyrrol-2-one

Prepared from 2,4-dimethylbenzaldehyde, 3-amino-6-methylpyridazine and 2-hydroxy-4-oxo-4-p-tolyl-but-2-enoic acid ethyl ester in 14% yield. After acidic washings, the solid was washed with Et₂O/MeOH (0.5%) to give the desired compound. ¹H-NMR (DMSO-d₆): δ (ppm) 2.07 (s, 3H); 2.34 (s, 3H); 2.50 (s, 3H); 2.68 (s, 3H); 6.60 (s, 1H); 6.78 (d, 1H); 6.84 (s, 1H); 7.02 (d, 1H); 7.26 (d, 2H); 7.54 (d, 1H); 7.65 (d, 2H); 8.28 (d, 1H); 11.73 (brs, 1H); MS (ESI+): m/z=414 [M+H]⁺; Melting point: 254° C.

### Example 58

### 3-hydroxy-4-(4-isopropyl-benzoyl)-1-(6-methyl-pyridazin-3-yl)-5-(4-morpholin-4-yl-phenyl)-1,5-dihydro-pyrrol-2-one

Prepared from 4-morpholinobenzaldehyde, 3-amino-6-methylpyridazine and 2-hydroxy-4-(4-isopropyl-phenyl)-4-oxo-but-2-enoic acid ethyl ester in 34% yield. After filtration of the mixture, the solid was washed with Et₂O/MeOH (0.5%) to give the desired compound. ¹H-NMR (DMSO-d₆): δ (ppm) 1.19 (d, 6H); 2.50 (s, 3H); 2.85-3.00 (m, 5H); 3.55-3.65 (m, 4H); 6.38 (s, 1H); 6.70 (d, 2H); 7.20 (d, 2H); 7.32 (d, 2H); 7.56 (d, 1H); 7.68 (d, 2H); 8.22 (d, 1H); 11.69 (brs, 1H); MS (ESI+): m/z=499 [M+H]⁺; Melting point: 293-294° C.

### Example 59

### 5-[4-(2-dimethylamino-ethoxy)-phenyl]-3-hydroxy-4-(4-methyl-benzoyl)-1-(6-methyl-pyridazin-3-yl)-1,5-dihydro-pyrrol-2-one

Prepared from 4-[2-(dimethylamino)ethoxy]benzaldehyde, 3-amino-6-methylpyridazine and 2-hydroxy-4-oxo-4-p-tolyl-but-2-enoic acid ethyl ester in 27% yield. After filtration of the mixture, the solid was washed with Et₂O/MeOH (0.5%) to give the desired compound. ¹H-NMR (DMSO-d₆): δ (ppm) 2.26 (s, 3H); 2.45 (s, 3H); 2.70 (s, 6H); 3.30 (m, 2H); 4.10-4.21 (m, 2H); 6.29 (s, 1H); 6.74 (d, 2H); 7.03 (d, 2H); 7.26 (d, 2H); 7.42 (d, 1H); 7.64 (d, 2H); 8.21 (d, 1H); MS (ESI+): m/z=473 [M+H]⁺; Melting point: 263-266° C.

### Example 60

### 3-hydroxy-5-(4-isopropyl-phenyl)-4-(4-methyl-benzoyl)-1-(5-trifluoromethyl-pyridin-2-yl)-1,5-dihydro-pyrrol-2-one

Prepared from 4(isopropyl)benzaldehyde, 5-(trifluoromethyl)pyridin-2-amine and 2-hydroxy-4-oxo-4-p-tolyl-but-2-enoic acid ethyl ester in 41% yield. The mixture was concentrated under vacuum and the residue was triturated in CH₂Cl₂/petroleum ether (50/50) to give the titled compound. ¹H-NMR (DMSO-d₆): δ (ppm) 1.05 (d, 6H); 2.34 (s, 3H); 2.72 (quint, 1H); 6.36 (s, 1H); 7.05 (d, 2H); 7.24-7.33 (m, 4H); 7.63 (d, 2H); 8.23 (dd, 1H); 8.38 (d, 1H); 8.69 (brs, 1H), 11.84 (brs, 1H); MS (ESI+): m/z=481 [M+H]⁺; Melting point: 255-257° C.

### Example 61

### 3-hydroxy-4-(4-isopropyl-benzoyl)-1-(6-methyl-pyridazin-3-yl)-5-(4-trifluoromethoxy-phenyl)-1,5-dihydro-pyrrol-2-one

Prepared from 4-(trifluoromethoxy)benzaldehyde, 3-amino-6-methylpyridazine and 2-hydroxy-4-(4-isopropyl-phenyl)-4-oxo-but-2-enoic acid ethyl ester in 33% yield. ¹H-NMR (DMSO-d₆): δ (ppm) 1.18 (d, 6H); 2.48 (s, 3H); 2.92 (quint, 1H); 6.46 (s, 1H); 7.16 (d, 2H); 7.31 (d, 2H); 7.52-7.61 (m, 5H); 8.32 (d, 1H); 12.07 (brs, 1H) MS (ESI+): m/z=498 [M+H]⁺; Melting point: 262-265° C.

### Example 62

### 3-hydroxy-4-(4-isopropyl-benzoyl)-5-(4-methoxy-phenyl)-1-(6-methyl-pyridazin-3-yl)-1,5-dihydro-pyrrol-2-one

Prepared from p-anisaldehyde, 3-amino-6-methylpyridazine and 2-hydroxy-4-(4-isopropyl-phenyl)-4-oxo-but-2-enoic acid ethyl ester in 14% yield. ¹H-NMR (DMSO-d₆): δ (ppm) 1.19 (d, 6H); 2.48 (s, 3H); 2.92 (quint 1H); 3.59 (s, 3H); 6.41 (s, 1H); 6.70 (d, 2H); 7.24-7.37 (m, 4H); 7.57 (d, 1H); 7.68 (d, 2H); 8.24 (d, 1H); 11.76 (brs, 1H); MS (ESI+): m/z=444 [M+H]⁺; Melting point: 263-267° C.

### Example 63

### 3-hydroxy-4-(4-isopropyl-benzoyl)-1-(6-methyl-pyridazin-3-yl)-5-(4-trifluoromethyl-phenyl)-1,5-dihydro-pyrrol-2-one

Prepared from 4-(trifluoromethyl)benzaldehyde, 3-amino-6-methylpyridazine and 2-hydroxy-4-(4-isopropyl-phenyl)-4-oxo-but-2-enoic acid ethyl ester in 32% yield. After filtration of the mixture, the solid was washed with Et₂O/MeOH (0.5%) to give the desired compound. ¹H-NMR (DMSO-d₆): δ (ppm) 1.18 (d, 6H); 2.50 (s, 3H); 2.92 (quint, 1H); 6.50 (s, 1H); 7.32 (d, 2H); 7.54-7.68 (m, 7H); 8.36 (d, 1H); 12.02 (brs 1H); MS (ESI+): m/z=482 [M+H]⁺; Melting point: 268-270° C.

### Example 64

### 5-{4-[4-(2-Dimethylamino-ethyl)-piperazin-1-yl]-phenyl}-3-hydroxy-4-(4-methyl-benzoyl)-1-(6-methyl-pyridazin-3-yl)-1,5-dihydro-pyrrol-2-one

Prepared from 4-[4-(2-dimethylamino-ethyl)-piperazin-1-yl]-benzaldehyde (Intermediate 7), 3-amino-6-methylpyridazine and 2-hydroxy-4-oxo-4-p-tolyl-but-2-enoic acid ethyl ester in 7% yield. After filtration of the mixture, the solid was washed with Et₂O/MeOH (0.5%), then, stirred in MeOH for 15 min and filtered. The solid was washed with Et₂O to give the desired compound. ¹H-NMR (DMSO-d₆): δ(ppm) 2.28 (s, 3H); 2.50 (s, 3H); 2.67 (s, 6H); 2.95-3.23 (m, 12H); 6.24 (s, 1H); 6.69 (d, 2H); 7.07 (d; 2H); 7.18 (d, 2H); 7.46 (d, 1H); 7.68 (d, 2H); 8.27 (d, 1H); MS (ESI+): m/z=541 [M+H]⁺; Melting point: 185.3° C.

### Example 89

### 3-hydroxy-5-(4-isopropoxy-phenyl)-4-(4-isopropyl-benzoyl)-1-(6-methyl-pyridazin-3-yl)-1,5-dihydro-pyrrol-2-one

Prepared from 4-isopropoxybenzaldehyde, 3-amino-6-methylpyridazine and 2-hydroxy-4-(4-isopropyl-phenyl)-4-oxo-but-2-enoic acid ethyl ester in 12% yield. ¹H-NMR (DMSO-d₆): δ(ppm) 1.14 (d, 6H); 1.20 (d, 6H); 2.50 (s, 3H); 2.93 (m, 1H); 4.43 (m, 1H); 6.40 (s, 1H); 6.68 (d, 2H); 7.26 (d, 2H); 7.33 (d, 2H); 7.59 (d, 1H); 7.69 (d, 2H); 8.25 (d, 1H); MS (ESI+): m/z=472 [M+H]⁺; Melting point: 280-281° C.

### Example 90

### 5-(4-tert-butoxyphenyl)-3-hydroxy-4-(4-isopropyl-benzoyl)-1-(6-methyl-pyridazin-3-yl)-1,5-dihydro-pyrrol-2-one

Prepared from 4-(tert-butoxy)benzaldehyde, 3-amino-6-methylpyridazine and 2-hydroxy-4-(4-isopropyl-phenyl)-4-oxo-but-2-enoic acid ethyl ester in 23% yield. ¹H-NMR (DMSO-d₆): δ (ppm) 1.15 (s, 9H); 1.18 (d, 6H); 2.50 (s, 3H); 2.92 (quint, 1H); 6.42 (s, 1H); 6.74 (d, 2H); 7.24 (d, 2H); 7.32 (d, 2H); 7.58 (d, 1H); 7.65 (d, 2H); 8.26 (d, 1H); MS (ESI+): m/z=486 [M+H]⁺; Melting point: 276-277° C.

### Example 93

### 3-hydroxy-1-(6-methyl-pyridazin-3-yl)-4-(4-pyridin-2-yl-benzoyl)-5-(4-trifluoromethoxy-phenyl)-1,5-dihydro-pyrrol-2-one

Prepared from 4-(trifluoromethoxy)benzaldehyde, 3-amino-6-methylpyridazine and 2-hydroxy-4-oxo-4-(4-pyridin-2-yl-phenyl)-but-2-enoic acid ethyl ester (Intermediate 9) in 23% yield. Note: After filtration of the mixture, the solid was washed with Et₂O then methanol to give the desired compound. ¹H-NMR (DMSO-d₆): δ (ppm) 2.36 (s, 3H); 6.36 (s, 1H); 6.48 (brs, 1H); 6.80 (d, 1H); 7.13 (d, ¹H); 7.21 (d, 1H); 7.37 (t, 1H); 7.52 (t, 2H); 7.88-8.05 (m, 5H); 8.39 (d, 1H); 8.68 (d, 1H); MS (ESI+): m/z=533 [M+H]⁺; Melting point: 247-249° C.

### Example 94

### 3-hydroxy-5-(4-isopropyl-phenyl)-1-(6-methyl-pyridin-3-yl)-4-(4-trifluoromethoxy-benzoyl)-1,5-dihydro-pyrrol-2-one

Prepared from 4-isopropylbenzaldehyde, 5-amino-2-methylpyridine and 2-hydroxy-4-oxo-4-(4-trifluoromethoxy-phenyl)-but-2-enoic acid ethyl ester in 28% yield. ¹H-NMR (DMSO-d₆): δ (ppm) 1.04 (d, 6H); 2.34 (s, 3H); 2.69 (quint, 1H); 6.08 (s, 1H); 6.95 (d, 2H); 7.11 (d, 2H); 7.17 (s, 1H); 7.22 (d, 2H); 7.78-7.90 (dd, 3H); 8.69 (d, 1H); MS (ESI+): m/z=497 [M+H]⁺

### Example 95

### 3-hydroxy-1-(6-methyl-pyridazin-3-yl)-4-(4-trifluoromethyl-benzoyl)-5-(4-trifluoromethyl-phenyl)-1,5-dihydro-pyrrol-2-one

Prepared from 4-(trifluoromethyl)benzaldehyde, 3-amino-6-methylpyridazine and 2-hydroxy-4-oxo-4-(4-trifluoromethyl-phenyl)-but-2-enoic acid ethyl ester in 2% yield. ¹H-NMR (DMSO-d₆): δ (ppm) 2.5 (s, 3H); 6.51 (s, 1H); 7.57-7.6 (m, 3H); 7.70 (d, 2H); 7.85 (m, 4H); 8.36 (d, 1H); MS (ESI+): m/z=508 [M+H]⁺

### Example 96

### 3-hydroxy-1-(6-methyl-pyridazin-3-yl)-4-(4-trifluoromethoxy-benzoyl)-5-(4-trifluoromethoxy-phenyl)-1,5-dihydro-pyrrol-2-one

Prepared from 4-(trifluoromethoxy)benzaldehyde, 5-amino-2-methylpyridine and 2-hydroxy-4-oxo-4-(4-trifluoromethoxy-phenyl)-but-2-enoic acid ethyl ester in 8% yield. ¹H-NMR (DMSO-d₆): δ (ppm) 2.5 (s, 3H); 6.47 (s, 1H); 7.19 (d, 2H); 7.44 (d, 2H); 7.56-7.62 (m, 3H); 7.86 (d, 2H); 8.33 (d, 1H); MS (ESI+): m/z=540 [M+H]⁺

### Example 98

### 3-hydroxy-4-(4-isopropyl-benzoyl)-1-(6-methyl-pyridazin-3-yl)-5-(6-trifluoromethyl-pyridin-3-yl)-1,5-dihydro-pyrrol-2-one

Prepared from 6-(trifluoromethyl)pyridine-3-carboxaldehyde, 3-amino-6-methylpyridazine and 2-hydroxy-4-(4-isopropyl-phenyl)-4-oxo-but-2-enoic acid ethyl ester in 6% yield. ¹H-NMR (DMSO-d₆): δ (ppm) 1.18 (d, 6H); 2.50 (s, 3H); 2.93 (quint, 1H); 6.50 (s, 1H); 7.31 (d, 2H); 7.62 (d, 1H); 7.72 (dd, 3H); 8.15 (dd, 1H); 8.42 (d, 1H); 8.90 (s, 1H); MS (ESI+): m/z=483 [M+H]⁺

### Example 99

### 5-(4-difluoromethoxy-phenyl)-3-hydroxy-4-(4-methoxy-benzoyl)-1-(6-methyl-pyridazin-3-yl)-1,5-dihydro-pyrrol-2-one

Prepared from 4-(difluoromethoxy)benzaldehyde, 3-amino-6-methylpyridazine and 2-hydroxy-4-(4-methoxy-phenyl)-4-oxo-but-2-enoic acid ethyl ester in 10% yield. ¹H-NMR (DMSO-d₆): δ (ppm) 2.5 (s, 3H); 3.82 (s, 3H); 6.45 (s, 1H); 6.83 (s, 0.3H); 6.95-7.00 (m, 4H); 7.13 (s, 0, 5H); 7.42 (s, 0, 2H); 7.46 (d, 2H); 7.60 (d, 1H); 7.75 (d, 2H); 8.30 (d, 1H); 11.80 (brs, 1H); MS (ESI+): m/z=468 [M+H]⁺

### Example 100

### 3-hydroxy-5-(4-isopropoxy-phenyl)-4-(4-methoxy-benzoyl)-1-(6-methyl-pyridazin-3-yl)-1,5-dihydro-pyrrol-2-one

Prepared from 4-isopropoxybenzaldehyde, 3-amino-6-methylpyridazine and 2-hydroxy-4-(4-methoxy-phenyl)-4-oxo-but-2-enoic acid ethyl ester in 17% yield. ¹H-NMR (DMSO-d₆): δ (ppm) 1.12 (d, 6H); 2.5 (s, 3H); 3.80 (s, 3H); 4.41 (quint, 1H); 6.39 (s, 1H); 6.66 (d, 2H); 6.97 (d, 2H); 7.24 (d, 2H); 7.57 (d, 1H); 7.75 (d, 2H); 8.24 (d, 1H); 11.65 (brs, 1H); MS (ESI+): m/z=460 [M+H]⁺

### Example 101

### 4-(4-ethoxy-benzoyl)-3-hydroxy-1-(6-methyl-pyridazin-3-yl)-5-(4-trifluoromethoxy-phenyl)-1,5-dihydro-pyrrol-2-one

Prepared from 4-(trifluoromethoxy)benzaldehyde, 3-amino-6-methylpyridazine and 4-(4-ethoxy-phenyl)-2-hydroxy-4-oxo-but-2-enoic acid ethyl ester in 17% yield. ¹H-NMR (DMSO-d₆): δ (ppm) 1.32 (t, 3H); 2.50 (s, 3H); 4.09 (q, 2H); 6.46 (s, 1H); 6.96 (d, 2H); 7.16 (d, 2H); 7.53 (d, 2H); 7.59 (d, 1H); 7.73 (d, 2H); 8.32 (d, 1H); 11.88 (brs, 1H); MS (ESI+): m/z=500 [M+H]⁺; Melting point: 255-257° C.

### Example 102

### 4-(4-ethyl-benzoyl)-3-hydroxy-1-(6-methyl-pyridazin-3-yl)-5-(4-trifluoromethoxy-phenyl)-1,5-dihydro-pyrrol-2-one

Prepared from 4-(trifluoromethoxy)benzaldehyde, 3-amino-6-methylpyridazine and 4-(4-ethyl-phenyl)-2-hydroxy-4-oxo-but-2-enoic acid ethyl ester in 32% yield. ¹H-NMR (DMSO-d₆): δ (ppm) 1.16 (t, 3H); 2.50 (s, 3H); 2.64 (q, 2H); 6.46 (s, 1H); 7.16 (d, 2H); 7.28 (d, 2H); 7.53-7.67 (m, 5H); 8.32 (d, 1H); MS (ESI+): m/z=484 [M+H]⁺; Melting point: 263-267° C.

### Example 103

### 3-hydroxy-1-(6-methyl-pyridazin-3-yl)-4-(pyridine-4-carbonyl)-5-(4-trifluoromethoxy-phenyl)-1,5-dihydro-pyrrol-2-one

Prepared from 4-(trifluoromethoxy)benzaldehyde, 3-amino-6-methylpyridazine and 2-hydroxy-4-oxo-4-pyridin-4-yl-but-2-enoic acid ethyl ester in 3% yield. ¹H-NMR (DMSO-d₆): δ (ppm) 2.50 (s, 3H); 6.34 (s, 1H); 7.13 (d, 2H); 7.47-7.60 (m, 5H); 8.38 (d, 1H); 8.58 (d, 2H); MS (ESI+): m/z=457 [M+H]⁺

### Example 104

### 5-(4-ethoxy-phenyl)-3-hydroxy-4-(4-isopropyl-benzoyl)-1-(6-methyl-pyridazin-3-yl)-1,5-dihydro-pyrrol-2-one

Prepared from 4-ethoxybenzaldehyde, 3-amino-6-methylpyridazine and 2-hydroxy-4-(4-isopropyl-phenyl)-4-oxo-but-2-enoic acid ethyl ester in 18% yield. ¹H-NMR (DMSO-d₆): δ(ppm) 1.19 (d, 9H); 2.50 (s, 3H); 2.93 (quint, 1H); 3.85 (q, 2H); 6.41 (s, 1H); 6.70 (d, 2H); 7.28 (d, 2H); 7.32 (d, 2H); 7.58 (d, 1H); 7.69 (d, 2H); 8.25 (d, 1H); MS (ESI+): m/z=458 [M+H]⁺; Melting point: 279° C.

### Example 105

### 5-(4-ethyl-phenyl)-3-hydroxy-4-(4-isopropyl-benzoyl)-1-(6-methyl-pyridazin-3-yl)-1,5-dihydro-pyrrol-2-one

Prepared from 4-ethylbenzaldehyde, 3-amino-6-methylpyridazine and 2-hydroxy-4-(4-isopropyl-phenyl)-4-oxo-but-2-enoic acid ethyl ester in 28% yield. ¹H-NMR (DMSO-d₆): δ(ppm) 1.03 (t, 3H); 1.19 (d, 6H); 2.42 (q, 2H); 2.50 (s, 3H); 2.92 (quint, 1H); 6.43 (s, 1H); 7.01 (d, 2H); 7.31 (t, 4H); 7.58 (d, 1H); 7.68 (d, 2H); 8.27 (d, 1H); MS (ESI+): m/z=442 [M+H]⁺; Melting point: 282-284° C.

### Example 106

### 3-hydroxy-4-(4-isopropyl-benzoyl)-1-(6-methyl-pyridazin-3-yl)-5-(5-trifluoromethyl-pyridin-2-yl)-1,5-dihydro-pyrrol-2-one

Prepared from 5-(trifluoromethyl)-2-pyridinecarboxyaldehyde, 3-amino-6-methylpyridazine and 2-hydroxy-4-(4-isopropyl-phenyl)-4-oxo-but-2-enoic acid ethyl ester in 11% yield. ¹H-NMR (DMSO-d₆): δ (ppm) 1.19 (d, 6H); 2.50 (s, 3H); 2.92 (quint, 1H); 6.57 (s, 1H); 7.30 (d, 2H); 7.58-7.63 (m, 3H); 7.89 (d, 1H); 8.14 (dd, 1H); 8.50 (d, 1H); 8.74 (brs, 1H); MS (ESI+): m/z=483 [M+H]⁺

### Example 108

### 3-hydroxy-1-(6-methyl-pyridazin-3-yl)-4-(5-methyl-pyridine-2-carbonyl)-5-(4-trifluoromethoxy-phenyl)-1,5-dihydro-pyrrol-2-one

Prepared from 4-(trifluoromethoxy)benzaldehyde, 3-amino-6-methylpyridazine and 2-hydroxy-4-(5-methyl-pyridin-2-yl)-4-oxo-but-2-enoic acid ethyl ester in 13% yield. ¹H-NMR (DMSO-d₆): δ (ppm) 2.48 (s, 3H); 2.50 (s, 3H); 6.46 (s, 1H); 7.15 (d, 2H); 7.46-7.59 (m, 3H); 8.02 (d, 2H); 8.36 (d, 1H); 8.76 (brs, 1H); MS (ESI+): m/z=471 [M+H]⁺

### Example 109

### 3-hydroxy-4-(4-isopropoxy-benzoyl)-5-(4-isopropoxy-phenyl)-1-(6-methyl-pyridazin-3-yl)-1,5-dihydro-pyrrol-2-one

Prepared from 4-isopropoxybenzaldehyde, 3-amino-6-methylpyridazine and 2-hydroxy-4-(4-isopropoxy-phenyl)-4-oxo-but-2-enoic acid ethyl ester in 9% yield. ¹H-NMR (DMSO-d₆): δ (ppm) 1.13 (d, 6H); 1.26 (d, 6H); 2.50 (s, 3H); 4.42 (quint, 1H); 4.72 (quint, 1H); 6.39 (s, 1H); 6.68 (d, 2H); 6.96 (d, 2H); 7.25 (d, 2H); 7.58 (d, 1H); 7.72 (d, 2H); 8.27 (d, 1H); 11.65 (brs, 1H); MS (ESI+): m/z=488 [M+H]⁺

### Example 110

### 5-(6-ethoxy-pyridin-3-yl)-3-hydroxy-4-(4-isopropyl-benzoyl)-1-(6-methyl-pyridazin-3-yl)-1,5-dihydro-pyrrol-2-one

Prepared from 6-ethoxynicotinaldehyde, 3-amino-6-methylpyridazine and 2-hydroxy-4-(4-isopropyl-phenyl)-4-oxo-but-2-enoic acid ethyl ester in 25% yield. ¹H-NMR (DMSO-d₆): δ (ppm) 1.15-1.19 (m, 9H); 2.50 (s, 3H); 2.92 (quint, 1H); 4.12 (q, 2H); 6.38 (s, 1H); 6.56 (d, 1H); 7.32 (d, 2H); 7.58 (d, 1H); 7.71 (d, 3H); 8.20 (d, 1H); 8.27 (d, 1H); MS (ESI+): m/z=459 [M+H]⁺; Melting point: 274-276° C.

### Example 111

### 3-Hydroxy-4-(4-isopropyl-benzoyl)-5-(5-isopropyl-pyridin-2-yl)-1-(6-methyl-pyridazin-3-yl)-1,5-dihydro-pyrrol-2-one

Prepared from 5-isopropyl-pyridine-2-carbaldehyde, 3-amino-6-methylpyridazine and 2-hydroxy-4-(4-isopropyl-phenyl)-4-oxo-but-2-enoic acid ethyl ester in 43% yield. ¹H-NMR (DMSO-d₆): δ(ppm) 1.09 (d, 6H); 1.19 (d, 6H); 2.50 (s, 3H); 2.78 (quint, 1H); 2.90 (quint, 1H); 6.50 (s, 1H); 7.32 (d, 2H); 7.46-7.72 (m, 5H); 8.23 (brs, 1H); 8.43 (d, 1H); MS (ESI+): m/z=457 [M+H]⁺

### Example 112

### 3-hydroxy-1-(6-methyl-pyridazin-3-yl)-4-(6-methyl-pyridine-3-carbonyl)-5-(4-trifluoromethoxy-phenyl)-1,5-dihydro-pyrrol-2-one

Prepared from 4-(trifluoromethoxy)benzaldehyde, 3-amino-6-methylpyridazine and 2-hydroxy-4-(6-methyl-pyridin-3-yl)-4-oxo-but-2-enoic acid ethyl ester in 15% yield. ¹H-NMR (DMSO-d₆): δ (ppm) 2.48 (s, 3H); 2.50 (s, 3H); 6.41 (s, 1H); 7.17 (d, 2H); 7.36 (d, 1H); 7.54-7.59 (m, 3H); 8.03 (d, 1H); 8.34 (d, 1H); 8.79 (brs, 1H); MS (ESI+): m/z=471 [M+H]⁺

### Example 113

### 3-hydroxy-4-(4-isopropyl-benzoyl)-1-(6-methyl-pyridazin-3-yl)-5-(5-methyl-thiazol-2-yl)-1,5-dihydro-pyrrol-2-one

Prepared from 5-methyl-thiazole-2-carbaldehyde, 3-amino-6-methylpyridazine and 2-hydroxy-4-(4-isopropyl-phenyl)-4-oxo-but-2-enoic acid ethyl ester in 10% yield. ¹H-NMR (DMSO-d₆): δ (ppm) 1.21 (d, 6H); 2.29 (s, 3H); 2.56 (s, 3H); 2.95 (quint, 1H); 6.77 (s, 1H); 7.22 (s, 1H); 7.36 (d, 2H); 7.64 (d, 1H); 7.70 (d, 2H); 8.35 (d, 1H); MS (ESI+): m/z=435 [M+H]⁺

### Example 114

### 5-(5-ethyl-pyridin-2-yl)-3-hydroxy-4-(4-isopropyl-benzoyl)-1-(6-methyl-pyridazin-3-yl)-1,5-dihydro-pyrrol-2-one

Prepared from 5-ethylpyridine-2-carbaldehyde, 3-amino-6-methylpyridazine and 2-hydroxy-4-(4-isopropyl-phenyl)-4-oxo-but-2-enoic acid ethyl ester in 37% yield. ¹H-NMR (DMSO-d₆): δ (ppm) 1.05 (t, 3H); 1.20 (d, 6H); 2.48 (q, 2H); 2.50 (s, 3H); 2.92 (quint, 1H); 6.49 (s, 1H); 7.31 (d, 2H); 7.48-7.63 (m, 5H); 8.18 (brs, 1H); 8.43 (d, 1H); MS (ESI+): m/z=443 [M+H]⁺

### Example 115

### 3-hydroxy-4-(4-isopropoxy-benzoyl)-1-(6-methyl-pyridazin-3-yl)-5-(4-trifluoromethoxy-phenyl)-1,5-dihydro-pyrrol-2-one

Prepared from 4-(trifluoromethoxy)benzaldehyde, 3-amino-6-methylpyridazine and 2-hydroxy-4-(4-isopropoxy-phenyl)-4-oxo-but-2-enoic acid ethyl ester in 18% yield. ¹H-NMR (DMSO-d₆): δ (ppm) 1.26 (d, 6H); 2.50 (s, 3H); 4.72 (quint, 1H); 6.46 (s, 1H); 6.94 (d, 2H); 7.17 (d, 2H); 7.53 (d, 2H); 7.60 (d, 1H); 7.72 (d, 2H); 8.32 (d, 1H); 11.90 (brs, 1H); MS (ESI+): m/z=514 [M+H]⁺

### Example 116

### 3-hydroxy-1-(6-methyl-pyridazin-3-yl)-4-(5-methyl-thiazole-2-carbonyl)-5-(4-trifluoromethoxy-phenyl)-1,5-dihydro-pyrrol-2-one

Prepared from 4-(trifluoromethoxy)benzaldehyde, 3-amino-6-methylpyridazine and 2-hydroxy-4-(5-methyl-thiazol-2-yl)-4-oxo-but-2-enoic acid ethyl ester (intermediate 10) in 6% yield. ¹H-NMR (DMSO-d₆): δ (ppm) 2.50 (s, 3H); 2.59 (s, 3H); 6.45 (s, 1H); 7.19 (d, 2H); 7.54 (d, 2H); 7.60 (d, 1H); 8.12 (brs, 1H); 8.35 (d, 1H); MS (ESI+): m/z=477 [M+H]⁺

### Example 117

### 3-hydroxy-4-(6-methoxy-pyridine-3-carbonyl)-1-(6-methyl-pyridazin-3-yl)-5-(4-trifluoromethoxy-phenyl)-1,5-dihydro-pyrrol-2-one

Prepared from 4-(trifluoromethoxy)benzaldehyde, 3-amino-6-methylpyridazine and 2-hydroxy-4-(6-methoxy-pyridin-3-yl)-4-oxo-but-2-enoic acid ethyl ester in 5% yield. ¹H-NMR (DMSO-d₆): δ (ppm) 2.5 (s, 3H); 3.92 (s, 3H); 6.46 (s, 1H); 6.88 (d, 1H); 7.18 (d, 2H); 7.56-7.63 (m, 3H); 8.00 (dd, 1H); 8.33 (d, 1H); 8.63 (d, 1H); MS (ESI+): m/z=487 [M+H]⁺; Melting point: 250-251° C.

### Example 118

### 1-(6-chloro-pyridazin-3-yl)-3-hydroxy-4-(4-isopropyl-benzoyl)-5-(4-trifluoromethoxy-phenyl)-1,5-dihydro-pyrrol-2-one

Prepared from 4-(trifluoromethoxy)benzaldehyde, 3-amino-6-chloropyridazine and 2-hydroxy-4-(4-isopropyl-phenyl)-4-oxo-but-2-enoic acid ethyl ester in 15% yield. ¹H-NMR (DMSO-d₆): δ (ppm) 1.19 (d, 6H); 2.93 (quint, 1H); 6.43 (s, 1H); 7.20 (d, 2H); 7.34 (d, 2H); 7.58 (d, 2H); 7.68 (d, 2H); 7.94 (d, 1H); 8.56 (d, 1H); 12.07 (brs, 1H); MS (ESI+): m/z=518 [M+H]⁺

### Example 120

### 5-(4-cyclopropoxy-phenyl)-3-hydroxy-4-(4-isopropyl-benzoyl)-1-(6-methyl-pyridazin-3-yl)-1,5-dihydro-pyrrol-2-one

Prepared from 4-cyclopropoxy-benzaldehyde, 3-amino-6-methylpyridazine and 2-hydroxy-4-(4-isopropyl-phenyl)-4-oxo-but-2-enoic acid ethyl ester in 10% yield. ¹H-NMR (DMSO-d₆): δ(ppm) 0.60 (dd, 4H); 1.20 (d, 6H); 2.50 (s, 3H); 2.94 (quint, 1H); 3.68 (brs, 1H); 6.43 (s, 1H); 6.84 (d, 2H); 7.31-7.35 (m, 4H); 7.59 (d, 1H); 7.70 (d, 2H); 8.26 (d, 1H); MS (ESI+): m/z=470 [M+H]⁺; Melting point: 285-288° C.

### Example 121

### 3-hydroxy-5-(5-isopropoxy-pyridin-2-yl)-4-(4-isopropyl-benzoyl)-1-(6-methyl-pyridazin-3-yl)-1,5-dihydro-pyrrol-2-one

Prepared from 5-isopropoxy-pyridine-2-carbaldehyde (Intermediate 11), 3-amino-6-methylpyridazine and 2-hydroxy-4-(4-isopropyl-phenyl)-4-oxo-but-2-enoic acid ethyl ester in 48% yield. ¹H-NMR (DMSO-d₆): δ(ppm) 1.15 (d, 6H); 1.19 (d, 6H); 2.50 (s, 3H); 2.92 (quint, 1H); 4.52 (quint, 1H); 6.47 (s, 1H); 7.25 (dd, 1H); 7.31 (d, 2H); 7.48 (d, 1H); 7.60 (t, 3H); 7.96 (d, 1H); 8.39 (d, 1H); 11.87 (brs, 1H); MS (ESI+): m/z=473 [M+H]⁺; Melting point: 243-245° C.

### Example 122

### 5-(5-ethoxy-pyridin-2-yl)-3-hydroxy-4-(4-isopropyl-benzoyl)-1-(6-methyl-pyridazin-3-yl)-1,5-dihydro-pyrrol-2-one

Prepared from 5-ethoxy-pyridine-2-carbaldehyde (Intermediate 12), 3-amino-6-methylpyridazine and 2-hydroxy-4-(4-isopropyl-phenyl)-4-oxo-but-2-enoic acid ethyl ester in 43% yield. ¹H-NMR (DMSO-d₆): δ (ppm) 1.18-1.26 (m, 9H); 2.5 (s, 3H); 2.92 (quint, 1H); 3.94 (q, 2H); 6.48 (s, 1H); 7.24 (d, 1H); 7.31 (d, 2H); 7.49 (d, 1H); 7.60 (t, 3H); 8.00 (brs, 1H); 8.40 (d, 1H); MS (ESI+): m/z=459 [M+H]⁺; Melting point: 198-199° C.

### Example 123

### 4-(4-tert-butyl-benzoyl)-3-hydroxy-1-(6-methyl-pyridazin-3-yl)-5-(4-trifluoromethoxy-phenyl)-1,5-dihydro-pyrrol-2-one

Prepared from 4-(trifluoromethoxy)benzaldehyde, 3-amino-6-methylpyridazine and 4-(4-tert-butyl-phenyl)-2-hydroxy-4-oxo-but-2-enoic acid ethyl ester in 17% yield. ¹H-NMR (DMSO-d₆): δ (ppm) 1.27 (s, 9H); 2.50 (s, 3H); 6.47 (s, 1H); 7.17 (d, 2H); 7.46 (d, 2H); 7.55 (d, 2H); 7.60 (d, 1H); 7.68 (d, 2H); 8.33 (d, 1H); MS (ESI+): m/z=512 [M+H]⁺; Melting point: 262-265° C.

### Example 124

### 5-(4-acetyl-phenyl)-3-hydroxy-4-(4-isopropyl-benzoyl)-1-(6-methyl-pyridazin-3-yl)-1,5-dihydro-pyrrol-2-one

Prepared from 4-acetylbenzaldehyde, 3-amino-6-methylpyridazine and 2-hydroxy-4-(4-isopropyl-phenyl)-4-oxo-but-2-enoic acid ethyl ester in 21% yield. ¹H-NMR (DMSO-d₆): δ (ppm) 1.18 (d, 6H); 2.44 (s, 3H); 2.5 (s, 3H); 2.92 (quint, 1H); 6.49 (s, 1H); 7.31 (d, 2H); 7.54 (d, 2H); 7.60 (d, 1H); 7.66 (d, 2H); 7.76 (d, 2H); 8.34 (d, 1H); 11.99 (brs, 1H); MS (ESI+): m/z=456 [M+H]⁺; Melting point: 259-266° C.

### Example 125

### 4-(4-cyclohexyl-benzoyl)-3-hydroxy-1-(6-methyl-pyridazin-3-yl)-5-(4-trifluoromethoxy-phenyl)-1,5-dihydro-pyrrol-2-one

Prepared from 4-(trifluoromethoxy)benzaldehyde, 3-amino-6-methylpyridazine and 4-(4-cyclohexyl-phenyl)-2-hydroxy-4-oxo-but-2-enoic acid ethyl ester in 36% yield. ¹H-NMR (DMSO-d₆): δ (ppm) 1.22-1.47 (m, 5H); 1.67-178 (m, 5H); 2.48-2.51 (m, 4H); 6.46 (s, 1H); 7.16 (d, 2H); 7.30 (d, 2H); 7.55 (d, 2H); 7.60 (d, 1H); 7.67 (d, 2H); 8.32 (d, 1H); 11.99 (brs, 1H); MS (ESI+): m/z=538 [M+H]⁺; Melting point: 283-286° C.

### Example 126

### 3-hydroxy-1-(6-methyl-pyridazin-3-yl)-5-(4-trifluoromethoxy-phenyl)-4-(6-trifluoromethyl-pyridine-3-carbonyl)-1,5-dihydro-pyrrol-2-one

Prepared from 4-(trifluoromethoxy)benzaldehyde, 3-amino-6-methylpyridazine and 2-hydroxy-4-oxo-4-(6-trifluoromethyl-pyridin-3-yl)-but-2-enoic acid ethyl ester (Intermediate 13) in 19% yield. ¹H-NMR (DMSO-d₆): δ (ppm) 2.50 (s, 3H); 6.34 (s, 1H); 7.13 (d, 2H); 7.46-7.54 (m, 3H); 7.83 (d, 1H); 8.23 (d, 1H); 8.40 (d, 1H); 8.88 (s, 1H); MS (ESI+): m/z=525 [M+H]⁺; Melting point: 172° C.

### Example 127

### 3-hydroxy-4-(4-isopropyl-benzoyl)-1-(6-methyl-pyridazin-3-yl)-5-pyridin-4-yl-1,5-dihydro-pyrrol-2-one

Prepared from 4-pyridinecarboxaldehyde, 3-amino-6-methylpyridazine and 2-hydroxy-4-(4-isopropyl-phenyl)-4-oxo-but-2-enoic acid ethyl ester in 10% yield. Note: After filtration of the mixture, the solid was washed with Et₂O then Et₂O/DMSO (10%) to give the desired compound. ¹H-NMR (DMSO-d₆): δ (ppm) 1.19 (d, 6H); 2.5 (s, 3H); 2.92 (quint, 1H); 6.38 (s, 1H); 7.30 (d, 2H); 7.43 (d, 2H); 7.61 (d, 1H); 7.68 (d, 2H); 8.40 (d, 3H); MS (ESI+): m/z=415 [M+H]⁺; Melting point: degradation at 250° C.

### Example 128

### 3-hydroxy-4-(4-isopropyl-benzoyl)-1-(5-methyl-pyrazin-2-yl)-5-(4-trifluoromethoxy-phenyl)-1,5-dihydro-pyrrol-2-one

Prepared from 4-(trifluoromethoxy)benzaldehyde, 2-amino-5-methylpyrazine and 2-hydroxy-4-(4-isopropyl-phenyl)-4-oxo-but-2-enoic acid ethyl ester in 17% yield. ¹H-NMR (DMSO-d₆): δ (ppm) 1.20 (d, 6H); 2.40 (s, 3H); 2.93 (quint, 1H); 6.30 (s, 1H); 7.18 (d, 2H): 7.33 (d, 2H); 7.54 (d, 2H); 7.68 (d, 2H); 8.25 (brs, 1H); 9.31 (d, 1H); 12.08 (brs, 1H); MS (ESI+): m/z=498 [M+H]⁺; Melting point: 266-268° C.

### Example 129

### 3-hydroxy-4-(4-isopropyl-benzoyl)-1-(6-methoxy-pyridazin-3-yl)-5-(4-trifluoromethoxy-phenyl)-1,5-dihydro-pyrrol-2-one

Prepared from 4-(trifluoromethoxy)benzaldehyde, 3-amino-6-methoxypyridazine and 2-hydroxy-4-(4-isopropyl-phenyl)-4-oxo-but-2-enoic acid ethyl ester in 21% yield. ¹H-NMR (DMSO-d₆): δ (ppm) 1.20 (d, 6H); 2.93 (quint, 1H); 3.94 (s, 3H); 6.40 (s, 1H); 7.19 (d, 2H); 7.29 (d, 1H); 7.33 (d, 2H); 7.55 (d, 2H); 7.68 (d, 2H); 8.37 (d, 1H); 12.07 (brs, 1H); MS (ESI+): m/z=514 [M+H]⁺; Melting point: 271° C.

### Example 130

### 3-[3-hydroxy-4-(4-isopropyl-benzoyl)-2-oxo-5-(4-trifluoromethoxy-phenyl)-2,5-dihydro-pyrrol-1-yl]-1-methyl-1H-pyridin-2-one

Prepared from 4-(trifluoromethoxy)benzaldehyde, 3-amino-1-methyl-1H-pyridin-2-one and 2-hydroxy-4-(4-isopropyl-phenyl)-4-oxo-but-2-enoic acid ethyl ester in 10% yield. ¹H-NMR (DMSO-d₆): δ (ppm) 1.19 (d, 6H); 2.92 (quint, 1H); 3.36 (s, 3H); 6.12 (t, 1H); 6.33 (s, 1H); 7.18 (d, 2H); 7.32 (d, 2H); 7.40-7.48 (m, 3H); 7.63-7.68 (m, 3H); 11.84 (brs, 1H); MS (ESI+): m/z=513 [M+H]⁺; Melting point: 207° C.

### Example 131

### 3-hydroxy-4-(4-isobutyl-benzoyl)-1-(6-methyl-pyridazin-3-yl)-5-(4-trifluoromethoxy-phenyl)-1,5-dihydro-pyrrol-2-one

Prepared from 4-(trifluoromethoxy)benzaldehyde, 3-amino-6-methylpyridazine and 2-hydroxy-4-(4-isobutyl-phenyl)-4-oxo-but-2-enoic acid ethyl ester in 35% yield. ¹H-NMR (DMSO-d₆): δ (ppm) 0.83 (d, 6H); 1.84 (quint, 1H), 2.48-2.51 (m, 5H); 6.47 (s, 1H); 7.16 (d, 2H); 7.23 (d, 2H); 755 (d, 2H); 7.60 (d, 1H); 7.65 (d, 2H); 8.37 (d, 1H); 11.99 (brs, 1H); MS (ESI+): m/z=512 [M+H]⁺; Melting point: 260° C.

### Example 132

### 3-hydroxy-4-(4-isopropyl-benzoyl)-5-(4-methanesulfonyl-phenyl)-1-(6-methyl-pyridazin-3-yl)-1,5-dihydro-pyrrol-2-one

Prepared from 4-methylsulphonyl benzaldehyde, 3-amino-6-methylpyridazine and 2-hydroxy-4-(4-isopropyl-phenyl)-4-oxo-but-2-enoic acid ethyl ester in 7% yield. ¹H-NMR (DMSO-d₆): δ (ppm) 1.19 (d, 6H); 2.50 (s, 3H); 2.93 (quint, 1H); 3.14 (s, 3H); 6.52 (s, 1H); 7.34 (d, 2H); 7.63 (d, 1H); 7.69 (d, 2H); 7.74-7.78 (m, 4H); 8.38 (d, 1H); MS (ESI+): m/z=492 [M+H]⁺

### Example 133

### 4-[4-hydroxy-3-(4-isopropyl-benzoyl)-1-(6-methyl-pyridazin-3-yl)-5-oxo-2,5-dihydro-1H-pyrrol-2-yl]-benzonitrile

Prepared from 4-cyanobenzaldehyde, 3-amino-6-methylpyridazine and 2-hydroxy-4-(4-isopropyl-phenyl)-4-oxo-but-2-enoic acid ethyl ester in 30% yield. ¹H-NMR (DMSO-d₆): δ (ppm) 1.19 (d, 6H); 2.50 (s, 3H); 2.93 (quint, 1H); 6.48 (s, 1H); 7.32 (d, 2H); 7.59-7.69 (m, 7H); 8.36 (d, 1H); MS (ESI+): m/z=439 [M+H]⁺

### Example 134

### 5-(4-fluoro-phenyl)-3-hydroxy-4-(4-isopropyl-benzoyl)-1-(6-methyl-pyridazin-3-yl)-1,5-dihydro-pyrrol-2-one

Prepared from 4-fluorobenzaldehyde, 3-amino-6-methylpyridazine and 2-hydroxy-4-(4-isopropyl-phenyl)-4-oxo-but-2-enoic acid ethyl ester in 20% yield. ¹H-NMR (DMSO-d₆): δ (ppm) 1.19 (d, 6H); 2.5 (s, 3H); 2.92 (quint, 1H); 6.44 (s, 1H); 6.99 (t, 2H); 7.32 (d, 2H); 7.44 (t, 2H); 7.59 (d, 1H); 7.68 (d, 2H); 8.29 (d, 1H); 11.91 (brs, 1H); MS (ESI+): m/z=432 [M+H]⁺; Melting point: 273-278° C.

### Example 135

### 5-(4-dimethylamino-phenyl)-3-hydroxy-4-(4-isopropyl-benzoyl)-1-(6-methyl-pyridazin-3-yl)-1,5-dihydro-pyrrol-2-one

Prepared from dimethylaminobenzaldehyde, 3-amino-6-methylpyridazine and 2-hydroxy-4-(4-isopropyl-phenyl)-4-oxo-but-2-enoic acid ethyl ester in 7% yield. ¹H-NMR (DMSO-d₆): δ (ppm) 1.19 (d, 6H); 2.5 (s, 3H); 2.74 (s, 6H); 2.93 (quint, 1H); 6.36 (s, 1H); 6.46 (d, 2H); 7.16 (d, 2H); 7.33 (d, 2H); 7.57 (d, 1H); 7.69 (d, 2H); 8.21 (d, 1H); 11.63 (brs, 1H); MS (ESI+): m/z=457 [M+H]⁺; Melting point: 283-286° C.

### Example 136

### 3-hydroxy-5-(4-isobutyl-phenyl)-4-(4-isopropyl-benzoyl)-1-(6-methyl-pyridazin-3-yl)-1,5-dihydro-pyrrol-2-one

Prepared from 4-isobutyl-benzaldehyde, 3-amino-6-methylpyridazine and 2-hydroxy-4-(4-isopropyl-phenyl)-4-oxo-but-2-enoic acid ethyl ester in 14% yield. ¹H-NMR (DMSO-d₆): δ (ppm) 0.71 (d, 6H); 1.20 (d, 6H); 1.67 (quint, 1H); 1.26 (d, 2H); 2.5 (s, 3H); 2.93 (quint, 1H); 6.44 (s, 1H); 7.95 (d, 2H); 7.26 (d, 2H); 7.32 (d, 2H); 7.59 (d, 1H); 7.66 (d, 2H); 8.28 (d, 1H); MS (ESI+): m/z=470 [M+H]⁺

### Example 137

### 5-(4-ethynyl-phenyl)-3-hydroxy-4-(4-isopropyl-benzoyl)-1-(6-methyl-pyridazin-3-yl)-1,5-dihydro-pyrrol-2-one

Prepared from 4-ethynyl-benzaldehyde, 3-amino-6-methylpyridazine and 2-hydroxy-4-(4-isopropyl-phenyl)-4-oxo-but-2-enoic acid ethyl ester in 16% yield. ¹H-NMR (DMSO-d₆): δ (ppm) 1.18 (d, 6H); 2.5 (s, 3H); 2.92 (quint, 1H); 4.08 (s, 1H); 6.44 (s, 1H); 7.29 (t, 4H); 7.40 (d, 2H); 7.60 (d, 1H); 7.67 (d, 2H); 8.32 (d, 1H); MS (ESI+): m/z=438 [M+H]+

### Example 139

### 3-hydroxy-5-(4-hydroxy-phenyl)-4-(4-isopropyl-benzoyl)-1-(6-methyl-pyridazin-3-yl)-1,5-dihydro-pyrrol-2-one

Prepared from 4-hydroxybenzaldehyde, 3-amino-6-methylpyridazine and 2-hydroxy-4-(4-isopropyl-phenyl)-4-oxo-but-2-enoic acid ethyl ester in 8% yield. ¹H-NMR (DMSO-d₆): δ (ppm) 1.20 (d, 6H); 2.5 (s, 3H); 2.93 (quint, 1H); 6.37 (s, 1H); 6.53 (d, 2H); 7.15 (d, 2H); 7.35 (d, 2H); 7.60 (d, 1H); 7.67 (d, 2H); 8.23 (d; 1H); 9.28 (brs, 1H); 11.7 (brs, 1H); MS (ESI+): m/z=430 [M+H]⁺; Melting point: 222-224° C.

### Example 140

### 3-hydroxy-4-(4-isopropyl-benzoyl)-1-(6-methyl-pyridazin-3-yl)-5-(4-tetrazol-1-ylmethyl-phenyl)-1,5-dihydro-pyrrol-2-one

Prepared from 4-tetrazol-1-ylmethyl-benzaldehyde (Intermediate 14), 3-amino-6-methylpyridazine and 2-hydroxy-4-(4-isopropyl-phenyl)-4-oxo-but-2-enoic acid ethyl ester in 19% yield. ¹H-NMR (DMSO-d₆): δ (ppm) 1.20 (d, 6H); 2.5 (s, 3H); 2.92 (quint, 1H); 5.57 (s, 2H); 6.44 (s, 1H); 7.10 (d, 2H); 7.31 (d, 2H); 7.43 (d, 2H); 7.58 (d, 1H); 7.66 (d, 2H); 8.29 (d, 1H); 9.44 (s, 1H); MS (ESI+): m/z=496 [M+H]⁺; Melting point>300° C.

### Example 141

### 3-hydroxy-4-(4-isopropyl-benzoyl)-1-(6-methyl-pyridazin-3-yl)-5-(4-[1,2,4]triazol-1-ylmethyl-phenyl)-1,5-dihydro-pyrrol-2-one

Prepared from 4-(1H-1,2,4-triazol-1-ylmethyl)benzaldehyde, 3-amino-6-methylpyridazine and 2-hydroxy-4-(4-isopropyl-phenyl)-4-oxo-but-2-enoic acid ethyl ester in 15% yield. ¹H-NMR (DMSO-d₆): δ (ppm) 1.18 (d, 6H); 2.5 (s, 3H); 2.94 (quint, 1H); 5.26 (s, 2H); 6.44 (s, 1H); 7.02 (d, 2H); 7.31 (d, 2H); 7.38 (d, 2H); 7.57 (d, 1H); 7.67 (d, 2H); 7.89 (s, 1H); 8.28 (d, 1H); 8.56 (s, 1H); MS (ESI+): m/z=495 [M+H]⁺; Melting point: 223° C.

### Example 142

### 3-hydroxy-4-(4-isopropenyl-benzoyl)-1-(6-methyl-pyridazin-3-yl)-5-(4-trifluoromethoxy-phenyl)-1,5-dihydro-pyrrol-2-one

Prepared from 4-(trifluoromethoxy)benzaldehyde, 3-amino-6-methylpyridazine and 2-hydroxy-4-(4-isopropenyl-phenyl)-4-oxo-but-2-enoic acid ethyl ester (intermediate 16) in 16% yield. ¹H-NMR (DMSO-d₆): δ (ppm) 2.12 (s, 3H); 2.50 (s, 3H); 5.22 (s, 1H); 5.55 (s, 1H); 6.48 (s, 1H); 7.18 (d, 2H); 7.52-7.65 (m, 5H); 7.73 (d, 2H); 8.33 (d, 1H); MS (ESI+): m/z=496 [M+H]⁺; Melting point: 253° C.

### Example 143

### 3-hydroxy-4-(4-isopropyl-benzoyl)-1-(6-methyl-pyridazin-3-yl)-5-[4-(tetrahydro-pyran-4-yloxy)-phenyl]-1,5-dihydro-pyrrol-2-one

Prepared from 4-(tetrahydropyran-4-yloxy)benzaldehyde, 3-amino-6-methylpyridazine and 2-hydroxy-4-(4-isopropyl-phenyl)-4-oxo-but-2-enoic acid ethyl ester in 10% yield. ¹H-NMR (DMSO-d₆): δ (ppm) 1.19 (d, 6H); 1.44 (m, 2H); 1.82 (m, 2H); 2.5 (s, 3H); 2.93 (quint, 1H); 3.39 (m; 2H); 3.75 (m, 2H); 4.40 (m, 1H); 6.42 (s, 1H); 7.75 (d, 2H); 7.30 (m, 4H); 7.60 (d, 1H); 7.69 (d, 2H); 8.26 (d, 1H); MS (ESI+): m/z=514 [M+H]⁺; Melting point: 275° C.

### Example 144

### 3-hydroxy-4-(4-isopropyl-benzoyl)-1-(6-methyl-pyridazin-3-yl)-5-(4-[1,2,4]triazol-1-yl-phenyl)-1,5-dihydro-pyrrol-2-one

Prepared from 4-(1H-1,2,4-triazol-1-yl)benzaldehyde, 3-amino-6-methylpyridazine and 2-hydroxy-4-(4-isopropyl-phenyl)-4-oxo-but-2-enoic acid ethyl ester in 38% yield. ¹H-NMR (DMSO-d₆): δ (ppm) 1.18 (d, 6H); 2.5 (s, 3H); 2.92 (quint, 1H); 6.50 (s, 1H); 7.32 (d, 2H); 7.65-7.75 (m, 7H); 8.14 (s, 1H); 8.33 (d, 1H); 9.14 (s, 1H); MS (ESI+): m/z=481 [M+H]⁺; Melting point: 284° C.

### Example 145

### 3-hydroxy-4-(4-isopropyl-benzoyl)-1-(6-methyl-pyridazin-3-yl)-5-(4-pyrazol-1-ylmethyl-phenyl)-1,5-dihydro-pyrrol-2-one

Prepared from 4-(1H-pyrazol-1-ylmethyl)benzaldehyde, 3-amino-6-methylpyridazine and 2-hydroxy-4-(4-isopropyl-phenyl)-4-oxo-but-2-enoic acid ethyl ester in 29% yield. ¹H-NMR (DMSO-d₆): δ (ppm) 1.19 (d, 6H); 2.50 (s, 3H); 2.93 (quint, 1H); 5.18 (s, 2H); 6.19 (s, 1H); 6.43 (s, 1H); 6.95 (d, 2H); 7.30-7.37 (m, 5H); 7.58 (d, 1H); 7.65-7.73 (m, 3H); 8.27 (d, 1H); MS (ESI+): m/z=494 [M+H]⁺; Melting point: 263° C.

### Example 146

### 3-hydroxy-4-(4-isopropyl-benzoyl)-1-(6-methyl-pyridazin-3-yl)-5-(4-[1,2,3]triazol-1-ylmethyl-phenyl)-1,5-dihydro-pyrrol-2-one

Prepared from 4-[1,2,3]triazol-1-ylmethyl-benzaldehyde (intermediate 17), 3-amino-6-methylpyridazine and 2-hydroxy-4-(4-isopropyl-phenyl)-4-oxo-but-2-enoic acid ethyl ester in 25% yield. ¹H-NMR (DMSO-d₆): δ (ppm) 1.20 (d, 6H); 2.50 (q, 3H); 2.93 (quint, 1H); 5.47 (s, 2H); 6.44 (s, 1H); 7.04 (d, 2H); 7.31 (d, 2H); 7.40 (d, 2H); 7.58 (d, 1H); 7.65-7.68 (m, 3H); 8.11 (s, 1H); 8.28 (d, 1H); 11.91 (brs, 1H); MS (ESI+): m/z=495 [M+H]⁺; Melting point: 293° C.

### Example 147

### 3-hydroxy-4-(4-isopropyl-benzoyl)-1-(6-methyl-pyridazin-3-yl)-5-(4-[1,2,3]triazol-2-ylmethyl-phenyl)-1,5-dihydro-pyrrol-2-one

Prepared from 4-[1,2,3]triazol-2-ylmethyl-benzaldehyde (Intermediate 18), 3-amino-6-methylpyridazine and 2-hydroxy-4-(4-isopropyl-phenyl)-4-oxo-but-2-enoic acid ethyl ester in 26% yield. ¹H-NMR (DMSO-d₆): δ (ppm) 1.20 (d, 6H); 2.50 (s, 3H); 2.92 (quint, 1H); 5.50 (s, 2H); 6.44 (s, 1H); 7.02 (d, 2H); 7.32 (d, 2H); 7.38 (d, 2H); 7.58 (d, 1H); 7.66 (d, 2H); 7.73 (s, 2H); 8.28 (d, 1H); 11.89 (brs, 1H); MS (ESI+): m/z=495 [M+H]⁺; Melting point: 285° C.

### Example 148

### 3-hydroxy-5-(4-itnidazol-1-ylmethyl-phenyl)-4-(4-isopropyl-benzoyl)-1-(6-methyl-pyridazin-3-yl)-1,5-dihydro-pyrrol-2-one

Prepared from 4-(1H-imidazol-1-ylmethyl)benzaldehyde, 3-amino-6-methylpyridazine and 2-hydroxy-4-(4-isopropyl-phenyl)-4-oxo-but-2-enoic acid ethyl ester in 41% yield. ¹H-NMR (DMSO-d₆): δ (ppm) 1.18 (d, 6H); 2.50 (s, 3H); 2.91 (quint, 1H); 5.11 (s, 2H); 6.39 (s, 1H); 7.03 (m, 3H); 7.26 (m, 3H); 7.39 (d, 2H); 7.55 (d, 1H); 7.69 (d, 2H); 8.09 (s, 1H); 8.30 (d, 1H); MS (ESI+): m/z=494 [M+H]⁺; Melting point: 222° C.

### Example 150

### 3-hydroxy-4-(4-isopropyl-benzoyl)-1-(6-methyl-pyridazin-3-yl)-5-(4-tetrazol-2-ylmethyl-phenyl)-1,5-dihydro-pyrrol-2-one

Prepared from 4-tetrazol-2-ylmethyl-benzaldehyde (Intermediate 15), 3-amino-6-methylpyridazine and 2-hydroxy-4-(4-isopropyl-phenyl)-4-oxo-but-2-enoic acid ethyl ester in 16% yield. ¹H-NMR (DMSO-d₆): δ (ppm) 1.19 (d, 6H); 2.50 (s, 3H); 2.93 (quint, 1H); 5.81 (s, 2H); 6.44 (s, 1H); 7.13 (d, 2H); 7.31 (d, 2H); 7.43 (d, 2H); 7.58 (d, 1H); 7.66 (d, 2H); 8.29 (d, 1H); 8.90 (s, 1H); MS (ESI+): m/z=496 [M+H]⁺; Melting point: 271° C.

### Example 151

### 3-hydroxy-1-(6-methyl-pyridazin-3-yl)-5-(4-tetrazol-2-ylmethyl-phenyl)-4-(4-trifluoromethoxy-benzoyl)-1,5-dihydro-pyrrol-2-one

Prepared from 4-tetrazol-2-ylmethyl-benzaldehyde (Intermediate 15), 3-amino-6-methylpyridazine and 2-hydroxy-4-oxo-4-(4-trifluoromethoxy-phenyl)-but-2-enoic acid ethyl ester in 14% yield. ¹H-NMR (DMSO-d₆): δ (ppm) 2.50 (s, 3H); 5.81 (s, 2H); 6.40 (s, 1H); 7.12 (d, 2H); 7.35-7.43 (m, 4H); 7.55 (d, 1H); 7.84 (d, 2H); 8.31 (d, 1H); 8.91 (s, 1H); MS (ESI+): m/z=538 [M+H]⁺; Melting point: 258° C.

### Example 152

### 3-hydroxy-1-(6-methyl-pyridazin-3-yl)-5-(4-[1,2,3]triazol-1-ylmethyl-phenyl)-4-(4-trifluoromethoxy-benzoyl)-1,5-dihydro-pyrrol-2-one

Prepared from 4-[1,2,3]triazol-1-ylmethyl-benzaldehyde (Intermediate 17), 3-amino-6-methylpyridazine and 2-hydroxy-4-oxo-4-(4-trifluoromethoxy-phenyl)-but-2-enoic acid ethyl ester in 21% yield. ¹H-NMR (DMSO-d₆): δ (ppm) 2.50 (s, 3H); 5.47 (s, 2H); 6.41 (s, 1H); 7.04 (d, 2H); 7.37-7.42 (m, 4H); 7.55 (d, 1H); 7.68 (brs, 1H); 7.84 (d, 2H); 8.11 (brs, 1H); 8.29 (d, 1H); MS (ESI+): m/z=537 [M+H]⁺; Melting point: 275° C.

### Example 153

### 3-hydroxy-1-(6-methyl-pyridazin-3-yl)-5-(4-[1,2,3]triazol-2-ylmethyl-phenyl)-4-(4-trifluoromethoxy-benzoyl)-1,5-dihydro-pyrrol-2-one

Prepared from 4-[1,2,3]triazol-2-ylmethyl-benzaldehyde (Intermediate 18), 3-amino-6-methylpyridazine and 2-hydroxy-4-oxo-4-(4-trifluoromethoxy-phenyl)-but-2-enoic acid ethyl ester in 15% yield. ¹H-NMR (DMSO-d₆): δ (ppm) 2.50 (s, 3H); 5.50 (s, 2H); 6.40 (s, 1H); 7.02 (d, 2H); 7.37-7.40 (d, 4H); 7.56 (d, 1H); 7.74 (brs, 2H); 7.84 (d, 2H); 8.28 (d, 1H); MS (ESI+): m/z=537 [M+H]⁺; Melting point: 277° C.

### Example 154

### 3-hydroxy-1-(6-methyl-pyridazin-3-yl)-5-(4-tetrazol-1-ylmethyl-phenyl)-4-(4-trifluoromethoxy-benzoyl)-1,5-dihydro-pyrrol-2-one

Prepared from 4-tetrazol-1-ylmethyl-benzaldehyde (Intermediate 14), 3-amino-6-methylpyridazine and 2-hydroxy-4-oxo-4-(4-trifluoromethoxy-phenyl)-but-2-enoic acid ethyl ester in 11% yield. ¹H-NMR (DMSO-d₆): δ (ppm) 2.50 (s, 3H); 5.57 (s, 2H); 6.40 (s, 1H); 7.10 (d, 2H); 7.36-7.44 (m, 4H); 7.55 (d, 1H); 7.84 (d, 2H); 8.30 (d, 1H); 9.45 (d, 1H); MS (ESI+): m/z=538 [M+H]⁺; Melting point>300° C.

### Example 156

### 3-hydroxy-1-(6-methyl-pyridazin-3-yl)-5-(4-[1,2,4]triazol-1-ylmethyl-phenyl)-4-(4-trifluoromethoxy-benzoyl)-1,5-dihydro-pyrrol-2-one

Prepared from 4-(1H-1,2,4-triazol-1-yl)benzaldehyde, 3-amino-6-methylpyridazine and 2-hydroxy-4-oxo-4-(4-trifluoromethoxy-phenyl)-but-2-enoic acid ethyl ester in 8% yield. ¹H-NMR (DMSO-d₆): δ (ppm) 2.50 (s, 3H); 5.28 (s, 2H); 6.44 (s, 1H); 7.04 (d, 2H); 7.41-7.45 (m, 4H); 7.60 (d, 1H); 7.83 (d, 2H); 7.91 (s, 1H); 8.27 (d, 1H); 8.58 (s, 1H); MS (ESI+): m/z=537 [M+H]⁺; Melting point: 224° C.

### Example 157

### 3-hydroxy-4-(4-isopropyl-benzoyl)-5-[4-(1-methyl-1H-pyrazol-3-yl)-phenyl]-1-(6-methyl-pyridazin-3-yl)-1,5-dihydro-pyrrol-2-one

Prepared from 4-(1-methyl-1H-pyrazol-3-yl)benzaldehyde, 3-amino-6-methylpyridazine and 2-hydroxy-4-(4-isopropyl-phenyl)-4-oxo-but-2-enoic acid ethyl ester in 28% yield. ¹H-NMR (DMSO-d₆): δ (ppm) 1.18 (d, 6H); 2.50 (s, 3H); 2.92 (quint, 1H); 3.79 (s, 3H); 6.46 (s, 1H); 6.54 (s, 1H); 7.31-7.40 (m, 4H); 7.55-7.70 (m, 6H); 8.31 (d, 1H); MS (ESI+): m/z=494 [M+H]⁺

### Example 158

### 5-[4-(1,1-difluoro-ethyl)phenyl]-3-hydroxy-4-(4-isopropyl-benzoyl)-1-(6-methyl-pyridazin-3-yl)-1,5-dihydro-pyrrol-2-one

Prepared from 4-(1,1-difluoro-ethyl)-benzaldehyde, 3-amino-6-methylpyridazine and 2-hydroxy-4-(4-isopropyl-phenyl)-4-oxo-but-2-enoic acid ethyl ester in 34% yield. ¹H-NMR (DMSO-d₆): δ (ppm) 1.18 (d, 6H); 1.84 (t, 3H). 2.50 (s, 3H); 2.92 (quint, 1H); 6.49 (s, 1H); 7.30-7.39 (m, 4H); 7.53 (d, 2H); 7.60 (d, 1H); 7.67 (d, 2H); 8.33 (d, 1H); MS (ESI+): m/z=478 [M+H]⁺

### Example 159

### 3-hydroxy-1-(6-hydroxy-pyridazin-3-yl)-4-(4-isopropyl-benzoyl)-5-(4-trifluoromethoxy-phenyl)-1,5-dihydro-pyrrol-2-one

Prepared from 4-(trifluoromethoxy)benzaldehyde, 6-amino-pyridazin-3-ol and 2-hydroxy-4-(4-isopropyl-phenyl)-4-oxo-but-2-enoic acid ethyl ester in 52% yield. ¹H-NMR (DMSO-d₆): δ (ppm) 1.19 (d, 6H); 2.92 (quint, 1H); 6.09 (s, 1H); 6.97 (d, 1H); 7.23-7.33 (m, 4H); 7.51-7.68 (m, 4H); 8.13 (d, 1H); 12.82 (brs, 1H); MS (ESI+): m/z=500 [M+H]⁺; Melting point: 255° C.

### Example 160

### 3-hydroxy-5-(4-isopropenyl-phenyl)-4-(4-isopropyl-benzoyl)-1-(6-methyl-pyridazin-3-yl)-1,5-dihydro-pyrrol-2-one

Prepared from 4-isopropenyl-benzaldehyde, 3-amino-6-methylpyridazine and 2-hydroxy-4-(4-isopropyl-phenyl)-4-oxo-but-2-enoic acid ethyl ester in 9% yield. ¹H-NMR (DMSO-d₆): δ (ppm) 1.19 (d, 6H); 1.97 (s, 3H); 2.50 (s, 3H); 2.93 (quint, 1H); 4.99 (s, 1H); 5.30 (s, 1H); 6.45 (s, 1H); 7.28-7.45 (m, 6H), 7.59 (d, 1H); 7.69 (d, 2H); 8.31 (d, 1H); 11.92 (brs, 1H); MS (ESI+): m/z=454 [M+H]⁺; Melting point: 274° C.

### Example 161

### 1-(6-benzyloxy-pyridazin-3-yl)-3-hydroxy-4-(4-isopropyl-benzoyl)-5-(4-trifluoromethoxy-phenyl)-1,5-dihydro-pyrrol-2-one

Prepared from 4-(trifluoromethoxy)benzaldehyde, 6-benzyloxy-pyridazin-3-ylamine and 2-hydroxy-4-(4-isopropyl-phenyl)-4-oxo-but-2-enoic acid ethyl ester in 34% yield. ¹H-NMR (DMSO-d₆): δ (ppm) 1.19 (d, 6H); 2.93 (quint, 1H); 5.40 (dd, 2H); 6.42 (s, 1H); 7.20 (d, 2H); 7.31-7.43 (m, 8H); 7.57 (d, 2H); 7.68 (d, 2H); 8.40 (d, 1H); MS (ESI+): m/z=590 [M+H]⁺; Melting point: 269° C.

### Example 162

### 3-hydroxy-1-(6-methyl-pyridazin-3-yl)-5-(4-trifluoromethoxy-phenyl)-4-(4-trifluoromethyl-benzoyl)-1,5-dihydro-pyrrol-2-one

Prepared from 4-(trifluoromethoxy)benzaldehyde, 3-amino-6-methylpyridazine and 2-hydroxy-4-oxo-4-(4-trifluoromethoxy-phenyl)-but-2-enoic acid ethyl ester in 26% yield. ¹H-NMR (DMSO-d₆): δ (ppm) 2.50 (s, 3H); 6.46 (s, 1H); 7.17 (d, 2H); 7.60 (m, 3H); 7.84 (d, 4H), 8.32 (d, 1H); MS (ESI+): m/z=524 [M+H]⁺

### Example 163

### 3-hydroxy-4-(4-isopropyl-benzoyl)-1-(6-methyl-pyridazin-3-yl)-5-[4-(2H-tetrazol-5-yl)-phenyl]-1,5-dihydro-pyrrol-2-one

Prepared from 4-(2H-tetrazol-5-yl)-benzaldehyde, 3-amino-6-methylpyridazine and 2-hydroxy-4-(4-isopropyl-phenyl)-4-oxo-but-2-enoic acid ethyl ester in 6% yield. ¹H-NMR (DMSO-d₆): δ (ppm) 1.18 (d, 6H); 2.38 (s, 3H); 2.91 (quint, 1H); 6.47 (s, 1H); 7.00 (d, 1H); 7.28 (d, 1H); 7.39 (d, 1H); 7.57-7.72 (m, 4H); 7.83 (d, 2H); 8.37 (d, 1H); MS (ESI+): m/z=482 [M+H]⁺

### Example 164

### 3-hydroxy-4-(4-isopropyl-benzoyl)-1-(6-methyl-pyridazin-3-yl)-5-[4-(pyridin-2-yloxy)-phenyl]-1,5-dihydro-pyrrol-2-one

Prepared from 4-(pyrid-2-yloxy)benzaldehyde, 3-amino-6-methylpyridazine and 2-hydroxy-4-(4-isopropyl-phenyl)-4-oxo-but-2-enoic acid ethyl ester in 25% yield. ¹H-NMR (DMSO-d₆): δ (ppm) 1.21 (d, 6H); 2.50 (s, 3H); 2.95 (quint, 1H); 6.49 (s, 1H); 6.94 (d, 3H); 7.08 (s, 1H); 7.35 (d, 2H); 7.43 (d, 2H); 7.63 (d, 1H); 7.72-7.79 (m, 3H); 8.08 (brs, 1H); 8.32 (d, 1H); 11.89 (brs, 1H); MS (ESI+): m/z=507 [M+H]⁺; Melting point: 280° C.

### Example 165

### 3-hydroxy-5-(6-isopropoxy-pyridin-3-yl)-4-(4-isopropyl-benzoyl)-1-(6-methyl-pyridazin-3-yl)-1,5-dihydro-pyrrol-2-one

Prepared from 6-isopropoxy-pyridine-3-carbaldehyde, 3-amino-6-methylpyridazine and 2-hydroxy-4-(4-isopropyl-phenyl)-4-oxo-but-2-enoic acid ethyl ester in 35% yield. ¹H-NMR (DMSO-d₆): δ (ppm) 1.13-1.26 (m, 12H); 2.50 (s, 3H); 2.93 (quint, 1H); 5.08 (quint, 1H); 6.39 (s, 1H); 6.51 (d, 1H); 7.33 (d, 2H); 7.58-7.74 (m, 4H); 8.21 (brs, 1H); 8.29 (d, 1H); MS (ESI+): m/z=473 [M+H]⁺; Melting point: 282° C.

### Example 166

### 3-hydroxy-4-(4-isopropyl-benzoyl)-5-[4-(5-methyl-[1,3,4]oxadiazol-2-yl)-phenyl]-1-(6-methyl-pyridazin-3-yl)-1,5-dihydro-pyrrol-2-one

Prepared from 4-(5-methyl-1,3,4-oxadiazol-2-yl)benzaldehyde, 3-amino-6-methylpyridazine and 2-hydroxy-4-(4-isopropyl-phenyl)-4-oxo-but-2-enoic acid ethyl ester in 19% yield. ¹H-NMR (DMSO-d₆): δ (ppm) 1.18 (d, 6H); 2.50 (s, 6H); 2.92 (quint, 1H); 6.50 (s, 1H); 7.32 (d, 2H); 7.59-7.63 (m, 3H); 7.68 (d, 2H); 7.78 (d, 2H); 8.37 (d, 1H); MS (ESI+): m/z=496 [M+H]⁺; Melting point: 293° C.

### Example 168

### 3-hydroxy-1-(6-methyl-pyridazin-3-yl)-5-[4-(2H-tetrazol-5-yl)-phenyl]-4-(4-trifluoromethoxy-benzoyl)-1,5-dihydro-pyrrol-2-one

Prepared from 4-(2H-tetrazol-5-yl)-benzaldehyde, 3-amino-6-methylpyridazine and 2-hydroxy-4-oxo-4-(4-trifluoromethoxy-phenyl)-but-2-enoic acid ethyl ester in 4% yield. ¹H-NMR (DMSO-d₆): δ (ppm) 2.50 (s, 3H); 6.50 (s, 1H); 7.11 (t, 1H); 7.44 (d, 2H); 7.58-7.69 (m, 3H); 7.86 (d, 4H); 8.35 (d, 1H); MS (ESI+): m/z=524 [M+H]⁺

### Example 169

### 3-hydroxy-5-(6-isopropoxy-pyridin-3-yl)-1-(6-methyl-pyridazin-3-yl)-4-(4-trifluoromethoxy-benzoyl)-1,5-dihydro-pyrrol-2-one

Prepared from 6-isopropoxy-pyridine-3-carbaldehyde, 3-amino-6-methylpyridazine and 2-hydroxy-4-oxo-4-(4-trifluoromethoxy-phenyl)-but-2-enoic acid ethyl ester in 2% yield. ¹H-NMR (DMSO-d₆): δ (ppm) 1.18 (d, 6H); 2.50 (s, 3H); 5.09 (quint, 1H); 6.36 (s, 1H); 6.51 (d, 1H); 7.11 (t, 1H); 7.40 (d, 2H); 7.59 (d, 1H); 7.66 (d, 1H); 7.89 (d, 2H); 8.22 (brs, 1H); 8.30 (d, 1H); MS (ESI+): m/z=515 [M+H]⁺

### Example 170

### 5-[4-(1-benzyl-1H-tetrazol-5-yloxy)-phenyl]-3-hydroxy-4-(4-isopropyl-benzoyl)-1-(6-methyl-pyridazin-3-yl)-1,5-dihydro-pyrrol-2-one

Prepared from 4-(1-benzyl-1H-tetrazol-5-yloxy)-benzaldehyde (Intermediate 19), 3-amino-6-methylpyridazine and 2-hydroxy-4-(4-isopropyl-phenyl)-4-oxo-but-2-enoic acid ethyl ester in 33% yield. ¹H-NMR (DMSO-d₆): δ (ppm) 1.20 (d, 6H); 2.50 (s, 3H); 2.93 (quint, 1H); 5.47 (s, 2H); 6.48 (s, 1H); 7.21 (d, 2H); 7.31-7.35 (m, 7H); 7.53 (d, 2H); 7.61 (d, 1H); 7.71 (d, 2H); 8.32 (d, 1H); 12.02 (brs, 1H); MS (ESI+): m/z=588 [M+H]⁺

### Example 172

### 5-(4-bromo-phenyl)-3-hydroxy-4-(4-isopropyl-benzoyl)-1-(6-methyl-pyridazin-3-yl)-1,5-dihydro-pyrrol-2-one

Prepared from 4-bromobenzaldehyde, 3-amino-6-methylpyridazine and 2-hydroxy-4-(4-isopropyl-phenyl)-4-oxo-but-2-enoic acid ethyl ester in 30% yield. ¹H-NMR (DMSO-d₆): δ (ppm) 1.19 (d, 6H); 2.50 (s, 3H); 2.93 (quint, 1H); 6.41 (s, 1H); 7.30-7.36 (m, 6H); 7.60 (d, 1H); 7.67 (d, 2H); 8.32 (d, 1H); 11.93 (brs, 1H); MS (ESI+): m/z=493 [M+H]⁺

### Example 173

### 3-hydroxy-4-(4-isopropyl-benzoyl)-5-(4-isopropylsulfanyl-phenyl)-1-(6-methyl-pyridazin-3-yl)-1,5-dihydro-pyrrol-2-one

Prepared from 4-isopropylsulfanyl-benzaldehyde, 3-amino-6-methylpyridazine and 2-hydroxy-4-(4-isopropyl-phenyl)-4-oxo-but-2-enoic acid ethyl ester in 27% yield. ¹H-NMR (DMSO-d₆): δ (ppm) 1.12 (d, 6H); 1.19 (d, 6H); 2.50 (s, 3H); 2.93 (quint, 1H); 3.37 (quint, 1H); 6.42 (s, 1H); 7.13 (d, 2H); 7.33 (d, 4H); 7.60 (d, 1H); 7.68 (d, 2H); 8.30 (d, 1H); 11.94 (brs, 1H); MS (ESI+): m/z=488 [M+H]⁺

### Example 174

### 5-[4-(1,1-difluoro-ethoxy)-phenyl]-3-hydroxy-4-(4-isopropyl-benzoyl)-1-(6-methyl-pyridazin-3-yl)-1,5-dihydro-pyrrol-2-one

Prepared from 4-(1,1-difluoro-ethoxy)-benzaldehyde (Intermediate 20), 3-amino-6-methylpyridazine and 2-hydroxy-4-(4-isopropyl-phenyl)-4-oxo-but-2-enoic acid ethyl ester in 26% yield. ¹H-NMR (DMSO-d₆): δ (ppm) 1.19 (d, 6H); 2.48 (s, 3H); 2.92 (quint, 1H); 4.16 (dt, 2H); 6.26 (t, 1H); 6.41 (s, 1H); 6.79 (d, 2H); 7.32 (d, 4H); 7.58 (d, 1H); 7.68 (d, 2H); 8.26 (d, 1H); 11.85 (brs, 1H); MS (ESI+): m/z=494 [M+H]⁺

### Example 175

### 4-[2-[4-(1,1-difluoro-ethyl)-phenyl]-4-hydroxy-1-(6-methyl-pyridazin-3-yl)-5-oxo-2,5-dihydro-1H-pyrrole-3-carbonyl]-benzonitrile

Prepared from 4-(1,1-difluoro-ethyl)-benzaldehyde, 3-amino-6-methylpyridazine and 4-(4-cyano-phenyl)-2,4-dioxo-butyric acid ethyl ester in 24% yield. ¹H-NMR (DMSO-d₆): δ (ppm) 1.86 (t, 3H); 2.50 (s, 3H); 6.46 (s, 1H); 7.37 (d, 2H); 7.53-7.61 (m, 3H); 7.86 (dd, 4H); 8.32 (d, 1H); MS (ESI+): m/z=461 [M+H]⁺

### Example 176

### 4-[4-hydroxy-1-(6-methyl-pyridazin-3-yl)-5-oxo-2-(4-trifluoromethoxy-phenyl)-2,5-dihydro-1H-pyrrole-3-carbonyl]-benzonitrile

Prepared from 4-(trifluoromethoxy)benzaldehyde, 3-amino-6-methylpyridazine and 4-(4-cyano-phenyl)-2,4-dioxo-butyric acid ethyl ester in 10% yield. ¹H-NMR (DMSO-d₆): δ(ppm) 2.50 (s, 3H); 6.46 (s, 1H); 7.18 (d, 2H); 7.58-7.61 (m, 3H); 7.88 (dd, 4H); 8.32 (d, 1H); MS (ESI+): m/z=481 [M+H]⁺

### Example 177

### 3-hydroxy-4-(4-methanesulfonyl-benzoyl)-1-(6-methyl-pyridazin-3-yl)-5-(4-trifluoromethoxy-phenyl)-1,5-dihydro-pyrrol-2-one

Prepared from 4-(trifluoromethoxy)benzaldehyde, 3-amino-6-methylpyridazine and 4-(4-methanesulfonyl-phenyl)-2,4-dioxo-butyric acid ethyl ester (Intermediate 21) in 13% yield. ¹H-NMR (DMSO-d₆): δ(ppm) 2.50 (s, 3H); 3.27 (s, 3H); 6.49 (s, ¹H); 7.20 (d, 2H); 7.60-7.65 (m, 3H); 7.97 (dd, 4H); 8.34 (d, 1H); MS (ESI+): m/z=534 [M+H]⁺

### Example 178

### 5-[4-(1,1-difluoro-ethyl)phenyl]-3-hydroxy-4-(4-isopropyl-benzoyl)-1-(5-methyl-pyrimidin-2-yl)-1,5-dihydro-pyrrol-2-one

Prepared from 4-(1,1-difluoro-ethyl)-benzaldehyde, 2-amino-5-methylpyrimidine and 2-hydroxy-4-(4-isopropyl-phenyl)-4-oxo-but-2-enoic acid ethyl ester in 3% yield. ¹H-NMR (DMSO-d₆): δ (ppm) 1.19 (d, 6H); 1.84 (t, 3H), 2.17 (s, 3H); 2.92 (quint, 1H); 6.32 (s, 1H); 7.32 (d, 2H); 7.39 (d, 2H); 7.49 (d, 2H); 7.68 (d, 2H); 8.54 (s, 2H); MS (ESI+): m/z=478 [M+H]⁺

### Example 179

### 5-(6-dimethylamino-pyridin-3-yl)-3-hydroxy-4-(4-isopropyl-benzoyl)-1-(6-methyl-pyridazin-3-yl)-1,5-dihydro-pyrrol-2-one

Prepared 6-(dimethylamino)nicotinaldehyde, 3-amino-6-methylpyridazine and 2-hydroxy-4-(4-isopropyl-phenyl)-4-oxo-but-2-enoic acid ethyl ester in 14% yield. ¹H-NMR (DMSO-d₆): δ (ppm) 1.21 (d, 6H); 2.50 (s, 3H); 2.88 (s, 6H); 2.94 (quint, 1H); 6.33 (s, 1H); 6.42 (d, 1H); 7.34 (d, 2H); 7.50 (d, 1H); 7.59 (d, 1H); 7.74 (d, 2H); 8.12 (brs, 1H); 8.25 (d, 1H); MS (ESI+): m/z=458 [M+H]⁺

### Example 180

### 3-hydroxy-4-(4-isopropyl-benzoyl)-1-(5-methyl-pyrimidin-2-yl)-5-(4-trifluoromethoxy-phenyl)-1,5-dihydro-pyrrol-2-one

Prepared from 4-(trifluoromethoxy)benzaldehyde, 2-amino-5-methylpyrimidine and 2-hydroxy-4-(4-isopropyl-phenyl)-4-oxo-but-2-enoic acid ethyl ester in 17% yield. ¹H-NMR (DMSO-d₆): δ (ppm) 1.27 (d, 6H); 2.24 (s, 3H); 3.00 (quint, 1H); 6.37 (s, 1H); 7.25 (d, 2H); 7.39 (d, 2H); 7.57 (d, 2H); 7.74 (d, 2H); 8.61 (s, 2H); 11.94 (brs, 1H); MS (ESI+): m/z=498 [M+H]⁺

### Example 181

### 5-[4-(1,1-difluoro-ethyl)-phenyl]-3-hydroxy-4-(4-methanesulfonyl-benzoyl)-1-(6-methyl-pyridazin-3-yl)-1,5-dihydro-pyrrol-2-one

Prepared from 4-(1,1-difluoro-ethyl)benzaldehyde, 3-amino-6-methylpyridazine and 4-(4-methanesulfonyl-phenyl)-2,4-dioxo-butyric acid ethyl ester (Intermediate 21) in 4% yield. ¹H-NMR (DMSO-d₆): δ (ppm) 1.86 (t, 3H); 2.50 (s, 3H); 3.27 (s, 3H); 6.50 (s, 1H); 7.42 (d, 2H); 7.57-7.64 (m, 3H); 7.93 (d, 2H); 8.01 (d, 2H); 8.32 (d, 1H); MS (ESI+): m/z=514 [M+H]⁺

### Example 183

### 5-[4-(1,1-difluoro-ethoxy)-phenyl]-3-hydroxy-1-(6-methyl-pyridazin-3-yl)-4-(4-pyrrolidin-1-yl-benzoyl)-1,5-dihydro-pyrrol-2-one

Prepared from 4-(1,1-difluoro-ethyl)benzaldehyde, 3-amino-6-methylpyridazine and 2,4-dioxo-4-(4-pyrrolidin-1-yl-phenyl)-butyric acid ethyl ester (Intermediate 22) in 18% yield. ¹H-NMR (DMSO-d₆): δ (ppm) 1.84 (t, 3H); 1.91-1.94 (m, 4H); 2.50 (s, 3H); 3.28-3.32 (m, 4H); 6.47-6.52 (m, 3H); 7.37 (dd, 4H); 7.62 (t, 3H); 8.34 (d, 1H); 1.44 (brs, 1H); MS (ESI+): m/z=521 [M+H]⁺

### Example 184

### 3-hydroxy-1-(6-methyl-pyridazin-3-yl)-4-(4-pyrrolidin-1-yl-benzoyl)-5-(4-trifluoromethoxy-phenyl)-1,5-dihydro-pyrrol-2-one

Prepared from 4-(trifluoromethoxy)benzaldehyde, 3-amino-6-methylpyridazine and 2,4-dioxo-4-(4-pyrrolidin-1-yl-phenyl)-butyric acid ethyl ester (Intermediate 22) in 12% yield. ¹H-NMR (DMSO-d₆): δ (ppm) 1.94 (m, 4H); 2.50 (s, 3H); 3.28-3.32 (m, 4H); 6.48 (m, 3H); 7.17 (d, 2H); 7.49 (d, 2H); 7.62 (t, 3H); 8.34 (d, 1H); MS (ESI+): m/z=525 [M+H]⁺

### Example 185

### 3-hydroxy-4-(4-isopropyl-benzoyl)-5-[4-(5-methyl-isoxazol-3-yloxy)-phenyl]-1-(6-methyl-pyridazin-3-yl)-1,5-dihydro-pyrrol-2-one

Prepared from 4-(5-methyl-isoxazol-3-yloxy)benzaldehyde (Intermediate 23), 3-amino-6-methylpyridazine and 2-hydroxy-4-(4-isopropyl-phenyl)-4-oxo-but-2-enoic acid ethyl ester in 34% yield. ¹H-NMR (DMSO-d₆): δ (ppm) 1.20 (d, 6H); 2.31 (s, 3H); 2.52 (s, 3H); 2.94 (quint, 1H); 6.04 (s, 1H); 6.47 (s, 1H); 7.05 (d, 2H); 7.34 (d, 2H); 7.46 (d, 2H); 7.61 (d, 1H); 7.70 (d, 2H); 8.31 (d, 1H); 11.94 (brs, 1H); MS (ESI+): m/z=511 [M+H]⁺

### Example 187

### 5-[6-(1,1-difluoro-ethyl)-pyridin-3-yl]-3-hydroxy-4-(4-isopropyl-benzoyl)-1-(6-methyl-pyridazin-3-yl)-1,5-dihydro-pyrrol-2-one

Prepared from 6-(1,1-difluoro-ethyl)-pyridine-3-carbaldehyde, 3-amino-6-methylpyridazine and 2-hydroxy-4-(4-isopropyl-phenyl)-4-oxo-but-2-enoic acid ethyl ester in 22% yield. ¹H-NMR (DMSO-d₆): δ (ppm) 1.19 (d, 6H); 1.89 (t, 3H); 2.50 (s, 3H); 2.92 (quint, 1H); 6.49 (s, 1H); 7.32 (d, 2H); 7.52 (d, 1H); 7.62 (d, 1H); 7.71 (d, 2H); 8.04 (d, 1H); 8.38 (d, 1H); 8.77 (brs, 1H), MS (ESI+): m/z=479 [M+H]⁺

### Example 200

### 3-hydroxy-4-(4-isopropyl-benzoyl)-1-(6-methyl-pyridazin-3-yl)-5-{4-[1-(2-trimethylsilanyl-ethoxymethyl)-1H-[1,2,4]triazol-3-yloxy]-phenyl}-1,5-dihydro-pyrrol-2-one

Prepared, following procedure C, from 4-[1-(2-trimethylsilanyl-ethoxymethyl)-1H-[1,2,4]triazol-3-yloxy]-benzaldehyde (Intermediate 25), 3-amino-6-methylpyridazine and 2-hydroxy-4-(4-isopropyl-phenyl)-4-oxo-but-2-enoic acid ethyl ester in 8% yield. ¹H-NMR (DMSO-d₆): δ (ppm)-0.14 (brs, 9H); 0.79 (t; 2H); 1.20 (d, 6H); 2.50 (s, 3H); 2.94 (quint, 1H); 3.55 (t, 2H); 5.32 (s, 2H); 6.49 (s, 1H); 7.15 (d, 2H); 7.34 (d, 2H); 7.49 (d, 2H); 7.59-7.72 (m, 4H); 8.31 (1H); MS (ESI+): m/z=627 [M+H]⁺

### General Procedure D: Oximes Formation:

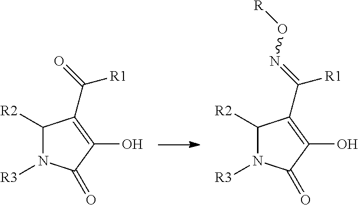

(R1, R2 R3 and R are as defined above).

To a solution of the relevant pyrrolidinone in pyridine (10 ml/mmol) was added oxime (1:1 by mass). The solution was stirred for 2 h or 4 h at 100° C. in micro-waves apparatus. The mixture was concentrated under vacuum. The crude product was diluted with water and extracted twice with ethyl acetate. The organic layers were combined, dried on anhydrous MgSO₄, filtered and concentrated in vacuum. The residue was purified by flash chromatography (silica gel) and it was triturated with Et₂O/MeOH (99/1). The following compounds were prepared according general procedure D:

### Example 53

### 3-hydroxy-5-(4-isopropyl-phenyl)-4-(methoxyimino-p-tolyl-methyl)-1-(6-methyl-pyridazin-3-yl)-1,5-dihydro-pyrrol-2-one

Prepared from 3-hydroxy-5-(4-isopropyl-phenyl)-4-(4-methyl-benzoyl)-1-(6-methyl-pyridazin-3-yl)-1,5-dihydro-pyrrol-2-one (Example 8) and methylhydroxylamine hydrochloride as E/Z mixture in 30% yield. ¹H-NMR (DMSO-d₆): δ (ppm) 1.08 (d, 6H); 2.42 (dd, 3H); 2.45 (dd, 3H); 2.69-2.75 (m, 1H); 3.48 (s, 3H); 5.94 (s, 1H); 6.46-6.49 (d, 2H); 6.87-6.9 (d, 2H); 7.05-7.08 (d, 2H); 7.25-7.28 (d, 2H); 7.50-7.54 (dd, 1H); 8.24-8.27 (dd, 1H); 13.93 (brs, 1H); MS (ESI+): m/z=457 [M+H]⁺

### Example 54

### 3-hydroxy-4-(hydroxyimino-p-tolyl-methyl)-5-(4-isopropyl-phenyl)-1-(6-methyl-pyridazin-3-yl)-1,5-dihydro-pyrrol-2-one

Prepared from 3-hydroxy-5-(4-isopropyl-phenyl)-4-(4-methyl-benzoyl)-1-(6-methyl-pyridazin-3-yl)-1,5-dihydro-pyrrol-2-one (Example 8) and hydroxylamine hydrochloride as E/Z mixture in 56% yield. ¹H-NMR (DMSO-d₆): δ (ppm) 1.16 (d, 6H); 2.37 (s, 3H); 2.48 (s, 3H); 2.82-2.87 (m, 1H); 4.25-4.27 (d, 1H); 5.64-5.65 (d, 1H); 7.24-7.35 (m, 6H); 7.45-7.48 (d, 2H); 7.56-7.6 (d, 1H); 8.32 (d, 1H); 8.82 (s, 1H); MS (ESI+): m/z=443 [M+H]⁺

### Example 55

### [1-[4-Hydroxy-2-(4-isopropyl-phenyl)-1-(6-methyl-pyridazin-3-yl)-5-oxo-2,5-dihydro-1H-pyrrol-3-yl]-1-(4-isopropyl-phenyl)-meth-ylideneaminooxy]-acetic acid

Prepared from 3-hydroxy-4-(4-isopropyl-benzoyl)-5-(4-isopropyl-phenyl)-1-(6-methyl-pyridazin-3-yl)-1,5-dihydro-pyrrol-2-one (Example 20) and carboxymethoxylamine hemihydrochloride in 28% yield. ¹H-NMR (DMSO-d₆): δ (ppm) 1.05 (d, 6H); 1.13 (d, 6H); 2.50 (s, 3H); 2.66-2.90 (m, 2H); 4.80 (s, 2H); 6.71 (s, 1H); 7.00-7.23 (m, 9H); 7.61 (d, 1H); 8.39 (d, 1H); MS (ESI+): m/z=529 [M+H]⁺; Melting point: 234° C.

### Example 83

### [[4-Hydroxy-1-(6-methyl-pyridazin-3-yl)-5-oxo-2-(4-trifluoromethoxy-phenyl)-2,5-dihydro-1H-pyrrol-3-yl]-(4-isopropyl-phenyl)-methyleneaminooxy]-acetic acid

Prepared from 3-hydroxy-4-(4-isopropyl-benzoyl)-1-(6-methyl-pyridazin-3-yl)-5-(4-trifluoromethoxy-phenyl)-1,5-dihydro-pyrrol-2-one (Example 61) and carboxymethoxylamine hemihydrochloride in 65% as a E/Z mixture. Note: after concentration in vacuum, the residue was triturated in Et₂O and after filtration the solid was washed with Et₂O to give the desired compound; ¹H-NMR (DMSO-d₆): δ (ppm) 1.16 (2d, 6H); 2.50 (2s, 3H); 2.83 (m, 1H); 4.59 (s, 0.5H); 4.82 (s, 1.4H); 6.33 (s, 0.2H); 6.76 (s, 0.8H); 7.08-7.21 (m, 6H); 7.45 (d, 2H); 7.61 (2d, 1H); 8.38 (2d, 1H); MS (ESI+): m/z=571 [M+H]⁺; Melting point: 227° C.

### Example 84

### 3-Hydroxy-4-{(4-isopropyl-phenyl)-[2-methoxy-ethoxyimino]-methyl}-1-(6-methyl-pyridazin-3-yl)-5-(4-trifluoromethoxy-phenyl)-1,5-dihydro-pyrrol-2-one

Prepared from 3-hydroxy-4-(4-isopropyl-benzoyl)-1-(6-methyl-pyridazin-3-yl)-5-(4-trifluoromethoxy-phenyl)-1,5-dihydro-pyrrol-2-one (Example 61) and O-(2-methoxy-ethyl)-hydroxylamine in 31% as a E/Z mixture; ¹H-NMR (DMSO-d₆): δ (ppm) 1.13 (d, 4H); 1.19 (d, 2H); 2.82 (m, 1H); 3.36 (s, 3H); 3.42-3.50 (m, 0.66H); 3.64-3.71 (m, 1.46H); 4.09 (m, 0.59H); 4.33 (t, 1.45H); 6.35 (s, 0.23H); 6.74 (s, 0.64H); 7.09-7.22 (m, 6H); 7.44 (dd, 2H); 7.58 (dd, 1H); 8.34 (dd, 1H); 10.75 (brs, 1H); MS (ESI+): m/z=571 [M+H]⁺; Melting point: 115-118° C.

### General Procedure E: Acetylation

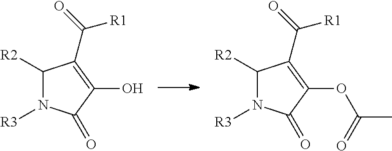

(R1, R2 and R3 are as defined above).

To a solution of the starting material (1 eq) in dry dichloromethane (9 ml/mmol) and under an atmosphere of nitrogen was added acetic anhydride (1.2 eq) and pyridine (1.5 eq). The solution was stirred for 6 h30 at room temperature. The mixture was concentrated under vacuum and the residue was triturated in Et₂O then filtered to give the titled compound. The following compounds were prepared according general procedure E:

### Example 56

### Acetic acid 4-(4-isopropyl-benzoyl)-5-(4-isopropyl-phenyl)-1-(6-methyl-pyridazin-3-yl)-2-oxo-2,5-hydro-1H-pyrrol-3-yl ester

Prepared from 3-hydroxy-4-(4-isopropyl-benzoyl)-5-(4-isopropyl-phenyl)-1-(6-methyl-pyridazin-3-yl)-1,5-dihydro-pyrrol-2-one (Example 20) and acetic anhydride in 49% yield. ¹H-NMR (DMSO-d₆): δ (ppm) 1.04 (d, 6H); 1.18 (d, 6H); 1.99 (s, 3H); 2.5 (s, 3H); 2.72 (quint, 1H); 2.95 (quint, 1H); 6.65 (s, 1H); 7.10 (d, 2H); 7.28 (d, 2H); 7.38 (d, 2H); 7.59-7.64 (m, 3H); 8.26 (d, 1H); MS (ESI+): m/z=498 [M+H]⁺; Melting point: 215° C.

### Example 76

### Acetic acid 4-(4-methyl-benzoyl)-1-(6-methyl-pyridazin-3-yl)-2-oxo-5-(4-trifluoromethoxy-phenyl)-2,5-dihydro-1H-pyrrol-3-yl ester

Prepared from 3-hydroxy-4-(4-methyl-benzoyl)-1-(6-methyl-pyridazin-3-yl)-5-(4-trifluoromethoxy-phenyl)-1,5-dihydro-pyrrol-2-one (Example 22) in 60% yield; ¹H-NMR (DMSO-d₆): δ(ppm) 2.04 (s, 3H); 2.37 (s, 3H); 2.50 (s, 3H); 6.71 (s, 1H); 7.23 (d, 2H); 7.34 (d, 2H); 7.52 (d, 2H); 7.63 (d, 3H); 8.32 (d, 1H); MS (ESI+): m/z=512 [M+H]⁺; Melting point: 165-166.5° C. Note: all the starting material was not consumed and the reaction was repeated under the same condition as described above (overnight at room temperature). The residue was purified by flash chromatography (silica gel) with the appropriate gradient determined by TLC to give the titled compound.

### Example 86

### Acetic acid 4-(4-isopropyl-benzoyl)-1-(6-methyl-pyridazin-3-yl)-2-oxo-5-(4-trifluoromethoxy-phenyl)-2,5-dihydro-1H-pyrrol-3-yl ester

Prepared from 3-hydroxy-4-(4-isopropyl-benzoyl)-1-(6-methyl-pyridazin-3-yl)-5-(4-trifluoromethoxy-phenyl)-1,5-dihydro-pyrrol-2-one (Example 61) in 64% yield. Note: 1.5 eq of acetic anhydride was added and the solution was stirred overnight at room temperature. The mixture was concentrated under vacuum and the residue was purified by flash chromatography (silica gel). NMR-1H (DMSO): δ (ppm) 1.21 (d, 6H); 2.00 (s, 3H); 2.5 (s, 3H); 2.96 (quint, 1H); 6.72 (s, 1H); 7.24 (d, 2H); 7.39 (d, 2H); 7.53 (d, 2H); 7.62 (d, 2H); 7.66 (d, 1H); 8.32 (d, 1H); MS (ESI+): m/z=540 [M+H]⁺

### Example 186

### Isobutyric acid 4-(4-isopropyl-benzoyl)-1-(6-methyl-pyridazin-3-yl)-2-oxo-5-(4-trifluoromethoxy-phenyl)-2,5-dihydro-1H-pyrrol-3-yl ester

Prepared from 3-hydroxy-4-(4-isopropyl-benzoyl)-1-(6-methyl-pyridazin-3-yl)-5-(4-trifluoromethoxy-phenyl)-1,5-dihydro-pyrrol-2-one (Example 61) and isobutyric anhydride in 61% yield. ¹H-NMR (DMSO-d₆): δ (ppm) 0.84 (dd, 6H); 1.17 (d, 6H); 2.52 (s, 3H); 2.56 (quint, 1H); 2.94 (quint, 1H); 6.72 (s, 1H); 7.26 (d, 2H); 7.38 (d, 2H); 7.53-7.68 (m, 5H); 8.33 (d, 1H); MS (ESI+): m/z=568 [M+H]⁺

### Procedure F: Preparation of Intermediate

### Intermediate 7

### 4-[4-(2-dimethylamino-ethyl)-piperazin-1-yl]-benzaldehyde

4-fluorobenzaldehyde (1 eq) and 1-[2-(dimethylamino)ethyl]piperazine (1 eq) were dissolved in DMF (4 ml/mmol). K₂CO₃ (1.5 eq) was added and the reaction mixture was stirred at reflux overnight then diluted with water. The aqueous layer was extracted twice with ethyl acetate. The combined organic layers were extracted with HCl 1N. The aqueous phase was basified with NaOH 1N then extracted twice with ethyl acetate. The organic layers were combined, dried on anhydrous MgSO₄, filtered and concentrated in vacuum to give the desired compound in 65% yield. ¹H-NMR (DMSO-d₆): δ (ppm) 2.26 (s, 6H); 2.44-2.53 (m, 4H); 2.59-2.63 (m, 4H); 3.38-3.42 (m, 4H); 6.89 (d, 2H); 7.73 (d, 2H); 9.76 (s, 1H)

### General Procedure G:

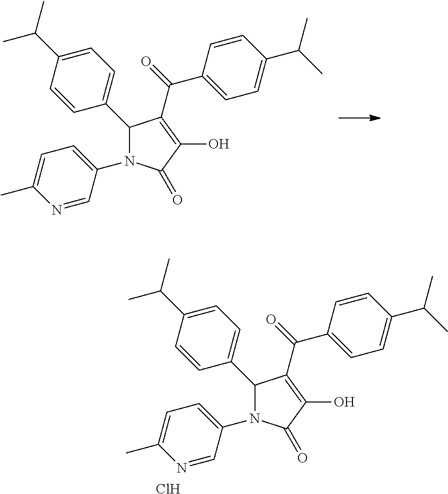

### Example 48

To a solution of the starting material (1 eq) in dichloromethane (18 ml/mmol) was added HCl 1M in Et₂O (1 eq). The solution was stirred for 5 h at room temperature. The mixture was concentrated under vacuum to give the titled compound. The following compounds were prepared according general procedure G:

### Example 66

### 5-[3-hydroxy-4-(4-isopropyl-benzoyl)-5-(4-isopropyl-phenyl)-2-oxo-2,5-dihydro-pyrrol-1-yl]-2-methyl-pyridinium; chloride

Prepared from 3-hydroxy-4-(4-isopropyl-benzoyl)-5-(4-isopropyl-phenyl)-1-(6-methyl-pyridin-3-yl)-1,5-dihydro-pyrrol-2-one (Example 48) in 70% yield. ¹H-NMR (DMSO-d₆): δ(ppm) 1.06 (d, 6H); 1.19 (d, 6H); 2.50 (s, 3H); 2.72 (quint, 1H); 2.93 (quint, 1H); 6.38 (s, 1H); 7.09 (d; 2H); 7.32-7.38 (m, 4H); 7.54 (d, 1H); 7.67 (d, 2H); 8.27 (d, 1H); 8.89 (brs, 1H); MS (ESI+): m/z=455 [M+H]⁺; Melting point: 165° C.

### Example 97

### 5-[3-hydroxy-5-(4-isopropyl-phenyl)-2-oxo-4-(4-trifluoromethoxy-benzoyl)-2,5-dihydro-pyrrol-1-yl]-2-methyl-pyridinium; chloride

Prepared from 3-hydroxy-5-(4-isopropyl-phenyl)-1-(6-methyl-pyridin-3-yl)-4-(4-trifluoromethoxy-benzoyl)-1,5-dihydro-pyrrol-2-one (Example 94) in 60% yield. ¹H-NMR (DMSO-d₆): δ (ppm) 1.04 (d, 6H); 2.46 (s, 3H); 2.72 (quint, 1H); 6.36 (s, 1H); 7.07 (d, 2H); 7.34-7.46 (m, 5H); 7.84 (d, 2H); 8.16 (d, 1H); 8.83 (brs, 1H); MS (ESI+): m/z=533 [M+H]⁺

### Example 155

### 2-ethoxy-5-[4-hydroxy-3-(4-isopropyl-benzoyl)-1-(6-methyl-pyridazin-3-yl)-5-oxo-2,5-dihydro-1H-pyrrol-2-yl]-pyridinium; chloride

Prepared from 5-(6-ethoxy-pyridin-3-yl)-3-hydroxy-4-(4-isopropyl-benzoyl)-1-(6-methyl-pyridazin-3-yl)-1,5-dihydro-pyrrol-2-one (Example 110) in 81% yield. ¹H-NMR (DMSO-d₆): δ (ppm) 1.21 (m, 9H); 2.55 (s, 3H); 2.94 (quint, 1H); 4.15 (q, 2H); 6.38 (s, 1H); 6.61 (d, 1H); 7.35 (d, 2H); 7.71-7.78 (m, 4H); 8.25 (d, 1H); 8.42 (d, 1H); MS (ESI+): m/z=459 [M+H]⁺; Melting point: 281-283° C.

### Example 167

### 3-hydroxy-5-(6-isopropoxy-pyridin-3-yl)-4-(4-isopropyl-benzoyl)-1-(6-methyl-pyridazin-3-yl)-1,5-dihydro-pyrrol-2-one; hydrochloride

Prepared, following from 3-hydroxy-5-(6-isopropoxy-pyridin-3-yl)-4-(4-isopropyl-benzoyl)-1-(6-methyl-pyridazin-3-yl)-1,5-dihydro-pyrrol-2-one (Example 165) in 98% yield. ¹H-NMR (DMSO-d₆): δ (ppm) 1.16-1.22 (m, 12H); 2.56 (s, 3H); 2.94 (quint, 1H); 5.08 (quint, 1H); 6.37 (s, 1H); 6.57 (d, 1H); 7.35 (d, 2H); 7.71-7.78 (m, 4H); 8.25 (d, 1H); 8.46 (d, 1H); MS (ESI+): m/z=473 [M+H]⁺; Melting point: 280° C.

### General Procedure H:

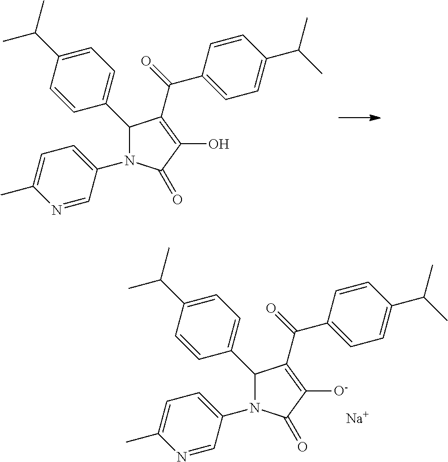

### Example 48

To a solution of the starting material (1 eq) in methanol (18 ml/mmol) was added MeONa [prepared in situ with Na (1 eq) in methanol (9.09 ml/mmol)]. The solution was stirred for 5 h at room temperature. The mixture was concentrated under vacuum to give the titled compound.

### Example 67

### Sodium; 4-(4-isopropyl-benzoyl)-5-(4-isopropyl-phenyl)-1-(6-methyl-pyridin-3-yl)-2-oxo-2,5-dihydro-1H-pyrrol-3-olate

Prepared from 3-hydroxy-4-(4-isopropyl-benzoyl)-5-(4-isopropyl-phenyl)-1-(6-methyl-pyridin-3-yl)-1,5-dihydro-pyrrol-2-one (Example 48) in quantitative yield. ¹H-NMR (DMSO-d₆): δ (ppm) 1.07 (d, 6H); 1.17 (d, 6H); 2.33 (s, 3H); 2.71 (quint, 1H); 2.86 (quint, 1H); 5.94 (s, 1H); 6.99 (d; 2H); 7.07-7.17 (m, 3H); 7.21 (d, 2H); 7.70 (d, 2H); 7.92 (dd, 1H); 8.67 (d, 1H); MS (ESI+): m/z=455 [M+H]⁺; Melting point: 215° C.

### General Procedure J:

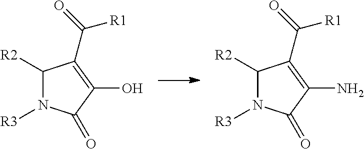

(R1, R2 and R3 are as defined above).

To a solution of the starting material (1 eq) in 2-methoxyethanol (7 ml/mmol) was added ammonium formate (1.8 eq). The solution was heated under reflux condition for 3 h. The mixture was concentrated under vacuum then the residue was triturated in Et₂O and after filtration the solid was washed with Et₂O to give the desired compound. The following examples were prepared according procedure J:

### Example 74

### 3-amino-4-(4-isopropyl-benzoyl)-5-(4-isopropyl-phenyl)-1-(6-methyl-pyridazin-3-yl)-1,5-dihydro-pyrrol-2-one

Prepared from 3-hydroxy-4-(4-isopropyl-benzoyl)-5-(4-isopropyl-phenyl)-1-(6-methyl-pyridazin-3-yl)-1,5-dihydro-pyrrol-2-one (Example 20) in 45% yield. Note: after 3 h, the reaction was not complete, ammonium formate (1.8 eq) was thus added and the reaction mixture was heated under reflux condition until consumption of the starting material. After trituration, the residue was purified by flash chromatography (silica gel) with the appropriate gradient determined by TLC. ¹H-NMR (DMSO-d₆): δ (ppm) 1.01 (d, 6H); 1.23 (d, 6H); 2.5 (s, 3H); 2.61 (m, 1H); 2.91 (quint, 1H); 6.46 (s, 1H); 6.53 (d, 2H); 6.68 (d, 2H); 7.13-7.22 (dd, 4H); 7.52 (d, 1H); 8.29 (d, 1H); 9.07 (brs, 1H); 10.23 (brs, 1H); MS (ESI+): m/z=455 [M+H]⁺

### Example 75

### 3-amino-4-(4-isopropyl-benzoyl)-1-(6-methyl-pyridazin-3-yl)-5-(4-trifluoromethoxy-phenyl)-1,5-dihydro-pyrrol-2-one

Prepared from 3-hydroxy-4-(4-isopropyl-benzoyl)-1-(6-methyl-pyridazin-3-yl)-5-(4-trifluoromethoxy-phenyl)-1,5-dihydro-pyrrol-2-one (Example 61) in 52% yield; ¹H-NMR (DMSO-d₆): δ (ppm) 1.21 (d, 6H); 2.48 (s, 3H); 2.91 (quint, 1H); 6.52 (s, 1H); 6.78 (brs, 4H); 7.17-7.24 (m, 4H); 7.55 (d, 1H); 8.32 (d, 1H); 9.14 (brs; 1H); 10.24 (brs, 1H); MS (ESI+): m/z=497 [M+H]⁺; Melting point: 258-260° C.

### General Procedure K: Preparation of Intermediates

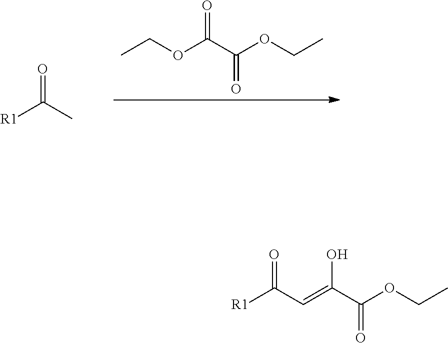

R1 is as defined above

To a solution of the starting material (1 eq) in dry toluene and under an atmosphere of nitrogen was added portionwise sodium hydride (2 eq). The mixture was heated at 45° C. and the diethyl oxalate (1.5 eq) in dry toluene was added dropwise. The mixture was refluxed for 10 min, then, concentrated in vacuum to give the crude product. It was purified by flash chromatography on silica gel. The product was dissolved in diethyl ether and washed with HCl 1N, the layers were separated and the organic layers were combined, dried on anhydrous MgSO₄, filtered and concentrated in vacuum to give the keto-ester. The following intermediate compounds were prepared according to general procedure K:

### Intermediate 8

### 2-hydroxy-4-[4-(4-methoxy-benzyloxy)-phenyl]-4-oxo-but-2-enoic acid ethyl ester

Prepared from 1-[4-(4-methoxy-benzyloxy)-phenyl]-ethanone and diethyl oxalate in 38% yield. Note: the crude product was purified by flash chromatography on silica gel to give the keto-ester. ¹H-NMR (CDCl₃): δ (ppm) 1.41 (t, 3H); 3.82 (s, 3H); 4.40 (q, 2H); 5.07 (s, 2H); 6.93 (d, 2H); 6.99-7.10 (m, 3H); 7.36 (d, 2H); 7.99 (d, 2H).

### Intermediate 13

### 2-hydroxy-4-oxo-4-(6-trifluoromethyl-pyridin-3-yl)-but-2-enoic acid ethyl ester

Prepared from 1-(6-trifluoromethyl-pyridin-3-yl)-ethanone and diethyl oxalate in 38% yield. Note: after 10 min at reflux, an excess of diethyl oxalate (4 eq) was added and the mixture was heated at reflux for 30 min. MS (ESI+): m/z=415 [M+H]⁺

### Intermediate 16

### 2-hydroxy-4-(4-isopropenyl-phenyl)-4-oxo-but-2-enoic acid ethyl ester

Prepared from 1-(4-isopropenyl-phenyl)-ethanone and diethyl oxalate in 57% yield. ¹H-NMR (DMSO-d₆): δ (ppm) 1.42 (t, 3H), 2.18 (s, 3H); 4.41 (q, 2H); 5.24 (s, 1H); 5.52 (s, 1H); 7.07 (s, 1H); 7.58 (d, 2H); 7.96 (d, 2H); MS (ESI+): m/z=261 [M+H]⁺

### General Procedure L: Diastereoisomers Formation

The diastereoisomers formation is an adapted procedure from Zou et al. Letters in Drug Design & Discovery, 2007, 4, 185-191.

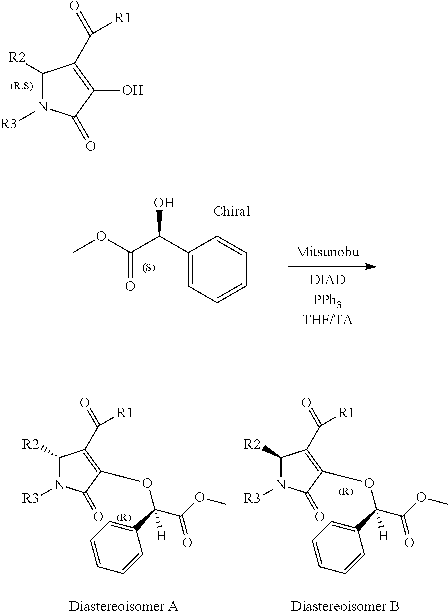

R1, R2 and R3 are as defined above.

To a solution of the starting material (1 eq) in dry THF (10 ml/mmol) at 0° C. and under an atmosphere of nitrogen was added triphenylphosphine (1.5 eq) and DIAD (1.5 eq). The solution was stirred 15 min at 0° C. then the methyl (S)-(+)-mandelate (1.5 eq) was added. The reaction mixture was stirred at room temperature overnight and concentrated under vacuum. The crude product was dissolved in EtOAc, washed with H₂O, sodium hydroxide 1N, H₂O and saturated sodium chloride, dried over anhydrous Na₂SO₄, filtered and concentrated under vacuum. Diastereoisomers A and B were separated and purified using flash chromatography (silica gel) with the appropriate gradient determined by TLC.

The following compounds were prepared according to general procedure L:

### Example 201

### (R)-[4-(4-isopropyl-benzoyl)-1-(6-methyl-pyridazin-3-yl)-2-oxo-5(R)-(4-trifluoromethoxy-phenyl)-2,5-dihydro-1H-pyrrol-3-yloxy]-phenyl-acetic acid methyl ester (Diastereoisomer A)

Prepared from 3-hydroxy-4-(4-isopropyl-benzoyl)-1-(6-methyl-pyridazin-3-yl)-5-(4-trifluoromethoxy-phenyl)-1,5-dihydro-pyrrol-2-one (Example 61) and methyl (S)-(+)-mandelate in 23%. ¹H-NMR (DMSO-d₆): δ (ppm) 1.21 (d, 6H); 2.52 (s, 3H); 2.97 (m, 1H); 3.78 (s, 3H); 6.57 (s, 1H); 6.73 (s, 1H); 7.10 (d, 2H); 7.20 (t, 2H); 7.30 (d, 3H); 7.38 (d, 2H), 7.52 (d, 2H); 7.62 (d, 1H); 7.82 (d, 2H); 8.29 (d, 1H); MS (ESI+): m/z=646 [M+H]⁺

### Example 202

### (R)-[4-(4-isopropyl-benzoyl)-1-(6-methyl-pyridazin-3-yl)-2-oxo-5(S)-(4-trifluoromethoxy-phenyl)-2,5-dihydro-1H-pyrrol-3-yloxy]-phenyl-acetic acid methyl ester (Diastereoisomer B)

Prepared from 3-hydroxy-4-(4-isopropyl-benzoyl)-1-(6-methyl-pyridazin-3-yl)-5-(4-trifluoromethoxy-phenyl)-1,5-dihydro-pyrrol-2-one (Example 61) and methyl (S)-(+)-mandelate in 10%. ¹H-NMR (DMSO-d₆): δ(ppm) 1.17 (d, 6H); 2.52 (s, 3H); 2.80 (quint, 1H); 3.60 (s, 3H); 6.57 (s, 1H); 6.70 (s, 1H); 7.03 (d, 2H); 7.14-7.34 (m, 7H); 7.59-7.67 (m, 5H); 8.27 (d, 1H); MS (ESI+): m/z=646 [M+H]⁺

### Example 203

### (R)-[4-(4-isopropyl-benzoyl)-1-(6-methyl-pyridazin-3-yl)-2-oxo-5(S)-(4-trifluoromethyl-phenyl)-2,5-dihydro-1H-pyrrol-3-yloxy]-phenyl-acetic acid methyl ester (diastereoisomer B)

Prepared from 3-hydroxy-4-(4-isopropyl-benzoyl)-1-(6-methyl-pyridazin-3-yl)-5-(4-trifluoromethyl-phenyl)-1,5-dihydro-pyrrol-2-one (Example 63) and methyl (S)-(+)-mandelate in 14%. ¹H-NMR (DMSO-d₆): δ(ppm) 1.10 (d, 6H); 2.50 (s, 3H); 2.78 (quint, 1H); 3.61 (s, 3H); 6.61 (s, 1H); 6.71 (s, 1H); 7.04 (d, 2H); 7.15 (d, 2H); 7.22-7.34 (m, 3H); 7.59-7.62 (m, 5H); 7.74 (d, 2H); 8.30 (d, 1H); MS (ESI+): m/z=630 [M+H]⁺

### Example 204

### (R)-[4-(4-isopropyl-benzoyl)-1-(6-methyl-pyridazin-3-yl)-2-oxo-5(R)-(4-trifluoromethyl-phenyl)-2,5-dihydro-1H-pyrrol-3-yloxy]-phenyl-acetic acid methyl ester (diastereoisomer A)

Prepared from 3-hydroxy-4-(4-isopropyl-benzoyl)-1-(6-methyl-pyridazin-3-yl)-5-(4-trifluoromethyl-phenyl)-1,5-dihydro-pyrrol-2-one (Example 63) and methyl (S)-(+)-mandelate in 31%. ¹H-NMR (DMSO-d₆): δ(ppm) 1.21 (d, 6H); 2.50 (s, 3H); 2.96 (quint, 1H); 3.78 (s, 3H); 6.60 (s, 1H); 6.72 (s, 1H); 7.09 (d, 2H); 7.19 (t, 2H); 7.29 (d, 1H); 7.37 (d, 2H); 7.58-7.69 (m, 5H); 7.80 (d, 2H); 8.31 (d, 1H); MS (ESI+): m/z=630 [M+H]⁺

### Example 205

### (R)-[5(R)-(6-ethoxy-pyridin-3-yl)-4-(4-isopropyl-benzoyl)-1-(6-methyl-pyridazin-3-yl)-2-oxo-2,5-dihydro-1H-pyrrol-3-yloxy]-phenyl-acetic acid methyl ester (diastereoisomer A)

Prepared from 5-(6-ethoxy-pyridin-3-yl)-3-hydroxy-4-(4-isopropyl-benzoyl)-1-(6-methyl-pyridazin-3-yl)-1,5-dihydro-pyrrol-2-one (Example 110) and methyl (S)-(+)-mandelate. ¹H-NMR (DMSO-d₆): δ(ppm) 1.23 (d, 9H); 2.53 (s, 3H); 2.97 (quint, 1H); 3.78 (s, 3H); 4.17 (q, 2H); 6.50 (s, 1H); 6.72 (d, 2H); 7.12 (d, 2H); 7.21 (t, 2H); 7.31 (t, 1H); 7.41 (d, 2H); 7.62 (d, 2H); 7.86 (d, 2H); 8.22-8.27 (m, 2H); MS (ESI+): m/z=607 [M+H]⁺

### Example 206

### (R)-[5(S)-(6-ethoxy-pyridin-3-yl)-4-(4-isopropyl-benzoyl)-1-(6-methyl-pyridazin-3-yl)-2-oxo-2,5-dihydro-1H-pyrrol-3-yloxy]-phenyl-acetic acid methyl ester (diastereoisomer B)

Prepared from 5-(6-ethoxy-pyridin-3-yl)-3-hydroxy-4-(4-isopropyl-benzoyl)-1-(6-methyl-pyridazin-3-yl)-1,5-dihydro-pyrrol-2-one (Example 110) and methyl (S)-(+)-mandelate. ¹H-NMR (DMSO-d₆): δ(ppm) 1.15 (d, 9H); 2.50 (s, 3H); 2.78 (quint, 1H); 3.61 (s, 3H); 4.16 (q, 2H); 6.50 (s, 1H); 6.61-6.68 (m, 2H); 7.04 (d, 2H); 7.16-7.34 (m, 5H); 7.63 (t, 3H); 7.84 (d, 1H); 8.23-8.28 (m, 2H); MS (ESI+): m/z=607 [M+H]⁺

### Example 207

### (R)-[4-(4-isopropyl-benzoyl)-1-(6-methyl-pyridazin-3-yl)-2-oxo-5(S)-(6-trifluoromethyl-pyridin-3-yl)-2,5-dihydro-1H-pyrrol-3-yloxy]-phenyl-acetic acid methyl ester (diastereoisomer B)

Prepared from 3-hydroxy-4-(4-isopropyl-benzoyl)-1-(6-methyl-pyridazin-3-yl)-5-(6-trifluoromethyl-pyridin-3-yl)-1,5-dihydro-pyrrol-2-one (Example 98) and methyl (S)-(+)-mandelate ¹H-NMR (DMSO-d₆): δ(ppm) 1.12 (d, 6H); 2.50 (s, 3H); 2.80 (quint, 1H); 3.63 (s, 3H); 6.67 (s, 1H); 6.72 (s, 1H); 7.04 (d, 2H); 7.15 (d, 2H); 7.25-7.38 (m, 3H); 7.64 (d, 3H); 7.81 (d, 1H); 8.32 (dd, 2H); 8.97 (brs, 1H); MS (ESI+): m/z=631 [M+H]⁺

### Example 208

### (R)-[4-(4-isopropyl-benzoyl)-1-(6-methyl-pyridazin-3-yl)-2-oxo-5(R)-(6-trifluoromethyl-pyridin-3-yl)-2,5-dihydro-1H-pyrrol-3-yloxy]-phenyl-acetic acid methyl ester (diastereoisomer A)

Prepared from 3-hydroxy-4-(4-isopropyl-benzoyl)-1-(6-methyl-pyridazin-3-yl)-5-(6-trifluoromethyl-pyridin-3-yl)-1,5-dihydro-pyrrol-2-one (Example 98) and methyl (S)-(+)-mandelate. ¹H-NMR (DMSO-d₆): δ(ppm) 1.21 (d, 6H); 2.50 (s, 3H); 2.95 (quint, 1H); 3.77 (s, 3H); 6.65 (s, 1H); 6.75 (s, 1H); 7.10 (d, 2H); 7.21 (t, 2H); 7.30 (d, 1H); 7.37 (d, 2H); 7.65 (d, 1H); 7.84 (dd, 3H); 8.07 (d, 1H); 8.38 (d, 1H); 8.89 (d, 1H); MS (ESI+): m/z=631 [M+H]⁺

### Example 209

### (R)-[5(R)[4-(1,1-difluoro-ethyl)-phenyl]-4-(4-isopropyl-benzoyl)-1-(6-methyl-pyridazin-3-yl)-2-oxo-2,5-dihydro-1H-pyrrol-3-yloxy]-phenyl-acetic acid methyl ester (diastereoisomer A)

Prepared from 5-[4-(1,1-difluoro-ethyl)-phenyl]-3-hydroxy-4-(4-isopropyl-benzoyl)-1-(6-methyl-pyridazin-3-yl)-1,5-dihydro-pyrrol-2-one (Example 158) and methyl (S)-(+)-mandelate. ¹H-NMR (DMSO-d₆): δ(ppm) 1.23 (d, 6H); 1.87 (t, 3H), 2.51 (s, 3H); 2.97 (quint, 1H); 3.79 (s, 3H); 6.59 (s, 1H); 6.74 (s, 1H); 7.10 (d, 2H); 7.20 (t, 2H); 7.31 (t, 1H); 7.40 (d, 2H); 7.51 (brs, 4H); 7.62 (d, 1H); 7.83 (d, 2H); 8.30 (d, 1H); MS (ESI+): m/z=626 [M+H]⁺

### Example 210

### (R)-[5(S)-[4-(1,1-difluoro-ethyl)-phenyl]-4-(4-isopropyl-benzoyl)-1-(6-methyl-pyridazin-3-yl)-2-oxo-2,5-dihydro-1H-pyrrol-3-yloxy]-phenyl-acetic acid methyl ester (diastereoisomer B)

Prepared from 5-[4-(1,1-difluoro-ethyl)-phenyl]-3-hydroxy-4-(4-isopropyl-benzoyl)-1-(6-methyl-pyridazin-3-yl)-1,5-dihydro-pyrrol-2-one (Example 158) and methyl (S)-(+)-mandelate. ¹H-NMR (DMSO-d₆): δ(ppm) 1.12 (d, 6H); 1.84 (t, 3H); 2.50 (s, 3H); 2.78 (quint, 1H); 3.60 (s, 3H); 6.58 (s, 1H); 6.72 (s, 1H); 7.02 (d, 2H); 7.16 (d, 2H); 7.24-7.33 (m, 3H), 7.41 (d, 2H), 7.59-7.65 (m, 5H); 8.27 (d, 1H); MS (ESI+): m/z=626 [M+H]⁺

### Example 211

### (R)-[4-(4-methyl-benzoyl)-1-(6-methyl-pyridazin-3-yl)-2-oxo-5(R)-(4-trifluoromethoxy-phenyl)-2,5-dihydro-1H-pyrrol-3-yloxy]-phenyl-acetic acid methyl ester (diastereoisomer A)

Prepared from 3-hydroxy-4-(4-methyl-benzoyl)-1-(6-methyl-pyridazin-3-yl)-5-(4-trifluoromethoxy-phenyl)-1,5-dihydro-pyrrol-2-one (Example 22) and methyl (S)-(+)-mandelate in 40%. ¹H-NMR (DMSO-d₆): δ(ppm) 2.39 (s, 3H); 2.52 (s, 3H); 3.76 (s, 3H); 6.56 (s, 1H); 6.68 (s, 1H); 7.07 (d, 2H); 7.19-7.33 (m, 8H); 7.50 (d, 2H); 7.62 (d, 1H), 7.74 (d, 2H); 8.28 (d, 1H). MS (ESI+): m/z=618 [M+H]⁺

### General Procedure M: Enantiomers Formation

The Enantiomers Formation is an adapted procedure from Zou et al. Letters in Drug Design & Discovery, 2007, 4, 185-191.

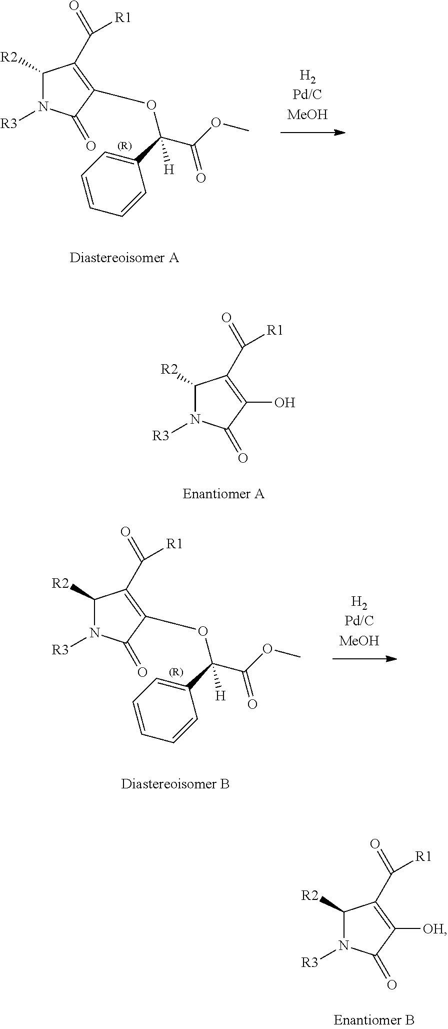

R1, R2 and R3 are as defined above.

Diastereoisomer A was dissolved in methanol (10 ml/mmol) and ethyl acetate (5 ml/mmol). Diastereoisomer B was dissolved in dichloromethane (16 ml/mmol) and methanol (11 ml/mmol). Each solution was purged under argon and the palladium on actived charcoal (10%) was added. Each reaction mixture was stirred, independently, at atmospheric pressure in a hydrogen atmosphere for 16 h at room temperature. After filtration of the mixture on Celite®, the solid was washed with CH₂Cl₂/MeOH (50/50) and the filtrate was concentrated under vacuum. Then, the residue was triturated in Et₂O and after filtration the solid was washed with Et₂O to give the corresponding enantiomer.

The following compounds were prepared according to general procedure M:

### Example 188

### 3-hydroxy-4-(4-isopropyl-benzoyl)-1-(6-methyl-pyridazin-3-yl)-5(R)-(4-trifluoromethoxy-phenyl)-1,5-dihydro-pyrrol-2-one

Prepared from (R)-[4-(4-isopropyl-benzoyl)-1-(6-methyl-pyridazin-3-yl)-2-oxo-5(R)-(4-trifluoromethoxy-phenyl)-2,5-dihydro-1H-pyrrol-3-yloxy]-phenyl-acetic acid methyl ester (Example 201, diastereoisomer A) in 43% yield. ¹H-NMR (DMSO-d₆): δ(ppm) 1.19 (d, 6H); 2.50 (s, 3H); 2.93 (quint, 1H); 6.47 (s, 1H); 7.17 (d, 2H); 7.32 (d, 2H); 7.53-7.58 (m, 2H); 7.62 (s, 1H); 7.68 (d, 2H); 8.32 (d; 1H); MS (ESI+): m/z=498 [M+H]⁺; Melting point: 217° C.; [α]_{D}=−29.41° (c=0.255 in DMSO)

### Example 189

### 3-hydroxy-4-(4-isopropyl-benzoyl)-1-(6-methyl-pyridazin-3-yl)-5(S)-(4-trifluoromethoxy-phenyl)-1,5-dihydro-pyrrol-2-one

Prepared from (R)-[4-(4-isopropyl-benzoyl)-1-(6-methyl-pyridazin-3-yl)-2-oxo-5(S)-(4-trifluoromethoxy-phenyl)-2,5-dihydro-1H-pyrrol-3-yloxy]-phenyl-acetic acid methyl ester (Example 202, diastereoisomer B) in 23% yield. ¹H-NMR (DMSO-d₆): δ(ppm) 1.19 (d, 6H); 2.50 (s, 3H); 2.93 (quint, 1H); 6.47 (s, 1H); 7.17 (d, 2H); 7.32 (d, 2H); 7.53-7.58 (m, 2H); 7.62 (s, 1H); 7.68 (d, 2H); 8.32 (d; 1H); MS (ESI+): m/z=498 [M+H]⁺; Melting point: 221° C.; [α]_{D}=+32.65° (c=0.245 in DMSO)

### Example 190

### 3-hydroxy-4-(4-isopropyl-benzoyl)-1-(6-methyl-pyridazin-3-yl)-5(S)-(4-trifluoromethyl-phenyl)-1,5-dihydro-pyrrol-2-one

Prepared from (R)-[4-(4-isopropyl-benzoyl)-1-(6-methyl-pyridazin-3-yl)-2-oxo-5(S)-(4-trifluoromethyl-phenyl)-2,5-dihydro-1H-pyrrol-3-yloxy]-phenyl-acetic acid methyl ester (Example 203, diastereoisomer B) in 38% yield. ¹H-NMR (DMSO-d₆): δ(ppm) 1.20 (d, 6H); 2.50 (s, 3H); 2.92 (quint, 1H); 6.49 (s, 1H); 7.30 (d, 2H); 7.48-7.81 (m, 7H); 8.38 (d, 1H); MS (ESI+): m/z=482 [M+H]⁺; Melting point: 252° C.; [α]_{D}=+39.48° C. (c=0.195 in DMSO)

### Example 191

### 3-hydroxy-4-(4-isopropyl-benzoyl)-1-(6-methyl-pyridazin-3-yl)-5(R)-(4-trifluoromethyl-phenyl)-1,5-dihydro-pyrrol-2-one

Prepared from (R)-[4-(4-isopropyl-benzoyl)-1-(6-methyl-pyridazin-3-yl)-2-oxo-5(R)-(4-trifluoromethyl-phenyl)-2,5-dihydro-1H-pyrrol-3-yloxy]-phenyl-acetic acid methyl ester (Example 204, diastereoisomer A) in 24% yield. ¹H-NMR (DMSO-d₆): δ(ppm) 1.19 (d, 6H); 2.50 (s, 3H); 2.92 (quint, 1H); 6.49 (s, 1H); 7.29 (d, 2H); 7.50-7.78 (m, 7H); 8.37 (d, 1H); MS (ESI+): m/z=482 [M+H]⁺; Melting point: 251° C.; [α]_{D}=−44° C. (c=0.225 in DMSO)

### Example 192

### 5(R)-(6-ethoxy-pyridin-3-yl)-3-hydroxy-4-(4-isopropyl-benzoyl)-1-(6-methyl-pyridazin-3-yl)-1,5-dihydro-pyrrol-2-one

Prepared from (R)-[5(R)-(6-ethoxy-pyridin-3-yl)-4-(4-isopropyl-benzoyl)-1-(6-methyl-pyridazin-3-yl)-2-oxo-2,5-dihydro-1H-pyrrol-3-yloxy]-phenyl-acetic acid methyl ester (Example 205, diastereoisomer A) in 39%. ¹H-NMR (DMSO-d₆): δ (ppm) 1.20 (d, 9H); 2.50 (s, 3H), 2.93 (quint, 1H); 4.15 (q, 2H); 6.40 (s, 1H); 6.57 (d, 1H); 7.32 (d, 2H); 7.60 (d, 1H); 7.68-7.74 (m, 3H); 8.21 (brs, 1H); 8.30 (d, 1H); MS (ESI+): m/z=459 [M+H]⁺; [α]_{D}=−20.59° (c=0.170 in DMSO).

### Example 193

### 5(S)-(6-ethoxy-pyridin-3-yl)-3-hydroxy-4-(4-isopropyl-benzoyl)-1-(6-methyl-pyridazin-3-yl)-1,5-dihydro-pyrrol-2-one

Prepared from (R)-[5(S)-(6-ethoxy-pyridin-3-yl)-4-(4-isopropyl-benzoyl)-1-(6-methyl-pyridazin-3-yl)-2-oxo-2,5-dihydro-1H-pyrrol-3-yloxy]-phenyl-acetic acid methyl ester (Example 206, diastereoisomer B) in 18%. ¹H-NMR (DMSO-d₆): δ(ppm) 1.20 (d, 9H); 2.50 (s, 3H), 2.93 (quint, 1H); 4.15 (q, 2H); 6.39 (s, 1H); 6.57 (d, 1H); 7.32 (d, 2H); 7.60 (d, 1H); 7.68-7.74 (m, 3H); 8.21 (brs, 1H); 8.28 (d, 1H); MS (ESI+): m/z=459 [M+H]⁺; [α]_{D}=+30.45° (c=0.220 in DMSO).

### Example 194

### 3-hydroxy-4-(4-isopropyl-benzoyl)-1-(6-methyl-pyridazin-3-yl)-5(S)-(6-trifluoromethyl-pyridin-3-yl)-1,5-dihydro-pyrrol-2-one

Prepared from (R)-[4-(4-isopropyl-benzoyl)-1-(6-methyl-pyridazin-3-yl)-2-oxoz-5(S)-(6-trifluoromethyl-pyridin-3-yl)-2,5-dihydro-1H-pyrrol-3-yloxy]-phenyl-acetic acid methyl ester (Example 207, diastereoisomer B) in 33%. ¹H-NMR (DMSO-d₆): δ(ppm) 1.19 (d, 6H); 2.50 (s, 3H); 2.93 (quint, 1H); 6.49 (s, 1H); 7.31 (d, 2H); 7.62 (d, 1H); 7.70-7.74 (m, 3H); 8.16 (d, 1H); 8.42 (d, 1H); 8.90 (brs, 1H); MS (ESI+): m/z=483 [M+H]⁺; [α]_{D}=+18.88° (c=0.180 in DMSO).

### Example 195

### 3-hydroxy-4-(4-isopropyl-benzoyl)-1-(6-methyl-pyridazin-3-yl)-5(R)-(6-trifluoromethyl-pyridin-3-yl)-1,5-dihydro-pyrrol-2-one

Prepared from (R)-[4-(4-isopropyl-benzoyl)-1-(6-methyl-pyridazin-3-yl)-2-oxo-5(R)-(6-trifluoromethyl-pyridin-3-yl)-2,5-dihydro-1H-pyrrol-3-yloxy]-phenyl-acetic acid methyl ester (Example 208, diastereoisomer A) in 26% yield.

¹H-NMR (DMSO-d₆): δ(ppm) 1.19 (d, 6H); 2.50 (s, 3H); 2.93 (quint, 1H); 6.50 (s, 1H); 7.31 (d, 2H); 7.62 (d, 1H); 7.70-7.74 (m, 3H); 8.16 (d, 1H); 8.41 (d, 1H); 8.90 (brs, 1H); MS (ESI+): m/z=483 [M+H]⁺; [α]_{D}=−25° (c=0.200 in DMSO).

### Example 196

### 5(R)-[4-(1,1-difluoro-ethyl)-phenyl]-3-hydroxy-4-(4-isopropyl-benzoyl)-1-(6-methyl-pyridazin-3-yl)-1,5-dihydro-pyrrol-2-one

Prepared from (R)-[5(R)-[4-(1,1-difluoro-ethyl)-phenyl]-4-(4-isopropyl-benzoyl)-1-(6-methyl-pyridazin-3-yl)-2-oxo-2,5-dihydro-1H-pyrrol-3-yloxy]-phenyl-acetic acid methyl ester (Example 209, diastereoisomer A) in 20% yield. ¹H-NMR (DMSO-d₆): δ(ppm) 1.20 (d, 6H); 1.84 (t, 3H); 2.50 (s, 3H); 2.92 (quint, 1H); 6.48 (s, 1H); 7.30-7.40 (m, 4H); 7.52 (d, 2H); 7.60 (d, 1H); 7.68 (d, 2H); 8.33 (d, 1H); MS (ESI+): m/z=478 [M+H]⁺; [α]_{D}=−52.22° (c=0.180 in DMSO).

### Example 197

### 5(S)-[4-(1,1-difluoro-ethyl)-phenyl]-3-hydroxy-4-(4-isopropyl-benzoyl)-1-(6-methyl-pyridazin-3-yl)-1,5-dihydro-pyrrol-2-one

Step 2: Prepared from (R)-[5(S)-[4-(1,1-difluoro-ethyl)-phenyl]-4-(4-isopropyl-benzoyl)-1-(6-methyl-pyridazin-3-yl)-2-oxo-2,5-dihydro-1H-pyrrol-3-yloxy]-phenyl-acetic acid methyl ester (Example 210, diastereoisomer B) in 29% yield. ¹H-NMR (DMSO-d₆): δ(ppm) 1.19 (d, 6H); 1.84 (t, 3H); 2.50 (s, 3H); 2.93 (quint, 1H); 6.49 (s, 1H); 7.30-7.40 (m, 4H); 7.53 (d, 2H); 7.60 (d, 1H); 7.68 (d, 2H); 8.33 (d, 1H); MS (ESI+): m/z=478 [M+H]⁺; [α]_{D}=+37.55° (c=0.245 in DMSO).

### Example 199

### 3-hydroxy-4-(4-methyl-benzoyl)-1-(6-methyl-pyridazin-3-yl)-5(R)-(4-trifluoromethoxy-phenyl)-1,5-dihydro-pyrrol-2-one

Prepared from (R)-[4-(4-methyl-benzoyl)-1-(6-methyl-pyridazin-3-yl)-2-oxo-5(R)-(4-trifluoromethoxy-phenyl)-2,5-dihydro-1H-pyrrol-3-yloxy]-phenyl-acetic acid methyl ester (Example 211, diastereoisomer A) in 28% yield. ¹H-NMR (DMSO-d₆): δ(ppm) 2.34 (s, 3H); 2.50 (s, 3H); 6.45 (s, 1H); 7.20 (dd, 4H); 7.52-7.65 (m, 5H); 8.33 (d; 1H); MS (ESI+): m/z=470 [M+H]⁺; Melting point: 215° C.; [α]_{D}=−38.14 (c=0.215 in DMSO)

### Example 198

### N-[4-(4-isopropyl-benzoyl)-1-(6-methyl-pyridazin-3-yl)-2-oxo-5(R)-(4-trifluoromethoxy-phenyl)-2,5-dihydro-1H-pyrrol-3-yl]-methanesulfonamide

Prepared from 3-chloro-4-(4-isopropyl-benzoyl)-1-(6-methyl-pyridazin-3-yl)-5(R)-(4-trifluoromethoxy-phenyl)-1,5-dihydro-pyrrol-2-one and methane sulphonamide (Example 212) in 21% yield. ¹H-NMR (CD₃OD): δ (ppm) 1.28 (d, 6H); 2.59 (s, 3H); 2.95 (quint, 1H); 3.03 (s, 3H); 6.45 (s, 1H); 6.83 (d, 2H); 6.91-6.98 (m, 2H); 7.09-7.15 (m, 2H); 7.21 (d, 2H); 7.72 (d, 1H); 8.65 (d, 1H); MS (ESI+): m/z=575 [M+H]⁺; [α]_{D}=−71.79° (c=0.195 in DMSO).

### General Procedure N

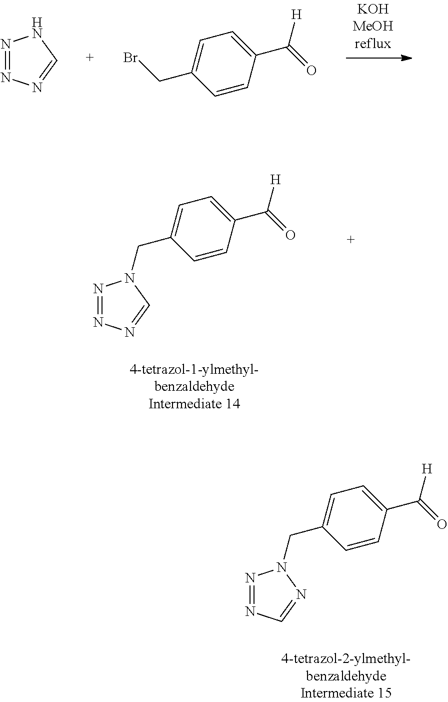

A mixture of 1H-tetrazole 3.89 mmol (1 eq), 4-(bromomethyl)benzaldehyde 3.89 mmol (1 eq) and potassium hydroxide 3.89 mmol (1 eq) in methanol (10 ml) was refluxed for 24 h, then, evaporated. The crude product was dissolved in dichloromethane and washed with H₂O then brine. The organic layers were dried on anhydrous Na₂SO₄, filtered and concentrated in vacuum. The residue was purified by flash chromatography on silica gel to give the aldehyde.

### Intermediate 14

### 4-tetrazol-1-ylmethyl-benzaldehyde

Prepared, following procedure N, from 1H-tetrazole and 4-(bromomethyl)benzaldehyde in 44% yield. ¹H-NMR (CDCl₃): δ (ppm) 5.70 (s, 2H); 7.44 (d, 2H); 7.93 (d, 2H); 8.62 (s, 1H); 10.03 (s, 1H)

### Intermediate 15

### 4-tetrazol-2-ylmethyl-benzaldehyde

Prepared, following procedure N, from 1H-tetrazole and 4-(bromomethyl)benzaldehyde in 26% yield. ¹H-NMR (CDCl₃): δ (ppm) 5.88 (s, 2H); 7.50 (d, 2H); 7.90 (d, 2H); 8.55 (s, 1H); 10.01 (s, 1H)

### General Procedure P

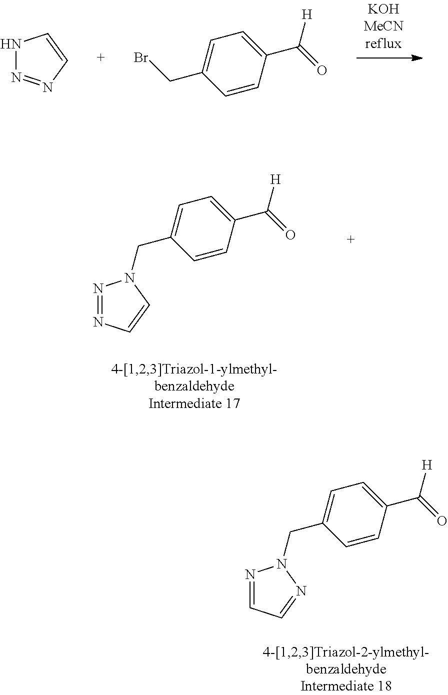

A mixture of 1H-triazole 1.7 mmol (1 eq), 4-(bromomethyl)benzaldehyde 1.7 mmol (1 eq) and potassium hydroxide 1.7 mmol (1 eq) in acetonitrile (5 ml) was refluxed for 7 h30. The mixture was diluted with H₂O and was extracted with dichloromethane. The organic layers were dried on anhydrous Na₂SO₄, filtered and concentrated in vacuum. The residue was purified by flash chromatography on silica gel to give the separated aldehydes.

### Intermediate 17

### 4-[1,2,3]triazol-1-ylmethyl-benzaldehyde

Prepared, following procedure P, from 1H-1,2,3-triazole and 4-(bromomethyl)benzaldehyde in 59% yield. ¹H-NMR (CDCl₃): δ (ppm) 5.66 (s, 2H); 7.39 (d, 2H); 7.54 (d, 1H); 7.75 (d, 1H); 7.88 (d, 2H); 10.01 (s, 1H)

### Intermediate 18

### 4-[1,2,3]triazol-2-ylmethyl-benzaldehyde

Prepared, following procedure P, from 1H-1,2,3-triazole and 4-(bromomethyl)benzaldehyde in 25% yield. ¹H-NMR (CDCl₃): δ (ppm) 5.69 (s, 2H); 7.41 (d, 2H); 7.66 (s, 2H); 7.86 (d, 2H); 9.99 (s, 1H)

### Other Procedures

The following compounds and intermediate compounds were prepared according to a particular process as described below:

### Example 91

### 3-methoxy-4-(4-methyl-benzoyl)-1-(6-methyl-pyridazin-3-yl)-5-(4-trifluoromethoxy-phenyl)-1,5-dihydro-pyrrol-2-one

NaH (1.5 eq) was added portionwise to a stirred solution of 3-hydroxy-4-(4-methyl-benzoyl)-1-(6-methyl-pyridazin-3-yl)-5-(4-trifluoromethoxy-phenyl)-1,5-dihydro-pyrrol-2-one (Example 22) in DMF (8 ml/mmol) at 0° C. After stirring 30 min at 0° C., iodomethane (1.5 eq) was added dropwise. The reaction mixture was stirred at room temperature overnight, then, diluted with water. The aqueous layer was extracted with ethyl acetate. The organic layer was washed with brine, dried over anhydrous MgSO₄, filtered and concentrated under vacuum. The residue was purified by flash chromatography (silica gel) to afford the titled compound in 7% yield. ¹H-NMR (DMSO-d₆): δ (ppm) 2.37 (s, 3H); 2.50 (s, 3H); 3.88 (s, 3H); 6.47 (s, 1H); 7.17 (d, 2H); 7.32 (d, 2H); 7.46 (d, 2H); 7.61 (d, 1H); 7.71 (d, 2H); 8.32 (d, 1H); MS (ESI+): m/z=484 [M+H]⁺; Melting point: 206-207° C.

### Example 92

### 5-(4-isopropyl-phenyl)-3-methoxy-4-(4-methyl-benzoyl)-1-(6-methyl-pyridazin-3-yl)-1,5-dihydro-pyrrol-2-one

Trimethylsilyl-diazomethane (solution 2N in hexane, 1.2 eq) was added at room temperature to a solution of 3-hydroxy-5-(4-isopropyl-phenyl)-4-(4-methyl-benzoyl)-1-(6-methyl-pyridazin-3-yl)-1,5-dihydro-pyrrol-2-one (Example 8) (1 eq) in dichloromethane (4 ml/mmol) and methanol (4 ml/mmol). The reaction mixture was stirred at room temperature for 5 h and concentrated under vacuum. The residue was purified by flash chromatography (silica gel) with the appropriate gradient determined by TLC to give the desired compound in 61% yield. ¹H-NMR (DMSO-d₆): δ (ppm) 1.05 (d, 6H); 2.36 (s, 3H); 2.50 (s, 3H); 2.71 (quint, 1H); 3.86 (s, 3H); 6.44 (s, 1H); 7.03 (d, 2H); 7.19 (d, 2H); 7.31 (d, 2H); 7.59 (d, 1H); 7.70 (d, 2H); 8.27 (d, 1H); MS (ESI+): m/z=442 [M+H]⁺; Melting point: 248-249° C.

### Example 119

### 3-hydroxy-4-(4-isopropyl-benzoyl)-1-[6-(4-nitro-phenylsulfanyl)-pyridazin-3-yl]-5-(4-trifluoromethoxy-phenyl)-1,5-dihydro-pyrrol-2-one

4-nitrothiophenol (2 eq) was added to 1 eq. of 1-(6-chloro-pyridazin-3-yl)-3-hydroxy-4-(4-isopropyl-benzoyl)-5-(4-trifluoromethoxy-phenyl)-1,5-dihydro-pyrrol-2-one (example 118) in pyridine (10 ml/mmol). The solution was refluxed overnight then diluted with water. The aqueous layer was extracted twice with dichloromethane. The organic layers were combined, dried on anhydrous Na₂SO₄, filtered and concentrated in vacuum. The solid was washed with Et₂O then filtered and the residue was purified by flash chromatography (silica gel) to give the titled compound in 61% yield. ¹H-NMR (DMSO-d₆): δ (ppm) 1.18 (d, 6H); 2.88 (quint, 1H); 6.34 (s, 1H); 7.05 (d, 2H); 7.14 (d, 2H); 7.31 (d, 2H); 7.63 (d, 4H); 7.72 (d, 1H); 8.20 (d, 2H), 8.54 (d, 1H); MS (ESI+): m/z=637 [M+H]⁺; Melting point: 194-209° C.

### Example 138

### 3-chloro-4-(4-isopropyl-benzoyl)-1-(6-methyl-pyridazin-3-yl)-5-(4-trifluoromethoxy-phenyl)-1,5-dihydro-pyrrol-2-one

Oxalyl chloride 6.03 mmol (3 eq) was added at 0° C. to a solution of example 61 (1 eq) in dry dichloromethane and dry DMF (20 ml/20 ml). The mixture was stirred 4 h at 0° C., then diluted with NaHCO₃ 10% and extracted twice with ethyl acetate. The combined organic layer was washed with H₂O then dried on anhydrous Na₂SO₄, filtered and concentrated in vacuum. The residue was purified by flash chromatography (silica gel) to give the titled compound, in 29% yield. ¹H-NMR (DMSO-d₆): δ (ppm) 1.21 (d, 6H); 2.56 (s, 3H); 2.96 (quint, 1H); 6.73 (s, 1H); 7.27 (d, 2H); 7.34 (d, 2H); 7.58 (d, 2H); 7.64 (d, 1H); 7.80 (d, 2H); 8.33 (d, 1H); MS (ESI+): m/z=516 [M+H]⁺; Melting point: 293° C.

### Example 149

### N-[4-(4-isopropyl-benzoyl)-1-(6-methyl-pyridazin-3-yl)-2-oxo-5-(4-trifluoromethoxy-phenyl)-2,5-dihydro-1H-pyrrol-3-yl]-methanesulfonamide

Sodium hydride (1.08 mmol, 1.2 eq) was added portionwise to a solution of the methane sulphonamide (1.2 eq) in dry DMF (10 ml) and under nitrogen. The mixture was stirred at room temperature for 30 min and Example 138 (1 eq) in dry DMF (10 ml) was added. The mixture was stirred at room temperature for 15 min then diluted with H₂O and extracted with dichloromethane. The organic layer was washed with brine then dried on anhydrous Na₂SO₄, filtered and concentrated in vacuum. The residue was purified by HPLC semi-preparative to give the titled compound in 8% yield. ¹H-NMR (CD₃OD): δ (ppm) 1.28 (d, 6H); 2.56 (s, 3H); 2.95 (quint, 1H); 3.04 (s, 3H); 6.49 (s, 1H); 6.83 (d, 2H); 6.91-6.95 (m, 2H); 7.09-7.13 (m, 2H); 7.22 (d, 2H); 7.62 (d, 1H); 8.53 (d, 1H); MS (ESI+): m/z=575 [M+H]⁺

### Example 171

### 3-Isopropylamino-4-(4-isopropyl-benzoyl)-1-(6-methyl-pyridazin-3-yl)-5-(4-trifluoromethoxy-phenyl)-1,5-dihydro-pyrrol-2-one

Sodium hydride (1.08 mmol, 1.2 eq) was added portionwise to a solution isopropylamine (1.2 eq) in dry DMF (10 ml) and under nitrogen. The mixture was stirred at room temperature for 40 min and 3-chloro-4-(4-isopropyl-benzoyl)-1-(6-methyl-pyridazin-3-yl)-5-(4-trifluoromethoxy-phenyl)-1,5-dihydro-pyrrol-2-one (Example 138) (1 eq) in dry DMF (10 ml) was added. The mixture was stirred at room temperature for 30 min, then, diluted with H₂O and extracted with dichloromethane. The organic layer was washed with brine then dried over anhydrous Na₂SO₄, filtered and concentrated in vacuum. The residue was purified by HPLC semi-preparative to give the titled compound in 17% yield. ¹H-NMR (DMSO-d₆): δ (ppm) 1.08 (d, 6H); 1.23 (d, 6H); 2.46 (s, 3H); 2.92 (quint, 1H); 3.50 (quint, 1H); 6.19 (s, 1H); 6.40 (d, 1H); 6.67 (d, 2H); 6.86 (dd, 2H), 7.58 (dd, 3H); 8.28 (d, 1H); 11.26 (d, 1H); MS (ESI+): m/z=539 [M+H]⁺

### Example 182

### 3-hydroxy-4-(4-isopropyl-benzoyl)-1-(6-methyl-pyridazin-3-yl)-5-{4-[1-(2-trimethylsilanyl-ethoxymethyl)-1H-[1,2,4]triazol-3-yloxy]-phenyl}-1,5-dihydro-pyrrol-2-one

HCl, 6N (21 ml/mmol), was added to a solution of 3-hydroxy-4-(4-isopropyl-benzoyl)-1-(6-methyl-pyridazin-3-yl)-5-{4-[1-(2-trimethylsilanyl-ethoxymethyl)-1H-[1,2,4]triazol-3-yloxy]-phenyl}-1,5-dihydro-pyrrol-2-one (Example 200) in EtOH (21 ml/mmol). The solution was stirred at room temperature overnight then diluted with sodium bicarbonate 10% and dichloromethane. The organic layer was dried on anhydrous Na₂SO₄, filtered and concentrated in vacuum. The residue was triturated in Et₂O with EtOH (2-3 drops). After filtration the solid was dried in vacuum to give 3-hydroxy-4-(4-isopropyl-benzoyl)-1-(6-methyl-pyridazin-3-yl)-5-[4-(1H-[1,2,4]triazol-3-yloxy)-phenyl]-1,5-dihydro-pyrrol-2-one in 18% yield. ¹H-NMR (DMSO-d₆): δ (ppm) 1.20 (d, 6H); 2.50 (s, 3H); 2.89 (quint, 1H); 6.39 (s, 1H); 6.90 (d, 2H); 7.30 (dd, 4H); 7.55 (d, 1H); 7.70 (d, 2H); 8.32 (brs, 2H); 13.64 (brs, 1H); MS (ESI+): m/z=497 [M+H]⁺

### Example 212

### 3-chloro-4-(4-isopropyl-benzoyl)-1-(6-methyl-pyridazin-3-yl)-5(R)-(4-trifluoromethoxy-phenyl)-1,5-dihydro-pyrrol-2-one

Oxalyl chloride 6.03 mmol (3 eq) was added at 0° C. to a solution of example 188 (1 eq) in dry dichloromethane and dry DMF (20 ml/20 ml). The mixture was stirred 4 h at 0° C., then diluted with NaHCO₃ 10% and extracted twice with ethyl acetate. The combined organic layer was washed with H₂O then dried on anhydrous Na₂SO₄, filtered and concentrated in vacuum. The residue was purified by flash chromatography (silica gel) to give the titled compound, in 41% yield. ¹H-NMR (DMSO-d₆): δ (ppm) 1.21 (d, 6H); 2.56 (s, 3H); 2.96 (quint, 1H); 6.73 (s, 1H); 7.27 (d, 2H); 7.34 (d, 2H); 7.58 (d, 2H); 7.64 (d, 1H); 7.80 (d, 2H); 8.33 (d, 1H); MS (ESI+): m/z=516 [M+H]⁺; [α]_{D}=+131.57° (c=0.285 in DMSO).

### Intermediate 11

### 5-isopropoxy-pyridine-2-carbaldehyde

Anhydrous potassium carbonate (1 eq) and 2-bromopropane (1 eq) were added to a solution of 5-hydroxy-pyridine-2-carbaldehyde (1 eq) in DMF (3.4 ml/mmol). The reaction mixture was stirred at 100° C. for 1 h then diluted with water. The aqueous layer was extracted twice with ethyl acetate. The organic layers were combined, dried on anhydrous Na₂SO₄ filtered and concentrated in vacuum to give the titled compound in 82% yield. ¹H-NMR (DMSO-d₆): δ (ppm) 1.40 (d, 6H); 4.70 (quint, 1H); 7.25 (dd, 1H); 7.94 (d, 1H); 8.38 (d, 1H); 9.97 (brs, 1H); MS (ESI+): m/z=166 [M+H]⁺

### Intermediate 12

### 5-ethoxy-pyridine-2-carbaldehyde

Anhydrous potassium carbonate (1 eq) and bromoethane (1 eq) were added to a solution of 5-hydroxy-pyridine-2-carbaldehyde (1 eq) in DMF (3.4 ml/mmol). The reaction mixture was stirred at 100° C. for 1 h then diluted with water. The aqueous layer was extracted twice with ethyl acetate. The organic layers were combined, dried on anhydrous Na₂SO₄ filtered and concentrated in vacuum to give the titled compound in 90% yield. ¹H-NMR (CDCl₃): δ (ppm) 1.48 (t, 3H); 4.16 (q, 2H); 7.27 (dd, 1H), 7.94 (d, 1H); 8.41 (d, 1H); 9.97 (s, 1H); MS (ESI+): m/z=152 [M+H]⁺

### Intermediate 19

### 4-(1-benzyl-1H-tetrazol-5-yloxy)-benzaldehyde

Potassium tert-butoxyde (1.13 eq) was added under argon atmosphere to a solution of 4-hydroxybenzaldehyde (1 eq) in dry DMF (2.5 ml/mmol). The mixture was stirred 15 min at room temperature. 1-benzyl-5-bromo-1H-tetrazole (1 eq) in dry DMF (2.5 ml/mmol) was added. The reaction mixture was refluxed for 5 hours and concentrated under vacuum. The crude product was diluted with water and extracted twice with dichloromethane. The organic layers were dried over anhydrous MgSO₄, filtered and concentrated. The residue was purified by flash chromatography (silica gel) to give intermediate 19 in 86% yield. 1H-NMR (CDCl₃): δ (ppm) 5.47 (s, 2H); 7.38 (brs, 5H); 7.53 (d, 2H); 7.95 (d, 2H); 10 (s, 1H); MS (ESI+): m/z=281 [M+H]⁺

### Intermediate 20

### 4-(1,1-difluoro-ethoxy)-benzaldehyde

Manganese (IV) oxide activated (5 eq) was added to a solution of Intermediate 27 in dioxane (3.6 ml/mmol). The reaction mixture was stirred at room temperature overnight. After filtration of the mixture on Celite® and filtration on SPE (2 g), the filtrate was concentrated under vacuum to give 4-(1,1-difluoro-ethoxy)-benzaldehyde (intermediate 20) in quantitative yield. 1H-NMR (CDCl₃): δ (ppm) 4.28 (dt, 2H); 5.91 (t, 0.25H); 6.13 (t, 0.5H); 6.35 (t, 0.25H); 7.04 (d, 2H); 7.87 (d, 2H); 9.92 (s, 1H); MS (ESI+): m/z=187 [M+H]+

### Intermediate 26

### 4-(1,1-difluoro-ethoxy)-benzoic acid methyl ester

Potassium fluoride (1 eq) was added to a solution of methyl-4-hydroxybenzoate (1 eq) in MeOH (10 ml/mmol). The mixture was stirred 15 min at room temperature. The reaction mixture was concentrated under vacuum. Et2O was added in the crude product and the solution was concentrated under vacuum. This mixture was dissolved in DMSO (10 ml/mmol) and 1,1-difluoro-2-iodoethane in DMSO (0.7 ml/mmol) was added. The solution was purged under argon and heated in a sealed tube at 120° C. for 21 hours. The reaction mixture was diluted with water and extracted (3×) with ethyl acetate. The organic layers were dried on anhydrous Na2SO4, filtered and concentrated in vacuum. The residue was purified by flash chromatography (silica gel) to give 4-(1,1-difluoro-ethoxy)-benzoic acid methyl ester in 43% yield. ¹H-NMR (CDCl₃): δ (ppm) 4.25 (dt, 2H); 5.90 (t, 0.25H); 6.12 (t, 0.5H); 6.34 (t, 0.25H); 6.94 (d, 2H); 8.02 (d, 2H); MS (ESI+): m/z=217 [M+H]+

### Intermediate 27

### [4-(1,1-difluoro-ethoxy)-phenyl]-methanol

LiAlH₄ (1.5 eq) was added dropwise to a solution of Intermediate 26 in dry THF (2.4 ml/mmol) under argon atmosphere at 0° C. The reaction mixture was stirred at room temperature for 1 hour. The reaction was quenched with water and ice then filtrated on Celite®. The filtrate was extracted twice with dichloromethane and washed with water and brine. The organic layers were dried over anhydrous Na₂SO₄, filtered and concentrated in vacuum to give [4-(1,1-difluoro-ethoxy)-phenyl]-methanol in 81% yield. 1H-NMR (CDCl₃): δ (ppm) 4.19 (dt, 2H); 4.64 (brs, 2H); 5.86 (t, 0.25H); 6.09 (t, 0.5H); 6.31 (t, 0.25H); 6.91 (d, 2H); 7.32 (d, 2H)

### Intermediate 21

### 4-(4-methanesulfonyl-phenyl)-2,4-dioxo-butyric acid ethyl ester

4-methylsulfonylacetophenone (1 eq) was added at 0° C. to a solution of EtONa (prepared in situ with Na (1.3 eq) and ethanol). The mixture was stirred 45 min then diethyl oxalate was added dropwise. The mixture was refluxed overnight then concentrated to give the crude compound, which was diluted in ethyl acetate and was washed with HCl 1N then water and brine. The organic layers were combined, dried on anhydrous Na₂SO₄, filtered and concentrated in vacuum. The residue was purified by flash chromatography (silica gel) to give the titled compound in 53% yield. 1H-NMR (DMSO-d₆): δ (ppm) 1.31 (t, 4H); 4.32 (q, 2H); 7.15 (s, 1H); 8.10 (d, 2H); 8.30 (d, 1H); MS (ESI+): m/z=299 [M+H]⁺

### Intermediate 22

### 2,4-dioxo-4-(4-pyrrolidin-1-yl-phenyl)-butyric acid ethyl ester

4-(1-pyrrolidino)acetophenone (1 eq) was added, at 0° C., to a solution of EtONa (prepared in situ with Na (1.3 eq) and ethanol). The mixture was stirred 45 min, then, diethyl oxalate was added dropwise. The mixture was refluxed overnight then concentrated in vacuum to give the crude product. It was diluted in ethyl acetate and was washed with HCl, 1N, then, water and brine. The organic layers were combined, dried on anhydrous Na₂SO₄, filtered and concentrated in vacuum. The residue was purified by flash chromatography (silica gel) to give the titled compound in 63% yield. 1H-NMR (CDCl₃): δ (ppm) 1.40 (t, 3H); 2.03-2.08 (m, 4H); 3.37-3.43 (m, 4H); 4.38 (q, 2H); 6.56 (d, 2H); 6.99 (s, 1H); 7.91 (d, 2H); 15.91 (brs, 1H); MS (ESI+): m/z=290 [M+H]+

### Intermediate 23

### Synthesis of 4-(5-methyl-isoxazol-3-yloxy)-benzaldehyde

5-methyl-isoxazol-3-ol (1.1 eq) and potassium carbonate (1 eq) were added to a solution of 4-fluorobenzaldehyde (1 eq) in N,N-dimethylacetamide (1 ml/mmol). The solution was stirred at reflux for 3 h. The reaction mixture was diluted with water and extracted twice with ethyl acetate. The organic layers were washed with NaOH, 1N, then, brine, dried over anhydrous Na₂SO₄, filtered and concentrated in vacuum. The residue was purified by flash chromatography (silica gel) then triturated in Et₂O. After filtration the filtrate was concentrated in vacuum to give 4-(5-methyl-isoxazol-3-yloxy)-benzaldehyde in 48% yield. ¹H-NMR (DMSO-d₆): δ (ppm) 2.40 (s, 3H); 6.28 (s, 1H); 7.44 (d, 2H); 7.99 (d, 2H); 9.98 (s, 1H)

### Intermediate 24

### 3-bromo-1-(2-tritnethylsilanyl-ethoxymethyl)-1H-[1,2,4]triazole

Sodium hydride (1.2 eq) was added to a solution of 3-bromo-1H-[1,2,4]triazole (1 eq) in dry DMF (2.3 ml/mmol) at 0° C. and under an atmosphere of nitrogen The solution was stirred at 0° C. for 20 min, then, SEM-Cl (1.2 eq) was added. The mixture was stirred at room temperature overnight and diluted with H2O and ethyl acetate. The organic layers was washed with H2O then brine, dried on anhydrous Na2SO4, filtered and concentrated in vacuum to give the titled compound. Crude compound 3-bromo-1-(2-trimethylsilanyl-ethoxymethyl)-1H-[1,2,4]triazole was engaged in step 2 without purification. ¹H-NMR (CDCl₃): δ (ppm) 0.00 (t, 9H); 0.92 (t, 2H); 3.64 (t, 2H); 5.44 (s, 2H); 8.13 (s, 1H)

### Intermediate 25

### 4-[1-(2-tritnethylsilanyl-ethoxymethyl)-1H-[1,2,4]triazol-3-yloxy]-benzaldehyde

Potassium tert-butoxyde (1.13 eq) was added to a solution of the 4-hydroxybenzaldehyde (1 eq) in dry DMF (2.3 ml/mmol) and under an atmosphere of nitrogen. The solution was stirred at room temperature for 30 min then intermediate 24 (1 eq) in dry DMF (2.3 ml/mmol) was added. The mixture was stirred at reflux for 6 h15 and diluted with H₂O and ethyl acetate. The aqueous layer was extracted with ethyl acetate. The organic layers were washed twice with H2O then brine, dried on anhydrous Na2SO4, filtered and concentrated in vacuum. The residue was purified by flash chromatography (silica gel) to give 4-[1-(2-trimethylsilanyl-ethoxymethyl)-1H-[1,2,4]triazol-3-yloxy]-benzaldehyde. 1H-NMR (CDCl₃): δ (ppm) 0.00 (t, 9H); 0.94 (t, 2H); 3.71 (t, 2H); 5.88 (s, 2H); 7.22 (d, 2H); 7.95 (d, 2H); 9.91 (s, 1H); 9.99 (s, 1H)

### II Biological Testing of the Compounds According to the Present Invention

The compounds according to the present invention were tested for their anti Hepatitis C activity as follow:

### Materials and Methods

### Cell Culture:

Human Hepatoma Huh-7 cell line was maintained in DMEM/HAMF-12 supplemented with 10% SVF, 4 mM glutamine, 0.5M Na pyruvate, 1% penistreptomycine. HCV replicon containing Huh-7 cell lines Huh-9.13 and Luc Neo ET (Reblikon) were maintained in DMEM supplemented with 10% SVF, 2 mM glutamine, and 1×NEAA, 100 U/ml penicillin, and 100 μg/ml streptomycine. Replicon cells were maintained in medium supplemented with 1 mg/ml G418 for replicon Huh-9.13 and 0.5 mg/ml for Luc Neo et replicon unless indicated otherwise. Huh-7 and HCV replicon cell lines were maintained at 37° C. and 5% CO₂ in a humidified atmosphere. Cells were dissociated at sub confluence with trypsin EDTA 1×.

### Plasmid Construction:

cDNA encoding HCV NS5B genotype 1b, was cloned in frame with Gal4-DNA Binding Domain. The protein was expressed with a 21 amino acid C-terminal deletion to remove transmembrane domain. Expression of NS5BΔ21/Gal4 DBD fusion protein was under control of SV40 early promoter. 3D-Sensor peptide was cloned in frame with VP16 activation domain. Expression of 3D-Sensor/VP16 AD fusion protein was under control of CMV promoter. Expression of the firefly luciferase reporter gene was inducible by the [Target protein/conformation sensitive peptide/VP16AD] complex.

### 3D-SCREEN Assay:

3D-SCREEN assay is a reporter gene assay designed to identify chemical entities that modify the 3D-structure of target proteins and hence inhibit their biological activity (WO2006/046134). It is a single-target, cell based assay. Briefly, expression of a reporter gene depends on the interaction of a short peptide, thereafter named 3D-Sensor, and native conformation of the target protein. Whenever the conformation of the target protein is modified, interaction between 3D-sensor and target protein is disrupted and reporter gene is not expressed anymore. Conformation modifiers are identified by loss of expression of reporter gene. NS5B 3D-Screen platform was generated in Huh-7 cell lines by transient transfection of three expression vectors encoding respectively

(i) HCV NS5BΔ21/Gal4-DBD fusion protein

(ii) the 3D-sensor peptide I4/VP16 fusion protein

(iii) the firefly luciferase reporter gene

Huh-7 cells were dissociated the day before transfection and seeded in T175 flasks at a density of 10⁷ cells in 30 ml culture medium. Equimolar ratios of vectors were transfected in cell according to optimized jetPEI transfection protocol (PolyPlus Transfection, Illkirch, France) and 10 μg total DNA/10⁶ cells. Transfection was performed for 2 hours at 37° C. and 5% CO₂ in a humidified atmosphere. After two hours cells were dissociated and seeded in 96 wells plates at a density of 25,000 cells per well and 90 μl culture medium. 10 μl of compounds to be tested were added 2 hours after seeding. Final concentration of DMSO was 1%. Cells were incubated in the presence of compounds for 24 hours after which expression of firefly luciferase reporter gene was quantified. Briefly, culture medium was removed and cells were lysed by addition of 100 μl of lysis buffer containing 125 mM Tris Phosphate ph 7.8, 10 mM EDTA, 5 mM DTT, 50% glycerol and 5% Triton. Plates were vortexed 10 min at 1300 rpm. Cell lysat was transferred in OpaqueWhite Assay 96 well Flat Bottom plates. 100 μl of luciferin solution 1× were added to each well. Luciferin solution contained 40 mM Tris Phosphate ph 7.8, 0.2 mM EDTA, 67 mM DTT, 2.14 mM MgCl2, 5.4 mM MgSO4, 4.7×10⁻⁴ M luciferin, 5.3×10⁻⁴ M ATP and 2.7×10⁻⁴ M Acetyl co enzyme A. Luminescence was immediately measured with Berthold Microlumat Plus LB 96V luminometer with an integration of 0.5 sec. Inhibition was calculated using the formula: % inhibition=(1−(read/average max))*100.
Average max=signal in absence of compound

### Replicon Assay

Replicon Luc Neo ET is a bicistronic expression constructs (Lohmann et al, 1999, Science 285, 110-113). In brief, the structural genes of the HCV genome were replaced by heterologous sequences; the gene encoding the neomycin phosphotransferase (NPT) and the internal ribosome entry site (IRES) of the encephalomyocarditis virus (EMCV). The bicistronic construct is therefore composed of the following elements: HCV-IRES nucleotides 1-389, the NPT gene, the EMCV-IRES directing translation of downstream HCV sequences from NS2 or NS3 up to the authentic 3′ end of the genome. HCV Polyprotein harbours the cell culture adaptive mutations E1202G, T1280I, K1846T. G418-resistance is only possible with cells containing high amounts of replicon.

### Luc Neo ET Replicon

Cells were dissociated the day before addition of compounds and seeded in 96 well-plates at a final concentration of 77 777.77 cells.ml⁻¹.well⁻² in 90 μl final volume of culture medium per well and were maintained at 37° C. and 5% CO₂ in a humidified atmosphere for 24 hours. 10 μl of compounds to be tested were added 24 hours after seeding. Final concentration of DMSO was 1%. Cells were incubated in the presence of compounds for 72 hours after which expression of firefly luciferase reporter gene was quantified. Briefly, culture medium was removed and cells were lysed by addition of 100 μl of lysis buffer containing 125 mM Tris Phosphate ph 7.8, 10 mM EDTA, 5 mM DTT, 50% glycerol and 5% Triton. Plates were vortexed 10 min at 1300 rpm. Cell lysat was transferred in OpaqueWhite Assay 96 well Flat Bottom plates. 100 μl of luciferin solution 1× were added to each well. Luciferin solution contained 40 mM Tris Phosphate ph 7.8, 0.2 mM EDTA, 67 mM DTT, 2.14 mM MgCl2, 5.4 mM MgSO4, 4.7×10⁻⁴ M luciferin, 5.3×10⁻⁴ M ATP and 2.7×10⁻⁴ M Acetyl co enzyme A. Luminescence was immediately measured with Berthold Microlumat Plus LB 96V luminometer with an integration of 0.5 sec Inhibition was calculated using the formula:

% inhibition=1−[(RLUsample−RLUbackground)/(RLUsignal−RLUbackground)]

### HCV NS5B RdRp Enzyme Assay

### Assay Conditions

The assay was performed in a total volume of 20 μl containing 20 mM Tris pH 7.5, 1 mM DTT, 17 U RNasin, 50 mM NaCl, 10% DMSO, 5 mM MgCl₂, 0.5 mM each of the 3 NTPs (ATP, CTP, GTP), 86 nM RNA template (341 nt from the 3′ end of HCV minus strand RNA), 50 nM of purified HCV NS5B with a deletion of the 21 C-terminal amino acids and 2 μCi [³H]UTP (46 Ci.mmol⁻¹). The reaction mixture was incubated for 2 h at 25-30° C. and the radiolabeled products were precipitated by the addition of 10% TCA. The radioactivity incorporated was quantified by counting in a Wallac scintillation counter. Increasing concentrations of tested compounds were added to the complete RdRp reaction mixture. After a two hour incubation period at 25-30° C., the amount of labeled product was determined as above. Two types of control reactions were done: a negative control corresponding to the complete mixture without enzyme and a positive control with enzyme but without compounds. In each experiment, test and control samples are in duplicate.

### Evaluation of Inhibitory Potential of Tested Compounds

The level of activity with each compound concentration was expressed with the formulae:

$$\% activity ( test tube ) = \frac{\begin{matrix}{}^{3} H cpm test tube - \\ {}^{3} H cpm negative control\end{matrix}}{\begin{matrix}{}^{3} H cpm positive control - \\ {}^{3} H cpm negative control\end{matrix}} \times 100$$

The IC₅₀ value was calculated as the compound concentration reducing polymerase activity by 50%.

The results are indicated in the following tables:

TABLE 1
3D-SCREEN assay results

| | EC50_3DS_NS5B |
|---|---|
| Example | (μM) |
| 2 | 9 |
| 7 | 9 |
| 8 | 2 |
| 9 | 7 |
| 10 | 3 |
| 18 | 1 |
| 20 | 0.4 |
| 22 | 0.6 |
| 23 | 1 |
| 24 | 3 |
| 25 | 0.5 |
| 29 | 6 |
| 33 | 2 |
| 35 | 3 |
| 37 | 9 |
| 38 | 10 |
| 41 | 7 |
| 42 | 4 |
| 44 | 2 |
| 45 | 4 |
| 48 | 0.7 |
| 49 | 1 |
| 53 | 5 |
| 56 | 0.5 |
| 58 | 5 |
| 61 | 0.07 |
| 62 | 0.8 |
| 63 | 0.06 |
| 66 | 2 |
| 67 | 3 |
| 69 | 0.5 |
| 76 | 0.6 |
| 79 | 0.5 |
| 80 | 2 |
| 83 | 8 |
| 84 | 3 |

TABLE 2
Replicon assay results

| | EC50_3DS_NS5B |
|---|---|
| Example | (μM) |
| 77 | <1 |
| 85 | <10 |
| 86 | <0.1 |
| 87 | <10 |
| 88 | <1 |
| 89 | <10 |
| 90 | <10 |
| 94 | <10 |
| 95 | <10 |
| 96 | <1 |
| 97 | <10 |
| 98 | <10 |
| 100 | <10 |
| 101 | <10 |
| 102 | <1 |
| 104 | <1 |
| 105 | <1 |
| 106 | <10 |
| 109 | <10 |
| 110 | <1 |
| 111 | <10 |
| 115 | <10 |
| 118 | <1 |
| 119 | <1 |
| 120 | <1 |
| 121 | <10 |
| 123 | <1 |
| 125 | <10 |
| 126 | <10 |
| 128 | <1 |
| 129 | <1 |
| 130 | <10 |
| 131 | <1 |
| 135 | <10 |
| 136 | <10 |
| 137 | <10 |
| 138 | <10 |
| 142 | <1 |
| 143 | <10 |
| 147 | <10 |
| 149 | <1 |
| 155 | <1 |
| 158 | <0.1 |
| 159 | <1 |
| 160 | <1 |
| 161 | <1 |
| 162 | <1 |
| 165 | <1 |
| 167 | <1 |
| 169 | <1 |
| 172 | <1 |
| 173 | <1 |
| 174 | <10 |
| 176 | <10 |
| 183 | <10 |
| 184 | <10 |
| 185 | <10 |
| 186 | <1 |
| 187 | <1 |
| 188 | <0.1 |
| 189 | <10 |
| 190 | <10 |
| 191 | <0.1 |
| 192 | <1 |
| 194 | <10 |
| 195 | <1 |
| 196 | <1 |
| 197 | <10 |
| 198 | <1 |
| 199 | <1 |

TABLE 3
RdRp enzyme assay results

| Example | EC50_Replicon LucneoET (μM) |
|---|---|
| 2 | <10 |
| 8 | 3 |
| 20 | 0.1 |
| 22 | 0.5 |
| 25 | 0.2 |
| 48 | 1 |
| 56 | 0.6 |
| 61 | 0.1 |
| 62 | 0.9 |
| 63 | 0.1 |
| 67 | <10 |
| 69 | 0.2 |
| 76 | <10 |
| 77 | <10 |
| 79 | <10 |
| 80 | <10 |
| 86 | <1 |
| 88 | <1 |
| 89 | <10 |
| 90 | <10 |
| 94 | <10 |
| 96 | <1 |
| 98 | <10 |
| 102 | <1 |
| 104 | <1 |
| 105 | <1 |
| 106 | <10 |
| 110 | <1 |
| 111 | <10 |
| 115 | <10 |
| 118 | <1 |
| 119 | <10 |
| 120 | <1 |
| 123 | <1 |
| 125 | <10 |
| 128 | <10 |
| 129 | <10 |
| 130 | <10 |
| 131 | <10 |
| 137 | <10 |
| 138 | <10 |
| 142 | <10 |
| 143 | <10 |
| 149 | <1 |
| 155 | <1 |
| 158 | <1 |
| 159 | <10 |
| 160 | <10 |
| 161 | <10 |
| 162 | <10 |
| 165 | <1 |
| 167 | <1 |
| 169 | <1 |
| 188 | <0.1 |
| 189 | <10 |
| 191 | <0.1 |
| 192 | <1 |
| 195 | <1 |
| 196 | <1 |
| 198 | <1 |
| 199 | <1 |

### Example 63

### 50% Inhibition at 10 μM

### BIBLIOGRAPHIC REFERENCES

Ago H, Adachi T, Yoshida A, Yamamoto M, Habuka N, Yatsunami K, Miyano M. Crystal structure of the RNA-dependent RNA polymerase of hepatitis C virus Structure. Fold. Des. 1999. 7(11):1417-26
Bressanelli S et al. Crystal structure of the RNA-dependent RNA polymerase of hepatitis C virus. Proc. Natl. Acad. Sci. USA. 1999. 96(23):13034-9
Fried Michael W. 2002. Side effects of therapy of hepatitis and their management. Hepatology Vo132:S237-244
Gordon, C. P. and P. A. Keller. 2005. Control of Hepatitis C: A Medicinal Chemistry Perspective. Journal of Medicinal Chemistry 48: 1-20
Hugle T, Cerny A. Current therapy and new molecular approaches toantiviral treatment and prevention of hepatitis C. Rev. Med. Virol. 2003. 13:361-71.
Ivashkina N, Wölk B, Lohmann V, Bartenschlager R, Blum H E, Penin F, Moradpour D. The hepatitis C virus RNA-dependent RNA polymerase membrane insertion sequence is a transmembrane segment. J. Virol. 2002 December; 76(24):13088-93.
Lake-Bakaar, G. 2003. Current and future therapy for chronic hepatitis C virusliver disease. Curr Drug Targets Infect Disord 3:247-53.
Lindenbach, B. D., M. J. Evans, A. J. Syder, B. et al. Complete Replication of Hepatitis C Virus in Cell Culture. Science. 2005. 309:623-626.
Lesburg C A, Cable M B, Ferrari E, Hong Z, Mannarino A F, Weber P C. Crystal structure of the RNA-dependent RNA polymerase from hepatitis C virus reveals a fully encircled active site. Nat. Struct. Biol. 1999. 6(10):937-43.
Moussalli J, Opolon P, Poynard T. Management of hepatitis C. J. Viral. Hepatitis. 1998. 5:73-82.
Moradpour D et al. Membrane association of the RNA-dependent RNA polymerase is essential for hepatitis C virus RNA replication. J. Virol. 2004. 78(23):13278-84.
Schmidt-Mende J, Bieck E, Hugle T, Penin F, Rice C M, Blum H E, Moradpour D. Determinants for membrane association of the hepatitis C virus RNA-dependent RNA polymerase. J Biol. Chem. 2001 Nov. 23; 276(47):44052-63.
Walker M. P and Z. Hong. HCV RNA-dependent RNA polymerase as a target for antiviral development. Cur. Op. Pharmacol. 2002. 2:1-7.

1.-16. (canceled)

17. A compound of the following formula I or a salt, solvate, tautomer, isotope, enantiomer, diastereoisomer or racemic mixture thereof: 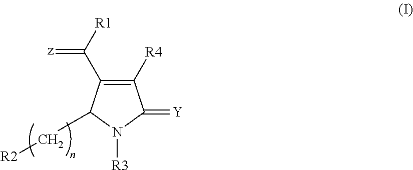
in which
n is an integer chosen between 0, 1 or 2;
Y represents an oxygen atom or a sulfur atom;
C═Z represents CH₂ or
Z represents an oxygen atom, a —CH—R group or a —N—OR group, in which R represents an hydrogen atom, a C₁-C₆ alkyl group, a C₃-C₆ cycloalkyl group, a 3-6 members heterocyclic group containing one or two heteroatoms selected in the group consisting of oxygen, nitrogen and sulfur atom, a (C₁-C₆ alkyl)COOH group, a (C₁-C₆ alkyl)O(C₁-C₆ alkyl) group or a O-protecting group;
R1 represents:
a phenyl group, or
a 5- or 6-members heteroaryl group containing one, two or three heteroatoms selected in the group consisting of oxygen, nitrogen and sulfur atom,
the phenyl group and the heteroaryl group being optionally substituted by:
a halogen atom;
a —CN group;
a —SO₂—(C₁-C₆) alkyl group in which the alkyl group is optionally substituted by one or more halogen atom;
a phenyl group;
a 5- or 6-members heteroaryl group containing one, two or three heteroatoms selected in the group consisting of oxygen, nitrogen and sulfur atom;
a C₁-C₆ alkyl group optionally substituted by one or more halogen atom;
a C₂-C₆ alkenyl group optionally substituted by one or more halogen atom;
a —O—(C₁-C₆)alkyl group in which the alkyl group is optionally substituted by one or more halogen atom;
a O—(C₁-C₆)alkyl-O—(C₁-C₆)alkyl group in which the alkyl group is optionally substituted by one or more halogen atom;
a —O—(C₁-C₆)alkyl-phenyl-O—(C₁-C₆)alkyl group;
a C₃-C₆ cycloalkyl group optionally substituted by one or more halogen atom;
a 5-, 6- or 7-members heterocyclic group containing one, two or three heteroatoms selected in the group consisting of nitrogen, sulfur and oxygen atom; or
a —NR′R″ group in which R′ and R″ represent independently of each other a hydrogen atom or a C₁-C₆ alkyl group;
R2 represents:
a phenyl group, or
a 5- or 6-members heteroaryl group containing one, two or three heteroatom (s) selected in the group consisting of oxygen, sulfur and nitrogen atom,
the phenyl group and the heteroaryl group being optionally substituted by one or more groups independently selected among:
a halogen atom;
a —OH group;
a —CN group;
a C₁-C₆ alkyl group optionally substituted by one or more halogen atom or by a 5-members heteroaryl group containing one, two, three or four heteroatom (s) selected in the group consisting of oxygen, sulfur and nitrogen atom;
a —O—(C₁-C₆)alkyl group in which the alkyl group is optionally substituted by one or more halogen atom;
a —CO—(C₁-C₆)alkyl group in which the alkyl group is optionally substituted by one or more halogen atom;
a —SO₂—(C₁-C₆)alkyl group in which the alkyl group is optionally substituted by one or more halogen atom;
a —S—(C₁-C₆)alkyl group in which the alkyl group is optionally substituted by one or more halogen atom;
a C₂-C₆ alkenyl group optionally substituted by one or more halogen atom;
a C₂-C₆ alkynyl group optionally substituted by one or more halogen atom;
a C₃-C₆ cycloalkyl group optionally substituted by one or more halogen atom;
a —O—(C₃-C₆)cycloalkyl group in which the cycloalkyl group is optionally substituted by one or more halogen atom;
a phenyl group;
a —O-phenyl group;
a 5-members heteroaryl group containing one, two, three or four heteroatom (s) selected in the group consisting of oxygen, sulfur and nitrogen atom, the heteroaryl group being optionally substituted by a C₁-C₆ alkyl group;
a —NR′R″ group in which R′ and R″ represent independently of each other a hydrogen atom or a C₁-C₆ alkyl group;
a —O-(6-members heterocyclic) group in which the heterocyclic group contains one, two or three heteroatoms selected in the group consisting of nitrogen, sulfur and oxygen atom;
a —O-(5- or 6-members heteroaryl) group in which the heteroaryl group contains one, two, three or four heteroatom (s) selected in the group consisting of oxygen, sulfur and nitrogen atom, the heteroaryl group being optionally substituted by a —(C₁-C₆ alkyl)-phenyl group, a —(C₁-C₆ alkyl) group or a —CH₂—O—CH₂—Si(CH₃)₃ group, the alkyl group being optionally substituted by a halogen atom;
a —O—((C₁-C₆)alkyl)-(6-members heterocyclic) group in which the heterocyclic group contains one, two or three heteroatoms selected in the group consisting of nitrogen, sulfur and oxygen atom;
a —O—((C₁-C₆)alkyl)-NR′R″ group in which R′ and R″ represent independently of each other a hydrogen atom or a C₁-C₆ alkyl group; and
a 6-members heterocyclic group containing one, two or three heteroatoms selected in the group consisting of nitrogen, sulfur and oxygen atom, the heterocyclic group being optionally substituted by a —((C₁-C₆)alkyl)-NR′R″ group in which R′ and R″ represent independently of each other a hydrogen atom or a C₁-C₆ alkyl group;
R3 represents
either
a group of the following formula II: 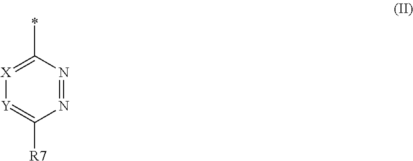
in which
X represents a nitrogen atom and Y represents a —C—R8 group or
X represents a —C—R9 group and Y represents a nitrogen atom or
X represents a —C—R9 group and Y represents a —C—R8 group,
R7, R8 and R9 represent independently of each other
a hydrogen atom;
a halogen atom;
a C₁-C₆ alkyl group optionally substituted by one or more halogen atom, by a —O—(C₁-C₆)alkyl group, by a —O—C₃-C₆ cycloalkyl group, by a —O-aryl group or by a —NR′R″ group in which R′ and R″ represent independently of each other a hydrogen atom, a C₁-C₆ alkyl group, a C₃-C₆ cycloalkyl group or an aryl group;
a C₃-C₆ cycloalkyl group optionally substituted by one or more halogen atom;
a —O—(C₁-C₆)alkyl group in which the alkyl group is optionally substituted by one or more halogen atom or by a phenyl group;
a —COON group;
a —OH group;
a —COO(C₁-C₆)alkyl group in which the alkyl group is optionally substituted by one or more halogen atom;
a —CN group;
a —S-phenyl-NO₂ group;
a —SO₂-phenyl-NO₂ group;
a —SO₂—(C₁-C₆)alkyl group;
a —SO₂-aryl group;
a —SO₂—NH—(C₁-C₆)alkyl group;
a —SO₂—NH-aryl group;
a 6-members heterocyclic group containing one or two heteroatoms selected in the group consisting of nitrogen, sulfur and oxygen atom; or
a —NR′R″ group in which R′ and R″ represent independently of each other a hydrogen atom or a C₁-C₆ alkyl group, and
* indicates the position involved in binding with another group.
or
a 6-members heteroaryl group containing as the only heteroatom, one nitrogen atom, said heteroaryl group being optionally substituted by a halogen atom; a C₁-C₆ alkyl group; a C₃-C₆ cycloalkyl group optionally substituted by one or more halogen atom; a —O—(C₁-C₆)alkyl group in which the alkyl group is optionally substituted by a phenyl group; a —OH group; a —COOH group; a —COO(C₃-C₆)alkyl group or a COOC₁alkyl group in which the alkyl group is optionally substituted by one or more halogen atom; a —CN group; a ═O group; a —SO₂-phenyl-NO₂ group; a —S-phenyl-NO₂ group; a —SO₂—(C₁-C₆)alkyl group; a —SO₂-aryl group; a —SO₂—NH—(C₁-C₆)alkyl group; a —SO₂—NH-aryl group; a 6-members heterocyclic group containing one or two heteroatoms selected in the group consisting of nitrogen, sulfur and oxygen atom; or a —NR′R″ group in which R′ and R″ represent independently of each other a hydrogen atom or a C₁-C₆ alkyl group,
R4 represents:
a —OH group or
a halogen atom or
a —O—C═O—(C₁-C₆alkyl) group or
a —O—(C₁-C₆)alkyl group or
a —O—(C₁-C₆)alkyl-CO—O—(C₁-C₆)alkyl group, in which the alkyl group is optionally substituted by a phenyl group;
a —NHR₅ group in which R₅ represents an hydrogen or a (C₁-C₆alkyl) group, or
a —NHSO₂R₆ group in which R₆ represents a hydrogen atom or a (C₁-C₆alkyl) group;
with the proviso that the compound of formula I does not correspond to the following one:

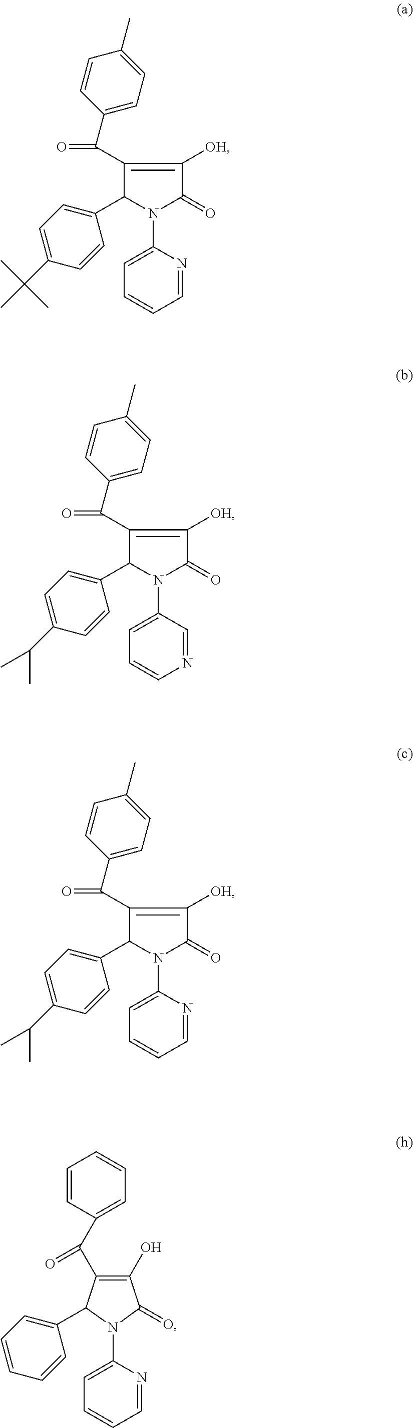

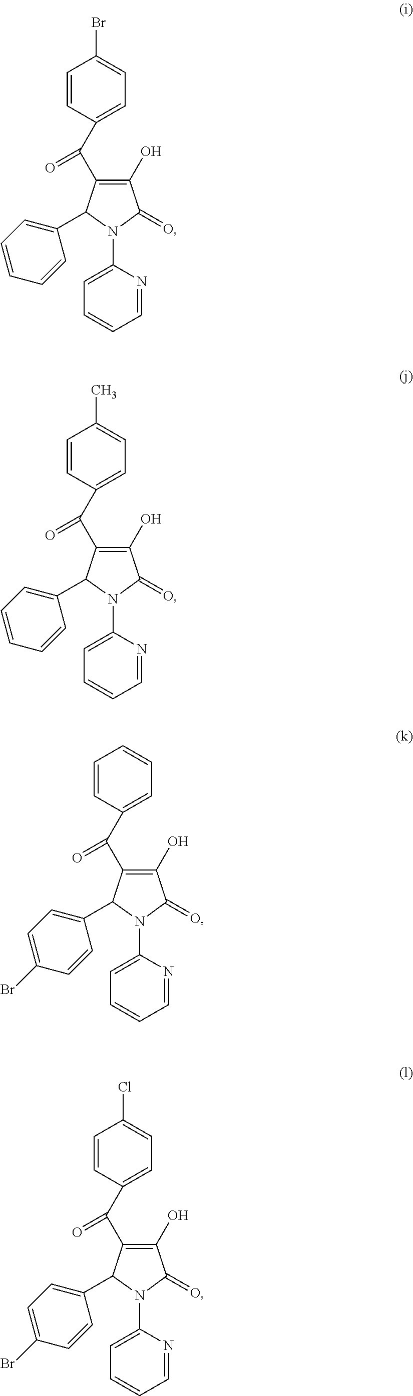

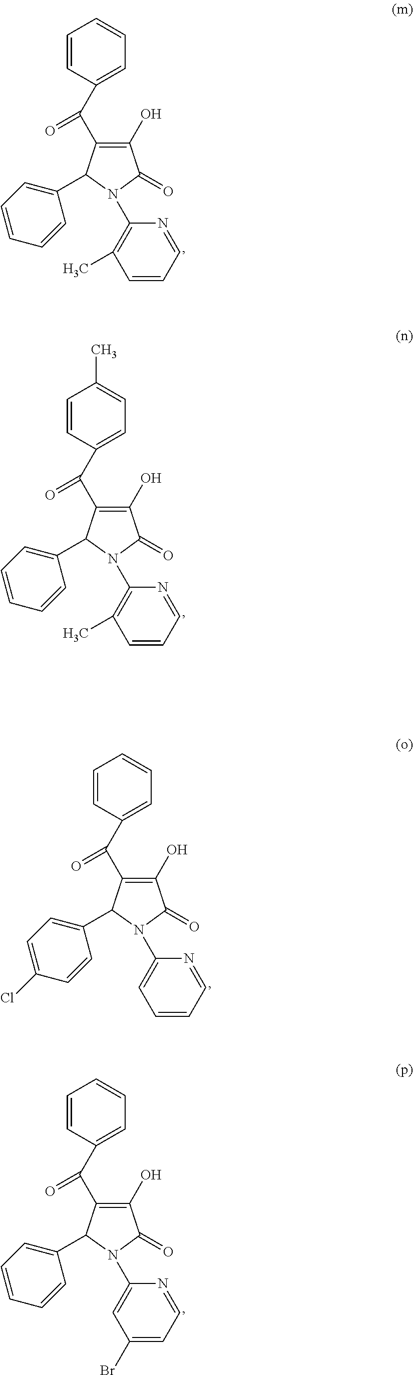

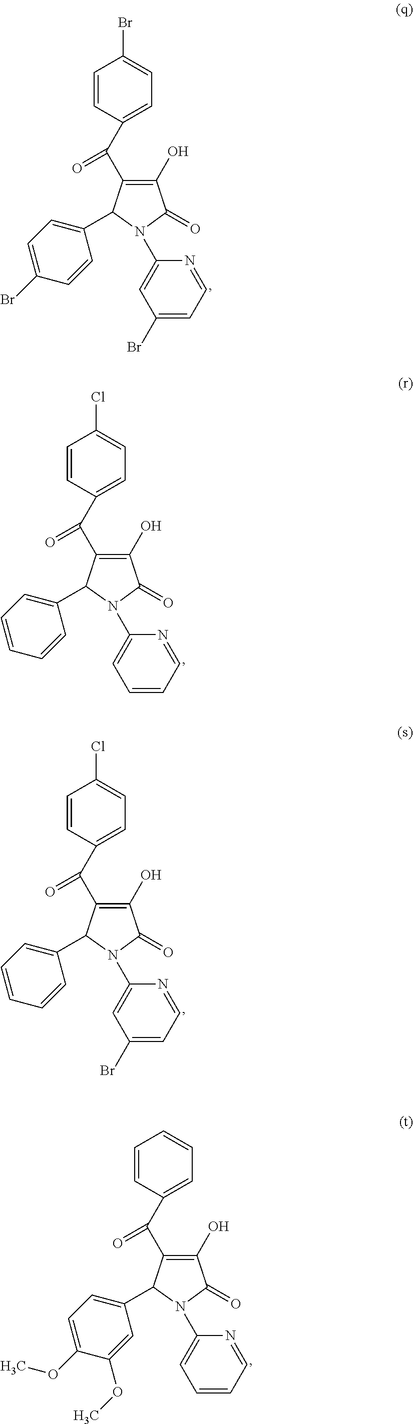

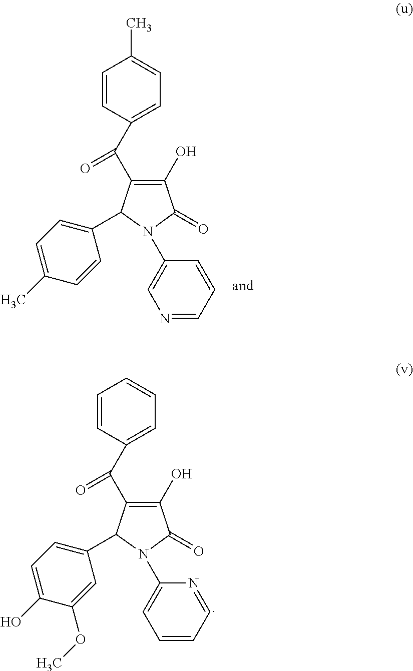

18. The compound according to claim 17 or a salt, solvate, tautomer, isotope, enantiomer, diastereoisomer or racemic mixture thereof, wherein n=0.

19. The compound according to claim 17 or a salt, solvate, tautomer, isotope, enantiomer, diastereoisomer or racemic mixture thereof, wherein Y represents an oxygen atom.

20. The compound according to claim 17 or a salt, solvate, tautomer, isotope, enantiomer, diastereoisomer or racemic mixture thereof, wherein Z represents an oxygen atom or a —N—OR group in which R represent a C₁-C₆ alkyl group, a (C₁-C₆ alkyl)O(C₁-C₆ alkyl) group or a (C₁-C₆ alkyl)COOH group.

21. The compound according to claim 17 or a salt, solvate, tautomer, isotope, enantiomer, diastereoisomer or racemic mixture thereof, wherein X represents a —C—R9 group and Y represents a —C—R8 group.

22. The compound according to claim 17 or a salt, solvate, tautomer, isotope, enantiomer, diastereoisomer or racemic mixture thereof, wherein R3 represents a group of the following formula II: 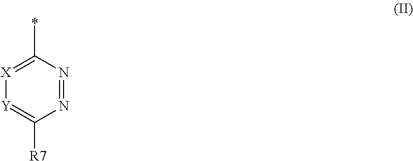
in which
X represents a nitrogen atom and Y represents a —C—R8 group or
X represents a —C—R9 group and Y represents a nitrogen atom or
X represents a —C—R9 group and Y represents a —C—R8 group, and
R7, R8 and R9 represent independently of each other
a hydrogen atom;
a halogen atom;
a C₁-C₆ alkyl group optionally substituted by one or more halogen atomby a —O—(C₁-C₆)alkyl group, by a —O—C₃-C₆ cycloalkyl group, by a —O-aryl group or by a —NR′R″ group in which R′ and R″ represent independently of each other a hydrogen atom, a C₁-C₆ alkyl group, a C₃-C₆ cycloalkyl group or an aryl group;
a C₃-C₆ cycloalkyl group optionally substituted by one or more halogen atom;
a —O—(C₁-C₆)alkyl group in which the alkyl group is optionally substituted by one or more halogen atom or by a phenyl group;
a —COOH group;
a —OH group;
a —COO(C₁-C₆)alkyl group in which the alkyl group is optionally substituted by one or more halogen atom;
a —CN group;
a —S-phenyl-NO₂ group;
a —SO₂-phenyl-NO₂ group;
a —SO₂—(C₁-C₆)alkyl group;
a —SO₂-aryl group;
a —SO₂—NH—(C₁-C₆)alkyl group;
a —SO₂—NH-aryl group;
a 6-members heterocyclic group containing one or two heteroatoms selected in the group consisting of nitrogen, sulfur and oxygen atom; or
a —NR′R″ group in which R′ and R″ represent independently of each other a hydrogen atom or a C₁-C₆ alkyl group, and
* indicates the position involved in binding with another group.

23. The compound according to claim 17 or a salt, solvate, tautomer, isotope enantiomer, diastereoisomer or racemic mixture thereof, wherein R7, R8 and R9 represent independently of each other a hydrogen atom; a C₁-C₆ alkyl group or a —NR′R″ group in which R′ and R″ represent independently of each other a hydrogen atom or a C₁-C₆ alkyl group.

24. The compound according to claim 17 or a salt, solvate, tautomer, isotope enantiomer, diastereoisomer or racemic mixture thereof, wherein R2 represents a phenyl group, optionally substituted, by one or more groups, independently selected among a halogen atom; a —OH group; a —CN group; a C₁-C₆ alkyl group optionally substituted by one or more halogen atom, or by a 5-members heteroaryl group containing one, two, three or four heteroatom (s) selected in the group consisting of oxygen, sulfur and nitrogen atom; a C₃-C₆ cycloalkyl group optionally substituted by one or more halogen atom; a —O—(C₃-C₆)cycloalkyl group in which the cycloalkyl group is optionally substituted by one or more halogen atom; a —O—(C₁-C₆)alkyl group in which the alkyl group is optionally substituted by one or more halogen atom; a —CO—(C₁-C₆)alkyl group in which the alkyl group is optionally substituted by one or more halogen atom; a —SO₇—(C₁-C₆)alkyl group in which the alkyl group is optionally substituted by one or more halogen atom; a —S—(C₁-C₆)alkyl group in which the alkyl group is optionally substituted by one or more halogen atom; a C₂-C₆ alkenyl group optionally substituted by one or more halogen atom; a C₂-C₆ alkynyl group optionally substituted by one or more halogen atom; a phenyl group; a —O-phenyl group; a 5-members heteroaryl group containing one, two, three or four heteroatom (s) selected in the group consisting of oxygen, sulfur and nitrogen atom, the heteroaryl group being optionally substituted by a C₁-C₆ alkyl group; a —NR′R″ group in which R′ and R″ represent independently of each other a hydrogen atom or a C₁-C₆ alkyl group, a —O-(6-members heterocyclic) group in which the heterocyclic group contains one, two or three heteroatoms selected in the group consisting of nitrogen, sulfur and oxygen atom; a —O-(5- or 6-members heteroaryl) group in which the heteroaryl group contains one, two, three or four heteroatom (s) selected in the group consisting of oxygen, sulfur and nitrogen atom, the heteroaryl group being optionally substituted by a —(C₁-C₆ alkyl)-phenyl group, a —(C₁-C₆ alkyl) group or a —CH₂—O—CH₂—Si(CH₃)₃ group, the alkyl group being optionally substituted by a halogen atom; a —O((C₁-C₆)alkyl)-(6-members heterocyclic) group in which the heterocyclic group contains one, two or three heteroatoms selected in the group consisting of nitrogen, sulfur and oxygen atom; a —O((C₁-C₆)alkyl)-NR′R″ group in which R′ and R″ represent independently of each other a hydrogen atom or a C₁-C₆ alkyl group, and a 6-members heterocyclic group containing one, two or three heteroatoms selected in the group consisting of nitrogen, sulfur and oxygen atom, the heterocyclic group being optionally substituted by a —((C₁-C₆)alkyl)-NR′R″ group in which R′ and R″ represent independently of each other a hydrogen atom or a C₁-C₆ alkyl group.

25. The compound according to claim 17 or a salt, solvate, tautomer, isotope, enantiomer, diastereoisomer or racemic mixture thereof, wherein R1 represents a phenyl group, optionally substituted by a halogen atom; a —CN group; a —SO₂—(C₁-C₆) alkyl group in which the alkyl group is optionally substituted by one or more halogen atom; a C₁-C₆ alkyl group optionally substituted by one or more halogen atom; a C₂-C₆ alkenyl group optionally substituted by one or more halogen atom; a C₃-C₆ cycloalkyl group optionally substituted by one or more halogen atom; a —O—(C₁-C₆)alkyl group in which the alkyl group is optionally substituted by one or more halogen atom; a O—(C₁-C₆)alkyl-O—(C₁-C₆)alkyl group in which the alkyl group is optionally substituted by one or more halogen atom; a —O—(C₁-C₆)alkyl-phenyl-O—(C₁-C₆)alkyl group; a phenyl group; a 5- or 6-members heteroaryl group containing one, two or three heteroatoms selected in the group consisting of oxygen, nitrogen and sulfur atom; a 6-members heterocyclic group containing one, two or three heteroatoms selected in the group consisting of nitrogen, sulfur and oxygen atom; or a —NR′R″ group in which R′ and R″ represent independently of each other a hydrogen atom or a C₁-C₆ alkyl group.

26. The compound according to claim 17 or a salt, solvate, tautomer, isotope, enantiomer, diastereoisomer or racemic mixture thereof, wherein R3 represents a 6-members heteroaryl group containing as the only heteroatom, one nitrogen atom, said heteroaryl group being optionally substituted by a halogen atom; a C₁-C₆ alkyl group; a C₃-C₆ cycloalkyl group optionally substituted by one or more halogen atom; a —O—(C₁-C₆)alkyl group in which the alkyl group is optionally substituted by a phenyl group; a —OH group; a —COOH group; a —COO(C₃-C₆)alkyl group or a COOC₁ alkyl group in which the alkyl group is optionally substituted by one or more halogen atom; a —CN group; a ═O group; a —SO₂-phenyl-NO₂ group; a —S-phenyl-NO₂ group; a —SO₂—(C₁-C₆)alkyl group; a —SO₂-aryl group; a —SO₂—NH—(C₁-C₆)alkyl group; a —SO₂—NH-aryl group; a 6-members heterocyclic group containing one or two heteroatoms selected in the group consisting of nitrogen, sulfur and oxygen atom; or a —NR′R″ group in which R′ and R″ represent independently of each other a hydrogen atom or a C₁-C₆ alkyl group.

27. The compound according to claim 17 or a salt, solvate, tautomer, isotope, enantiomer, diastereoisomer or racemic mixture thereof, wherein R4 represents a —OH group or a —O—C═O—(C₁-C₆alkyl) group.

28. The compound according to claim 17 or a salt, solvate, tautomer, isotope, enantiomer, diastereoisomer or racemic mixture thereof, wherein it is chosen from the following compounds:

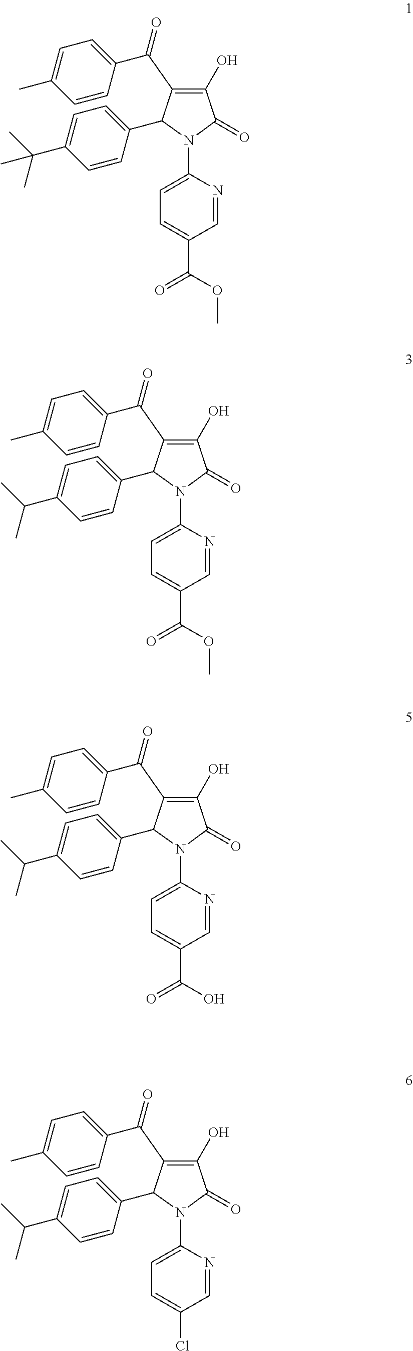

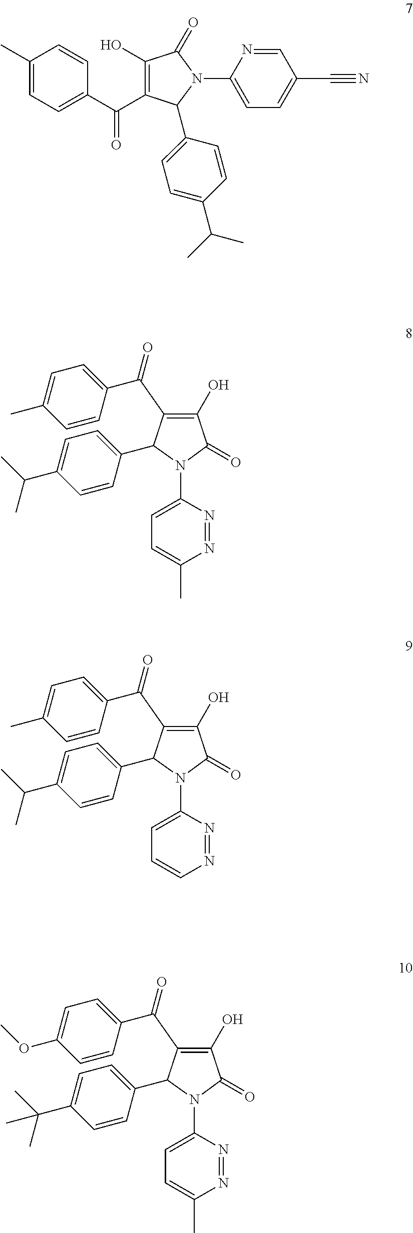

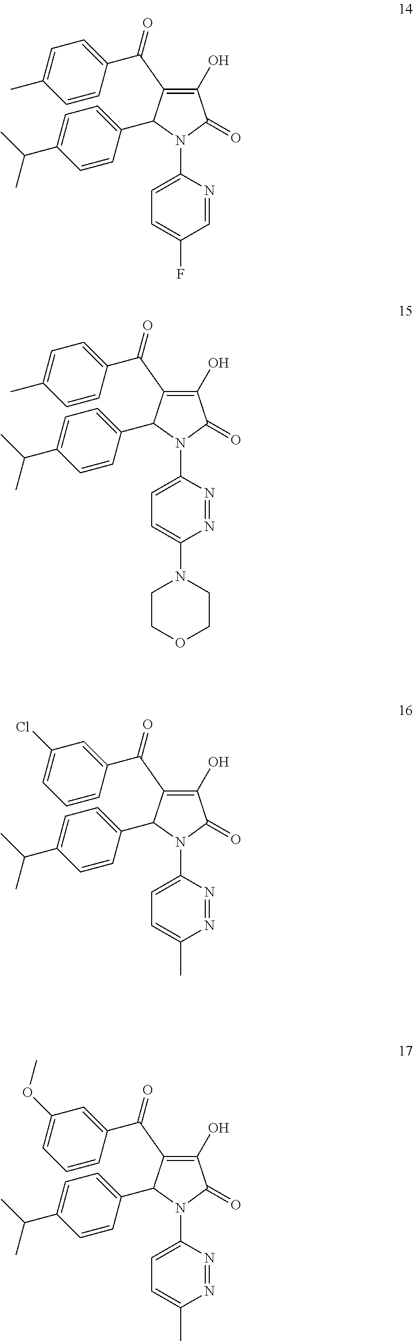

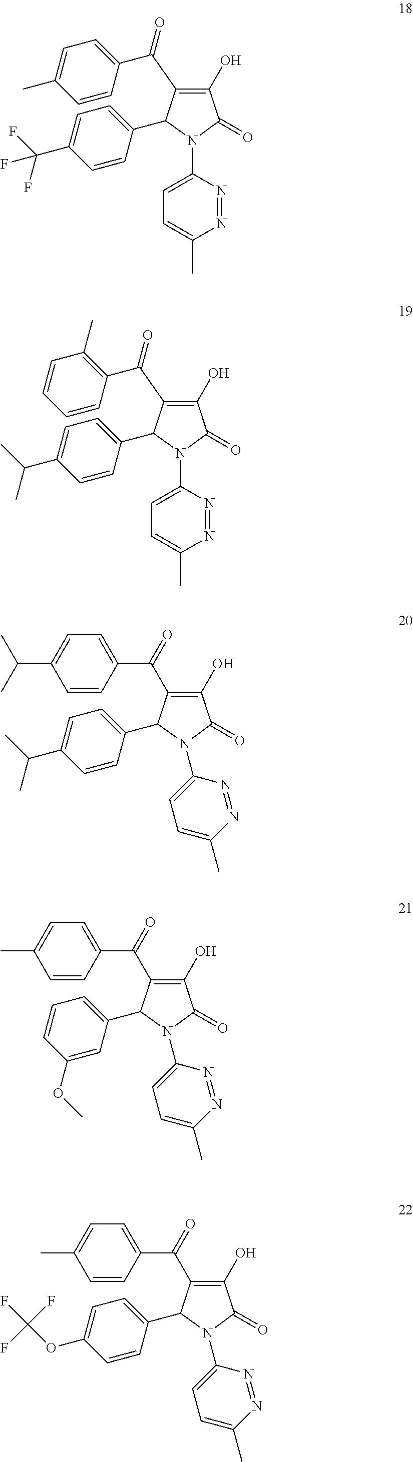

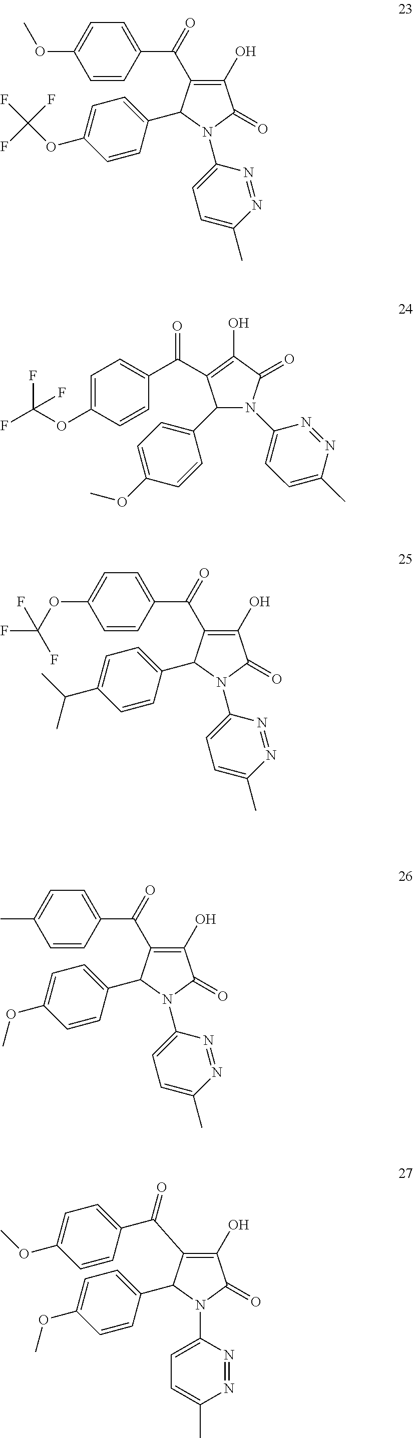

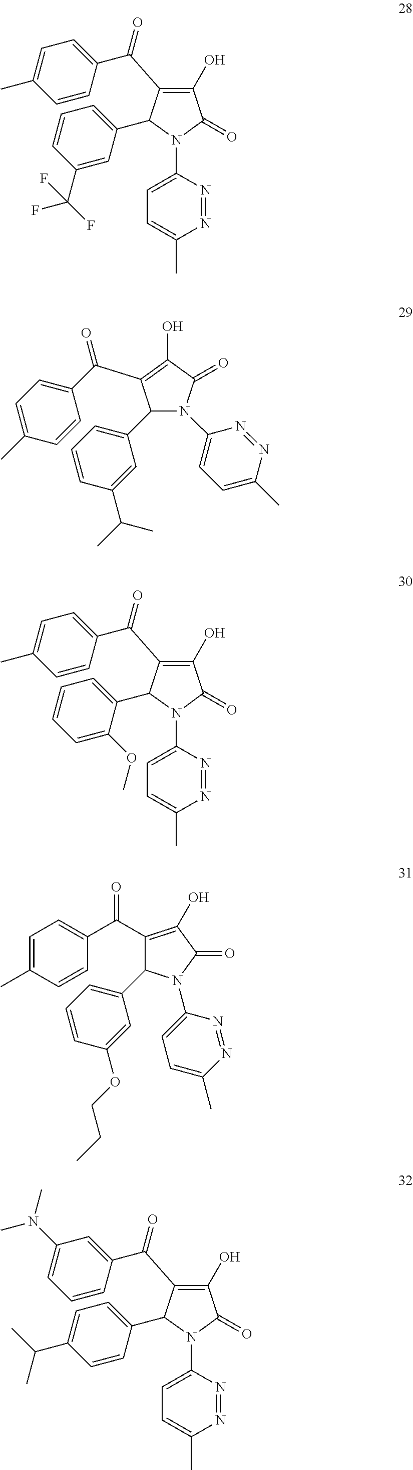

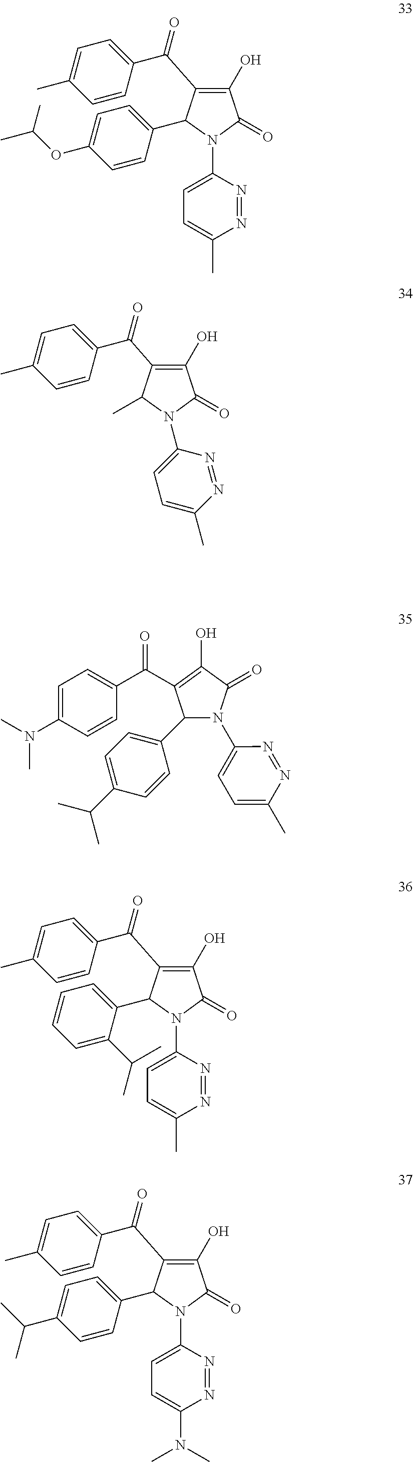

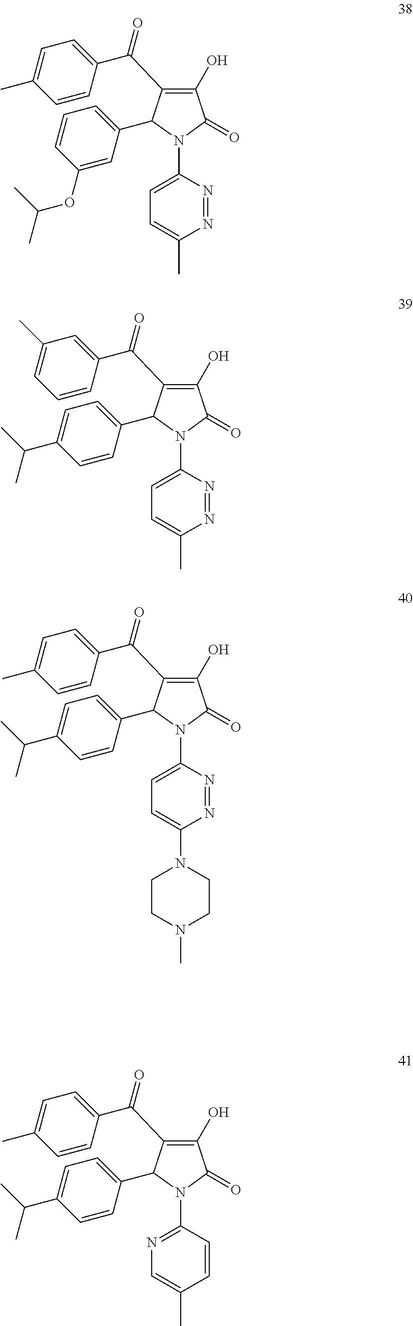

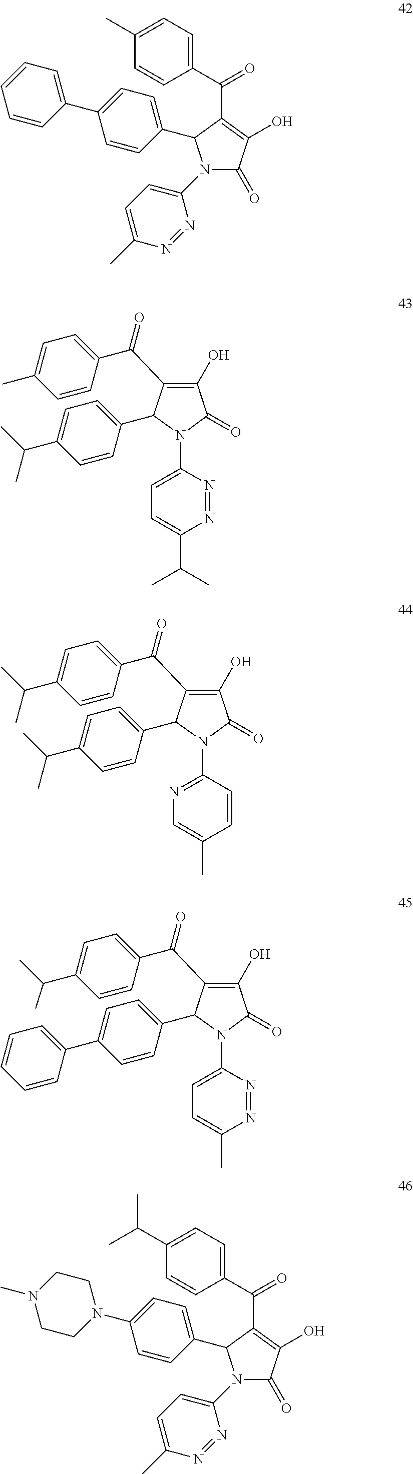

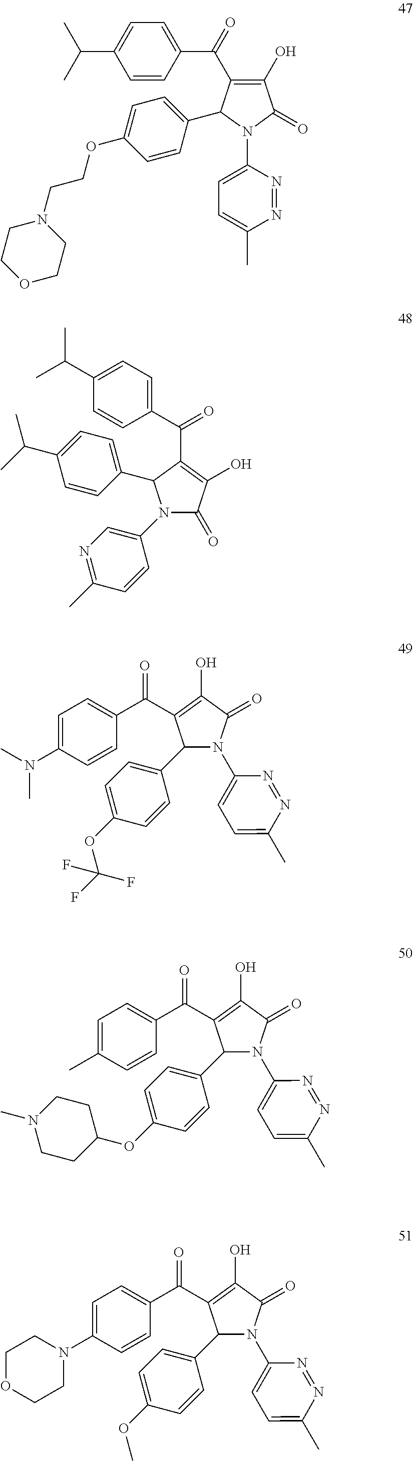

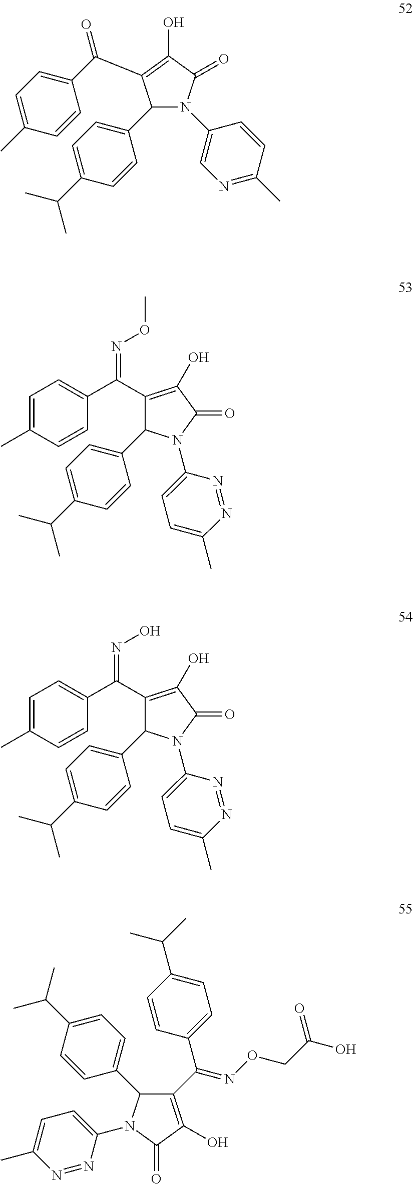

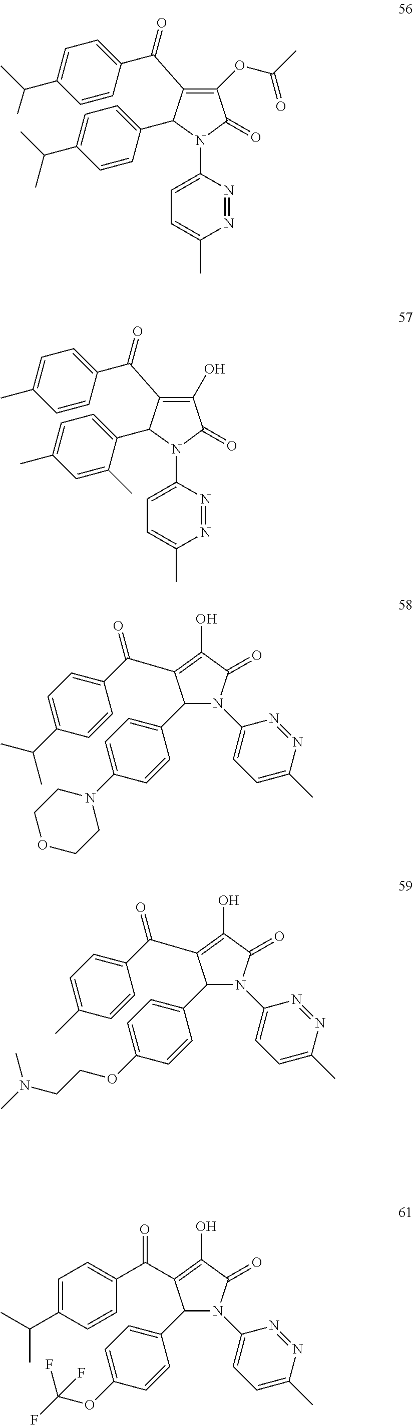

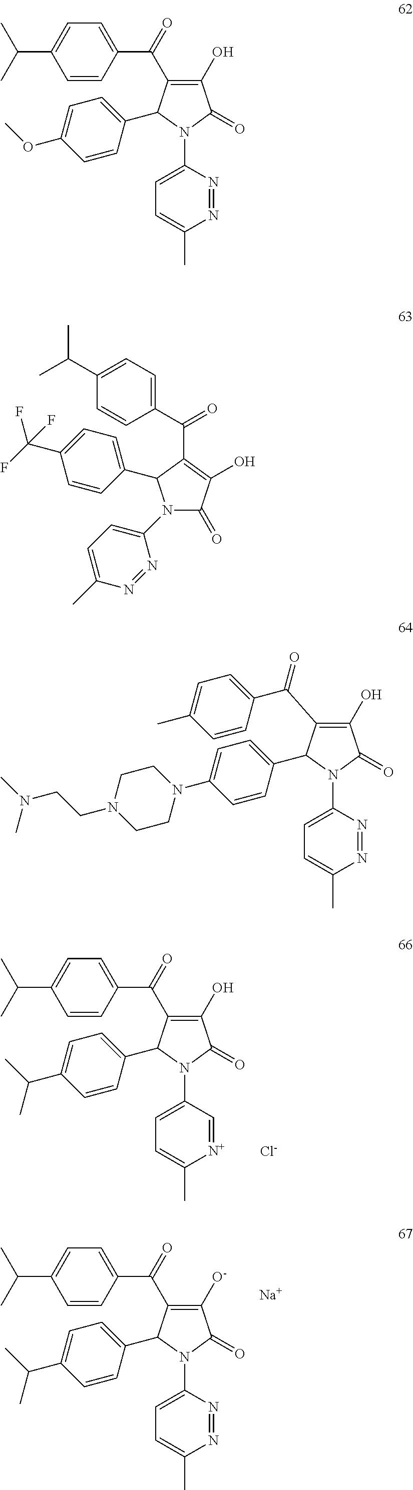

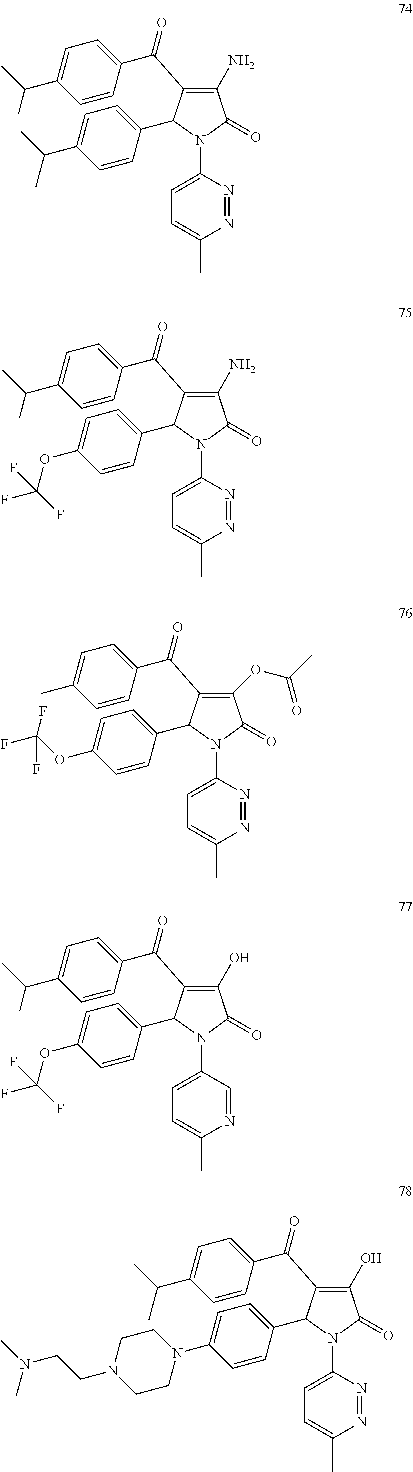

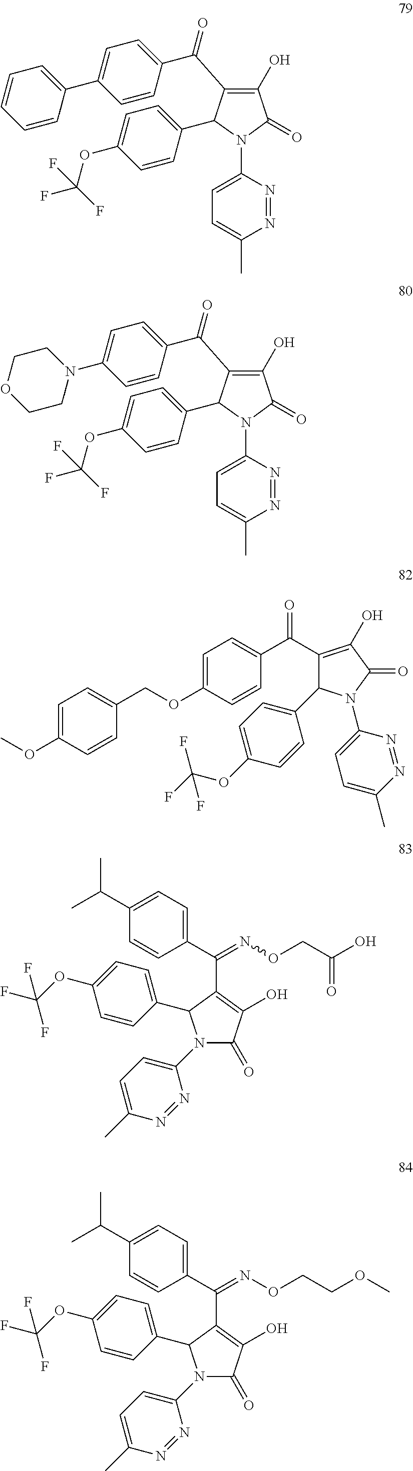

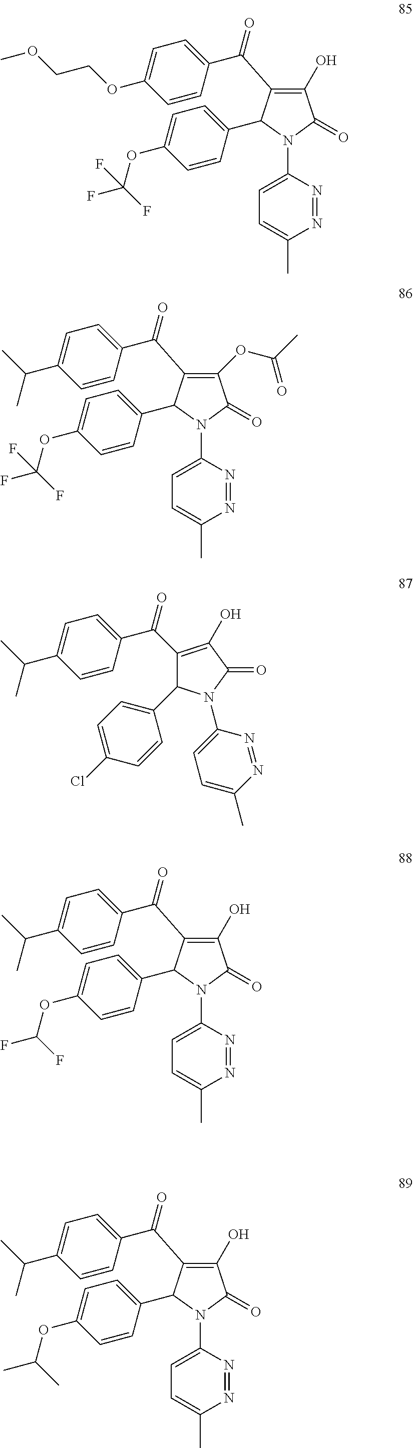

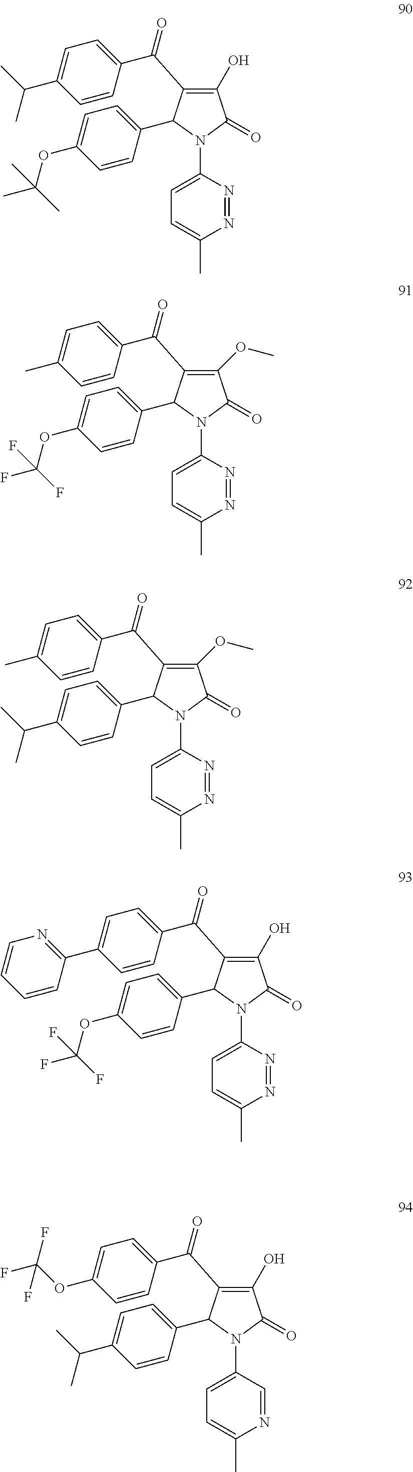

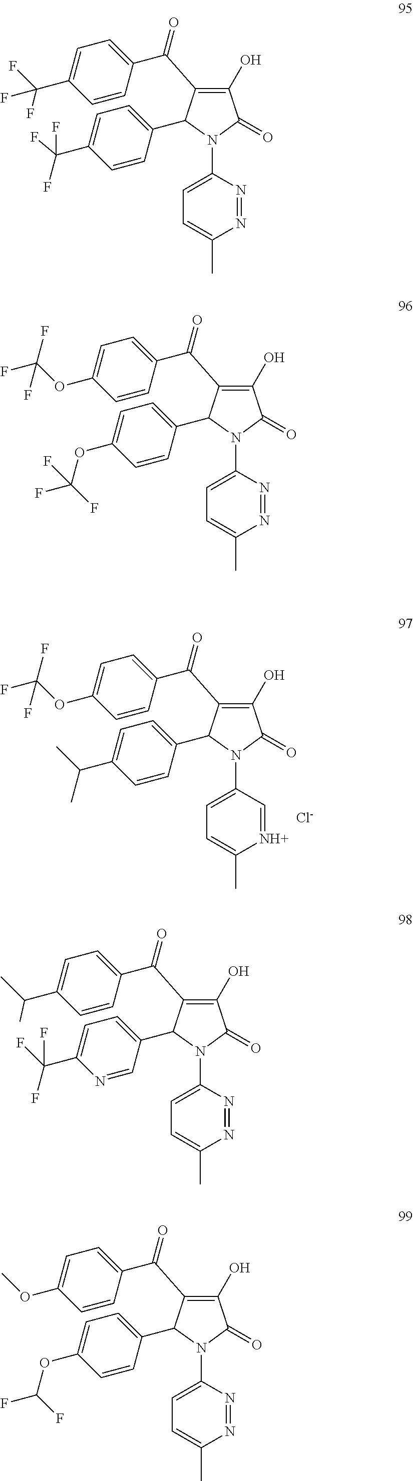

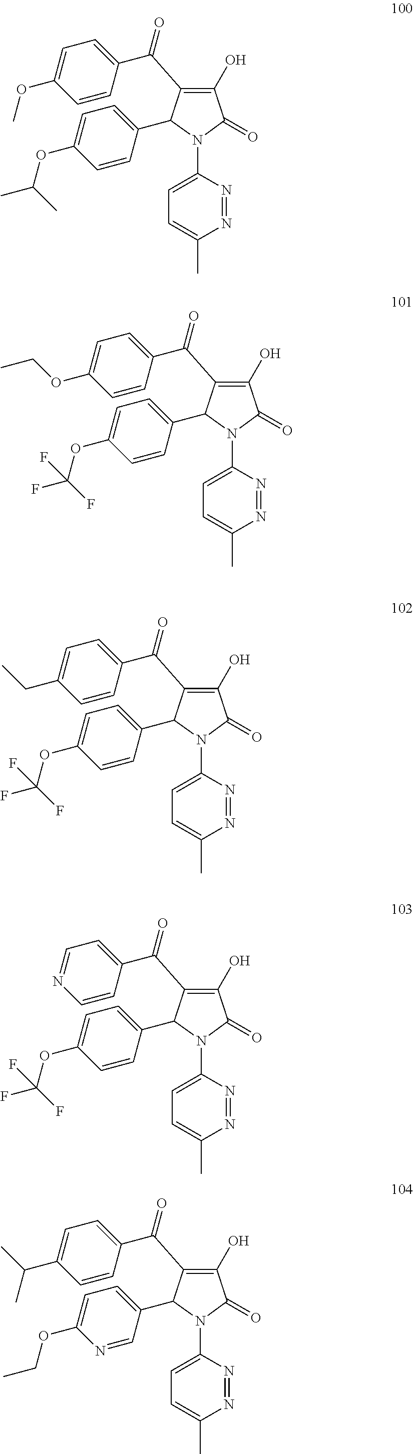

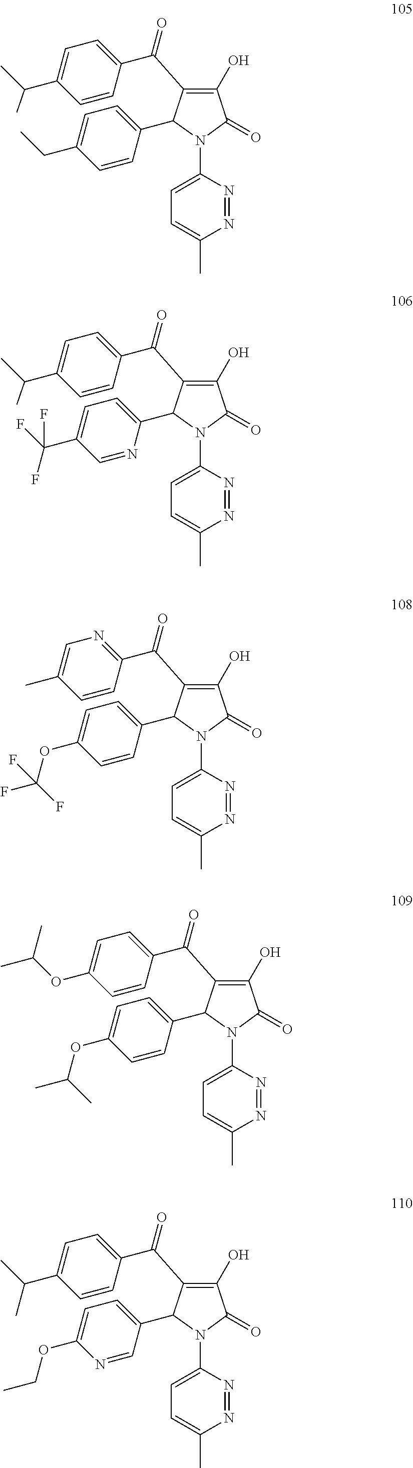

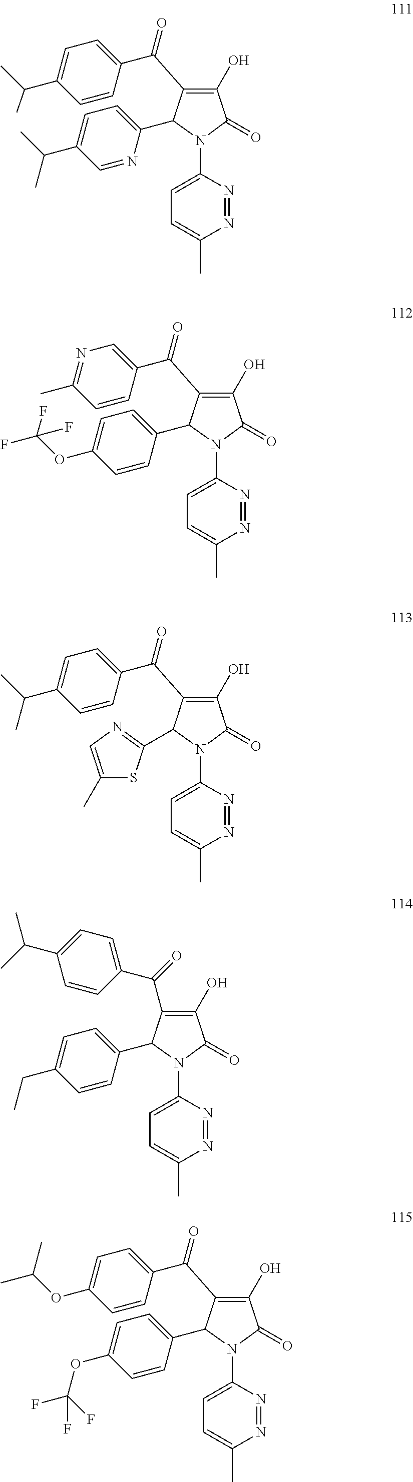

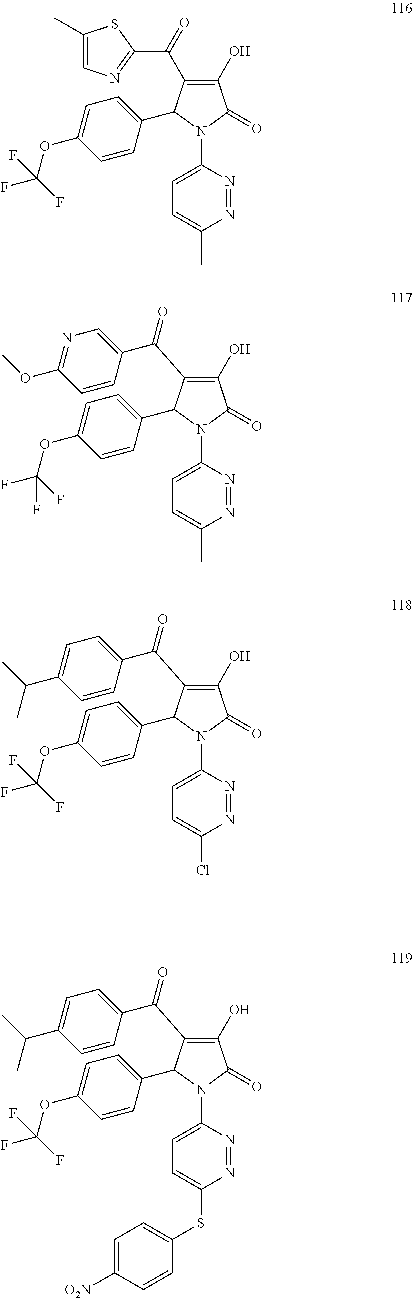

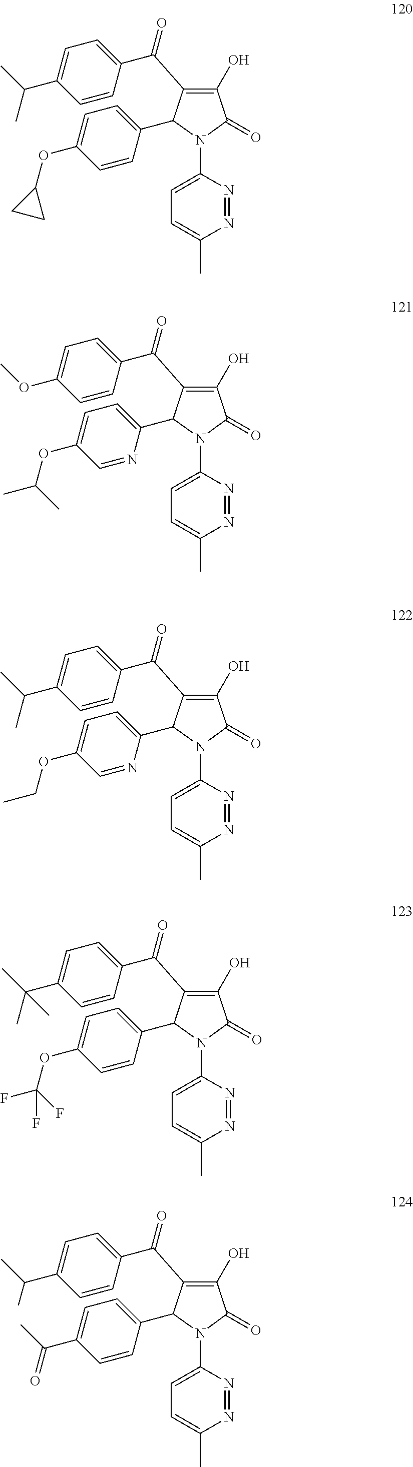

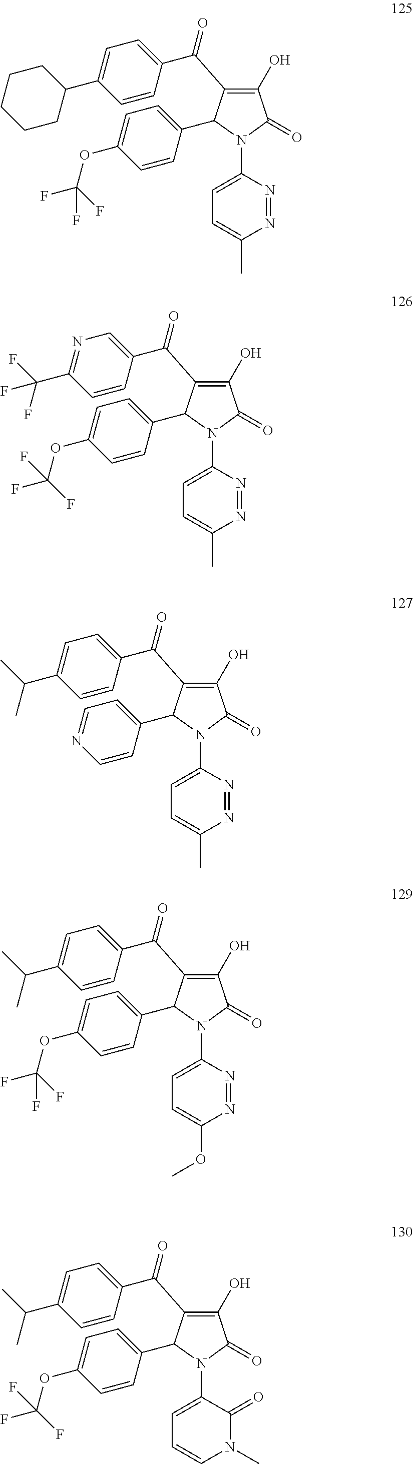

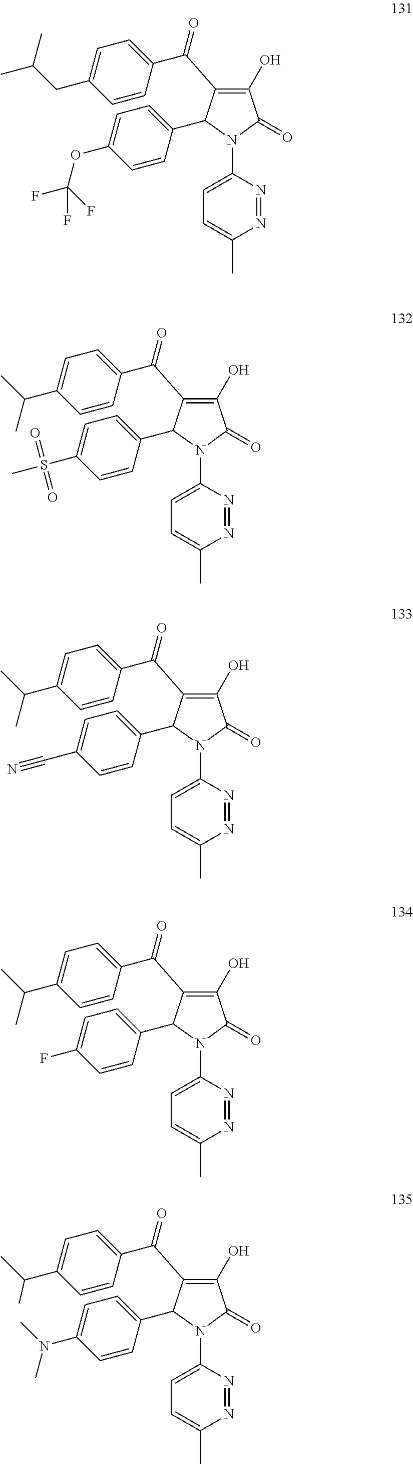

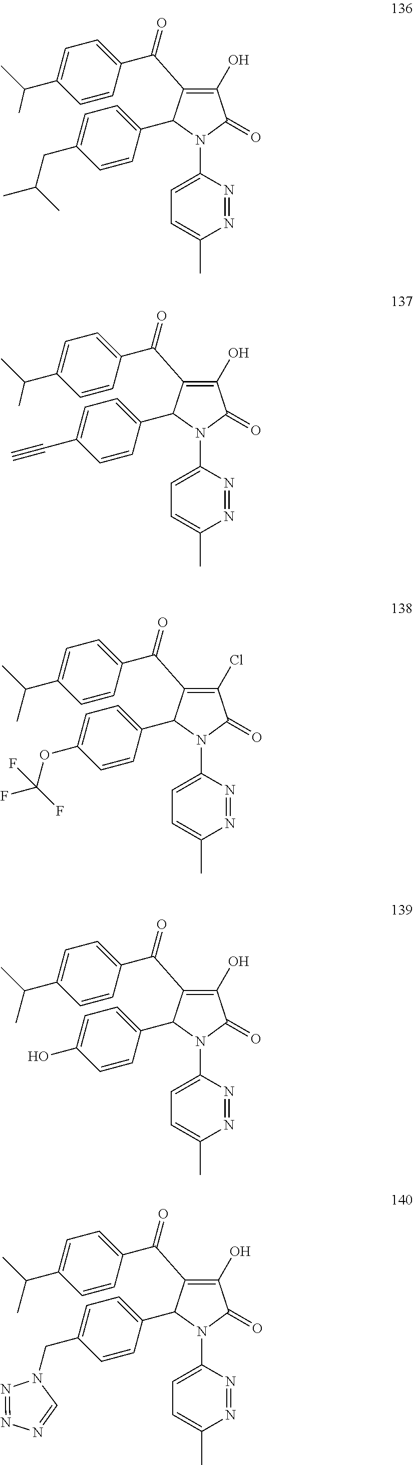

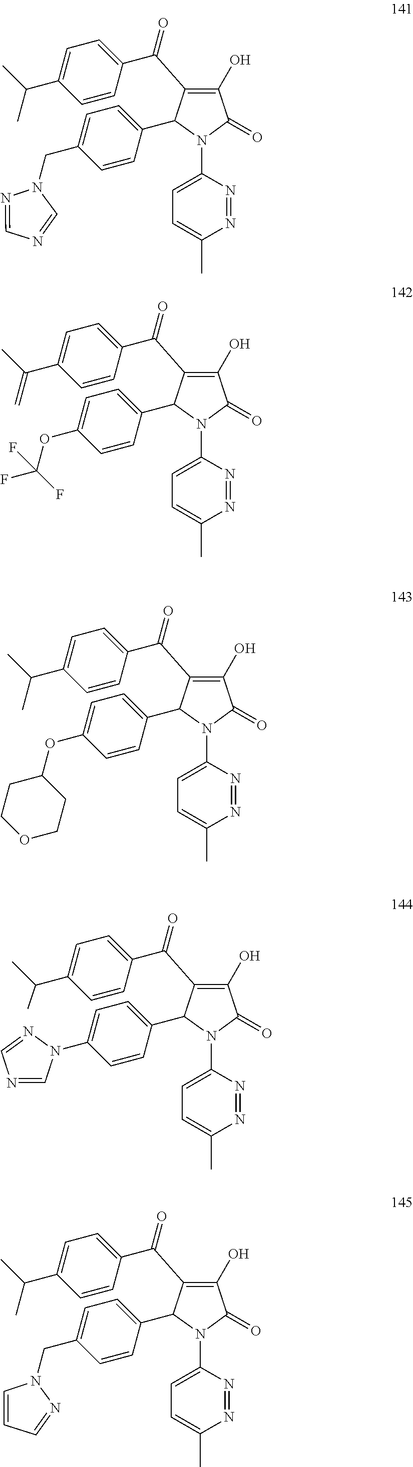

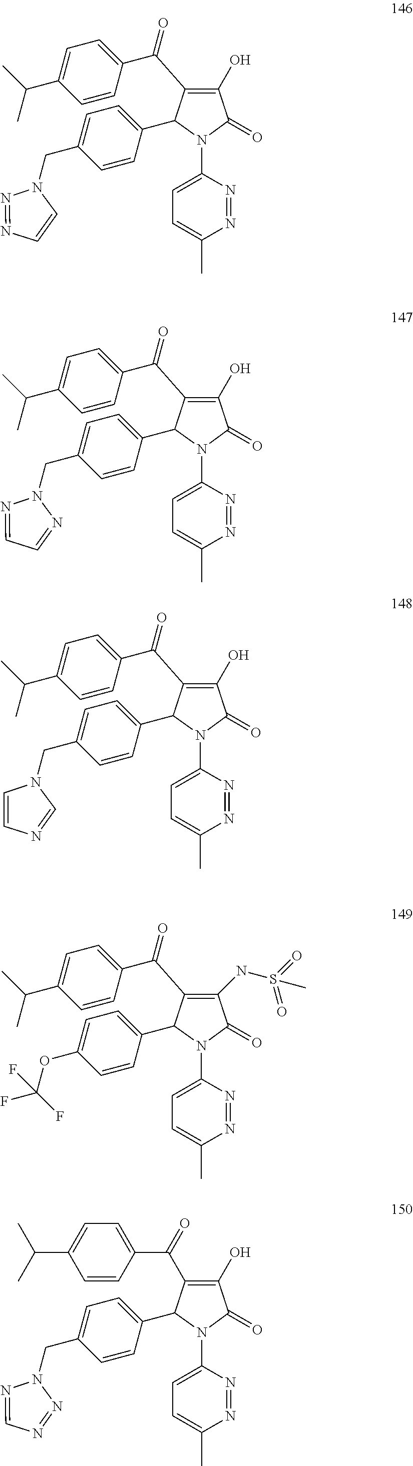

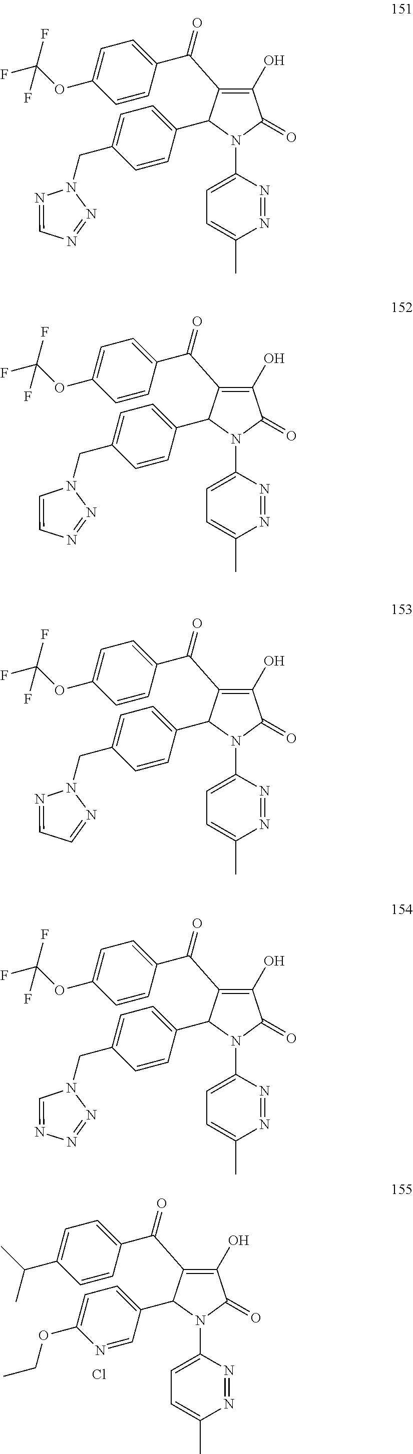

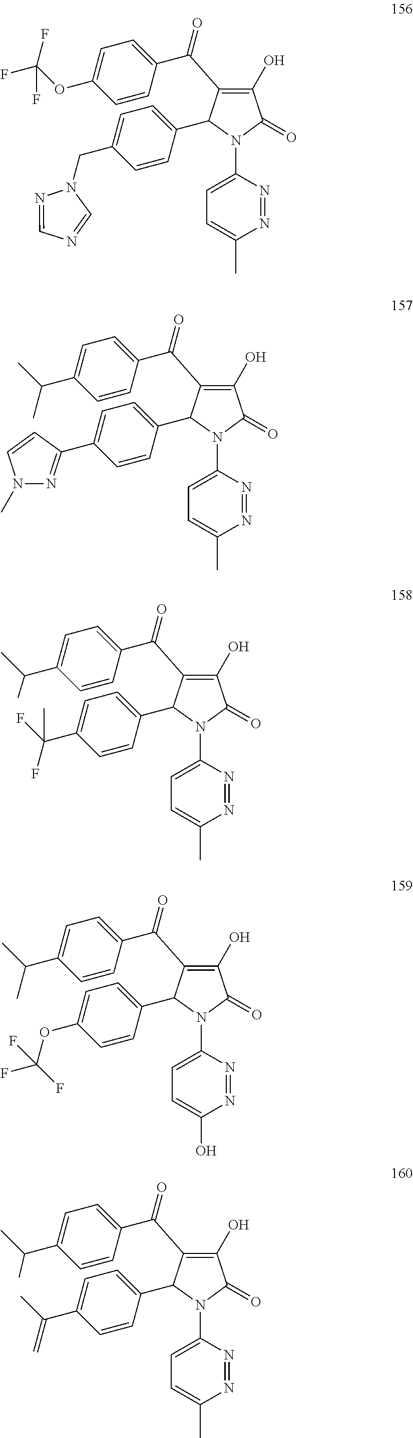

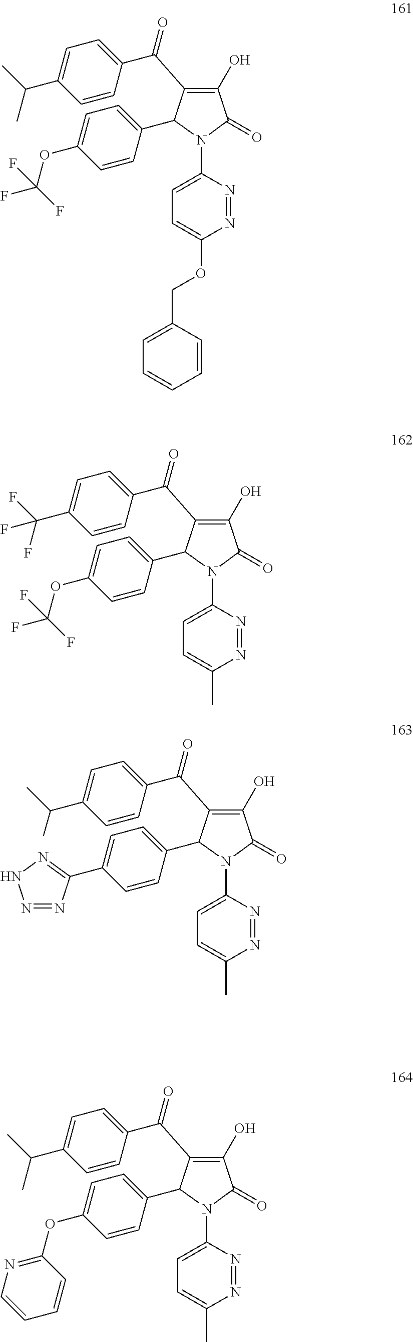

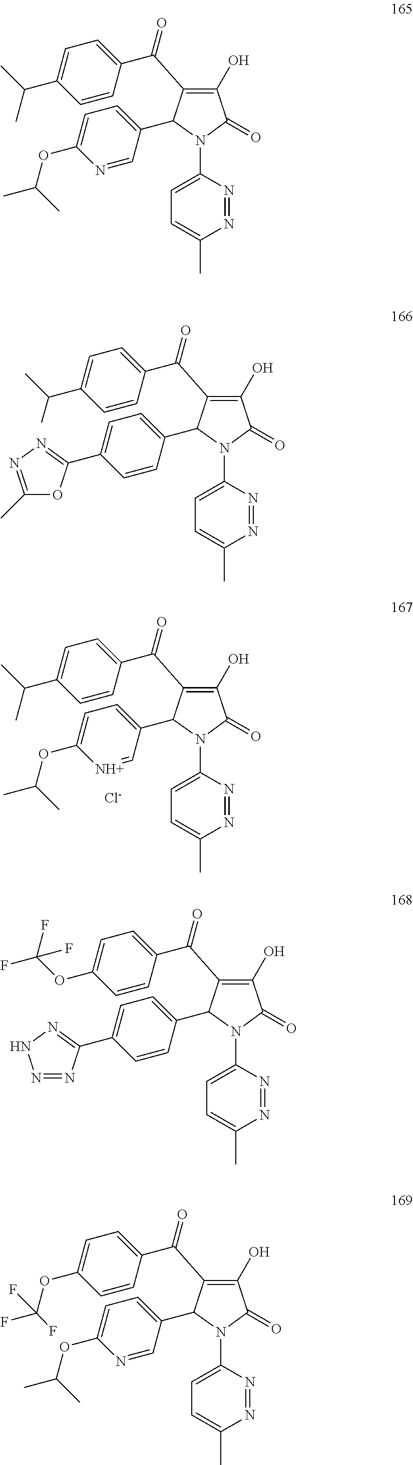

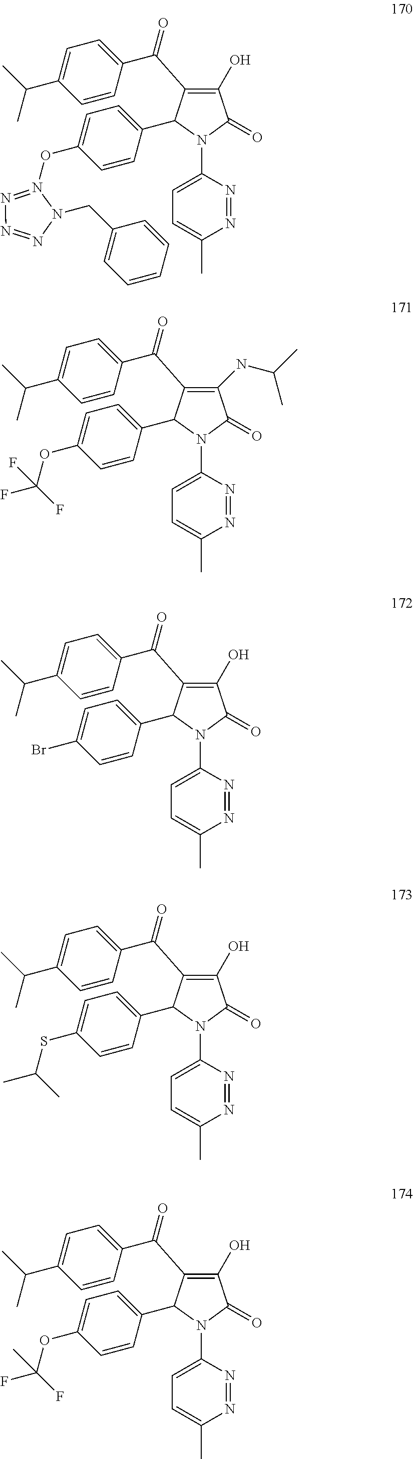

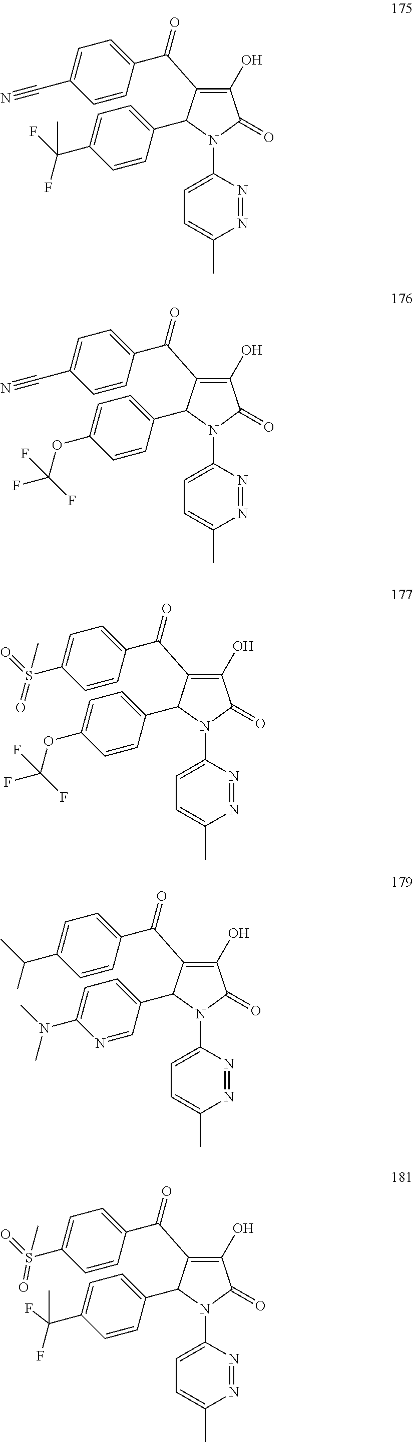

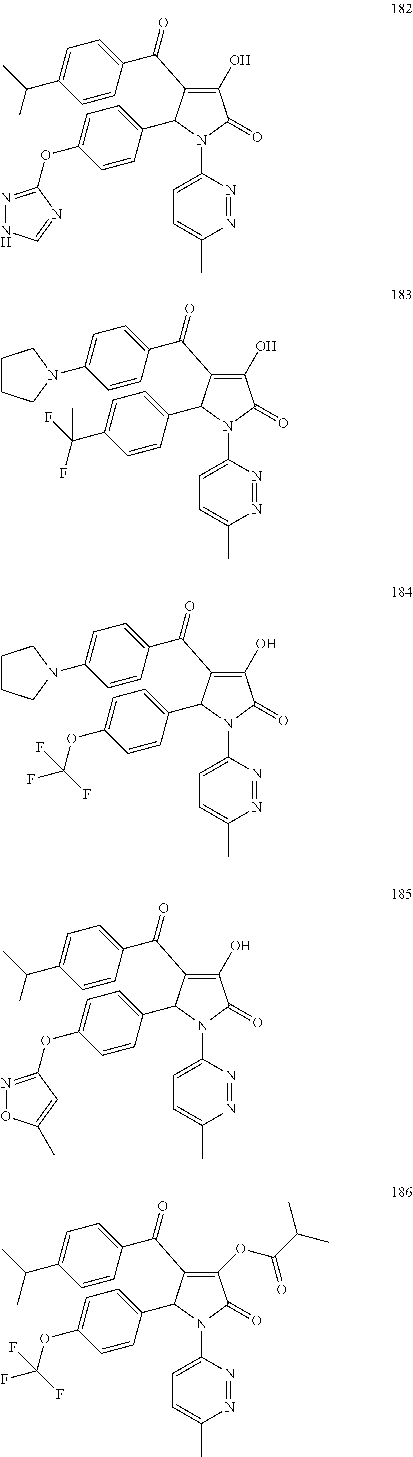

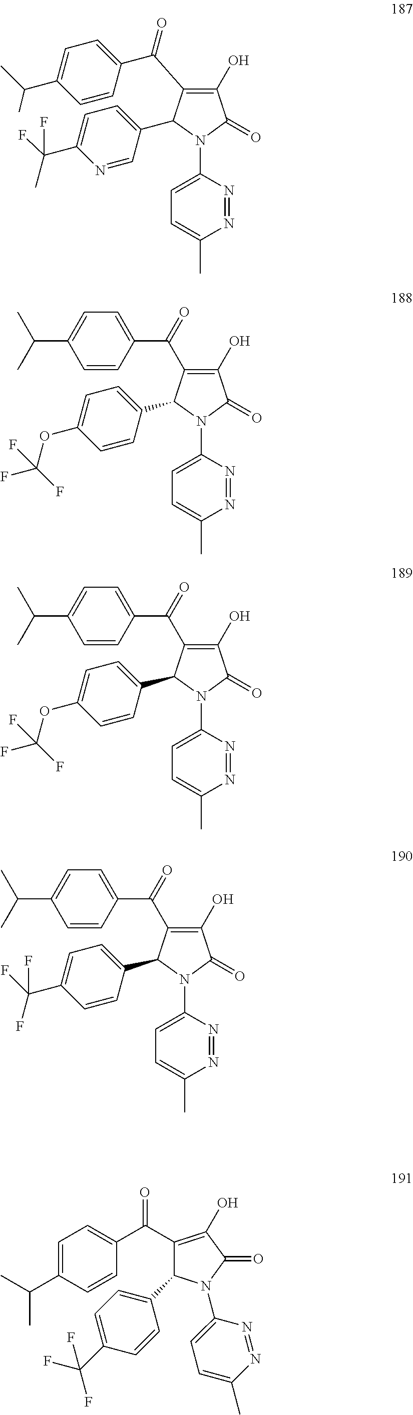

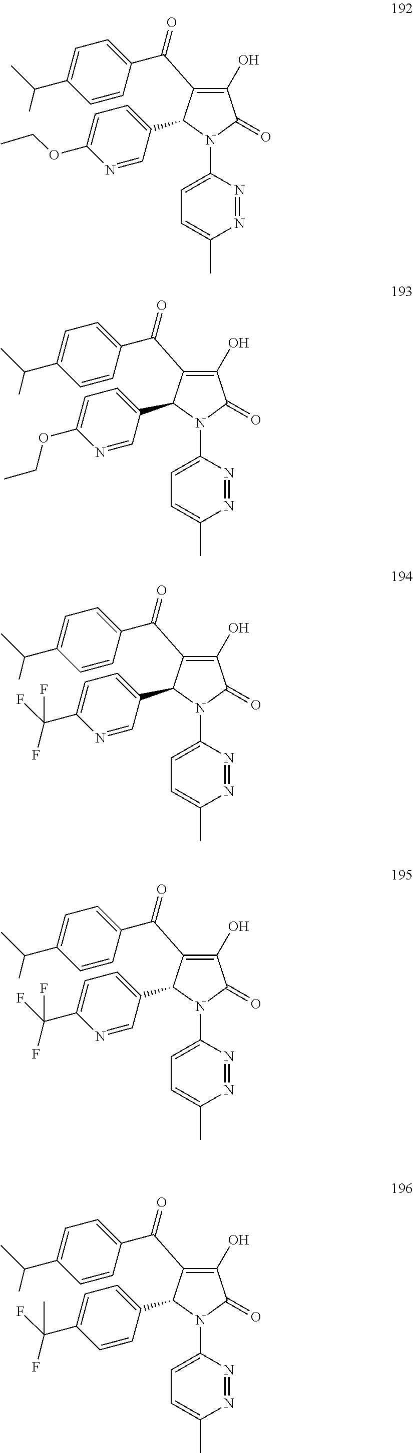

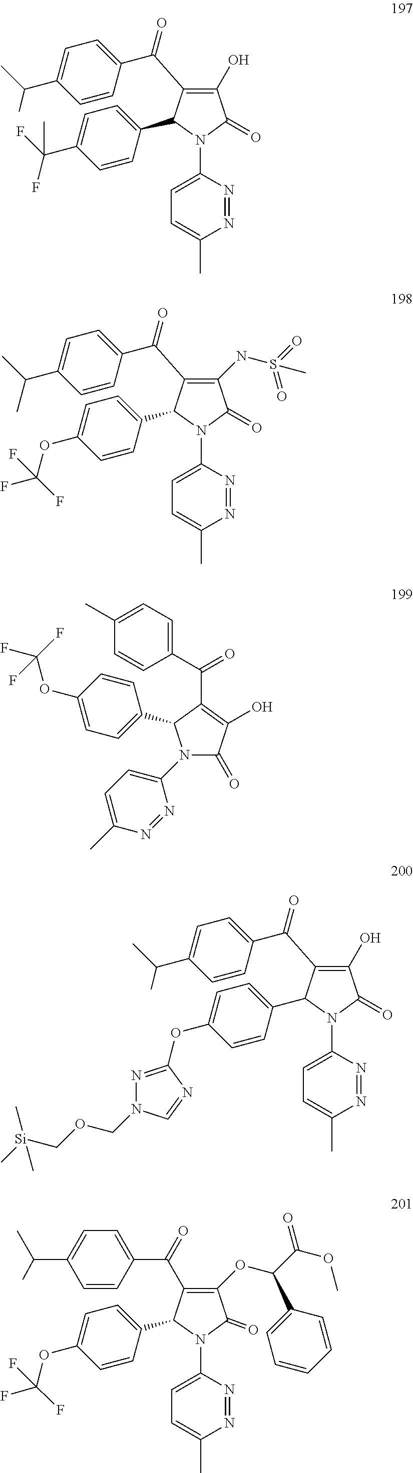

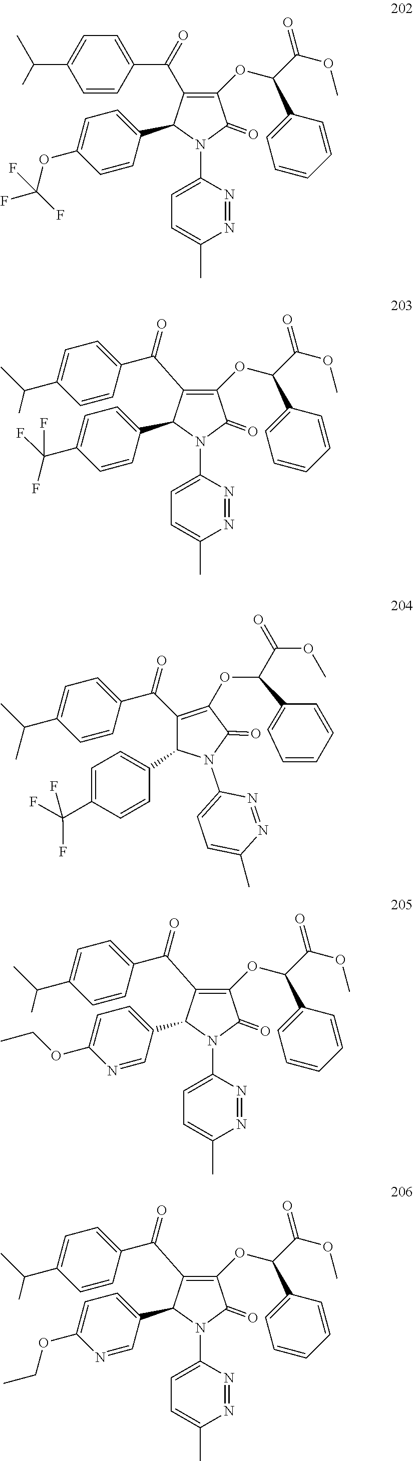

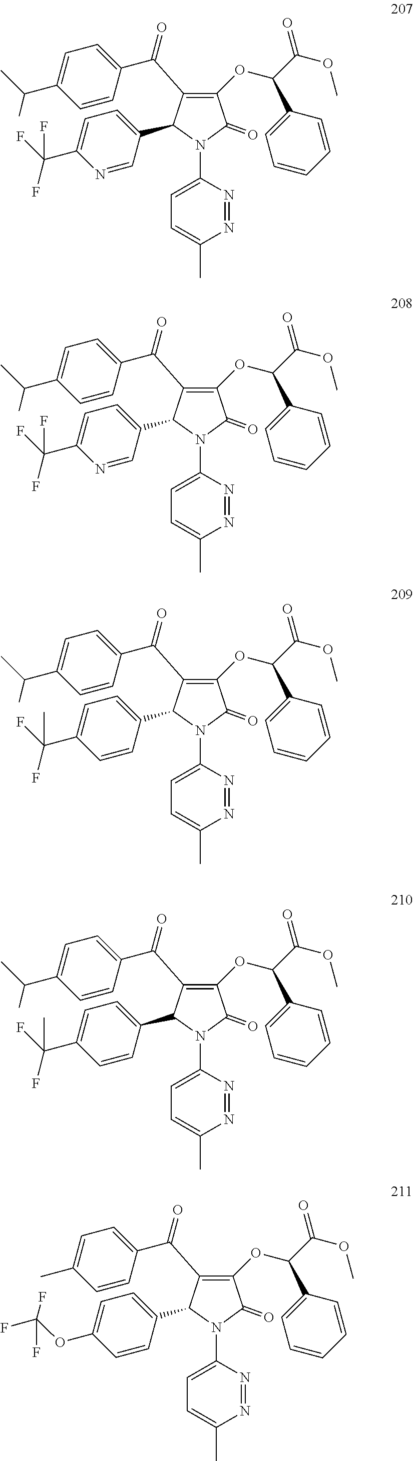

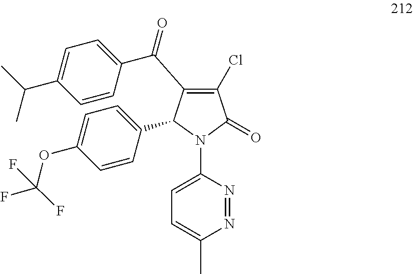

29. A pharmaceutical composition comprising a compound according to claim 17 or a salt, solvate, tautomer, isotope, enantiomer, diastereoisomer or racemic mixture thereof and a pharmaceutically acceptable diluent or carrier.

30. The pharmaceutical composition according to claim 29 containing a further antiviral agent.

31. The pharmaceutical composition according to claim 30, wherein the further antiviral agent is selected in the group consisting of ribavirin, interferon, inhibitors of HCV helicase, inhibitors of HCV protease, inhibitors of HCV NS4A, inhibitors of HCV NS5B, inhibitors of HCV NS5A, anti-HIV agent and mixture thereof.

32. A method to treat or prevent a viral infection comprising the administration to a person in need thereof of an effective amount of a compound of the following formula I or a salt, solvate, tautomer, isotope, enantiomer, diastereoisomer or racemic mixture thereof: 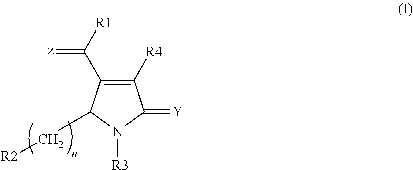
in which
n is an integer chosen between 0, 1 or 2;
Y represents an oxygen atom or a sulfur atom;
C═Z represents CH₂ or
Z represents an oxygen atom, a —CH—R group or a —N—OR group, in which R represents an hydrogen atom, a C₁-C₆ alkyl group, a C₃-C₆ cycloalkyl group, a 3-6 members heterocyclic group containing one or two heteroatoms selected in the group consisting of oxygen, nitrogen and sulfur atom, a (C₁-C₆ alkyl)COOH group, a (C₁-C₆ alkyl)O(C₁-C₆ alkyl) group or a O-protecting group;
R1 represents:
a phenyl group, or
a 5- or 6-members heteroaryl group containing one, two or three heteroatoms selected in the group consisting of oxygen, nitrogen and sulfur atom,
the phenyl group and the heteroaryl group being optionally substituted by:
a halogen atom;
a —CN group;
a —SO₂—(C₁-C₆) alkyl group in which the alkyl group is optionally substituted by one or more halogen atom;
a phenyl group;
a 5- or 6-members heteroaryl group containing one, two or three heteroatoms selected in the group consisting of oxygen, nitrogen and sulfur atom;
a C₁-C₆ alkyl group optionally substituted by one or more halogen atom;
a C₂-C₆ alkenyl group optionally substituted by one or more halogen atom;
a —O—(C₁-C₆)alkyl group in which the alkyl group is optionally substituted by one or more halogen atom;
a O—(C₁-C₆)alkyl-O—(C₁-C₆)alkyl group in which the alkyl group is optionally substituted by one or more halogen atom;
a —O—(C₁-C₆)alkyl-phenyl-O—(C₁-C₆)alkyl group;
a C₃-C₆ cycloalkyl group optionally substituted by one or more halogen atom;
a 5-, 6- or 7-members heterocyclic group containing one, two or three heteroatoms selected in the group consisting of nitrogen, sulfur and oxygen atom; or
a —NR′R″ group in which R′ and R″ represent independently of each other a hydrogen atom or a C₁-C₆ alkyl group;
R2 represents:
a phenyl group, or
a 5- or 6-members heteroaryl group containing one, two or three heteroatom (s) selected in the group consisting of oxygen, sulfur and nitrogen atom,
the phenyl group and the heteroaryl group being optionally substituted by one or more groups, independently selected among:
a halogen atom;
a —OH group;
a —CN group;
a C₁-C₆ alkyl group optionally substituted by one or more halogen atom or by a 5-members heteroaryl group containing one, two, three or four heteroatom (s) selected in the group consisting of oxygen, sulfur and nitrogen atom;
a —O—(C₁-C₆)alkyl group in which the alkyl group is optionally substituted by one or more halogen atom;
a —CO—(C₁-C₆)alkyl group in which the alkyl group is optionally substituted by one or more halogen atom;
a —SO₂—(C₁-C₆)alkyl group in which the alkyl group is optionally substituted by one or more halogen atom;
a —S—(C₁-C₆)alkyl group in which the alkyl group is optionally substituted by one or more halogen atom;
a C₂-C₆ alkenyl group optionally substituted by one or more halogen atom;
a C₂-C₆ alkynyl group optionally substituted by one or more halogen atom;
a C₃-C₆ cycloalkyl group optionally substituted by one or more halogen atom;
a —O—(C₃-C₆)cycloalkyl group in which the cycloalkyl group is optionally substituted by one or more halogen atom;
a phenyl group;
a —O-phenyl group;
a 5-members heteroaryl group containing one, two, three or four heteroatom (s) selected in the group consisting of oxygen, sulfur and nitrogen atom, the heteroaryl group being optionally substituted by a C₁-C₆ alkyl group;
a —NR′R″ group in which R′ and R″ represent independently of each other a hydrogen atom or a C₁-C₆ alkyl group;
a —O-(6-members heterocyclic) group in which the heterocyclic group contains one, two or three heteroatoms selected in the group consisting of nitrogen, sulfur and oxygen atom;
a —O-(5- or 6-members heteroaryl) group in which the heteroaryl group contains one, two, three or four heteroatom (s) selected in the group consisting of oxygen, sulfur and nitrogen atom, heteroaryl group being optionally substituted by a —(C₁-C₆ alkyl)-phenyl group, a —(C₁-C₆ alkyl) group or a —CH₂—O—CH₂—Si(CH₃)₃ group, the alkyl group being optionally substituted by a halogen atom;
a —O—((C₁-C₆)alkyl)-(6-members heterocyclic) group in which the heterocyclic group contains one, two or three heteroatoms selected in the group consisting of nitrogen, sulfur and oxygen atom;
a —O—((C₁-C₆)alkyl)-NR′R″ group in which R′ and R″ represent independently of each other a hydrogen atom or a C₁-C₆ alkyl group; and
a 6-members heterocyclic group containing one, two or three heteroatoms selected in the group consisting of nitrogen, sulfur and oxygen atom, the heterocyclic group being optionally substituted by a —((C₁-C₆)alkyl)-NR′R″ group in which R′ and R″ represent independently of each other a hydrogen atom or a C₁-C₆ alkyl group;
R3 represents a 6-members heteroaryl group containing as the only heteroatom(s), one, two or three nitrogen atoms, the heteroaryl group being optionally substituted by
a halogen atom;
a C₁-C₆ alkyl group optionally substituted by one or more halogen atom, by a —O—(C₁-C₆)alkyl group, by a —O—C₃-C₆ cycloalkyl group, by a —O-aryl group or by a —NR′R″ group in which R′ and R″ represent independently of each other a hydrogen atom, a C₁-C₆ alkyl group, a C₃-C₆ cycloalkyl group or an aryl group;
a C₃-C₆ cycloalkyl group optionally substituted by one or more halogen atom;
a —O—(C₁-C₆)alkyl group in which the alkyl group is optionally substituted by one or more halogen atom or by a phenyl group;
a —OH group;
a —COOH group;
a —COO(C₁-C₆)alkyl group in which the alkyl group is optionally substituted by one or more halogen atom;
a —CN group;
a ═O group;
a —SO₂-phenyl-NO₂ group;
a —S-phenyl-NO₂ group;
a —SO₂—(C₁-C₆)alkyl group;
a —SO₂-aryl group,
a —SO₂—NH—(C₁-C₆)alkyl group;
a —SO₂—NH-aryl group;
a 6-members heterocyclic group containing one or two heteroatoms selected in the group consisting of nitrogen, sulfur and oxygen atom; or
a —NR′R″ group in which R′ and R″ represent independently of each other a hydrogen atom or a C₁-C₆ alkyl group;
R4 represents:
a —OH group or
a halogen atom or
a —O—C═O—(C₁-C₆alkyl) group or
a —O—(C₁-C₆)alkyl group or
a —O—(C₁-C₆)alkyl-CO—O—(C₁-C₆)alkyl group, in which the alkyl group is optionally substituted by a phenyl group;
a —NHR₅ group in which R₅ represents an hydrogen or a (C₁-C₆alkyl) group, or
a —NHSO₂R₆ group in which R₆ represents a hydrogen atom or a (C₁-C₆alkyl) group.

33. The method according to claim 32, wherein the viral infection is hepatitis C.

34. The method according to claim 32, wherein the compound of formula (I) is chosen from the group consisting of the following compounds:

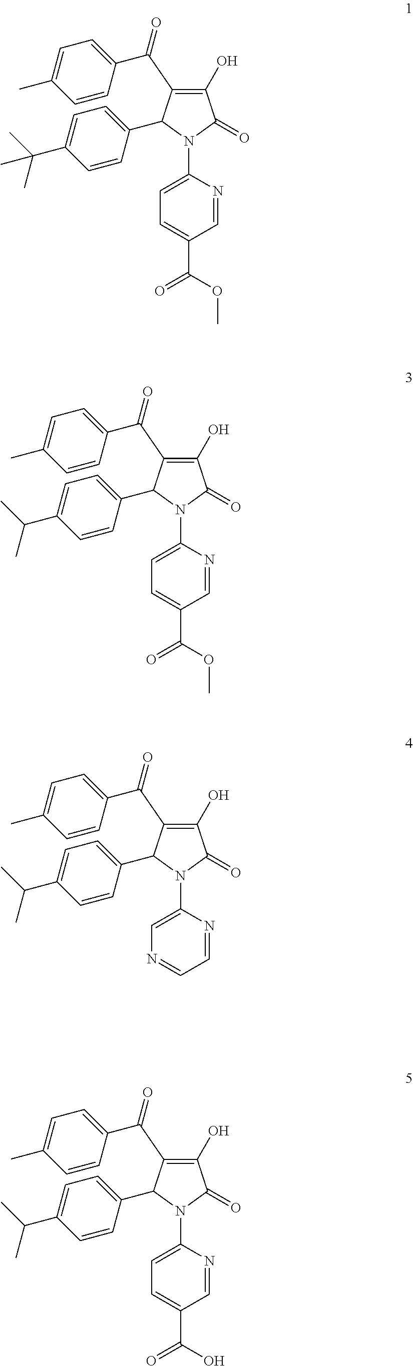

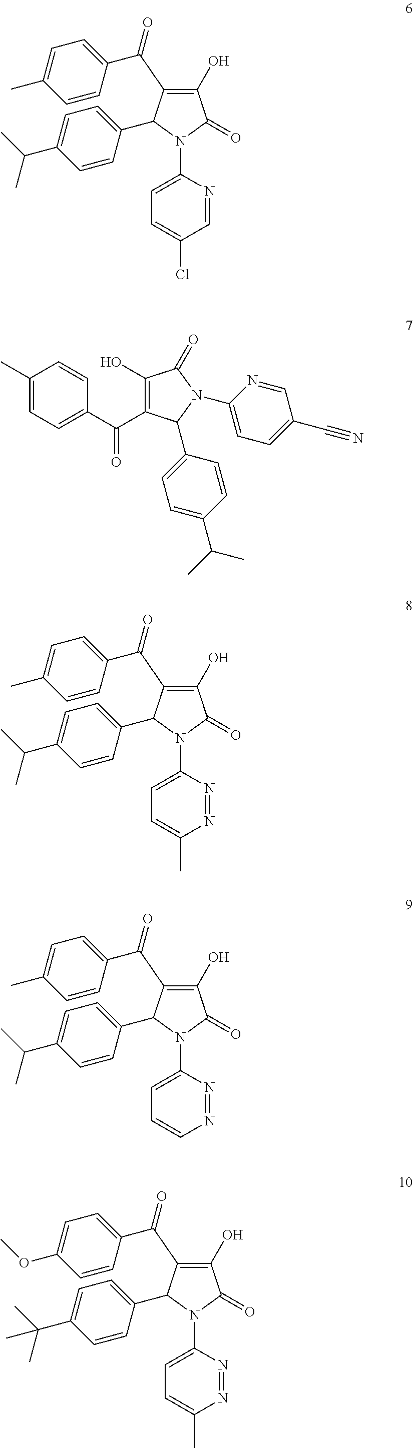

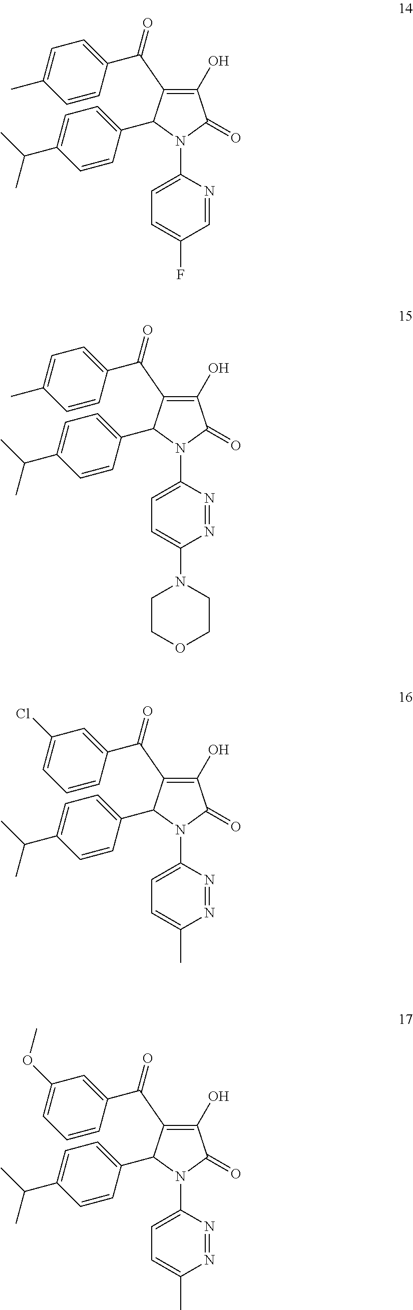

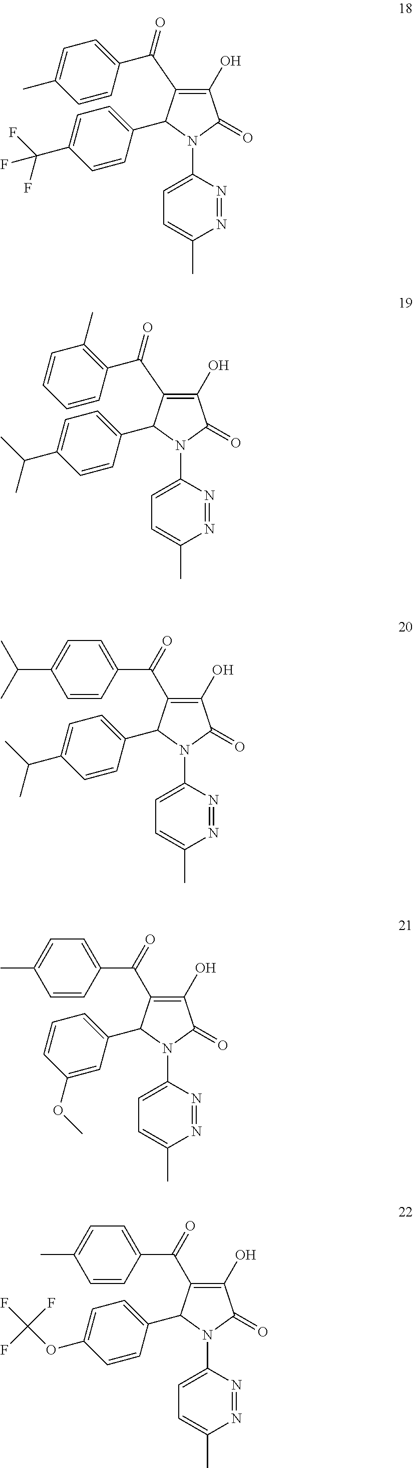

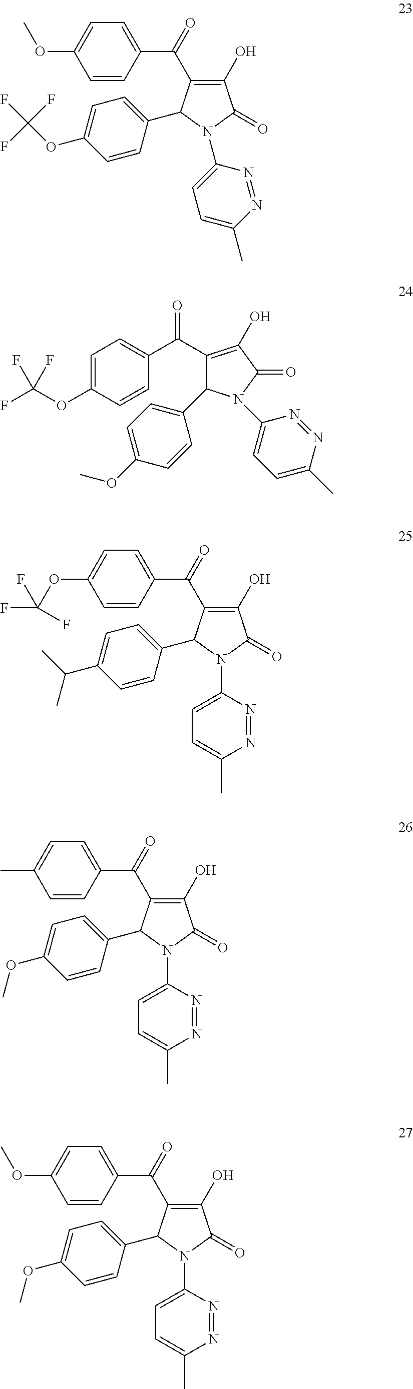

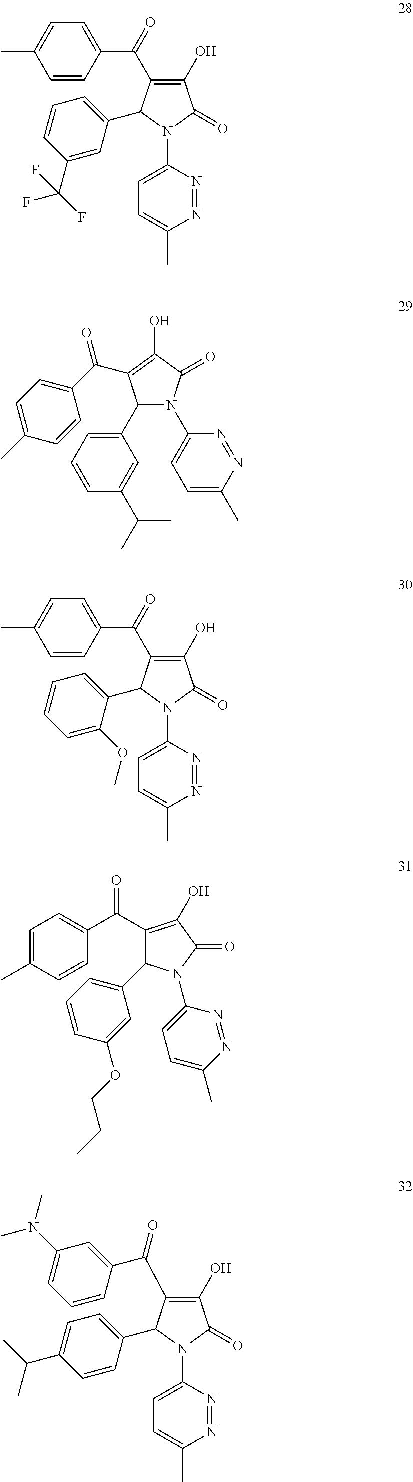

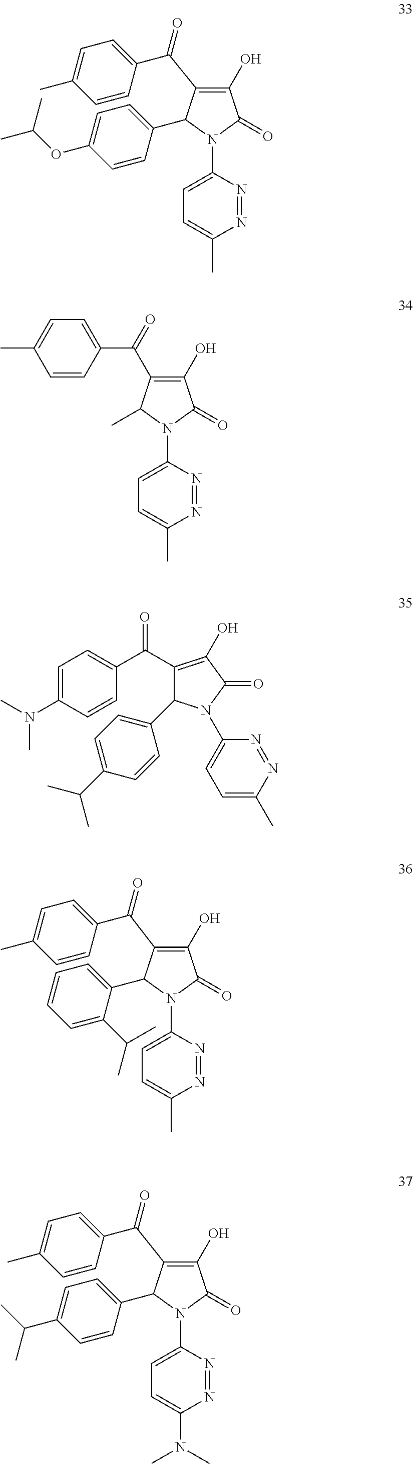

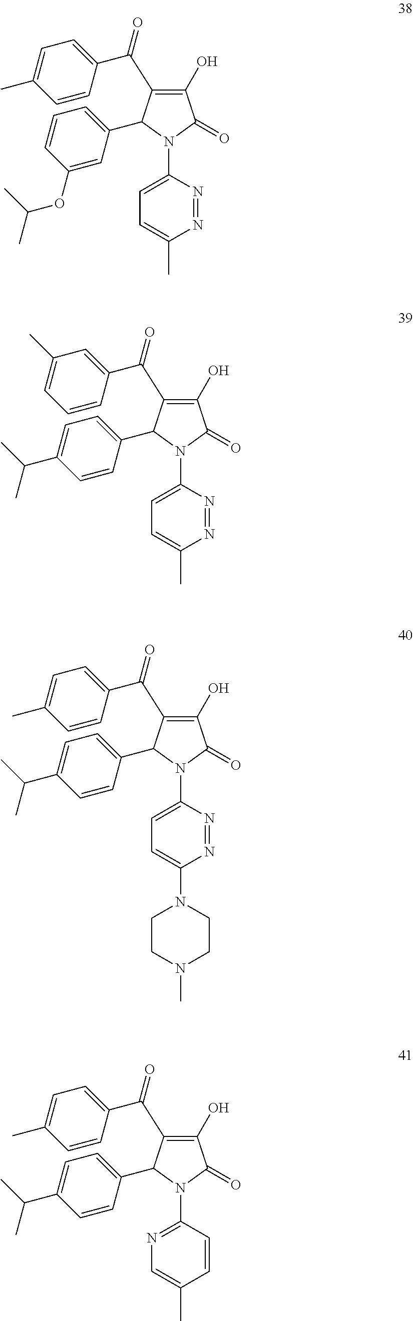

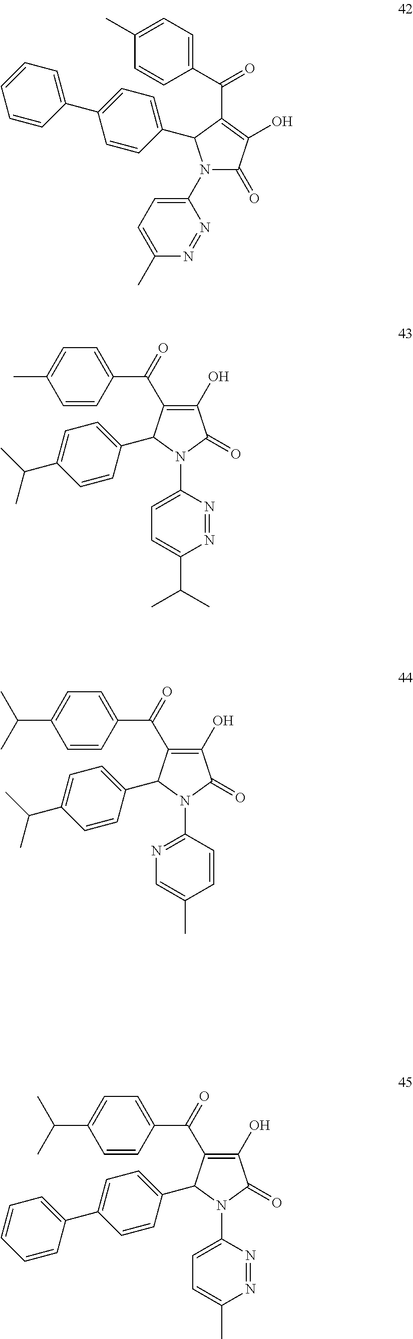

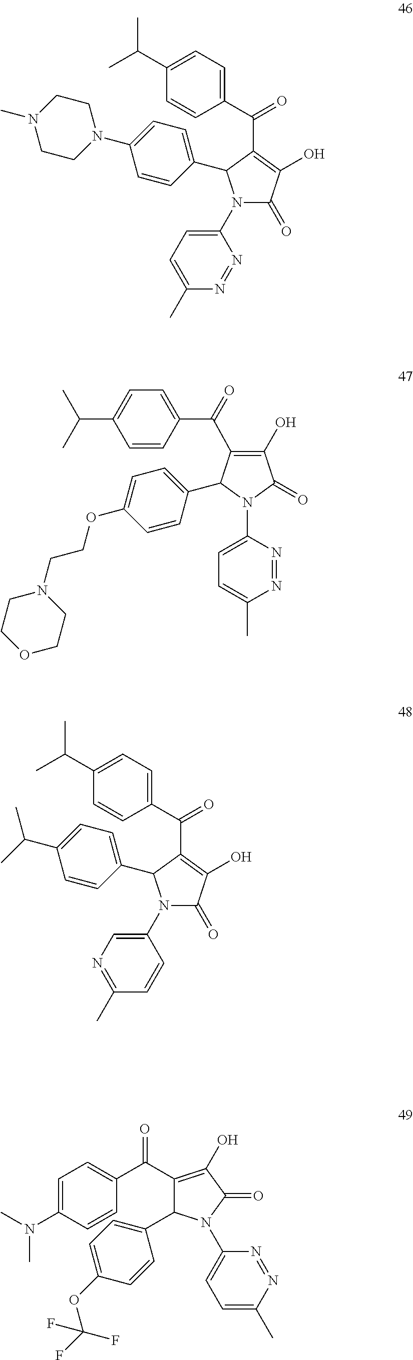

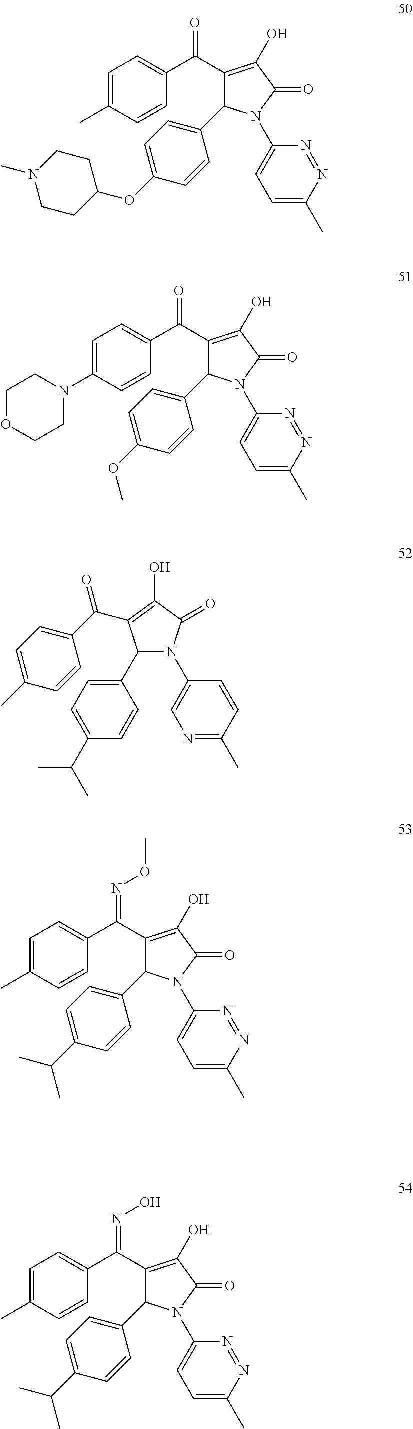

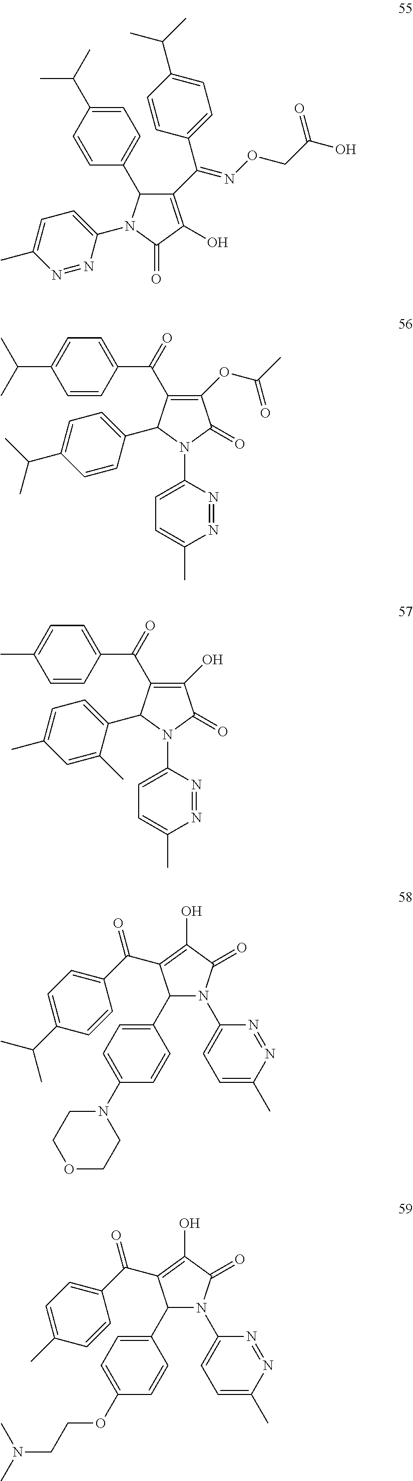

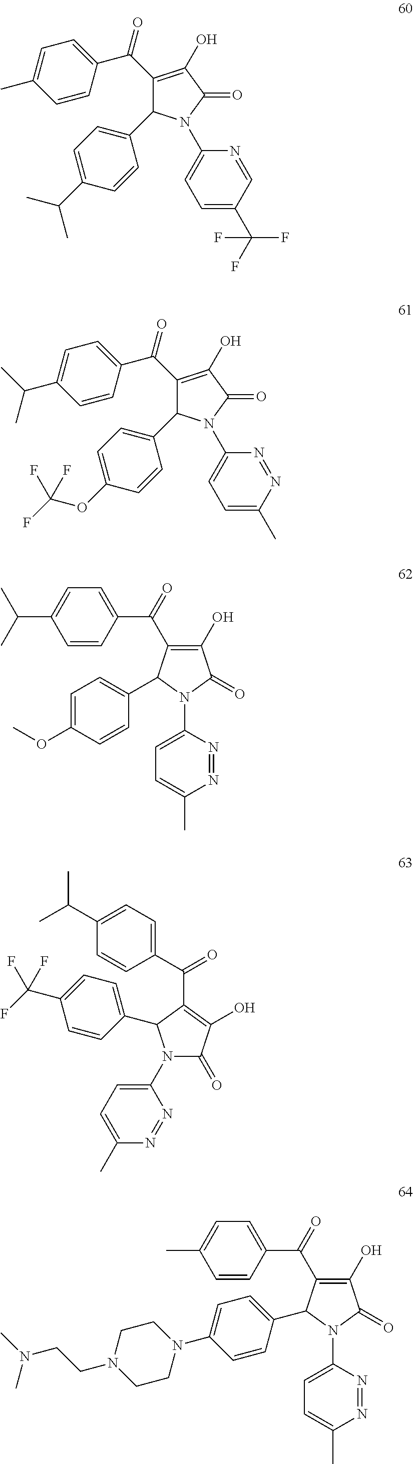

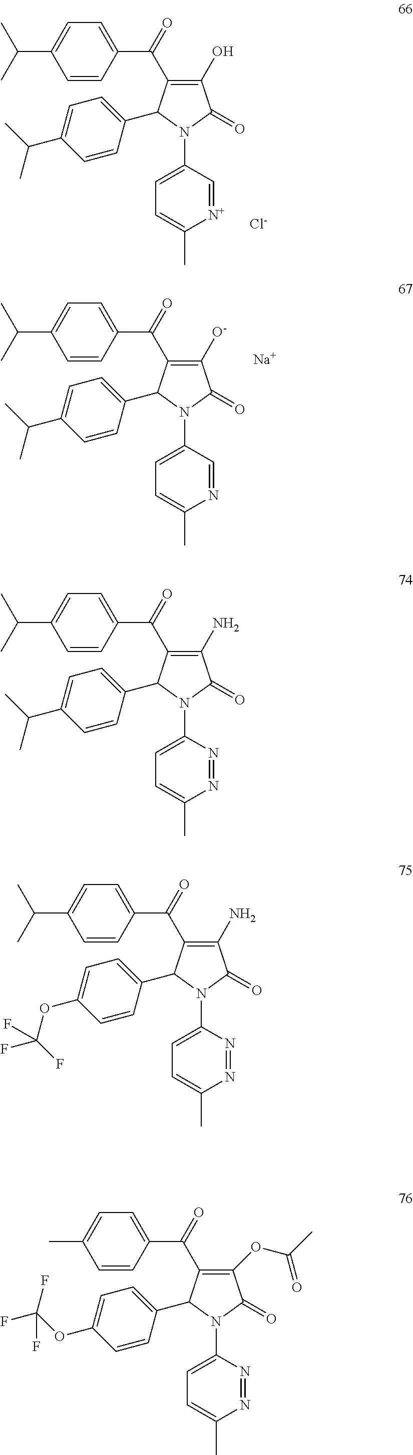

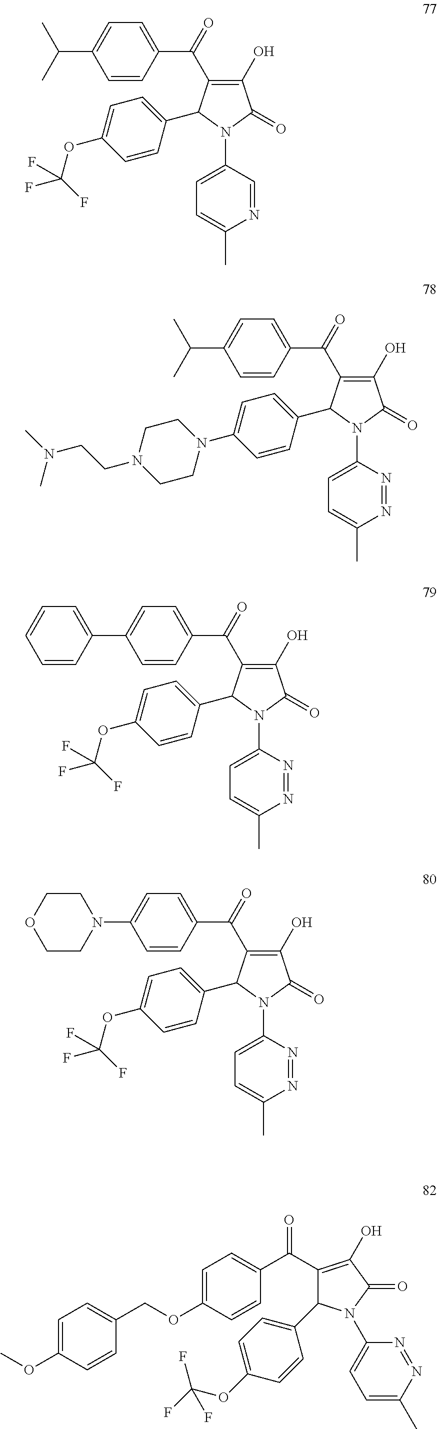

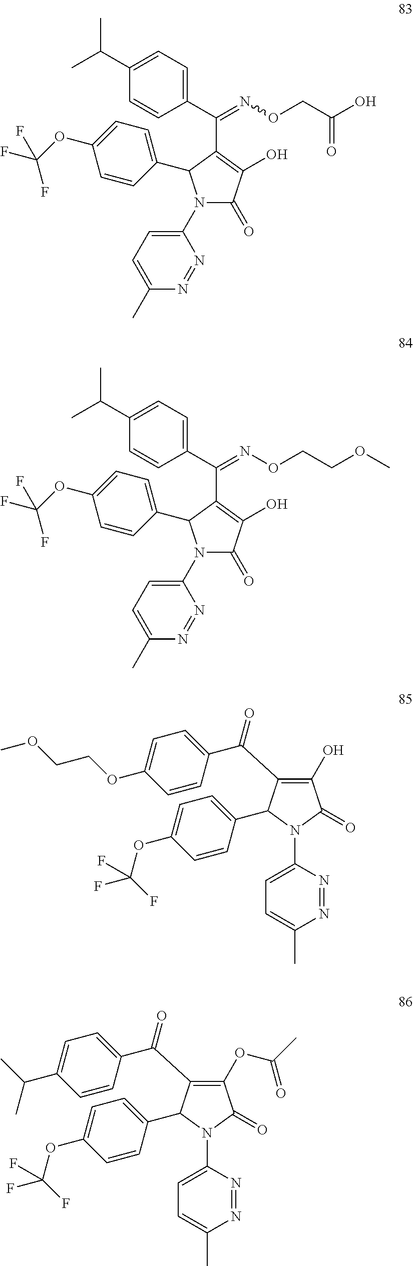

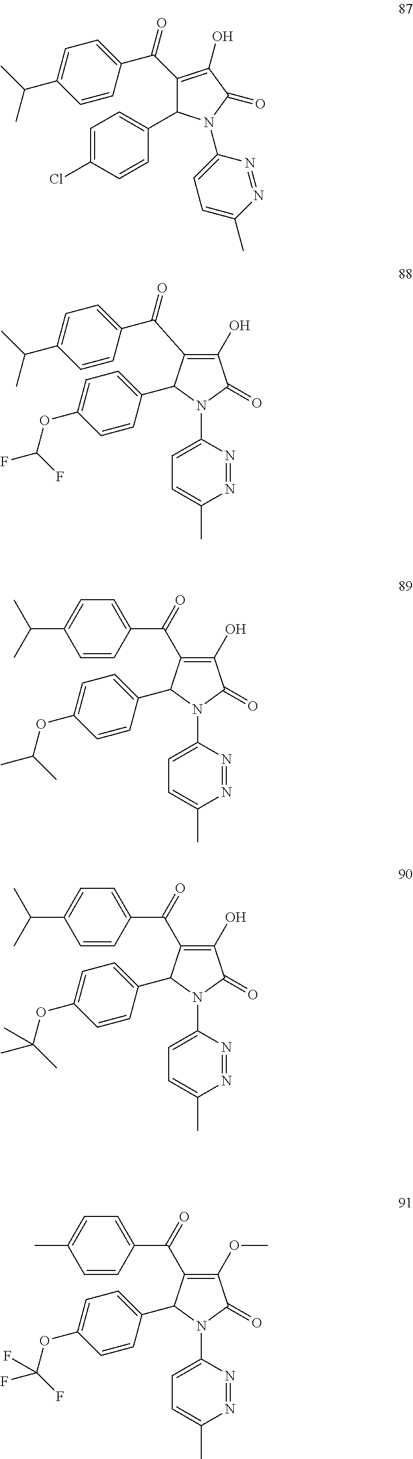

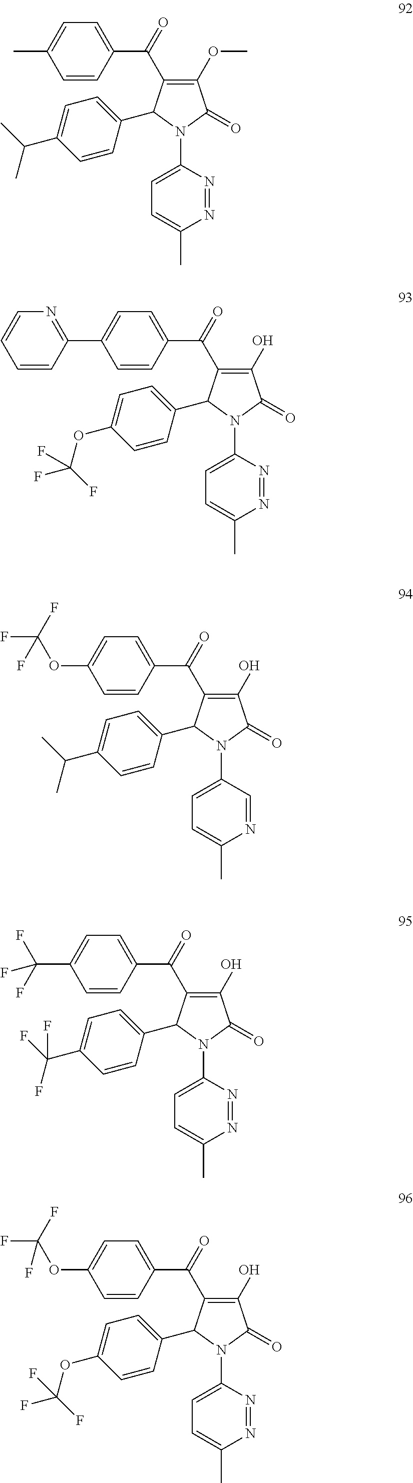

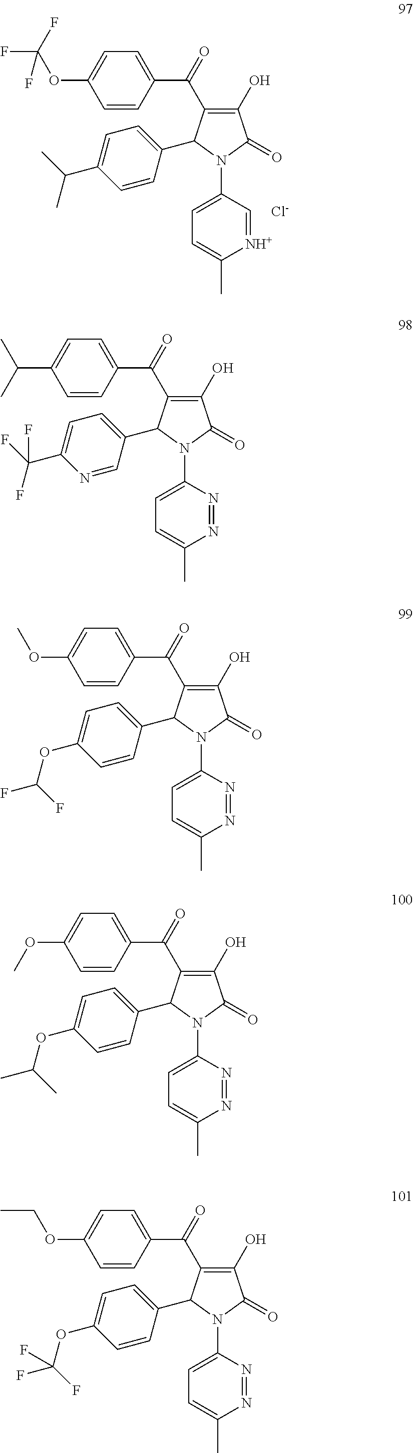

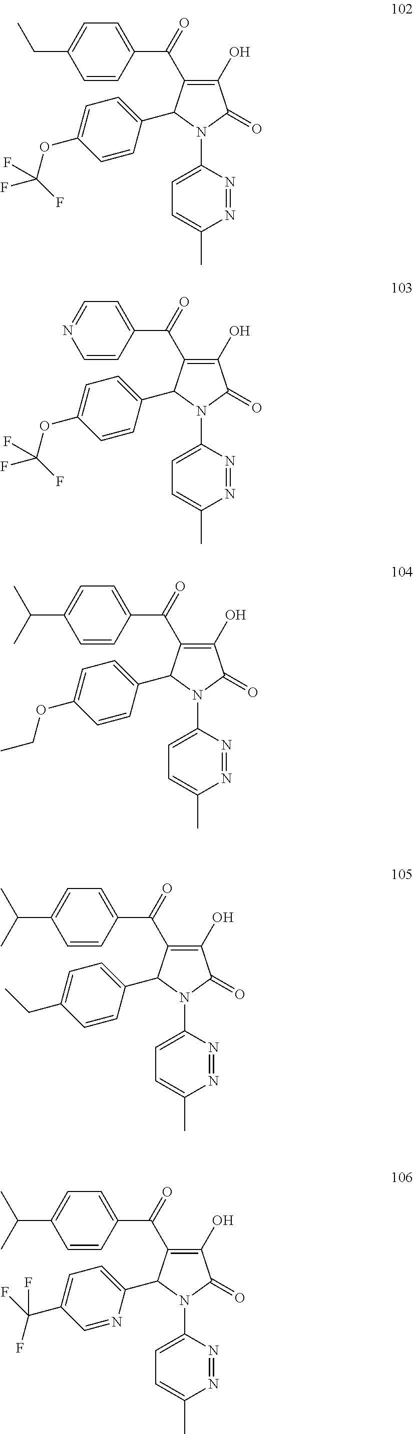

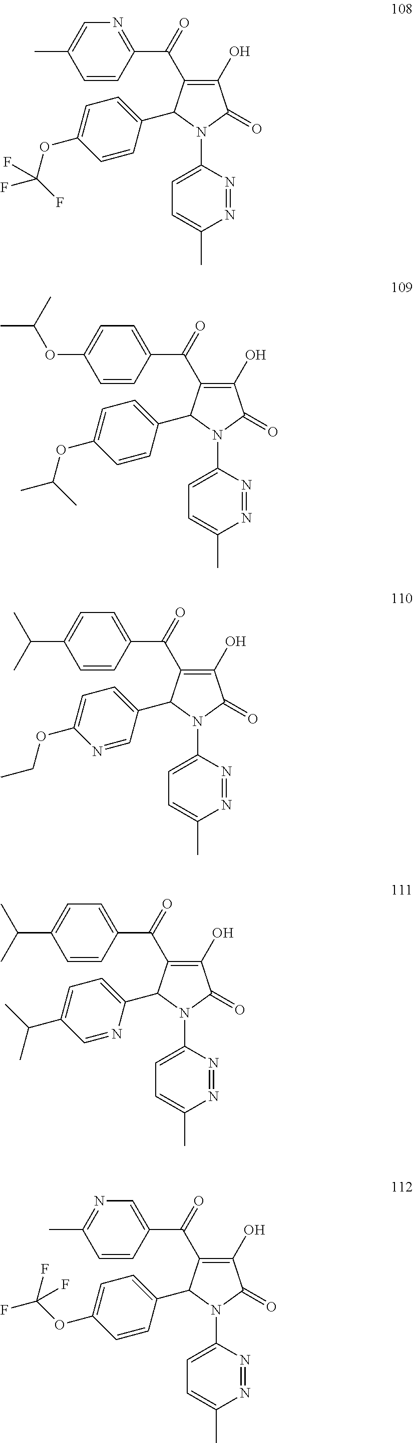

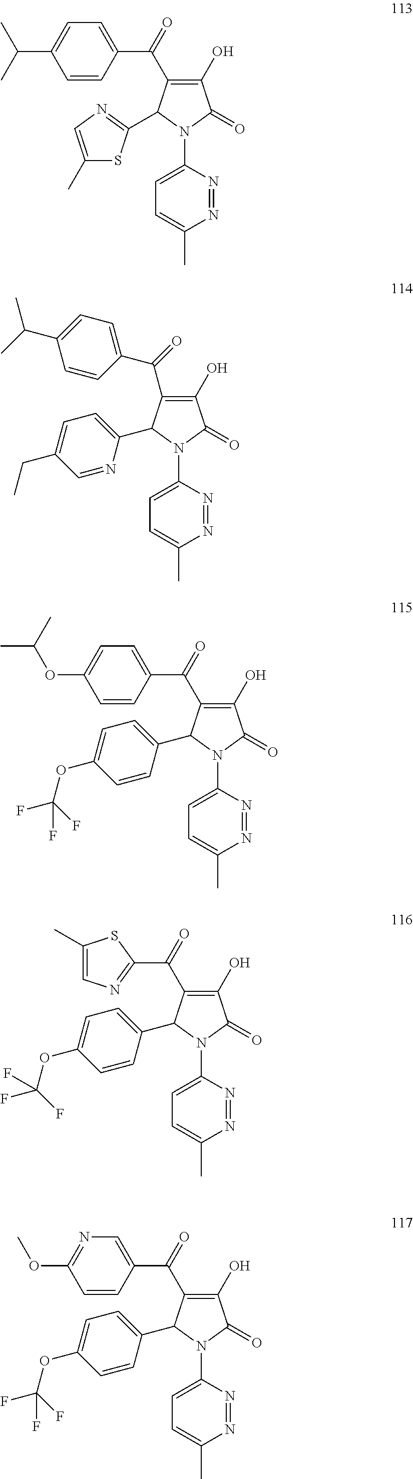

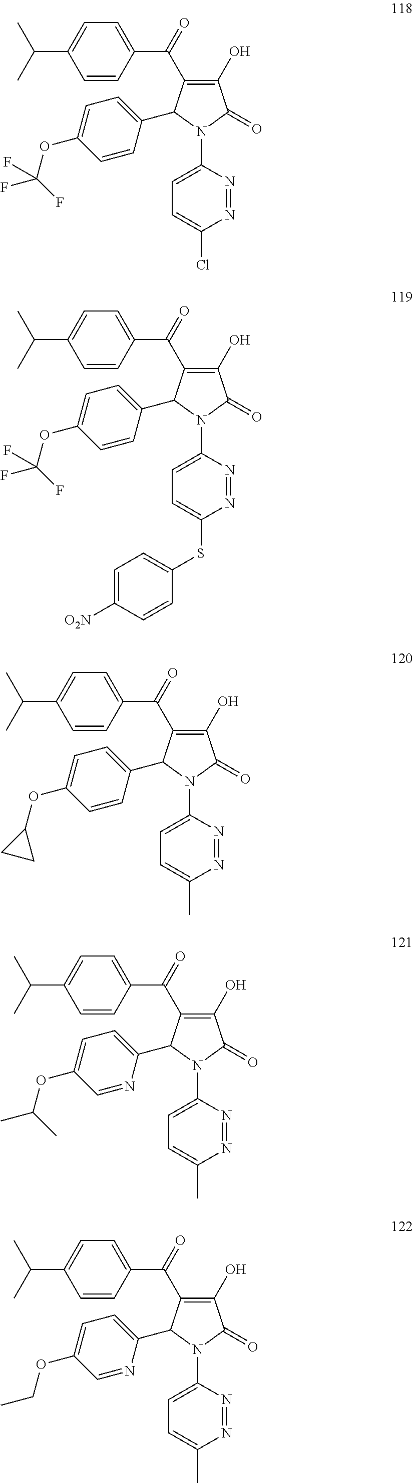

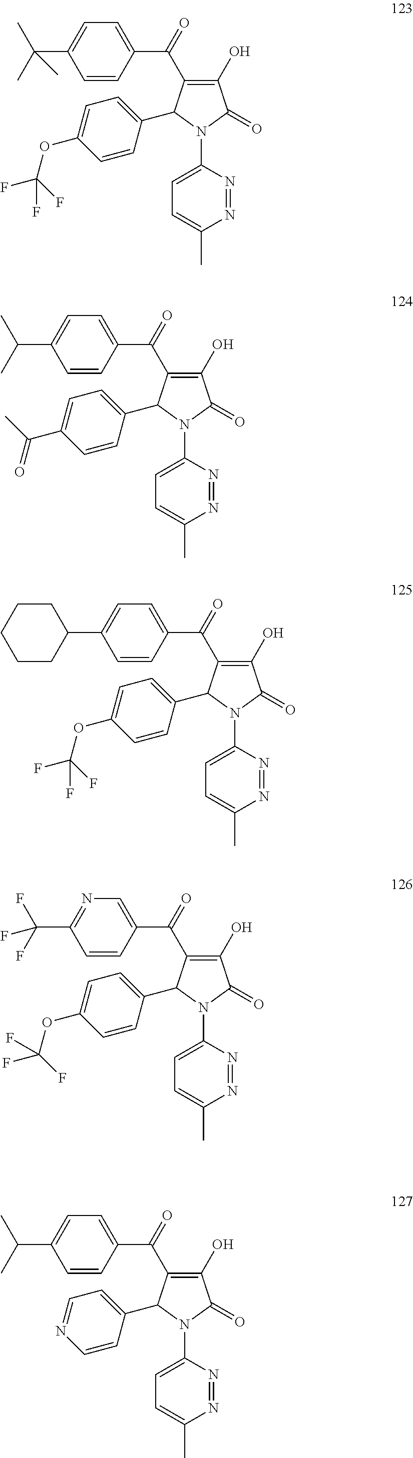

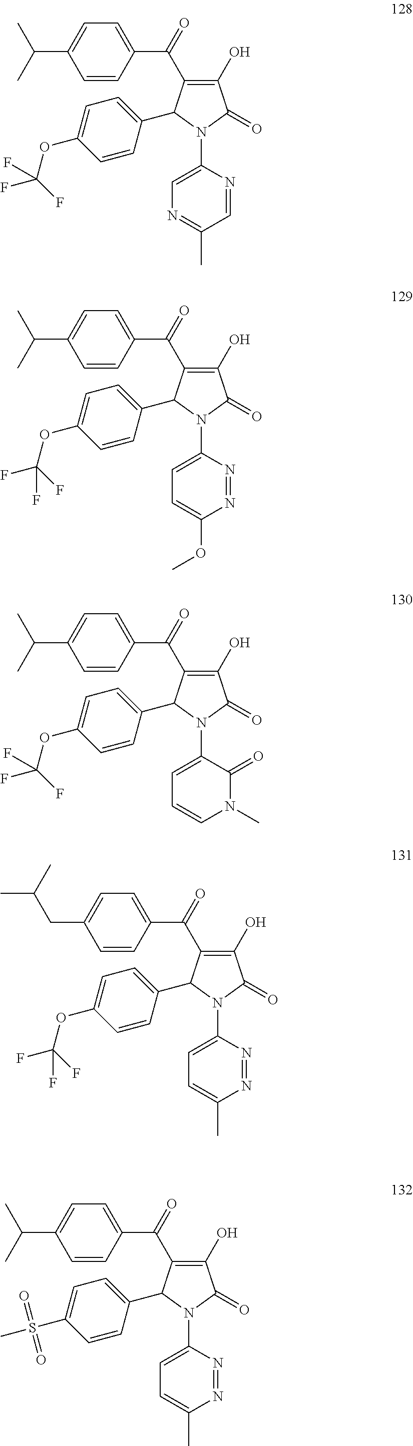

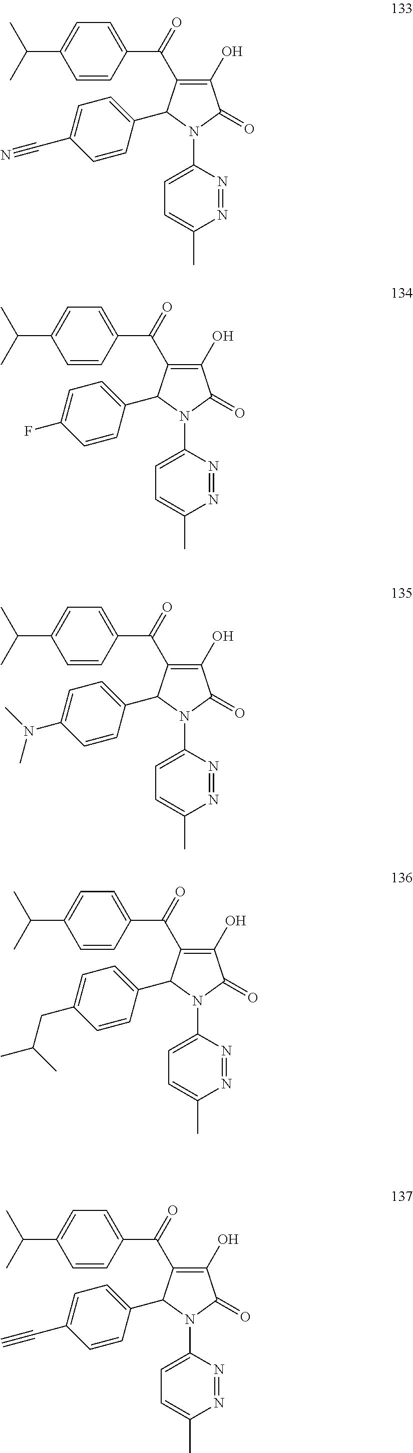

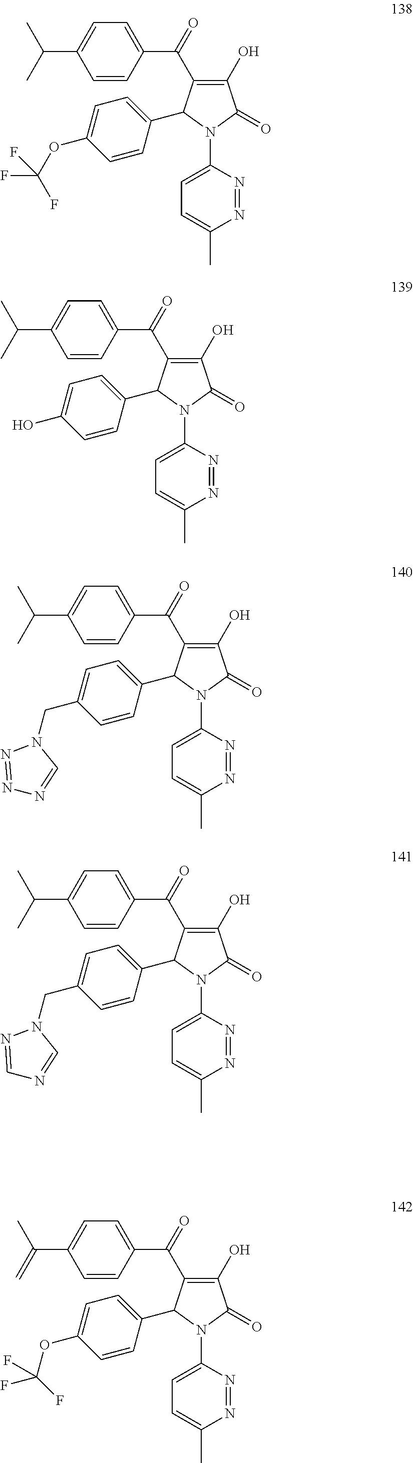

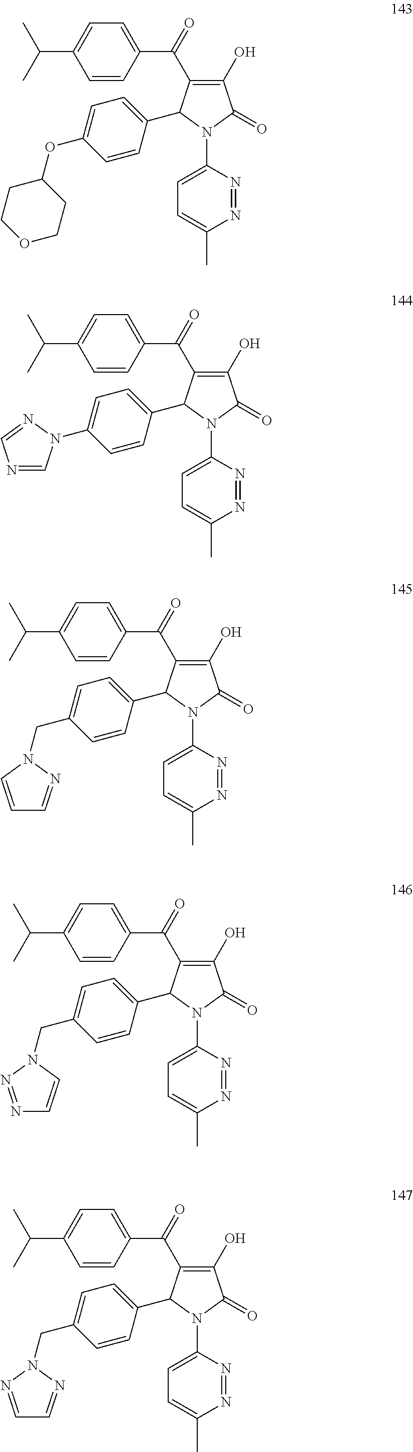

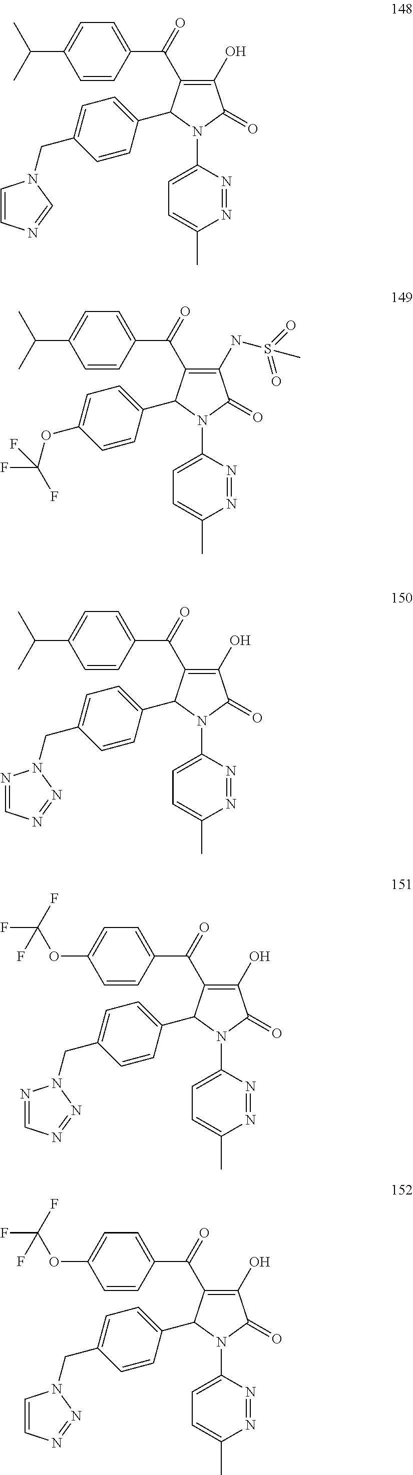

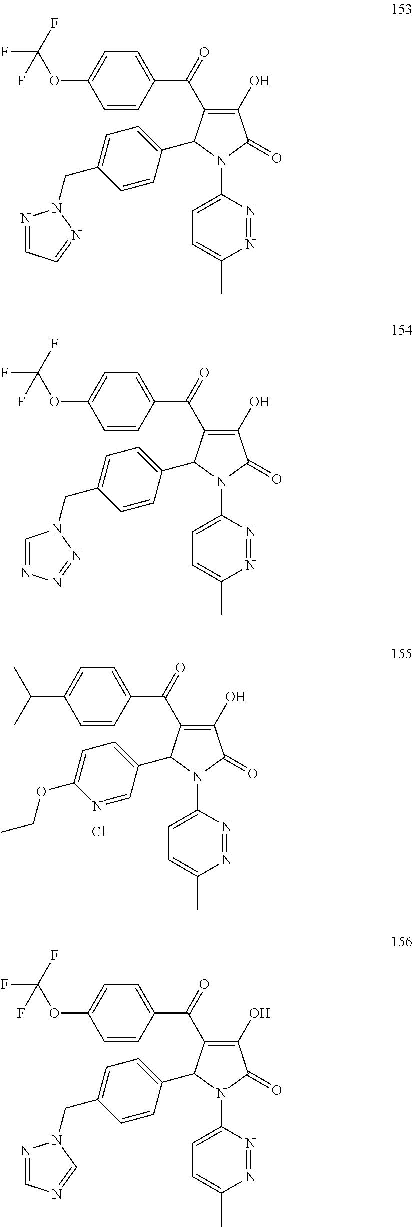

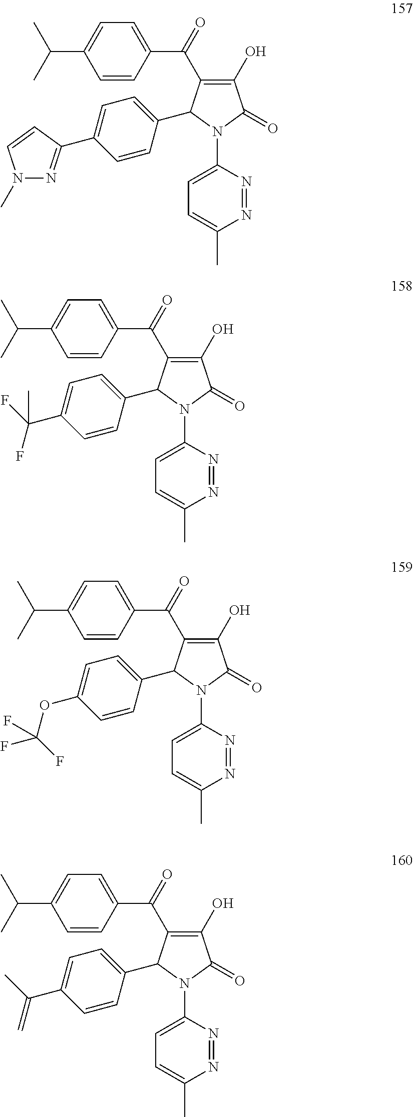

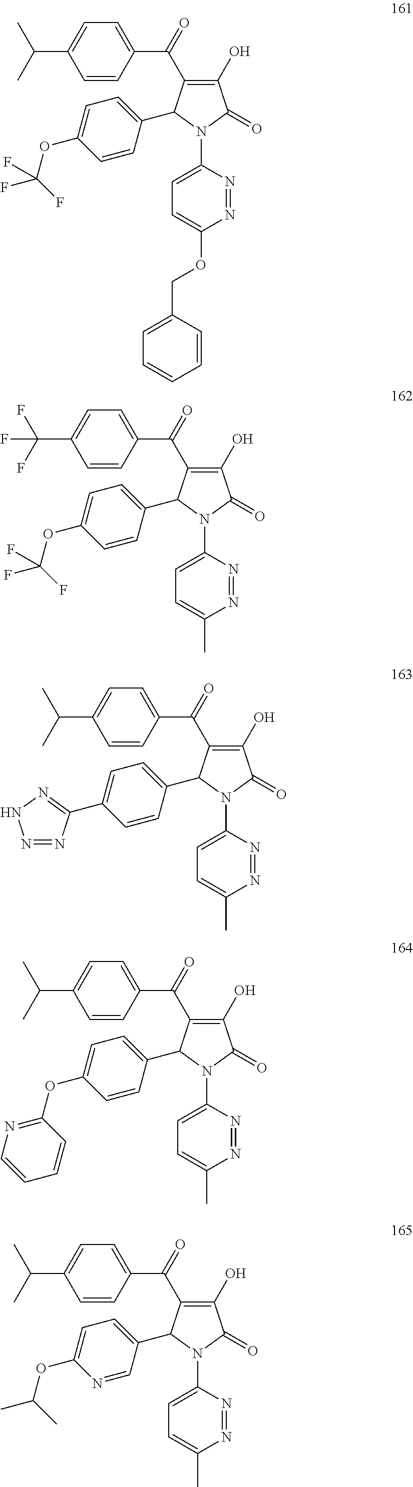

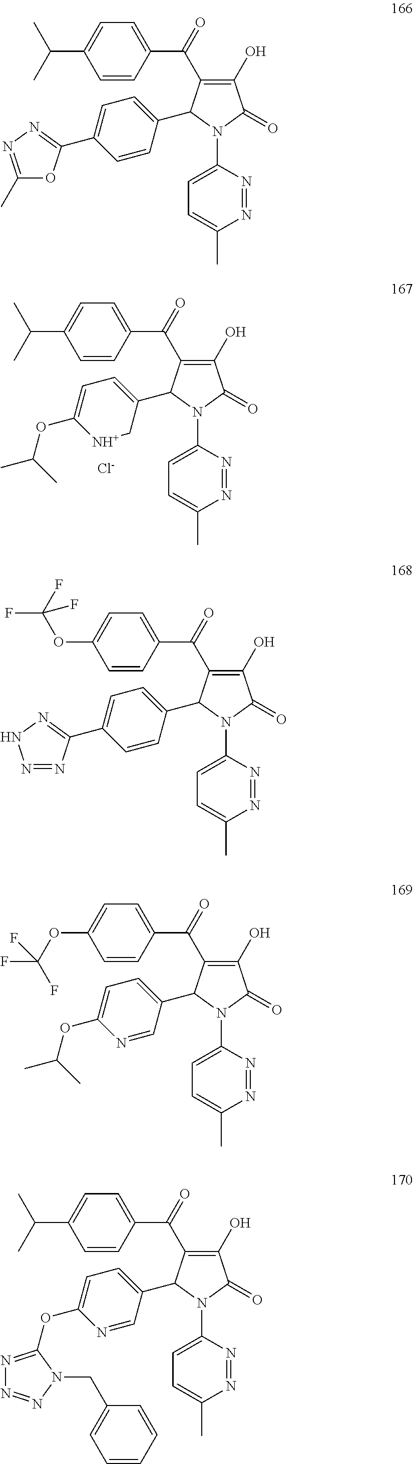

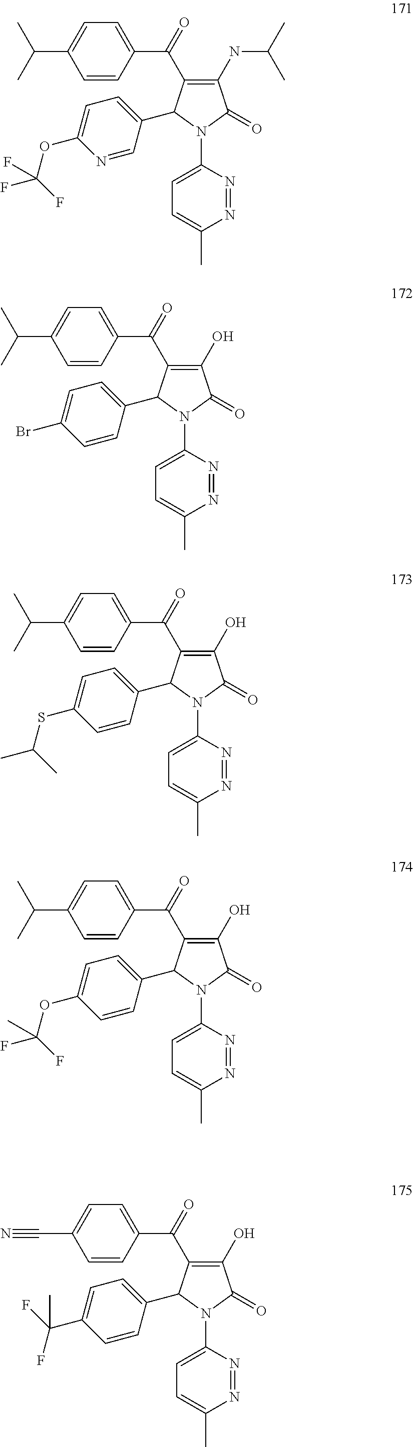

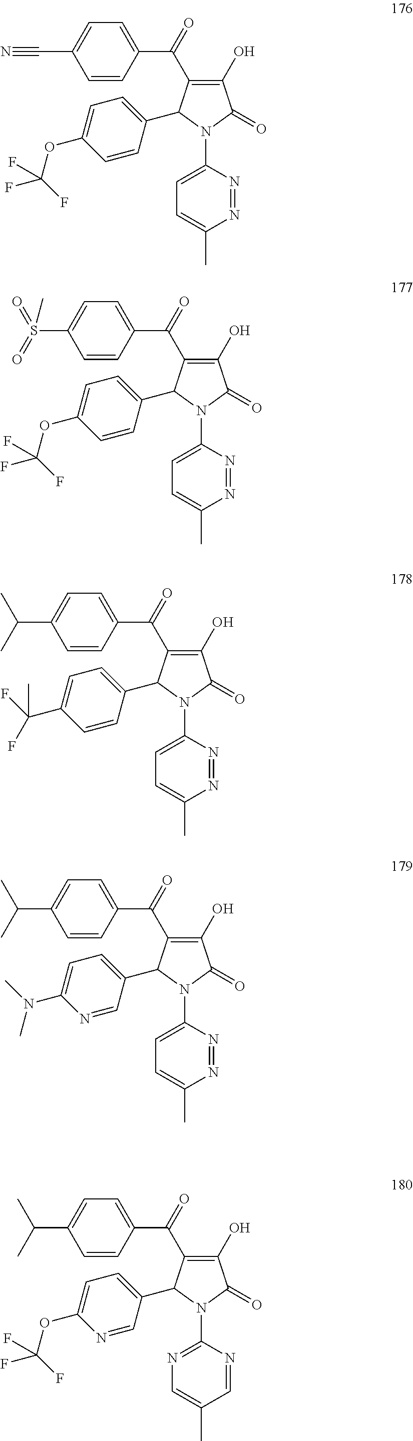

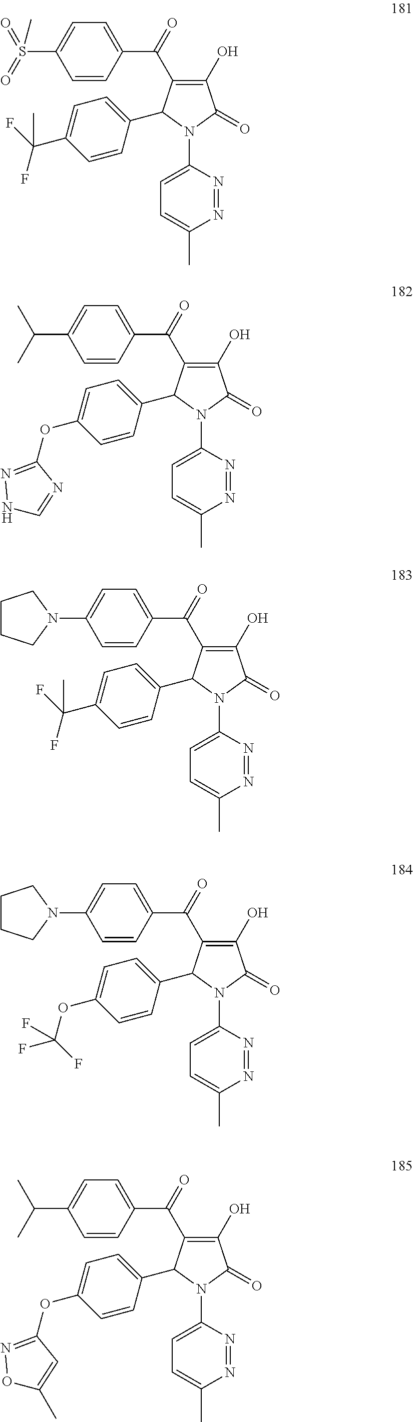

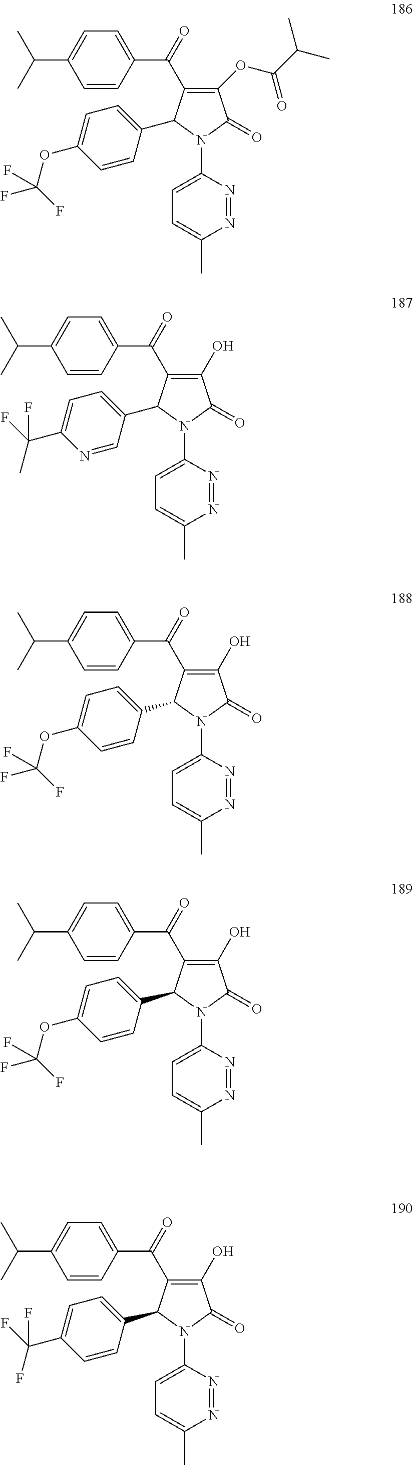

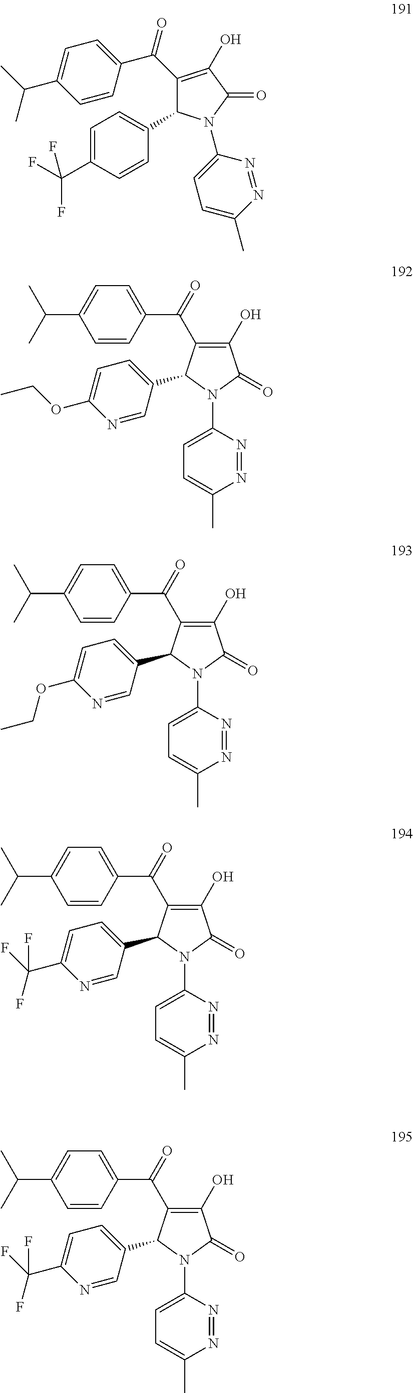

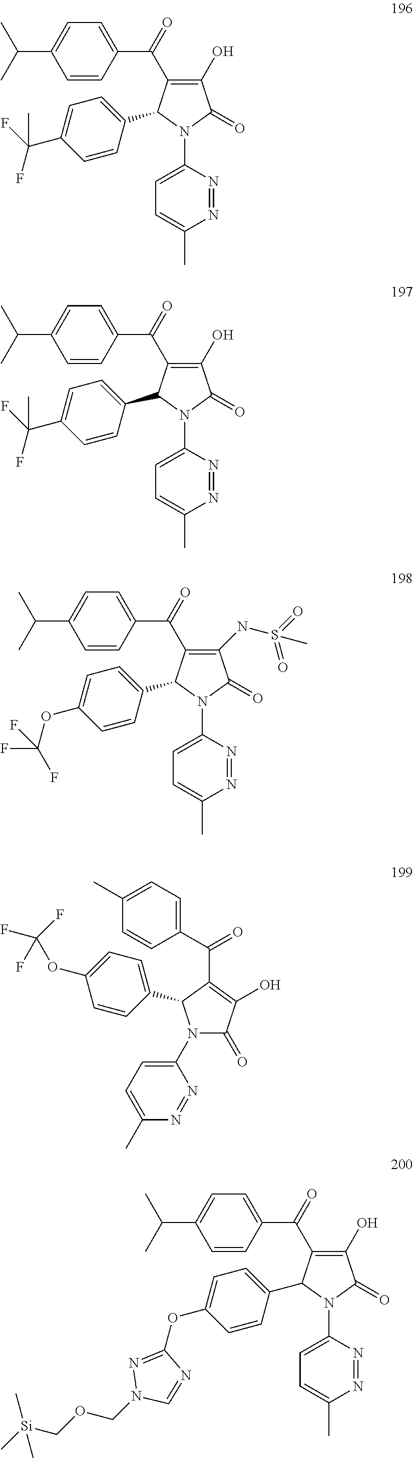

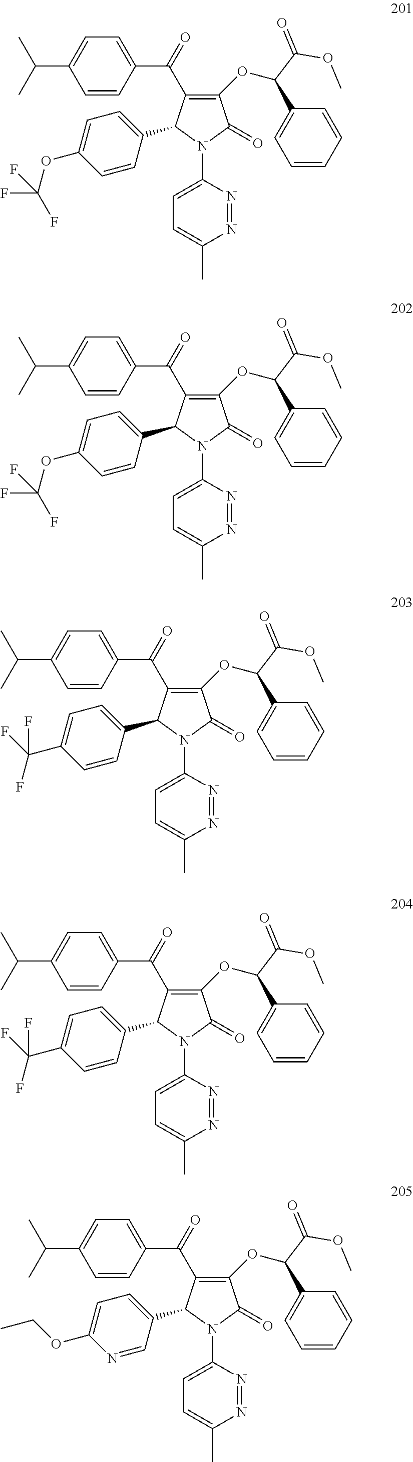

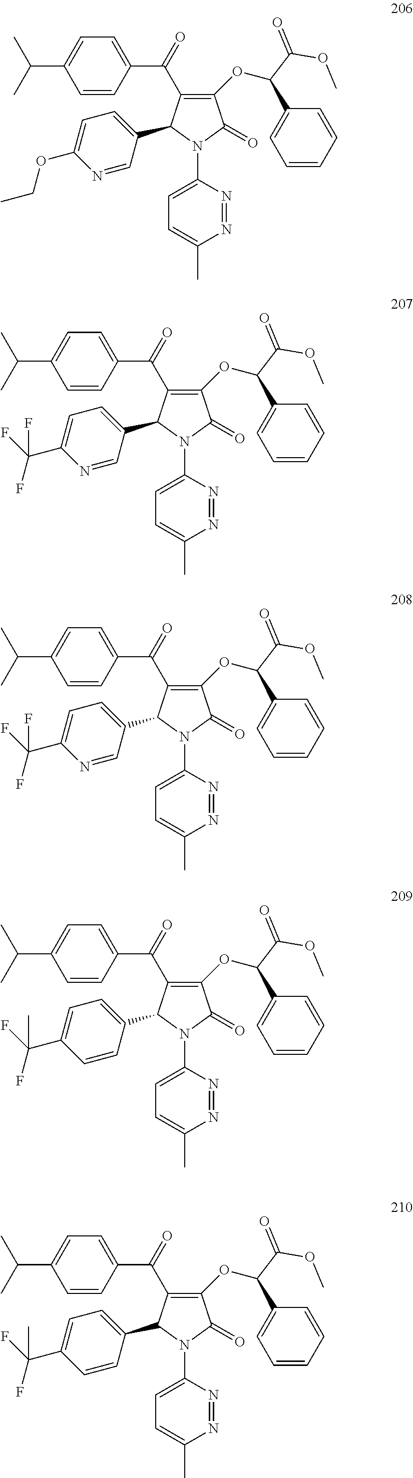

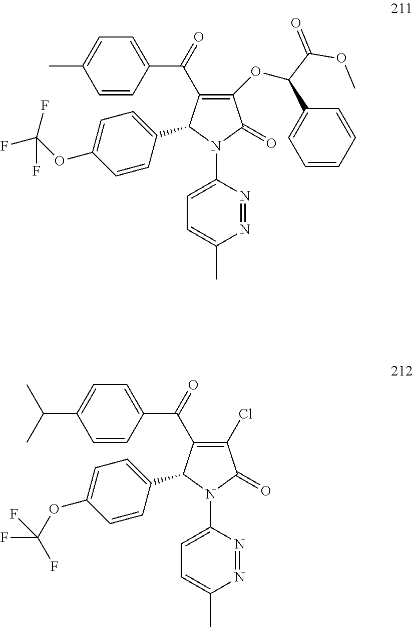

35. A method to inhibit the hepatitis C virus replication using a compound of formula I as defined in claim 32 or a salt, solvate, tautomer, isotope, enantiomer, diastereoisomer or racemic mixture thereof or a compound chosen from the group consisting of the compounds of formula 1, 3-10, 14-64, 66, 67, 74-80, 82-106 and 108-212 as defined in claim 34.

36. A method to treat hepatitis C comprising the simultaneous, separate or sequential administration to a person in need thereof of an effective amount of a compound of formula I as defined in claim 32 or a salt, solvate, tautomer, isotope, enantiomer, diastereoisomer or racemic mixture thereof and at least another antiviral agent.

37. The method according to claim 36, wherein the other antiviral agent is selected in the group consisting of ribavirin, interferon, inhibitors of HCV helicase, inhibitors of HCV protease, inhibitors of HCV NS4A, inhibitors of HCV NS5B, inhibitors of HCV NS5A, inhibitors of HCV polymerase, anti-HIV agent and mixture thereof.

38. The method according to claim 36, wherein the person has the HIV disease.
